(19) United States
(12) Patent Application Publication
(10) Pub. No.: US 2021/0180059 A1
(43) Pub. Date: Jun. 17, 2021

(54) COMPOSITIONS AND METHODS FOR IMPROVING THE EFFICACY OF CAS9-BASED KNOCK-IN STRATEGIES

(71) Applicant: ASTRAZENECA AB, SODERTALJE (SE)

(72) Inventors: MARCELLO MARESCA, SODERTALJE (SE); AMIR TAHERI-GHAHFAROKHI, SODERTALJE (SE); FREDERIK KARLSSON, SODERTALJE (SE); MOHAMMAD BOHLOOLY-YEGANEH, SODERTALJE (SE); LORENZ MARTIN MAYR, CAMBRIDGE (GB)

(21) Appl. No.: 16/763,809

(22) PCT Filed: Nov. 16, 2018
(86) PCT No.: PCT/US2018/061680

§ 371 Date: May 13, 2020

Related U.S. Application Data
(60) Provisional application No. 62/693,690, filed on Jul. 3, 2018.
(60) Provisional application No. 62/587,029, filed on Nov. 16, 2017.

(51) Int. Cl. C12N 15/113; C12N 15/10; C12N 9/22

(52) U.S. Cl. CPC C12N 15/113; C12N 15/102; C12N 9/22; C12N 2810/40; C12N 2800/24; C12N 2800/22; C12N 2310/20

(57) ABSTRACT

The present disclosure provides a non-naturally occurring CRISPR-Cas system comprising: a Cas9 effector protein capable of generating cohesive ends (stiCas9), and a guide polynucleotide that forms a complex with the stiCas9 and comprising a guide sequence, wherein the guide sequence hybridizes with a target sequence in a eukaryotic cell but does not hybridize to a sequence in a bacterial cell, and wherein the complex does not occur in nature. The present disclosure also provides a method of introducing a sequence of interest into a chromosome of a cell. Finally, the present disclosure provides for a method of modifying one or more nucleotides using seamless mutagenesis.

### SEQUENCE LISTING

The instant application contains a Sequence Listing which has been submitted electronically in ASCII format and is hereby incorporated by reference in its entirety. Said ASCII copy, created on Nov. 16, 2018, is named 0098-0002WO1_SL.txt and is 1,105,014 bytes in size.

### FIELD OF THE INVENTION

The present disclosure provides a non-naturally occurring CRISPR-Cas system comprising: a Cas9 effector protein capable of generating cohesive ends (stiCas9), and a guide polynucleotide that forms a complex with the stiCas9 and comprising a guide sequence, wherein the guide sequence hybridizes with a target sequence in a eukaryotic cell but does not hybridize to a sequence in a bacterial cell, and wherein the complex does not occur in nature.

### BACKGROUND

Clustered Regularly Interspaced Short Palindromic Repeats (CRISPR) and CRISPR-associated (Cas) systems are prokaryotic immune systems first discovered by Ishino in E. coli (Ishino et al., Journal of Bacteriology 169(12): 5429-5433 (1987), incorporated by reference herein in its entirety). This immune system provides immunity against viruses and plasmids by targeting the nucleic acids of the viruses and plasmids in a sequence-specific manner. See also Soret et al., “CRISPR—a widespread system that provides acquired resistance against phages in bacteria and archaea”, Nature Reviews Microbiology 6(3): 181-186 (2008), incorporated by reference herein in its entirety. CRISPR-Cas systems have been classified into three main types: Type I, Type II, and Type III. The main defining features of the separate Types are the various cas genes, and the respective proteins they encode, that are employed. The cas1 and cas2 genes appear to be universal across the three main Types, whereas cas3, cas9, and cas10 are thought to be specific to the Type I, Type II, and Type III systems, respectively. See, e.g., Barrangou and Marraffini, “CRISPR-Cas systems: prokaryotes upgrade to adaptive immunity”, Cell 54(2): 234-244 (2014), incorporated by reference herein in its entirety.

There are two main stages involved in this immune system: the first is acquisition, and the second is interference. The first stage involves cutting the genome of invading viruses and plasmids and integrating segments of this into the CRISPR locus of the organism. The segments that are integrated into the genome are known as protospacers and help in protecting the organism from subsequent attack by the same virus or plasmid. The second stage involves attacking an invading virus or plasmid. This stage relies upon the protospacers being transcribed to RNA, this RNA, following some processing, then hybridizes with a complementary sequence in the DNA of an invading virus or plasmid while also associating with a protein, or protein complex that effectively cleaves the DNA.

Depending on the bacterial species, CRISPR RNA processing proceeds differently. For example, in the Type II system, originally described in the bacterium Streptococcus pyogenes, the transcribed RNA is paired with a trans-activating RNA (tracrRNA) before being cleaved by RNase III to form an individual CRISPR-RNA (crRNA). The crRNA is further processed after binding by the Cas9 nuclease to produce the mature crRNA. The crRNA/Cas9 complex subsequently binds to DNA containing sequences complementary to the captured regions (termed protospacers). The Cas9 protein then cleaves both strands of DNA in a site-specific manner, forming a double-strand break (DSB). This provides a DNA-based “memory”, resulting in rapid degradation of viral or plasmid DNA upon repeat exposure and/or infection. The native CRISPR system has been comprehensively reviewed (see, e.g., Barrangou and Marraffini, 2014).

Since its original discovery, multiple groups have done extensive research around potential applications of the CRISPR system in genetic engineering, including gene editing (Jinek et al., “A programmable dual-RNA-guided DNA endonuclease in adaptive bacterial immunity”, Science 337(6096): 816-821 (2012); Cong et al., “Multiplex genome engineering using CRISPR/Cas systems”, Science 339(6121): 819-823 (2013); and Mali et al., “RNA-guided human genome engineering via Cas9”, Science 339(6121): 823-826 (2013); each of which is incorporated by reference herein in its entirety). One major development was utilization of a chimeric RNA to target the Cas9 protein, designed around individual units from the CRISPR array fused to the tracrRNA. This creates a single RNA species, called a small guide RNA (gRNA) where modification of the sequence in the protospacer region can target the Cas9 protein site-specifically. Considerable work has been done to understand the nature of the base-pairing interaction between the chimeric RNA and the target site, and its tolerance to mismatches, which is highly relevant in order to predict and assess off-target effects (see, e.g., Fu et al., “Improving CRISPR-Cas nucleases using truncated guide RNAs”, Nature Biotechnology 32(3): 279-284 (2014), including supporting materials, which is incorporated by reference herein in its entirety).

The CRISPR-Cas9 gene editing system has been used successfully in a wide range of organisms and cell lines, both in order to induce DSB formation using the wild type Cas9 protein or to nick a single DNA strand using a mutant protein termed Cas9n/Cas9 D10A (see, e.g., Mali et al., 2013 and Sander and Joung, “CRISPR-Cas systems for editing, regulating and targeting genomes”, Nature Biotechnology 32(4): 347-355 (2014), each of which is incorporated by reference herein in its entirety). While DSB formation results in creation of small insertions and deletions (indels) that can disrupt gene function, the Cas9n/Cas9 D10A nickase avoids indel creation (the result of repair through non-homologous end-joining) while stimulating the endogenous homologous recombination machinery. Thus, the Cas9n/Cas9 D10A nickase can be used to insert regions of DNA into the genome with high-fidelity.

In addition to genome editing, the CRISPR system has a multitude of other applications, including regulating gene expression, genetic circuit construction, and functional genomics, amongst others (reviewed in Sander and Joung, 2014).

Various publications are cited herein, the disclosures of which are incorporated by reference herein in their entireties.

### SUMMARY OF THE INVENTION

In some embodiments, the present disclosure provides a non-naturally occurring CRISPR-Cas system comprising: a Cas9 effector protein capable of generating cohesive ends (stiCas9), and a guide polynucleotide that forms a complex with the stiCas9 and comprises a guide sequence, wherein the guide sequence hybridizes with a target sequence in a eukaryotic cell but does not hybridize to a sequence in a bacterial cell, wherein the complex does not occur in nature.

In some embodiments, the present disclosure provides a non-naturally occurring CRISPR-Cas system comprising: a Cas9 effector protein capable of generating cohesive ends (stiCas9) and comprises a nuclear localization sequence (NLS), and a guide polynucleotide that forms a complex with the stiCas9 and comprises a guide sequence, wherein the complex does not occur in nature.

In some embodiments, the present disclosure provides a non-naturally occurring CRISPR-Cas system comprising: one or more nucleotide sequences encoding a Cas9 effector protein capable of generating cohesive ends (stiCas9), and a nucleotide sequence encoding a guide polynucleotide that forms a complex with the stiCas9 and comprises a guide sequence, wherein the guide sequence hybridizes with a target sequence in a eukaryotic cell but does not hybridize to a sequence in a bacterial cell, and wherein the complex does not occur in nature.

In some embodiments, the present disclosure provides a non-naturally occurring CRISPR-Cas system comprising: (a) one or more nucleotide sequences encoding a Cas9 effector protein capable of generating cohesive ends (stiCas9), and (b) a nucleotide sequence encoding a guide polynucleotide that forms a complex with the stiCas9 and comprises a guide sequence, wherein the nucleotide sequences of (a) and (b) are under control of a eukaryotic promoter, and wherein the complex does not occur in nature.

In some embodiments, the CRISPR-Cas systems of the present disclosure further comprise a polynucleotide comprising a tracrRNA sequence. In some embodiments, the guide polynucleotide, tracrRNA sequence and the stiCas9 of the CRISPR-Cas systems are capable of forming a complex, and the complex does not occur in nature.

In some embodiments, the present disclosure provides a non-naturally occurring CRISPR-Cas system comprising one or more vectors comprising: a regulatory element operably linked to one or more nucleotide sequences encoding a Cas9 effector protein capable of generating cohesive ends (stiCas9), and a guide polynucleotide that forms a complex with the stiCas9 and comprises a guide sequence, wherein the guide sequence hybridizes with a target sequence in a eukaryotic cell but does not hybridize to a sequence in a bacterial cell, wherein the complex does not occur in nature.

In some embodiments, the present disclosure provides a non-naturally occurring CRISPR-Cas system comprising one or more vectors comprising: a regulatory element operably linked to one or more nucleotide sequences encoding a Cas9 effector protein capable of generating cohesive ends (stiCas9), wherein the regulatory element is a eukaryotic regulatory element, and a guide polynucleotide that forms a complex with the stiCas9 and comprises a guide sequence, wherein the complex does not occur in nature.

In some embodiments, the guide polynucleotide further comprises a tracrRNA sequence. In some embodiments, the non-naturally occurring vector of the present disclosure further comprises a nucleotide sequence comprising a tracrRNA sequence.

In some embodiments of the CRISPR-Cas system, the complex is capable of cleaving at a site within 10 nucleotides of a Protospacer Adjacent Motif (PAM). In some embodiments of the CRISPR-Cas system, the complex is capable of cleavage at a site within 5 nucleotides of a Protospacer Adjacent Motif (PAM). In some embodiments of the CRISPR-Cas system, the complex is capable of cleavage at a site within 3 nucleotides of a Protospacer Adjacent Motif (PAM).

In some embodiments of the CRISPR-Cas system, the target sequence is 5′ of a Protospacer Adjacent Motif (PAM) and the PAM comprises a 3′ G-rich motif. In embodiments of the CRISPR-Cas system, the target sequence is 5′ of a Protospacer Adjacent Motif (PAM) and the PAM sequence is NGG, wherein N is A, C, G, or T.

In some embodiments of the CRISPR-Cas system, the cohesive ends comprise a single-stranded polynucleotide overhang of 3 to 40 nucleotides. In some embodiments of the CRISPR-Cas system, the cohesive ends comprise a single-stranded polynucleotide overhang of 4 to 20 nucleotides. In some embodiments of the CRISPR-Cas system, the cohesive ends comprise a single-stranded polynucleotide overhang of 5 to 10 nucleotides.

In some embodiments of the CRISPR-Cas system, the stiCas9 is derived from a bacterial species having a Type II-B CRISPR system. In some embodiments of the CRISPR-Cas system, the stiCas9 comprises a domain having at least 80% identity, 85% identity, 90% identity or 95% identity to any of SEQ ID NOs: 10-97 or 192-195. In some embodiments, the stiCas9 comprises a domain that matches a TIGR03031 protein family with an E-value cut-off of 1E-5. In some embodiments, the stiCas9 comprises a domain that matches a TIGR03031 protein family with an E-value cut-off of 1E-10.

In some embodiments of the CRISPR-Cas system, the bacterial species from which the stiCas9 is derived is Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1_47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonassp . S-B4-1U, Tamilnaduibacter salinus, Vibrio natriegens, Arcobacter skirrowii, Francisella philomiragia, Francisella hispaniensis, or Parendozoicomonas haliclonae.

In some embodiments of the CRISPR-Cas system, the target sequence is 5′ of a Protospacer Adjacent Motif (PAM) and the PAM sequence is YG, wherein Y is a pyrimidine, and the stiCas9 is derived from the bacterial species F. novicida.

In some embodiments of the CRISPR-Cas system, the stiCas9 comprises one or more nuclear localization signals. In some embodiments of the CRISPR-Cas system, the eukaryotic cell is an animal or human cell. In some embodiments of the CRISPR-Cas system, the eukaryotic cell is a human cell. In some embodiments of the CRISPR-Cas system, the eukaryotic cell is a plant cell.

In some embodiments of the CRISPR-Cas system, the guide sequence is linked to a direct repeat sequence.

In some embodiments, a delivery particle comprises the CRISPR-Cas system of the present disclosure. In some embodiments, the stiCas9 and the guide polynucleotide are in a complex within the delivery particle.

In some embodiments, the guide polynucleotide further comprises a tracrRNA sequence. In some embodiments, the complex within the delivery particle further comprises a polynucleotide comprising a tracrRNA sequence.

In some embodiments, the delivery particle further comprises a lipid, a sugar, a metal, or a protein.

In some embodiments, a vesicle comprises the CRISPR-Cas system of the present disclosure.

In some embodiments, the stiCas9 and the guide polynucleotide are in a complex within the vesicle.

In some embodiments, the complex within the vesicle further comprises a polynucleotide comprising a tracrRNA sequence. In some embodiments, the vesicle is an exosome or a liposome.

In some embodiments of the CRISPR-Cas system, the one or more nucleotide sequences encoding the stiCas9 is codon optimized for expression in a eukaryotic cell.

In some embodiments of the CRISPR-Cas system, the nucleotide encoding a Cas9 effector protein and the guide polynucleotide are on a single vector.

In some embodiments of the CRISPR-Cas system, the nucleotide encoding a Cas9 effector protein and the guide polynucleotide are a single nucleic acid molecule.

In some embodiments, a viral vector comprises the CRISPR-Cas system of the present disclosure. In some embodiments, the viral vector is of an adenovirus, a lentivirus, or an adeno-associated virus.

In some embodiments, the present disclosure provides a eukaryote cell comprising a CRISPR-Cas system comprising: a Cas9 effector protein capable of generating cohesive ends (stiCas9), and a guide polynucleotide that forms a complex with the stiCas9 and comprises a guide sequence, wherein the guide sequence is capable of hybridizing with a target sequence in a eukaryotic cell, wherein the complex does not occur in nature.

In some embodiments, the present disclosure provides a eukaryote cell comprising a CRISPR-Cas system comprising a Cas9 effector protein capable of generating cohesive ends (stiCas9), wherein the Cas9 effector protein is derived from a bacterial species having a Type II-B CRISPR system.

In some embodiments, the present disclosure provides a method for providing site-specific modification of a target sequence in a eukaryotic cell, the method comprising: (1) introducing into the cell: (a) a Cas9 effector protein capable of generating cohesive ends (stiCas9), and (b) a guide polynucleotide that forms a complex with the stiCas9 and comprises a guide sequence, wherein the guide sequence is capable of hybridizing with the target sequence in the eukaryotic cell but does not hybridize to a sequence in a bacterial cell, wherein the complex does not occur in nature; (2) generating cohesive ends in the target sequence with the Cas9 effector protein and the guide polynucleotide; and (3) ligating (a) the cohesive ends together, or (b) a polynucleotide sequence of interest (SoI) to the cohesive ends, thereby modifying the target sequence.

In some embodiments, the present disclosure provides a method for providing site-specific modification of a target sequence in a eukaryotic cell, the method comprising: (1) introducing into the cell: (a) a nucleotide sequence encoding a Cas9 effector protein capable of generating cohesive ends (stiCas9), and (b) a guide polynucleotide that forms a complex with the stiCas9 and comprises a guide sequence, wherein the guide sequence is capable of hybridizing with the target sequence in the eukaryotic cell but does not hybridize to a sequence in a bacterial cell, wherein the complex does not occur in nature; (2) generating cohesive ends in the target sequence with the Cas9 effector protein and the guide polynucleotide; and (3) ligating: (a) the cohesive ends together, or (b) a polynucleotide sequence of interest (SoI) to the cohesive ends, thereby modifying the target sequence.

In some embodiments, the methods for providing site-specific modification of a target sequence in a eukaryotic cell further comprise introducing into the cell a polynucleotide comprising a tracrRNA sequence.

In some embodiments of the method, the guide polynucleotide, tracrRNA sequence, and the stiCas9 are capable of forming a complex, and wherein the complex does not occur in nature.

In some embodiments of the method, the complex is capable of cleaving at a site within 10 nucleotides of a Protospacer Adjacent Motif (PAM). In some embodiments of the method, the complex is capable of cleaving at a site within 5 nucleotides of a Protospacer Adjacent Motif (PAM). In some embodiments of the method, the complex is capable of cleaving at a site within 3 nucleotides of a Protospacer Adjacent Motif (PAM).

In some embodiments of the method, the target sequence is 5′ of a Protospacer Adjacent Motif (PAM) and the PAM comprises a 3′ G-rich motif. In some embodiments of the method, the target sequence is 5′ of a PAM and the PAM sequence is NGG, wherein N is A, C, G, or T.

In some embodiments of the method, the cohesive ends comprise a single-stranded polynucleotide overhang of 3 to 40 nucleotides. In some embodiments of the method, the cohesive ends comprise a single-stranded polynucleotide overhang of 4 to 20 nucleotides. In some embodiments of the method, the cohesive ends comprise a single-stranded polynucleotide overhang of 5 to 10 nucleotides.

In some embodiments of the method, the stiCas9 is derived from a bacterial species having a Type II-B CRISPR system.

In some embodiments of the method, the eukaryotic cell is an animal or human cell. In some embodiments of the method, the eukaryotic cell is a human cell. In some embodiments of the method, the eukaryotic cell is a plant cell.

In some embodiments of the method, the modification is deletion of at least part of the target sequence. In embodiments of the method, the modification is mutation of the target sequence. In some embodiments of the method, the modification is inserting a sequence of interest into the target sequence.

In some embodiments, the method further comprises introducing an exonuclease to remove overhangs generated from the stiCas9.

In some embodiments of the method, the exonuclease is Cas4, Artemis, or TREX4. In some embodiments of the method, the Cas4 is derived from a bacterial species having a Type II-B CRISPR system.

In some embodiments of the method, a polynucleotide encoding components of the complex is introduced on one or more vectors.

In some embodiments, the disclosure is directed to a method of introducing a sequence of interest (SoI) into a chromosome in a cell, wherein the chromosome comprises a target sequence (TSC) comprising region 1 and region 2, the method comprising introducing into the cell:
(a) a vector comprising a target sequence (TSV), the TSV comprising region 2 and region 1 and the SoI;
(b) a first Cas9-endonuclease dimer capable of generating cohesive ends in the TSC, wherein a first monomer of the first Cas9-endonuclease dimer cleaves at region 1 and a second monomer of the first Cas9-endonuclease dimer cleaves at region 2 of the TSC; and
(c) a second Cas9-endonuclease dimer capable of generating cohesive ends in the TSV, wherein a first monomer of the second Cas9-endonuclease dimer cleaves at region 2 and a second monomer of the second Cas9-endonuclease dimer cleaves at region 1 of the TSV;
wherein introduction of the vector of (a), the first Cas9-endonuclease dimer of (b) and the second Cas9-endonuclease dimer of (c) results in insertion of the SoI into the chromosome of the cell.

In some embodiments, the disclosure is directed to a method of introducing a sequence of interest (SoI) into a chromosome in a cell, wherein the chromosome comprises a target sequence (TSC) comprising region 1 and region 2, the method comprising introducing into the cell:
(a) a vector comprising a target sequence (TSV), the TSV comprising region 2 and region 1 and the SoI, wherein the vector comprises cohesive ends;
(b) a first Cas9-endonuclease dimer capable of generating cohesive ends in the TSC, wherein a first monomer of the first Cas9-endonuclease dimer cleaves at region 1 and a second monomer of the first Cas9-endonuclease dimer cleaves at region 2 of the TSC;
wherein introduction of the vector of (a) and the first Cas9-endonuclease dimer of (b) results in insertion of the SoI into the chromosome of the cell.

In some embodiments, the first and second Cas9-endonuclease dimers are the same. In some embodiments, the first and second Cas9-endonuclease dimers are different.

In some embodiments, the method further comprises introducing into the cell a first guide polynucleotide that forms a complex with the first monomer of the first Cas9-endonuclease dimer and comprises a first guide sequence, wherein the first guide sequence hybridizes to the TSC comprising region 1 but does not hybridize to the vector.

In some embodiments, the method further comprises introducing into the cell a first guide polynucleotide that forms a complex with the first monomer of the first Cas9-endonuclease dimer and comprises a first guide sequence, wherein the first guide sequence hybridizes to the TSC and the TSV.

In some embodiments, the method further comprises introducing into the cell a second guide polynucleotide that forms a complex with the second monomer of the first Cas9-endonuclease dimer and comprises a second guide sequence, wherein the second guide sequence hybridizes to the TSC comprising region 2 but does not hybridize to the vector.

In some embodiments, the method further comprises introducing into the cell a second guide polynucleotide that forms a complex with the second monomer of the first Cas9-endonuclease dimer and comprises a second guide sequence, wherein the second guide sequence hybridizes to the TSC and the TSV.

In some embodiments, the method further comprises introducing into the cell a third guide polynucleotide that forms a complex with the first monomer of the second Cas9-endonuclease dimer and comprises a third guide sequence, wherein the third guide sequence hybridizes to the TSV comprising region 2 but does not hybridize to the chromosome.

In some embodiments, the method further comprises introducing into the cell a third guide polynucleotide that forms a complex with the first monomer of the second Cas9-endonuclease dimer and comprises a third guide sequence, wherein the third guide sequence hybridizes to the TSC and the TSV.

In some embodiments, the method further comprises introducing into the cell a fourth guide polynucleotide that forms a complex with the second monomer of the second Cas9-endonuclease dimer and comprises a fourth guide sequence, wherein the fourth guide sequence hybridizes to the TSV comprising region 1 but does not hybridize to the chromosome.

In some embodiments, the method further comprises introducing into the cell a fourth guide polynucleotide that forms a complex with the second monomer of the second Cas9-endonuclease dimer and comprises a fourth guide sequence, wherein the fourth guide sequence hybridizes to the TSC and the TSV.

In some embodiments, the method comprises introducing into the cell the first, second, third, and fourth guide polynucleotides.

In some embodiments, the method further comprises introducing into the cell a polynucleotide comprising a tracrRNA sequence.

In some embodiments, the endonucleases in the first monomer and the second monomer of the first Cas9-endonuclease dimer are Type IIS endonucleases. In some embodiments, the endonucleases in the first monomer and the second monomer of the second Cas9-endonuclease dimer are Type IIS endonucleases.

In some embodiments, the endonucleases in the first Cas9-endonuclease dimer and the second Cas9-endonuclease dimer are Type IIS endonucleases. In some embodiments, the endonucleases in the first Cas9-endonuclease dimer and the second Cas9-endonuclease dimer, are independently selected from the group consisting of BbvI, BgcI, BfuAI, BmpI, BspMI, CspCI, FokI, MboII, MmeI, NmeAIII, and PleI. In some embodiments, the endonucleases in the first Cas9-endonuclease dimer and the second Cas9-endonuclease dimer are FokI. In some embodiments, the first and second Cas9-endonuclease dimers are introduced into the cell as a polynucleotide encoding the first and second Cas9-endonuclease dimer.

In some embodiments, the polynucleotides encoding the first and second Cas9-endonuclease dimers are on one vector. In some embodiments, the polynucleotides encoding the first and second Cas9-endonuclease dimers are on more than one vector.

In some embodiments, the first, second or both Cas9-endonuclease dimers comprise a modified Cas9. In some embodiments, the first, second or both Cas9-endonuclease dimers comprise a catalytically inactive Cas9. In some embodiments, the endonuclease in the first, second or both Cas9-endonuclease dimers is FokI. In some embodiments, the first, second or both Cas9-endonuclease dimers comprise a Cas9 having nickase activity. In some embodiments, the endonuclease in the first, second or both Cas9-endonuclease dimers is FokI.

In some embodiments, the Cas9-endonuclease dimer comprises a single amino-acid substitution in Cas9 relative to a wild-type Cas9. In some embodiments, the endonuclease in the first, second or both Cas9-endonuclease dimers is FokI. In some embodiments, the single amino-acid substitution is D10A or H840A. In some embodiments, the single amino-acid substitution is D10A. In some embodiments, the single amino-acid substitution is H840A. In some embodiments, the Cas9-endonuclease dimer comprises a double amino-acid substitution relative to a wild-type Cas9. In some embodiments, the double amino-acid substitution is D10A and H840A.

In some embodiments, the wild-type Cas9 is derived from Streptococcus pyogenes, Staphylococcus aureus, Staphylococcus pseudintermedius, Planococcus antarcticus, Streptococcus sanguinis, Streptococcus thermophilus, Streptococcus mutans, Coribacterium glomerans, Lactobacillus farciminis, Catenibacterium mitsuokai, Lactobacillus rhamnosus, Bifidobacterium bifidum, Oenococcus kitahara, Fructobacillus fructosus, Finegoldia magna, Veillonella atyipca, Solobacterium moorei, Acidaminococcus sp. D21, Eubacterium yurri, Coprococcus catus, Fusobacterium nucleatum, Filifactor alocis, Peptoniphilus duerdenii, or Treponema denticola.

In some embodiments, the cohesive ends comprise a 5′ overhang. In some embodiments, the cohesive ends comprise a 3′ overhang. In some embodiments, the first, second or both Cas9-endonuclease dimers generate cohesive ends comprising a single-stranded polynucleotide of 3 to 40 nucleotides. In some embodiments, the first, second or both Cas9-endonuclease dimers generate cohesive ends comprising a single-stranded polynucleotide of 4 to 20 nucleotides. In some embodiments, the first, second or both Cas9-endonuclease dimers generate cohesive ends comprising a single-stranded polynucleotide of 5 to 15 nucleotides.

In some embodiments of the method, upon the insertion, the target sequence in the chromosome and the target sequence in the plasmid are not reconstituted.

In some embodiments, the cell is a eukaryotic cell. In some embodiments, the cell is an animal or human cell. In some embodiments, the cell is a plant cell.

In some embodiments of the method of introducing a sequence of interest (SoI) into a chromosome in a cell, the vector of (a), the first Cas9-endonuclease dimer of (b), the second Cas9-endonuclease dimer of (c) or combinations thereof are introduced into the cell via delivery particles, vesicles, or viral vectors. In some embodiments, the vector of (a), the first Cas9-endonuclease dimer of (b), the second Cas9-endonuclease dimer of (c) or combinations thereof are introduced into the cell via delivery particles. In some embodiments, the delivery particles comprise a lipid, a sugar, a metal, or a protein.

In some embodiments of the method of introducing a sequence of interest (SoI) into a chromosome in a cell, the vector of (a), the first Cas9-endonuclease dimer of (b), the second Cas9-endonuclease dimer of (c) or combinations thereof are introduced into the cell via vesicles. In some embodiments, the vesicles are exosomes or liposomes.

In some embodiments of the method of introducing a sequence of interest (SoI) into a chromosome in a cell, polynucleotides capable or expressing the vector of (a), the first Cas9-endonuclease dimer of (b), the second Cas9-endonuclease dimer of (c) or combinations thereof are introduced into the cell via a viral vector. In some embodiments, the vector of (a) is a viral vector. In some embodiments, the viral vector is an adenovirus, lentivirus, or adeno-associated virus.

In some embodiments, the first monomer of the first Cas9-endonuclease dimer forms a complex with the first guide polynucleotide, and the second monomer of the first Cas9-endonuclease dimer forms a complex with the second guide polynucleotide. In some embodiments, the first monomer of the second Cas9-endonuclease dimer forms a complex with the third guide polynucleotide, and the second monomer of the second Cas9-endonuclease dimer forms a complex with the fourth guide polynucleotide. In some embodiments, the first monomer of the first Cas9-endonuclease dimer forms a complex with the first guide polynucleotide sequence and a tracrRNA sequence, and the second monomer of the first Cas9-endonuclease dimer forms a complex with the second guide polynucleotide sequence and a tracrRNA sequence. In some embodiments, the first monomer of the second Cas9-endonuclease dimer forms a complex with the third guide polynucleotide sequence and a tracrRNA sequence, and the second monomer of the second Cas9-endonuclease dimer forms a complex with the fourth guide polynucleotide sequence and a tracrRNA sequence. In some embodiments, the first, second or both Cas9-endonuclease dimers comprise a nuclear localization signal.

In some embodiments of the method of introducing a sequence of interest (SoI) into a chromosome in a cell, the cell comprises a stem cell or stem cell line.

In some embodiments, the disclosure is directed to a method of modifying one or more nucleotides in a target polynucleotide sequence in a cell, the method comprising:
(a) introducing into the cell a vector comprising an insertion cassette (IC), the IC comprising, in a 5′ to 3′ direction,
   (i) a first region homologous to part of the target polynucleotide sequence,
   (ii) a second region comprising a mutation of the target polynucleotide sequence of one or more nucleotides,
   (iii) a first nuclease binding site,
   (iv) a polynucleotide sequence encoding a marker gene,
   (v) a second nuclease binding site,
   (vi) a third region comprising a mutation of the target polynucleotide sequence of one or more nucleotides, and
   (vii) a fourth region homologous to part of the target polynucleotide sequence, wherein the first region and the fourth region are 95%-100% identical to the target polynucleotide sequence;
(b) inserting the IC into the target polynucleotide sequence via homologous recombination to generate a first modified target polynucleotide;
(c) selecting a cell which expresses the marker gene;
(d) subjecting the first modified target polynucleotide to a site-specific nuclease to generate a second modified target polynucleotide having cohesive ends; and
(e) subjecting the second modified target polynucleotide having cohesive ends to a ligase, wherein the ligase ligates the cohesive ends at the second region and the third region to create a ligated modified target nucleic acid comprising one or more modified nucleotides when compared to the target polynucleotide sequence.

In some embodiments of a method of modifying one or more nucleotides in a target polynucleotide sequence in a cell, the first modified target nucleic acid is isolated from the cell after (c).

In some embodiments, the site-specific nuclease is exogenous to the cell. In some embodiments, the ligase is exogenous to the cell. In some embodiments, the first modified target protein is in the cell after (c). In some embodiments, the site-specific nuclease is introduced into the cell as a polynucleotide encoding the site-specific nuclease. In some embodiments, the ligase is introduced into the cell as a polynucleotide encoding a ligase.

In some embodiments, the site-specific nuclease is a recombinant site-specific nuclease. In some embodiments, the ligase is a recombinant ligase. In some embodiments, the site-specific nuclease is a Cas9 effector protein. In some embodiments, the Cas9 effector protein is a Type II-B Cas9. In some embodiments, the site-specific nuclease is a Cas9-endonuclease fusion protein. In some embodiments, the endonuclease in the Cas9-endonuclease fusion protein is a Type IIS endonuclease. In some embodiments, the endonuclease in the Cas9-endonuclease fusion protein is FokI.

In some embodiments, the Cas9-endonuclease fusion protein comprises a modified Cas9. In some embodiments, the modified Cas9 comprises a catalytically inactive Cas9. In some embodiments, the catalytically inactive Cas9 is fused to FokI endonuclease.

In some embodiments, the Cas9-endonuclease fusion protein comprises a Cas9 having nickase activity, and the endonuclease is FokI. In some embodiments, the Cas9-endonuclease fusion protein comprises a Cas9 having a D10A substitution. In some embodiments, the Cas9-endonuclease fusion protein comprises a Cas9 having a H840A substitution.

In some embodiments, the site-specific nuclease is a Cpf1 effector protein. In some embodiments, the site-specific nuclease is Cas9, Cpf1, or Cas9-FokI.

In some embodiments of a method of modifying one or more nucleotides in a target polynucleotide sequence in a cell, the cohesive ends of the second modified target polynucleotide of (d) comprise a 5′ overhang. In some embodiments, the cohesive ends of the second modified target polynucleotide of (d) comprise a 3′ overhang. In some embodiments, the site-specific nuclease is capable of generating cohesive ends comprising a single-stranded polynucleotide of 3 to 40 nucleotides. In some embodiments, the nuclease is capable of generating cohesive ends comprising a single-stranded polynucleotide of 4 to 20 nucleotides. In some embodiments, the nuclease is capable of generating cohesive ends comprising a single-stranded polynucleotide of 5 to 15 nucleotides.

In some embodiments of a method of modifying one or more nucleotides in a target polynucleotide sequence in a cell, the target polynucleotide sequence is in a plasmid. In some embodiments, the target polynucleotide sequence is in a chromosome.

In some embodiments, the disclosure is directed to an engineered guide RNA that forms a complex with a stiCas9 protein, comprising: (a) a guide sequence capable of hybridizing to a target sequence in a eukaryotic cell; and (b) a tracrRNA sequence capable of binding to the Cas9 protein, wherein the tracrRNA differs from a naturally-occurring tracrRNA sequence by at least 10 nucleotides, wherein the engineered guide RNA improves nuclease efficiency of the Cas9 protein. In some embodiments, the tracrRNA sequence has at least 10 fewer nucleotides than a naturally-occurring tracrRNA. In some embodiments, the tracrRNA sequence has at least 10 more nucleotides than a naturally-occurring tracrRNA. In some embodiments, the guide sequence comprises at least 90% sequence identity to any one of SEQ ID NOs: 104-125 or 196-199. In some embodiments, the tracrRNA sequence comprises at least 90% sequence identity to any one of SEQ ID NOs: 148-171. In some embodiments, the guide RNA comprises at least 90% sequence identity to any one of SEQ ID NOs: 172-191.

In some embodiments, the disclosure is directed to a CRISPR-Cas system comprising an engineered guide RNA as described herein. In some embodiments, the system does not comprise a tracrRNA sequence.

In some embodiments, the disclosure is directed to an engineered Cas9-guide RNA complex, comprising any combination of Cas9, guide sequence, and tracrRNA sequence as found in FIG. 40B. In some embodiments, the disclosure is directed to a method of producing an engineered guide RNA that binds to a Cas9 protein, comprising: (a) providing a guide sequence capable of hybridizing to a target sequence in a eukaryotic cell; (b) modifying a naturally-occurring tracrRNA sequence by removing at least ten nucleotides from the tracrRNA sequence to form a modified tracrRNA sequence; and (c) linking the guide sequence to the modified tracrRNA sequence to generate the engineered guide RNA. In some embodiments, the disclosure is directed to a non-naturally occurring CRISPR-Cas system comprising: (a) a Cas9 effector protein capable of generating cohesive ends (stiCas9); and (b) a guide RNA that forms a complex with the stiCas9 and comprises a guide sequence, wherein the guide sequence is capable of hybridizing with a target sequence in a eukaryotic cell but does not hybridize to a sequence in a bacterial cell; wherein the complex does not occur in nature, and wherein the system does not comprise a tracrRNA sequence.

### BRIEF DESCRIPTION OF THE FIGURES

FIG. 1 is a schematic of different mechanisms of repair by Cas9. FIG. 1a represents gene knock-outs. FIG. 1b represents base editing. FIG. 1c represents gene knock-ins by the Non-Homologous End Joining (NHEJ) pathway. FIG. 1d represents gene knock-ins by the Homology-Directed Recombination (HDR) pathway.

FIG. 2 is a schematic of different mechanisms of gene insertion by Cas9. Homology-Directed Recombination (HDR) is shown on the left. Non-Homologous End Joining (NHEJ) is shown on the right.

FIG. 3 is a schematic and representation of results for gene insertion using different Cas9 effector proteins. FIG. 3a-b show gene insertion mediated by Cas9 generating blunt ends. FIG. 3c-d show gene insertion mediated by Cas9 generating overhangs (i.e., “sticky ends”). The lower panel of FIG. 3 is a representation of the gene insertion frequency by the different Cas9 proteins in 3a-3f, using Homology-Independent Targeted Insertion (HITI).

FIG. 4 is described by Shmakov et al., Nature Reviews Microbiology 15:169-182 (2017). FIG. 4A is a phylogeny tree of different types of CRISPR systems and representative bacterial species having each type of CRISPR system. FIG. 4B shows a close-up of the Type II and Type V CRISPR systems, with arrows indicating operons that contain a cas4 gene.

FIG. 5 is described by Chylinski et al., Nucleic Acids Research 42(10):6091-6105 (2014). FIG. 5A-D represent a phylogeny tree of Type II CRISPR systems. FIG. 5E shows the different signature genes associated with each subfamily of Type II CRISPR systems.

FIG. 6A represents the results obtained for DNA cleavage using the Cas9 protein from Francisella novicida. Mutation signatures for a genomic locus in an engineered HEK293 cell line targeted with Cas9 from Francisella novicida and Cas9 from Streptococcus pyogenes are compared. FIG. 6A discloses SEQ ID NOS 204-205 and 284, respectively, in order of appearance. FIG. 6B-C is a phylogenetic tree of Type II CRISPR systems. Cas9 proteins chosen for in vitro validation are indicated in italics.

FIG. 7 is a schematic representation of the ObLiGaRe method for gene insertion, using zinc-finger nucleases (ZFN) as described in U.S. Pat. No. 9,567,608.

FIG. 8 is a schematic representation of the Cas9-PiTCH method for gene insertion as described by Sakuma et al., Nature Protocols 11(1): 118-133 (2016).

FIG. 9 is a schematic representation of three different Cas9-FokI fusion proteins. FIG. 9a: fusion of enzymatically inactivated Cas9 (deadCas9) with FokI; FIG. 9b: fusion of Cas9 with D10A mutation (Cas9n^{D10A}) with FokI; FIG. 9c: fusion of Cas9 with H840A (Cas9n^{H840A}) with FokI. FIGS. 9a-c disclose SEQ ID NO: 206.

FIG. 10 is a schematic representation of the different DNA breaks generated by the different Cas9-FokI fusion proteins in FIGS. 9 and 10. FIG. 10 discloses SEQ ID NO: 206 as “TCCCCTCCACCCCACAGTGGGGCCACTAGGGACAGGATTGGTGACAGAAAAGCCCC ATCCTTAGGCCT” and the cleaved sequences as SEQ ID NOS 285-289, respectively, in order of appearance.

FIG. 11 is a schematic representation of the cleavage site generated by Cas9n^{D10A}-FokI.

FIG. 11 discloses SEQ ID NO: 206.

FIG. 12 is a schematic representation of a gene insertion method using Cas9n^{D10A}-FokI. gRNA: guide RNA; PAM; protospacer adjacent motif. FIG. 12 discloses the “GENOME” sequences as SEQ ID NOS 206-208, the “VECTOR” sequences as SEQ ID NOS 209-211 and the “Knockin” sequence as SEQ ID NO: 212, all respectively, in order of appearance.

FIG. 13 is a schematic representation of the cleavage site generated by Cas9n^{H840A}-FokI. FIG. 13 discloses SEQ ID NO: 206.

FIG. 14 is a schematic representation of a gene insertion method using Cas9n^{H840A}-FokI. gRNA: guide RNA; PAM; protospacer adjacent motif. FIG. 14 discloses the “GENOME” sequences as SEQ ID NOS 206 and 213-214, the “VECTOR” sequences as SEQ ID NOS 215-217 and the “Knockin” sequence as SEQ ID NO: 218, all respectively, in order of appearance.

FIGS. 15-18 relate to the experiments set forth in Example 1.

FIG. 15 is a schematic representation of a gene insertion method using Cas9n^{D10A}-FokI (FIG. 15) and Cas9n^{H840A}-FokI (FIG. 15). FIGS. 15a-b disclose SEQ ID NO: 206.

FIG. 16 represents the target site (AAVS1 locus). “PlanA” refers to the gene insertion method using Cas9n^{D10A}-FokI; “PlanB” refers to the gene insertion method using Cas9n^{H840A}-FokI. FIG. 16 discloses SEQ ID NO: 219.

FIG. 17 shows representative resulting sequences from the gene insertion method using Cas9n^{D10A}-FokI. FIG. 17 discloses SEQ ID NOS 220-235, respectively, in order of appearance.

FIG. 18 shows representative resulting sequences from the gene insertion method using Cas9n^{H840A}-FokI. FIG. 18 discloses SEQ ID NOS 236-258, respectively, in order of appearance.

FIGS. 19-22 relate to the experiments set forth in Example 2.

FIG. 19 shows the design of a set of 10 guide RNAs (gRNA) used to target the AAVS1 locus.

FIG. 20 is a plasmid map of the “donor” plasmid containing the gene to be inserted into the AAVS1 locus using the gRNAs in FIG. 20.

FIG. 21 is a schematic of the procedure for selecting cells containing a correctly inserted gene (mCherry+ cells).

FIG. 22 shows results of gene insertion frequency with spacers of different lengths.

FIGS. 23-24 relate to the experiments set forth in Example 3.

FIG. 23 is a plasmid map of the “donor” plasmid containing the gene to be inserted into the SERPINA1 locus.

FIG. 24 is a schematic representation of a gene insertion method using deadCas9-FokI. FIG. 24 discloses SEQ ID NO: 206.

FIG. 25 is a comparison of the efficiency of the different methods used for targeted gene insertions, as set forth in Examples 2-4.

FIGS. 26-29 relate to the experiments set forth in Example 4.

FIG. 26 is a schematic of a seamless mutagenesis.

FIG. 27 is a schematic of the first step of seamless mutagenesis: recombination of a cassette containing a resistance marker into a target sequence using homology arms.

FIG. 28 is a schematic of the cassette integrated into the target sequence: a resistance marker flanked on both sides by nuclease binding sites and nuclease cutting sites.

FIG. 29 is a schematic of the second step of seamless mutagenesis: nuclease digestion at the cutting sites (shown in FIG. 28) and subsequent ligation, resulting in removal of the resistance marker and a seamlessly-generated mutation.

FIG. 30 includes amino acid sequences of Cas9 proteins from various sequenced bacteria, including: Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1-1_47, Bacteroidetes oral taxon 274 str. F0058, and Wolinella succinogenes. (SEQ ID NOS: 10-80.)

FIG. 31 includes amino acid sequences of Cas9 proteins from various sequenced bacteria, including: Burkholderiales bacterium, Campylobacter sp., Turicimonas muris, Salinivibrio sharmensis, Leptospira sp., Moritella sp., Endozoicomonas sp., Tamilnaduibacter salinus, Vibrio natriegens, Ruminobacter amylophilus, Vibrio sagaiensis, Arcobacter porcinus, Desulfofustis sp., and Succinatimonas sp. (SEQ ID NOS: 81-97.)

FIG. 32 includes nucleotide sequences of a guide RNA sequence, a tracrRNA sequence, and a crRNA sequence used in the experiments set forth in Example 8 on a Cas9 protein from MH0245_GL0161830_1 (SEQ ID NOS: 101-103).

FIG. 33A shows an exemplary 4-nucleotide 5′ overhang generated by a Type II-B Cas9 protein. FIG. 33A discloses SEQ ID NO: 259. FIG. 33B shows an exemplary Type II-B cas operon. cas9, cas 1, cas2, and cas4 genes are represented by arrows. A CRISPR array is marked downstream of the operon.

FIG. 34 relates to the experiments set forth in Example 7. FIG. 34A shows an electrophoresis gel image that demonstrates in vitro nuclease activity of a Cas9 protein from Francisella novicida (FnCas9). FIG. 34B shows a Sanger sequencing plot indicating that FnCas9 generates cohesive ends with a 5′ overhang. FIG. 34B discloses SEQ ID NOS 204-205 and 284, respectively, in order of appearance. FIG. 34C shows a RIMA comparison of the mutation patterns between Streptococcus pyogenes Cas9 protein (SpyCas9) and FnCas9.

FIGS. 35-36 relate to the experiments set forth in Example 8.

FIG. 35A shows an electrophoresis gel image that demonstrates in vitro nuclease activity of a Cas9 protein from the sequence gut metagenome MH0245 (MHCas9). FIG. 35B shows a Sanger sequencing plot indicating that MHCas9 generates cohesive ends with a 5′ overhang. FIG. 35B discloses SEQ ID NOS 260-262, respectively, in order of appearance. FIG. 35C shows an electrophoresis gel image that demonstrates MHCas9 activity in HEK293-REMINDEL cells, validated by a Cell1 assay.

FIG. 36A shows the sequence of the crRNA and tracrRNA from MHCas9. FIG. 36A discloses SEQ ID NO: 263. FIG. 36B shows a scheme of the crRNA/tracrRNA secondary structures. FIG. 36C shows a truncated phylogenetic tree with Cas9 proteins from Sulfurospirillum sp. SCADC (ssCas9), Wolinella succinogenes (WsCas9), Legionella pneumophila (LpCas9), Francisella novicida (FnCas9), and MH0245 (MHCas9).

FIG. 37 is a phylogenetic tree generated from the amino acid sequences of Cas9 proteins from various bacterial species, as described herein. Sequence alignment was performed using the MUSCLE algorithm, CLC Genomics Workbench v.9.

FIG. 38 is a phylogenetic tree generated from the amino acid sequences of Cas9 proteins from various species of the genus Campylobacter. Sequence alignment was performed using the MUSCLE algorithm, CLC Genomics Workbench v.9.

FIG. 39 includes nucleotide sequences of crRNA for various Cas9 proteins described herein (SEQ ID NOS: 104-147).

FIG. 40A includes nucleotide sequences of tracrRNA for various Cas9 proteins described herein (SEQ ID NOS: 148-171).

FIG. 40B includes various combinations of Cas9 proteins, crRNA(+), crRNA(−) and tracrRNA.

FIGS. 41A-T illustrate various sgRNAs (also termed “chimeric gRNA”) designed by the method described in Example 9, including sequences of the sgRNAs (SEQ ID NOs: 172-191). FIG. 41A also discloses the hairpin sequence as SEQ ID NO: 264.

FIGS. 42A-L illustrate the optimization and trimming of sgRNAs described in Example 9, and possible target sites for further modifications. FIG. 42A discloses SEQ ID NOS 265-266, respectively, in order of appearance. FIG. 42B discloses SEQ ID NOS 267-268, respectively, in order of appearance. FIG. 42C discloses SEQ ID NOS 269 and 173, respectively, in order of appearance. FIG. 42D discloses SEQ ID NOS 270-271, respectively, in order of appearance. FIG. 42E discloses SEQ ID NOS 178 and 272, respectively, in order of appearance. FIG. 42F discloses SEQ ID NOS 179 and 273, respectively, in order of appearance. FIG. 42G discloses SEQ ID NOS 180 and 274, respectively, in order of appearance. FIG. 42H discloses SEQ ID NOS 176 and 275, respectively, in order of appearance. FIG. 42I discloses SEQ ID NOS 174 and 276, respectively, in order of appearance. FIG. 42J discloses SEQ ID NOS 191 and 277, respectively, in order of appearance. FIG. 42K discloses SEQ ID NOS 184 and 278, respectively, in order of appearance. FIG. 42L discloses SEQ ID NOS 279-280, respectively, in order of appearance.

FIG. 43 illustrates a bi-directional expression construct of a Type II-B CRISPR-Cas system. As shown in the inset, the top strand expresses the crRNA and spacer for a single-guide RNA that does not include a tracrRNA. The bottom strand expresses the crRNA and spacer for a dual-guide RNA that includes a tracrRNA. FIG. 43 discloses SEQ ID NOS 137, 281 and 191, respectively, in order of appearance.

FIG. 44 shows predicted secondary structures of single-guide RNA scaffolds for Cas9 proteins described herein. FIG. 44 discloses SEQ ID NOS 137, 139, 282, 122, 110, 129, 120, 124 and 104, respectively, in order of appearance.

FIG. 45 generically describes four different engineered RNAs, and the cutting efficiency of each with MHCas9.

FIG. 46 demonstrates the cutting efficiency and functionality of Guide RNA of lengths 19, 20, 21, 22 and 23 with three different Cas9 systems SpyCas9, C11Cas9 and MHCas9.

FIG. 47 includes amino acid sequences of Cas9 proteins from various sequenced bacteria, including: Arcobacter skirrowii, Francisella philomiragia, Francisella hispaniensis, and Parendozoicomonas haliclonae (SEQ ID NOS: 192-195).

FIG. 48 includes nucleotide sequence of crRNA for various Cas9 proteins described herein (SEQ ID NOS: 196-203).

FIG. 49 relates to Example 11. FIG. 49A shows an exemplary method for determining the PAM sequence of a Cas9 protein. FIG. 49A discloses SEQ ID NO: 283. FIG. 49B shows the preferred PAM sequences for SpCas9 (top) and MHCas9 (bottom), as determined by the method shown in FIG. 49A.

FIGS. 50 and 51 relate Example 12.

FIG. 50A shows the schematic of a Cas9 cut repaired precisely. FIG. 50B shows the schematics of a Cas9 cut, coupled with end processing by exonucleases such as TREX2 or Artemis, resulting in imprecise repair and increased modifications.

FIG. 51A shows an overview of the method for testing the effects of adding an end processing enzyme (FnCas4 or TREX2) to various Cas9 (SpCas9, FnCas9, C11Cas9, or MHCas9), with three different guide RNAs. FIG. 51B shows the results for each of the Cas9 proteins, with either mock end processing enzyme, FnCas4, or TREX2, and with each of the three guide RNA's.

FIGS. 52 and 53 relate to Example 13.

FIGS. 52A, 52B, and 52C show the different types of mutations generated by SpCas9, C11Cas9, or MHCas9, respectively, when all three Cas9 proteins cut at the same sequence. FIGS. 52A-C disclose SEQ ID NO: 290.

FIG. 53A shows a schematic of the RuvC and HNH domains of a Type II-A Cas9 protein cutting a double-stranded DNA sequence complexed with a guide RNA, which generates blunt or single nucleotide overhangs. FIG. 53B shows a schematic of the RuvC and HNH domains of a Type II-B Cas9 protein cutting a double-stranded DNA sequence complexed with a guide RNA, which generates sticky ends with a 3- or 4-nucleotide overhang.

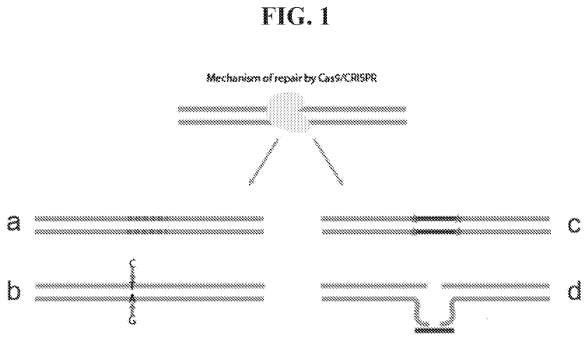

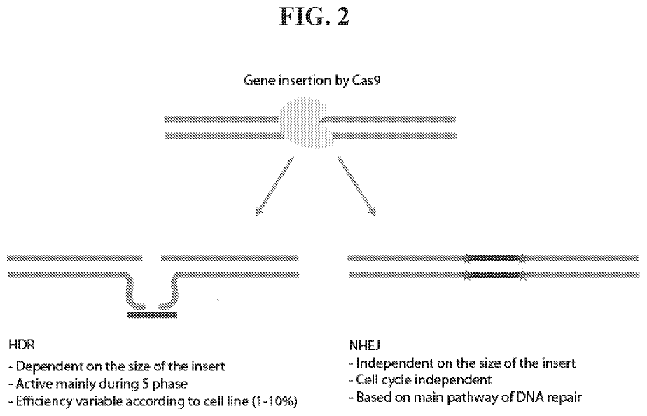

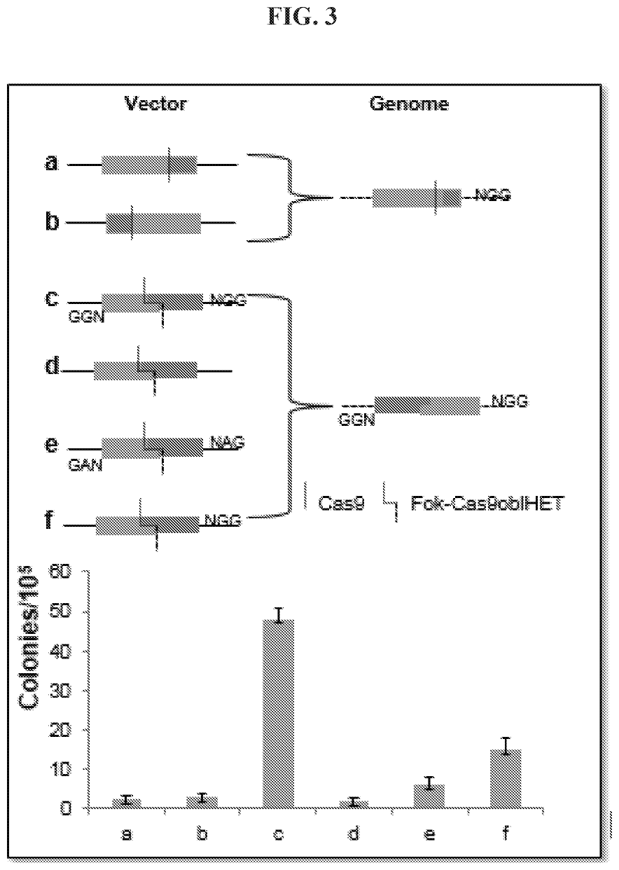

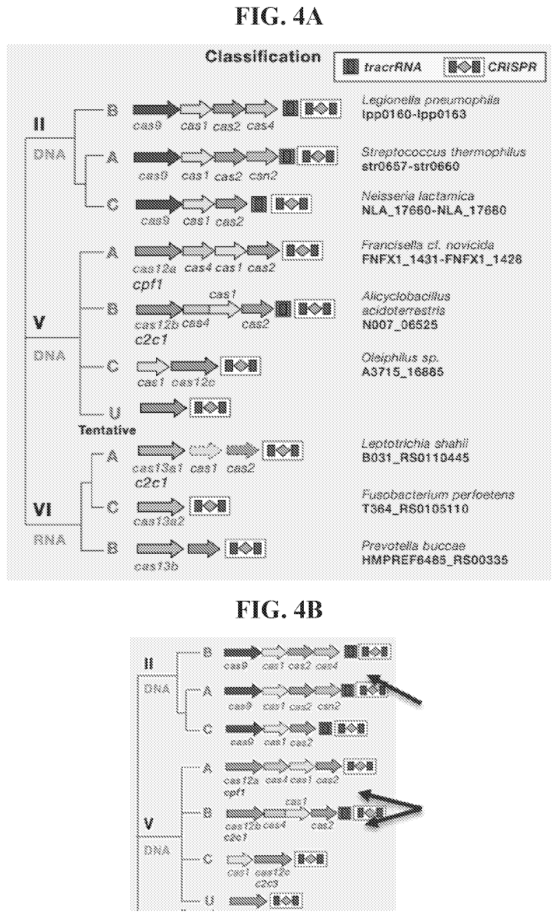

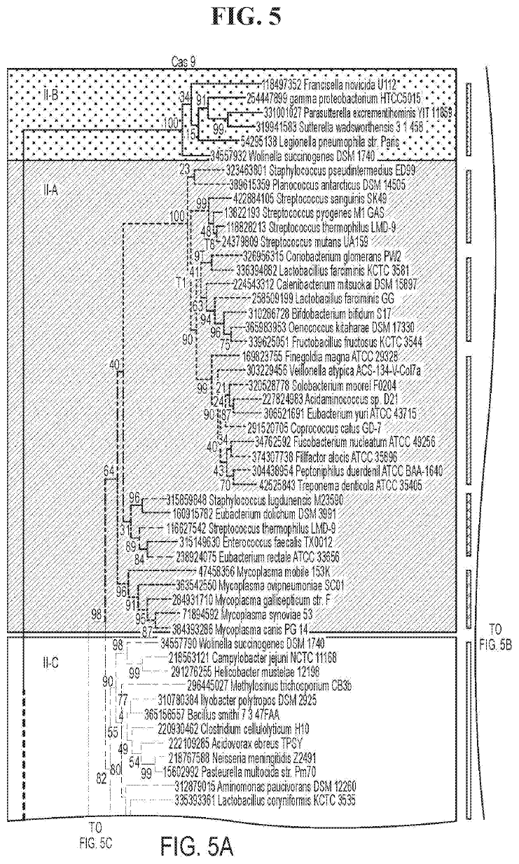

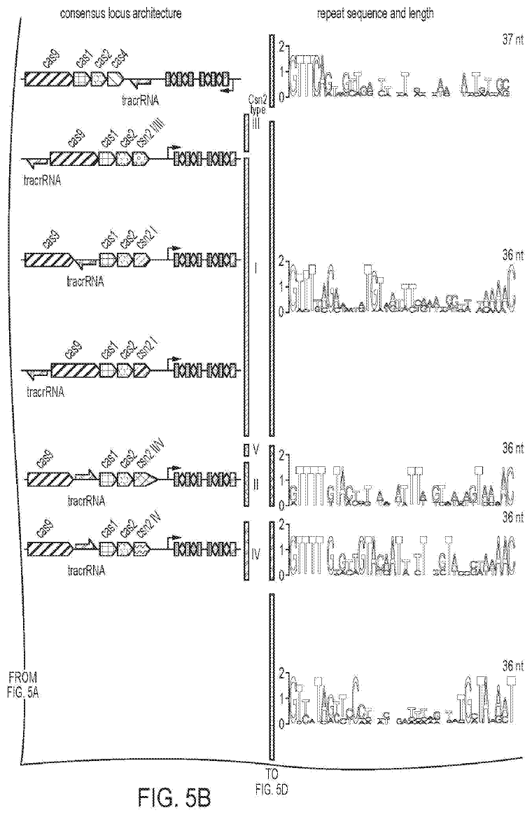

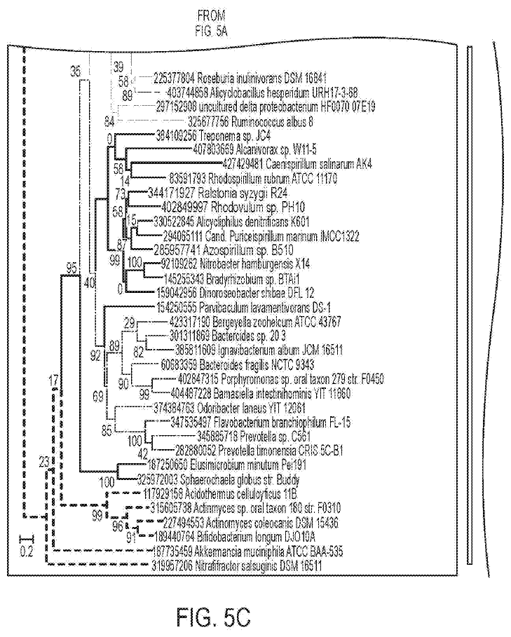

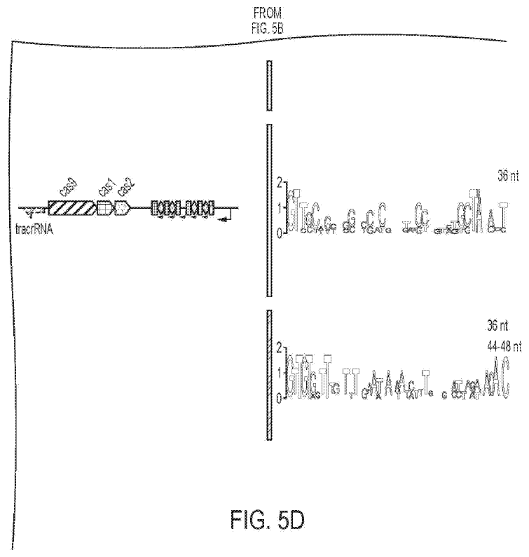

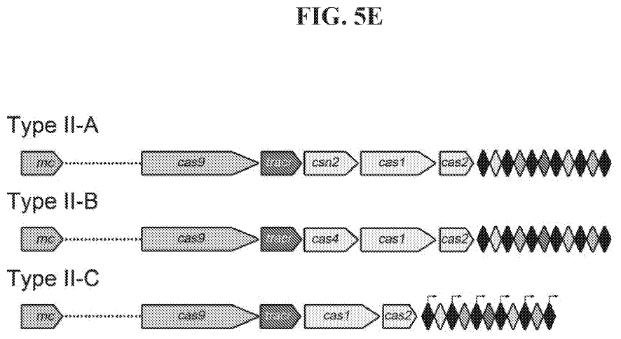

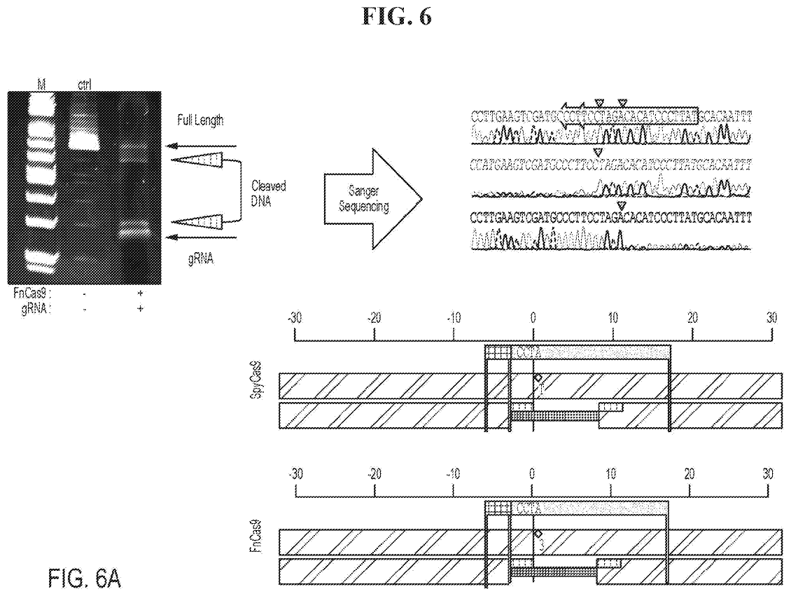

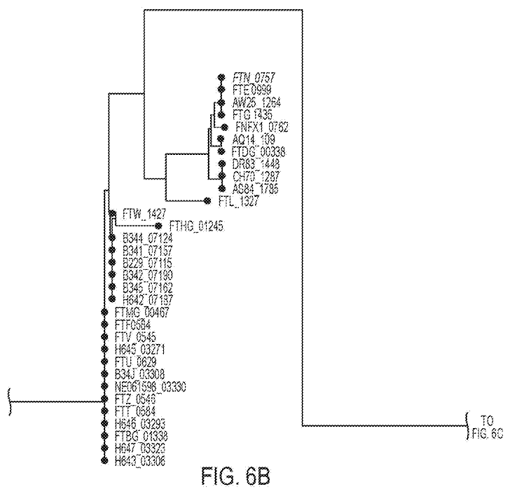

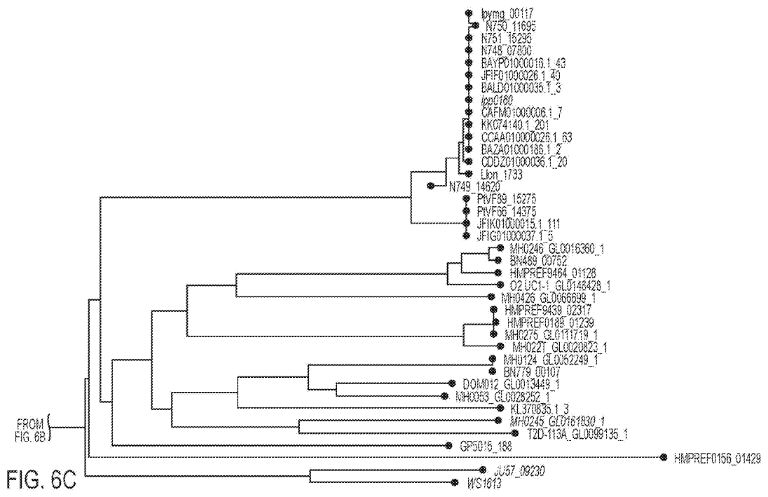

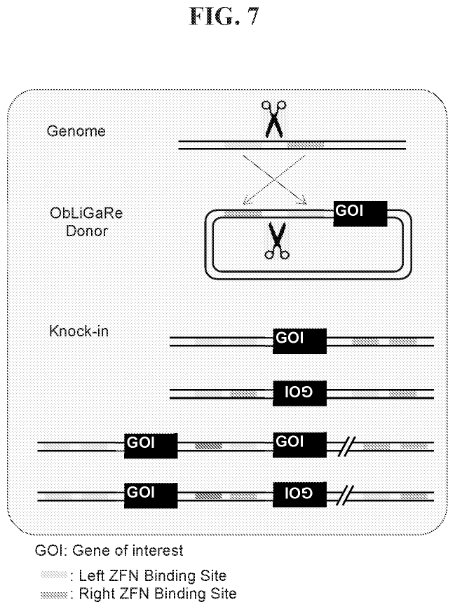

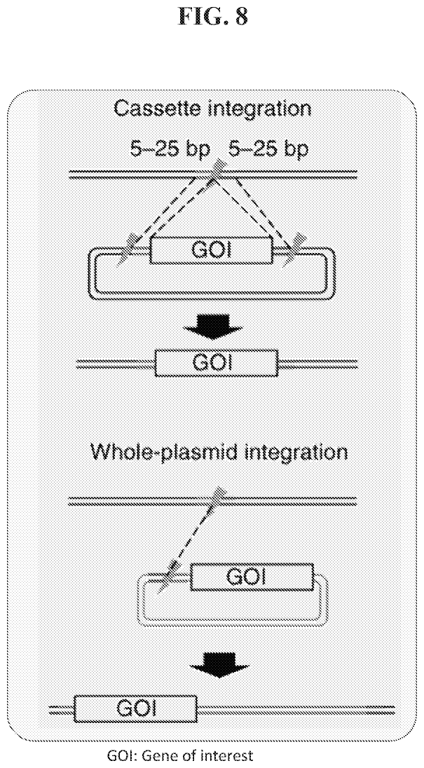

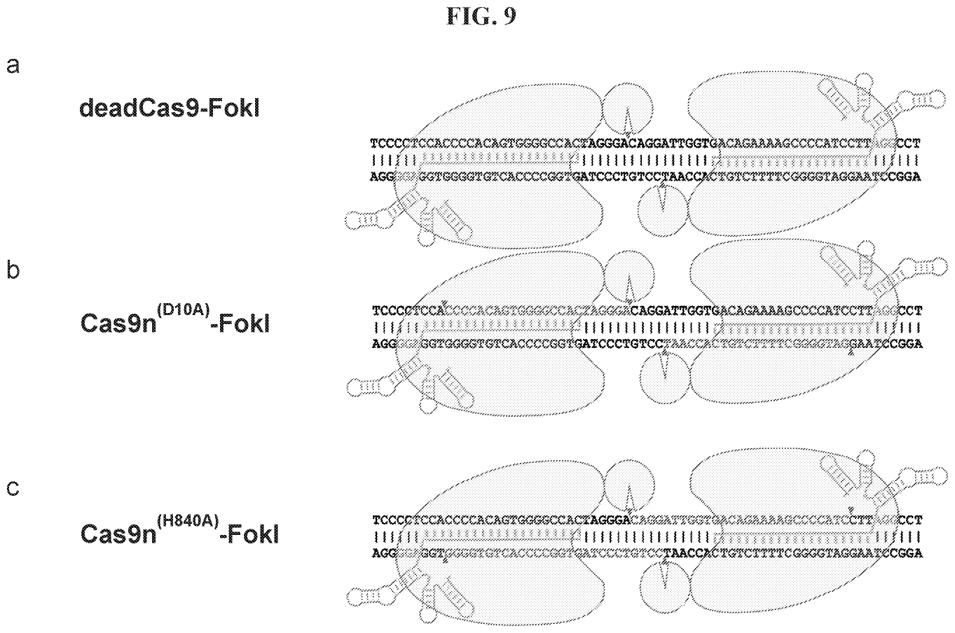

### DETAILED DESCRIPTION OF THE INVENTION

CRISPR-Cas9 systems are widely used in gene editing because of their ability to form targeted double-stranded breaks. Cas9 proteins are known to generate blunt ends upon cleavage, which provides less specificity compared with cohesive ends for inserting and/or modifying target sequences. Cas9 proteins capable of generating cohesive ends, also termed stiCas9, are described herein. Advantages of using stiCas9 proteins for inserting and/or modifying target sequences are described herein.

The present disclosure provides non-naturally occurring CRISPR-Cas systems; eukaryotic cells comprising CRISPR-Cas systems; methods for providing site-specific modification of a target sequence; methods of introducing a sequence of interest into a chromosome in a cell; and methods of modifying one or more nucleotides in a target polynucleotide sequence in a cell.

### Definitions

As used herein, “a” or “an” may mean one or more. As used herein in the specification and claims, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the present disclosure. Furthermore, compositions, systems, host cells, and/or vectors of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

A “nucleic acid,” “nucleic acid molecule,” “nucleotide,” “nucleotide sequence,” “oligonucleotide,” or “polynucleotide” means a polymeric compound comprising covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a polynucleotide encoding any one of the polypeptides disclosed herein, e.g., is directed to a polynucleotide encoding a Cas protein or a variant thereof.

A “gene” refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence.

A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SCC. High stringency hybridization conditions correspond to the highest Tm, e.g., 50% formamide, 5× or 6×SCC. Hybridization requires that the two nucleic acids contain complementary sequences, although depending on the stringency of the hybridization, mismatches between bases are possible.

The term “complementary” is used to describe the relationship between nucleotide bases that are capable of hybridizing to one another. For example, with respect to DNA, adenosine is complementary to thymine and cytosine is complementary to guanine. Accordingly, the present disclosure also includes isolated nucleic acid fragments that are complementary to the complete sequences as disclosed or used herein as well as those substantially similar nucleic acid sequences.

A DNA “coding sequence” is a double-stranded DNA sequence that is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon such as an ATG or AUG, and a termination codon and can be potentially translated into a polypeptide sequence.

The term “homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Methods known in the art may be used to propagate a polynucleotide according to the disclosure herein. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

As used herein, “promoter,” “promoter sequence,” or “promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments of the present disclosure the vector is an episomal vector, which is removed/lost from a population of cells after a number of cellular generations, e.g., by asymmetric partitioning. The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, adeno-associated virus, pox, baculovirus, vaccinia, herpes simplex, Epstein-Barr, adenovirus, geminivirus, and caulimovirus vectors. Non-viral vectors include, but are not limited to, plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

Vectors may be introduced into the desired host cells by well-known methods, including, but not limited to, transfection, transduction, cell fusion, and lipofection. Vectors can comprise various regulatory elements including promoters. In some embodiments, vector designs can be based on constructs designed by Mali et al., “Cas9 as a versatile tool for engineering biology,” Nature Methods 10: 957-63 (2013). In some embodiments, the present disclosure provides an expression vector comprising any of the polynucleotides described herein, e.g., an expression vector comprising polynucleotides encoding a Cas protein or variant thereof. In some embodiments, the present disclosure provides an expression vector comprising polynucleotides encoding a Cas9 protein or variant thereof.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

“Transfection” as used herein means the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transformed,” or “transgenic” organisms. In some embodiments, the present disclosure provides a host cell comprising any of the expression vectors described herein, e.g., an expression vector comprising a polynucleotide encoding a Cas protein or variant thereof. In some embodiments, the present disclosure provides a host cell comprising an expression vector comprising a polynucleotide encoding a Cas9 protein or variant thereof.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

An “amino acid” as used herein refers to a compound containing both a carboxyl (—COOH) and amino (—NH₂) group. “Amino acid” refers to both natural and unnatural, i.e., synthetic, amino acids. Natural amino acids, with their three-letter and single-letter abbreviations, include: Alanine (Ala; A); Arginine (Arg, R); Asparagine (Asn; N); Aspartic acid (Asp; D); Cysteine (Cys; C); Glutamine (Gln; Q); Glutamic acid (Glu; E); Glycine (Gly; G); Histidine (His; H); Isoleucine (Ile; I); Leucine (Leu; L); Lysine (Lys; K); Methionine (Met; M); Phenylalanine (Phe; F); Proline (Pro; P); Serine (Ser; S); Threonine (Thr; T); Tryptophan (Trp; W); Tyrosine (Tyr; Y); and Valine (Val; V).

An “amino acid substitution” refers to a polypeptide or protein comprising one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. Substitution mutants may be described using an abbreviated system. For example, a substitution mutation in which the fifth (5^{th}) amino acid residue is substituted may be abbreviated as “X5Y” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also to be understood that “isolated” polypeptides, proteins, peptides, or nucleic acids may be formulated with excipients such as diluents or adjuvants and still be considered isolated.

The term “recombinant” when used in reference to a nucleic acid molecule, peptide, polypeptide, or protein means of, or resulting from, a new combination of genetic material that is not known to exist in nature. A recombinant molecule can be produced by any of the well-known techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function. In some embodiments, a Cas9 domain matches a TIGR03031 protein family with an E-value cut-off of 1E-5. In some embodiments, a Cas9 domain matches a TIGR03031 protein family with an E-value cut-off of 1E-10. In some embodiments, a Cas9 domain is a RuvC domain. In some embodiments, a Cas9 domain is an HNH domain.

As used herein, the terms “sequence similarity” or “% similarity” refers to the degree of identity or correspondence between nucleic acid sequences or amino acid sequences. As used herein, “sequence similarity” refers to nucleic acid sequences wherein changes in one or more nucleotide bases results in substitution of one or more amino acids, but do not affect the functional properties of the protein encoded by the DNA sequence. “Sequence similarity” also refers to modifications of the nucleic acid, such as deletion or insertion of one or more nucleotide bases that do not substantially affect the functional properties of the resulting transcript. It is therefore understood that the present disclosure encompasses more than the specific exemplary sequences. Each of the proposed modifications is well within the routine skill in the art, as is determination of retention of biological activity of the encoded products.

Moreover, the skilled artisan recognizes that similar sequences encompassed by this disclosure are also defined by their ability to hybridize, under stringent conditions, with the sequences exemplified herein. Similar nucleic acid sequences of the present disclosure are those nucleic acids whose DNA sequences are at least 70%, at least 80%, at least 90%, at least 95%, or at least 99% identical to the DNA sequence of the nucleic acids disclosed herein. Similar nucleic acid sequences of the present disclosure are those nucleic acids whose DNA sequences are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the DNA sequence of the nucleic acids disclosed herein.

As used herein, “sequence similarity” refers to two or more amino acid sequences wherein greater than about 40% of the amino acids are identical, or greater than about 60% of the amino acids are functionally identical. Functionally identical or functionally similar amino acids have chemically similar side chains. For example, amino acids can be grouped in the following manner according to functional similarity:
Positively-charged side chains: Arg, His, Lys;
Negatively-charged side chains: Asp, Glu;
Polar, uncharged side chains: Ser, Thr, Asn, Gln;
Hydrophobic side chains: Ala, Val, Ile, Leu, Met, Phe, Tyr, Trp;
Other: Cys, Gly, Pro.

In some embodiments, similar amino acid sequences of the present disclosure have at least 40%, at least 50%, at least 60%, at least 70%, at least 80%, at least 90%, or at least 99% identical amino acids.

In some embodiments, similar amino acid sequences of the present disclosure have at least 60%, at least 70%, at least 80%, at least 90%, or at least 95% functionally identical amino acids. In some embodiments, similar amino acid sequences of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids.

In some embodiments, similar amino acid sequences of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

Sequence similarity is determined by sequence alignment using routine methods in the art, such as, for example, BLAST, MUSCLE, Clustal (including ClustalW and ClustalX), and T-Coffee (including variants such as, for example, M-Coffee, R-Coffee, and Expresso).

The terms “sequence identity” or “% identity” in the context of nucleic acid sequences or amino acid sequences refers to the percentage of residues in the compared sequences that are the same when the sequences are aligned over a specified comparison window. In some embodiments, only specific portions of two or more sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. “Percent identity” or “% identity” when referring to amino acid sequences can be determined by methods known in the art. For example, in some embodiments, “percent identity” of two amino acid sequences is determined using the algorithm of Karlin and Altschul, Proceedings of the National Academy of Sciences USA 87: 2264-2268 (1990), modified as in Karlin and Altschul, Proceedings of the National Academy of Sciences USA 90: 5873-5877 (1993). Such an algorithm is incorporated into the BLAST programs, e.g., BLAST+ or the NBLAST and XBLAST programs described in Altschul et al., Journal of Molecular Biology, 215: 403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Research 25(17): 3389-3402 (1997). When utilizing BLAST and Gapped BLAST programs, the default parameters of the respective programs (e.g., XBLAST and NBLAST) can be used.

In some embodiments, polypeptides or nucleic acid molecules have 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or nucleic acid molecule, respectively (or a fragment of the reference polypeptide or nucleic acid molecule). In some embodiments, polypeptides or nucleic acid molecules have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or nucleic acid molecule, respectively (or a fragment of the reference polypeptide or nucleic acid molecule).

### CRISPR-Cas Systems

In some embodiments, the disclosure provides a non-naturally occurring CRISPR-Cas system comprising: (a) a Cas9 effector protein capable of generating cohesive ends (“sticky-end Cas9” or “stiCas9”); and (b) a guide polynucleotide that forms a complex with the stiCas9 and comprises a guide sequence, wherein the guide sequence hybridizes with a target sequence in a eukaryotic cell but does not hybridize to a sequence in a bacterial cell; wherein the complex does not occur in nature.

In general, a CRISPR or CRISPR-Cas system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide polynucleotide is designed to target, e.g. have complementarity, where hybridization between a target sequence and a guide polynucleotide promotes the formation of a CRISPR complex. The section of the guide polynucleotide through which complementarity to the target sequence can be important for cleavage activity is referred to herein as the guide sequence. A target sequence may comprise any polynucleotide, such as DNA or RNA polynucleotides and can be located within a target locus of interest. In some embodiments, a target sequence is located in the nucleus or cytoplasm of a cell. In some embodiments, the target sequence is located on the chromosome (TSC). In some embodiments, the target sequence is located on a vector (TSV).

As described herein, Cas proteins are components of the CRISPR-Cas system, which can be used for, inter alia, genome editing, gene regulation, genetic circuit construction, and functional genomics. While the Cas1 and Cas2 proteins appear to be universal to all the presently identified CRISPR systems, the Cas3, Cas9, and Cas10 proteins are thought to be specific to the Type I, Type II, and Type III CRISPR systems, respectively.

Following initial publications around the CRISPR-Cas9 system (Type II system), Cas9 variants have been identified in a range of bacterial species and a number have been functionally characterized. See, e.g., Chylinski et al., “Classification and evolution of type II CRISPR-Cas systems”, Nucleic Acids Research 42(10): 6091-6105 (2014), Ran et al., “In vivo genome editing using Staphylococcus aureus Cas9”, Nature 520(7546): 186-91 (2015), and Esvelt et al., “Orthogonal Cas9 proteins for RNA-guided gene regulation and editing”, Nature Methods 10(11): 1116-1121 (2013), each of which is incorporated by reference herein in its entirety.

The present disclosure encompasses novel effector proteins of Type II CRISPR-Cas systems, of which Cas9 is an exemplary effector protein. Hence, the terms “Cas9,” “Cas 9 protein” and “Cas9 effector protein” are interchangeable and are used herein to describe effector proteins which are capable of providing cohesive ends when used in the CRISPR-Cas9 system. In some embodiments, the term Cas9 refers to a Type II-B Cas9. In some embodiments, the term Cas9 refers to engineered Cas9 variants, such as, e.g., deadCas9-FokI, Cas9n^{D10A}-FokI, and Cas9n^{H840A}-FokI.

In some embodiments, the Cas9 effector protein is functional in prokaryotic or eukaryotic cells for in vitro, in vivo, or ex vivo applications.

The term Cas9 effector protein can refer to effector proteins having Cas9-like function, generally having both RuvC and HNH nuclease domains. In some embodiments, the RuvC domain and HNH domain of a Cas9 effector protein each cleave one strand of a double-stranded target DNA. Thus, for example, if the RuvC domain and the HNH domain cleaves each strand at the same position, the result of the cleavage will be a double-stranded target DNA with blunt ends. If the RuvC domain and the HNH domain cleaves each strand at different positions (i.e., cut at an “offset”), the result of the cleavage will be a double-stranded target DNA with overhangs. In embodiments, the RuvC and HNH domains of the stiCas9 protein cut at a 3-nucleotide offset. In embodiments, the RuvC and HNH domains of the stiCas9 protein cut at a 4-nucleotide offset. In embodiments, the RuvC and HNH domains of the stiCas9 protein cut at a 5-nucleotide offset. In embodiments, the RuvC and HNH domains of the stiCas9 protein cut at an offset of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides.

In some embodiments, the term Cas9 effector protein refers to a Cas9 with a RuvC domain and an HNH domain, wherein the RuvC domain and the HNH domain cleaves at different positions on each strand of the double-stranded target DNA. In some embodiments, the RuvC domain of the Cas9 effector protein cleaves one strand of the double-stranded target DNA (which can be referred to, for example, as the “non-target strand”) at from about −10, about −9, about −8, about −7, or about −6 nucleotides from the PAM, and the HNH domain of the Cas9 effector protein cleaves the other strand of the double-stranded target DNA (which can be referred to, for example, as the “target strand”) at −5, about −4, about −3, about −2, or about −1 nucleotides from the PAM.

In some embodiments, the RuvC domain cleaves one strand of the double-stranded target DNA at about −8 nucleotides from the PAM. In some embodiments, the RuvC domain cleaves one strand of the double-stranded target DNA at about −7 nucleotides from the PAM. In some embodiments, the RuvC domain cleaves one strand of the double-stranded target DNA at about −6 nucleotides from the PAM. In some embodiments, the HNH domain cleaves one strand of the double-stranded target DNA at about −4 nucleotides from the PAM. In some embodiments, the HNH domain cleaves one strand of the double-stranded target DNA at about −3 nucleotides from the PAM. In some embodiments, the HNH domain cleaves one strand of the double-stranded target DNA at about −2 nucleotides from the PAM.

In some embodiments, the term Cas9 effector protein refers to a Cas9 with the TIGR03031 protein family as identified by a HMMER search, specifically, the program hmmscan (HMMER version 3.1b2). The present disclosure also relates to the identification and engineering of effector proteins associated with Type II CRISPR-Cas systems. In some embodiments, the effector protein comprises a single-subunit effector module. In some embodiments, the wild-type Cas9 effector or an engineered version of Cas9 protein is fused to one or multiple functional domains, such as, e.g., Nuclear Localization Signals (NLS) and FokI nuclease. The present disclosure encompasses computational methods and algorithms to predict new Type II-B CRISPR-Cas systems and identify the components therein.

In some embodiments, a computational method of identifying novel Type II-B CRISPR-Cas loci comprises methods described below and previously described in Shmakov et al., Nature Reviews Microbiology 15, 169-182 (2017). The presence and location of a CRISPR-Cas locus in a given nucleotide sequence can be identified by using the protein sequence of one of the known Cas proteins as seeds, e.g. Cas1, in a TBLASTN against nucleotide sequences using, for example, an E-value cutoff of 0.01. Another approach to identify the presence and location of CRISPR-Cas locus is to search CRISPR arrays in the nucleotide sequence by use of programs such as, e.g., CRISPRfinder or PILER-CR with default parameters. Once a CRISPR-Cas locus is identified, sequences including up to 10 kbp upstream and downstream of the CRISPR-Cas locus can be extracted. The presence of genes in the extracted nucleotide sequences can be performed with software such as GeneMark or MetaGeneMark using default parameters. Identified genes are then translated into protein sequences and annotated to indicate their predicted function using homology searches such as RPS-BLAST, BLAST, or HMMR to databases of proteins with known functions (i.e., Cas1, Cas2, Cas4, Cas9, etc.).

CRISPR-Cas loci identified with the methodology above were investigated for the presence of both Cas9 and Cas4 proteins in the same CRISPR-Cas loci because these are highly likely to contain Cas9 of Type IIB. To further increase the probability of a Type-IIB Cas9, the Cas9 proteins were searched with hmmscan for belonging to the TIGRFAM: TIGR03031 family.

In some embodiments, a method of identifying novel Type II-B CRISPR-Cas loci comprises identifying Cas9 proteins in the same loci as a Cas4 protein. In some embodiments, a method of identifying novel Type II-B CRISPR-Cas loci comprises translation of publicly available metagenomic gene catalogs into amino acid sequences, scanning each amino acid sequence with the TIGR03031 protein family profile to identify matches above a pre-defined cut-off E-value such as, e.g., 1E-5 to 1E-10.

TIGRFAMs are a collection of protein families featuring curated multiple sequence alignments, Hidden Markov Models, and associated information designed to support the automated functional identification of proteins by sequence homology. Hidden Markov Models (HMMs) as applied to sequence alignments refer to a statistical model for successive columns of protein multiple sequence alignments. Typically, protein profile HMMs are developed from curated multiple sequence alignments with position-based scoring for each of the amino acid, insertion, and deletion over the length of the sequence. Scores are reported both in bits of information and as an E-value. An E-value below a “trusted cut-off” or “trusted limit” such as, e.g., 0.001, is recognized as a positive “hit” or a positive identification. Thus, sequences identified with a low E-value cut-off are likely to belong to a specified protein family. In some embodiments, the E-value cut-off is 1E-10. In some embodiments, the E-value cut-off is 1E-5. In some embodiments, the trusted cut-off E-value is at least 1E-10, at least 1E-9, at least 1E-8, at least 1E-7, at least 1E-6, at least 1E-5, at least 1E-4, at least 1E-3, at least 1E-2, or at least 1E-1.

In some embodiments, the identification of all predicted protein coding genes is carried out by comparing the identified genes with Cas protein-specific profiles and annotating them according to NCBI Conserved Domain Database (CDD), which is a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM). Protein databases are described in, e.g., Finn et al., Nucleic Acids Research Database Issue 44: D279-D285 (2016); Letunic et al., Nucleic Acids Research, doi: gkx922 (2017); Tatusov et al., Science 278(5338): 631-637 (1997); and Haft et al., Nucleic Acids Research Database Issue 41: D387-D395 (2013), each of which is incorporated herein in its entirety.

In some embodiments, novel Type II-B CRISPR-Cas loci are identified using HMMER (or any version of HMMER such as HMMER2 or HMMER3) to search for conserved domains. HMMER is free and commonly used software package for sequence analysis, identification of homologous protein or nucleotide sequences, and sequence alignments. HMMER implements probabilistic models called profile hidden Markov models. HMMER can be used with a profile database such as Pfam, SMART, COG, PRK, or TIGRFAM. HMMER can also be used with query sequences, for example, searching a protein query sequence against a database (i.e., phmmer) or an iterative search (i.e., jackhmmer). In some embodiments, novel Type II-B CRISPR-Cas loci are identified by searching for the presence of a specific domain in a specific protein family. In some embodiments, the TIGRFAM protein family is TIGRFAM: TIGR03031. In some embodiments, the specific domain matches the TIGR03031 protein family with an E-value cut-off of at least 1E-0 10, at least 1E-9, at least 1E-8, at least 1E-7, at least 1E-6, at least 1E-5, at least 1E-4, at least 1E-3, at least 1E-2, or at least 1E-1. In some embodiments, the specific domain has at least 60%, at least 70%, at least 80%, at least 90%, at least 95%, at least 96%, at least 97%, at least 98%, at least 99%, or about 100% sequence similarity to any of the TIGR03031 domains identified herein. In some embodiments, the specific domain has at least 60%, at least 70%, at least 80%, at least 90%, at least 95%, at least 96%, at least 97%, at least 98%, at least 99%, or about 100% sequence similarity to any one of SEQ ID NOs: 10-97 or 192-195. In some embodiments, the specific domain has at least 40%, at least 50%, at least 60%, at least 70%, at least 80%, at least 90%, at least 95%, at least 96%, at least 97%, at least 98%, at least 99%, or about 100% sequence identity to any one of SEQ ID NOs: 10-97 or 192-195.

In some embodiments, the stiCas9 is derived from a bacterial species having a Type II-B CRISPR system. In some embodiments, the Type II-B CRISPR system includes a cas4 gene. As discussed herein, CRISPR systems have been classified as Type I, Type II, and Type III. All Type II CRISPR systems include the cas1, cas2, and cas9 genes on the cas operon. Type II CRISPR systems are further categorized into Type II-A, Type II-B, and Type II-C. In some embodiments, Type II-B CRISPR systems are identified by the presence of a cas4 gene on the cas operon. A cas4 gene is not found in Type II-A or Type II-C CRISPR systems.

Type II CRISPR systems can also be classified according to the sequence of individual cas genes, for example, the sequence and/or domains of cas9. Protein domains may be identified by conserved sequences or conserved motifs and classified into families, super families, and subfamilies. For example, protein domains can be classified according to PFAMs or TIGRFAMs. Accordingly, Cas proteins can be identified and classified with protein domains. For example, Type II-A Cas9 proteins, including Cas9 from Streptococcus pyogenes, are of the TIGR01865 TIGRFAM protein family. In contrast, Type II-B Cas9 proteins are of the TIGR03031 TIGRFAM protein family.

Thus, in some embodiments, the stiCas9 of the present disclosure comprises a domain having at least 95% sequence similarity to any of SEQ ID NOs: 10-97 or 192-195. In some embodiments, the stiCas9 of the present disclosure comprises a domain having at least 10%, at least 20%, at least 30%, at least 40%, at least 50%, at least 60%, at least 70%, at least 80%, at least 90%, at least 95%, at least 96%, at least 97%, at least 98%, at least 99%, or about 100% sequence similarity to any of SEQ ID NOs: 10-97 or 192-195. In some embodiments, the stiCas9 of the present disclosure comprises a domain that matches the TIGR03031 protein family with an E-value cut-off of at least 1E-10, at least 1E-9, at least 1E-8, at least 1E-7, at least 1E-6, at least 1E-5, at least 1E-4, at least 1E-3, at least 1E-2, or at least 1E-1.

In some embodiments, the Type II-B Cas9 is derived from any species having a Type II-B CRISPR system. In some embodiments, the Type II-B Cas9 is derived from the following bacterial species: Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1_47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonassp. S-B4-1U, Tamilnaduibacter salinus, Vibrio natriegens, Arcobacter skirrowii, Francisella philomiragia, Francisella hispaniensis, or Parendozoicomonas haliclonae.

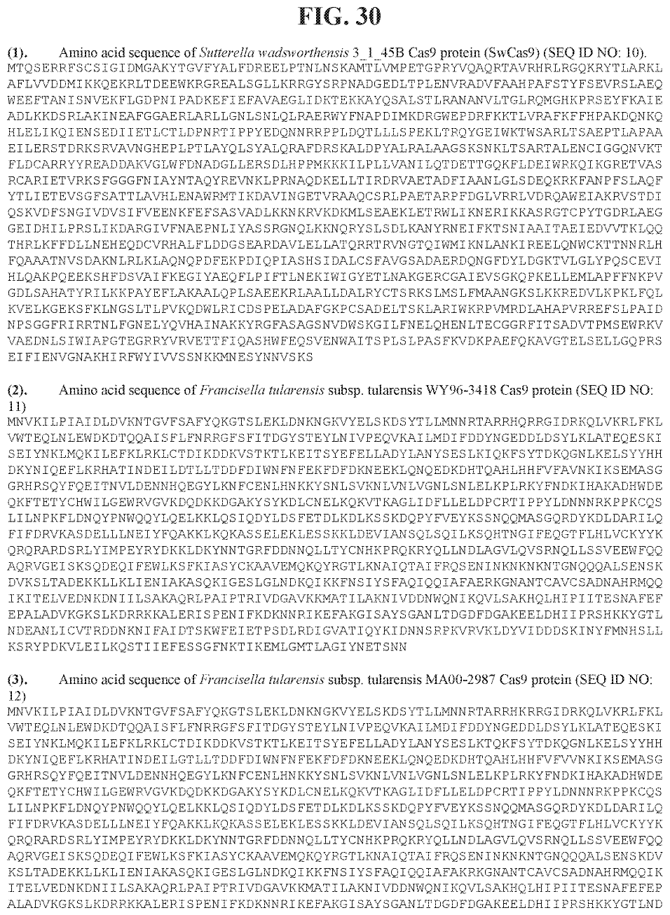

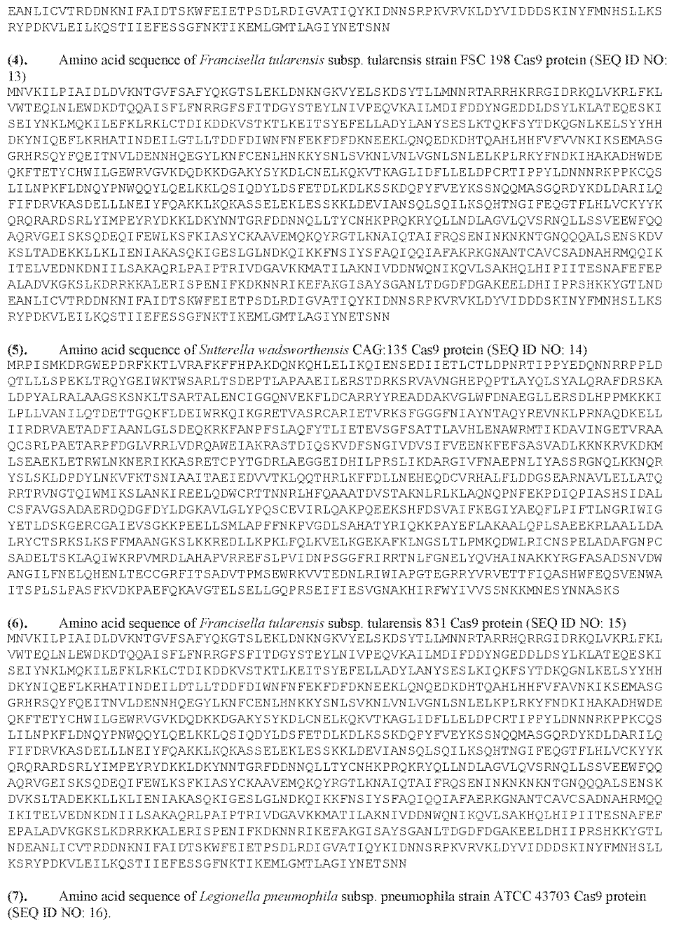

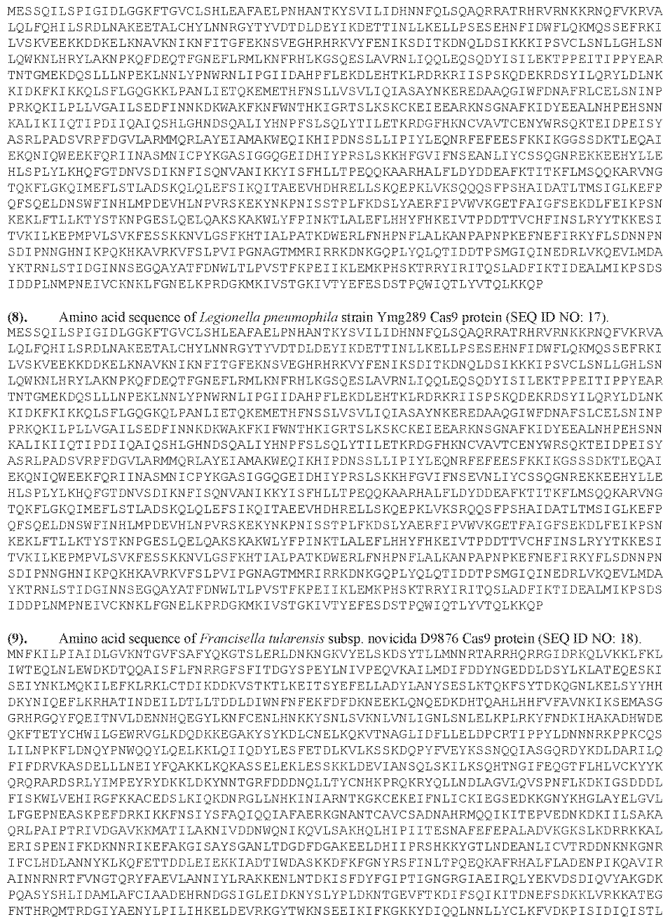

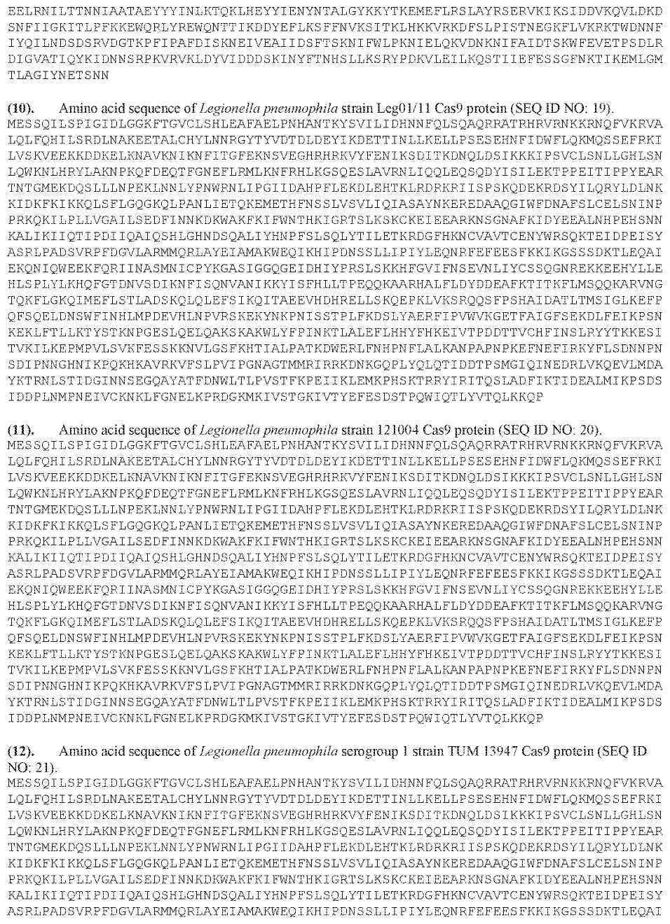

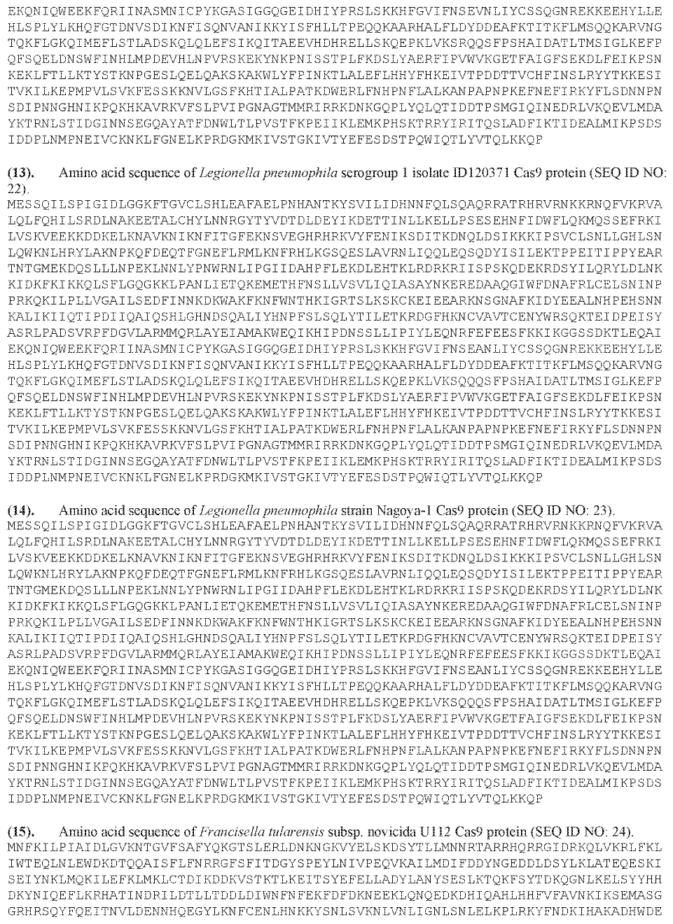

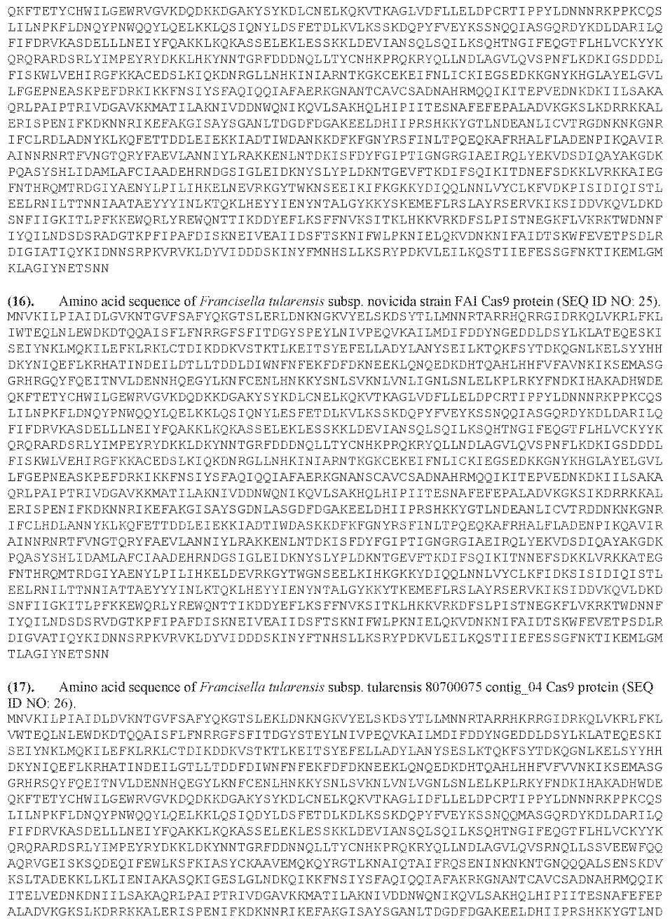

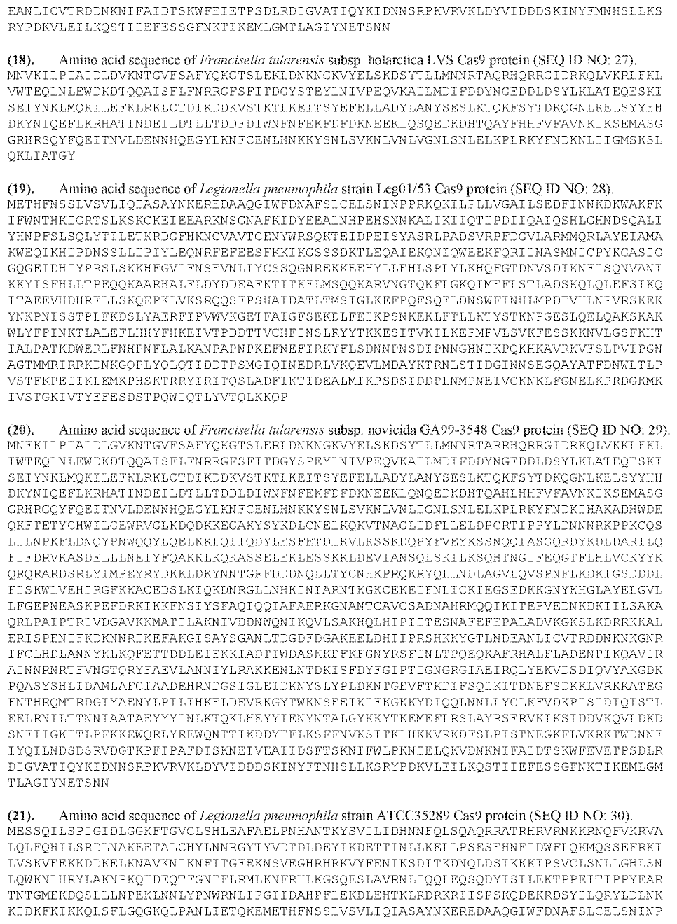

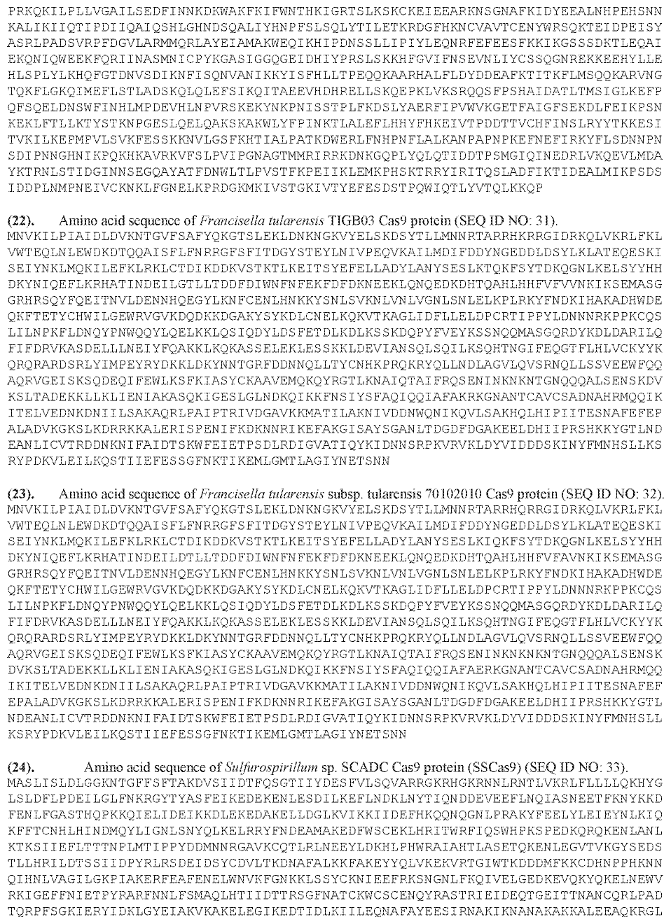

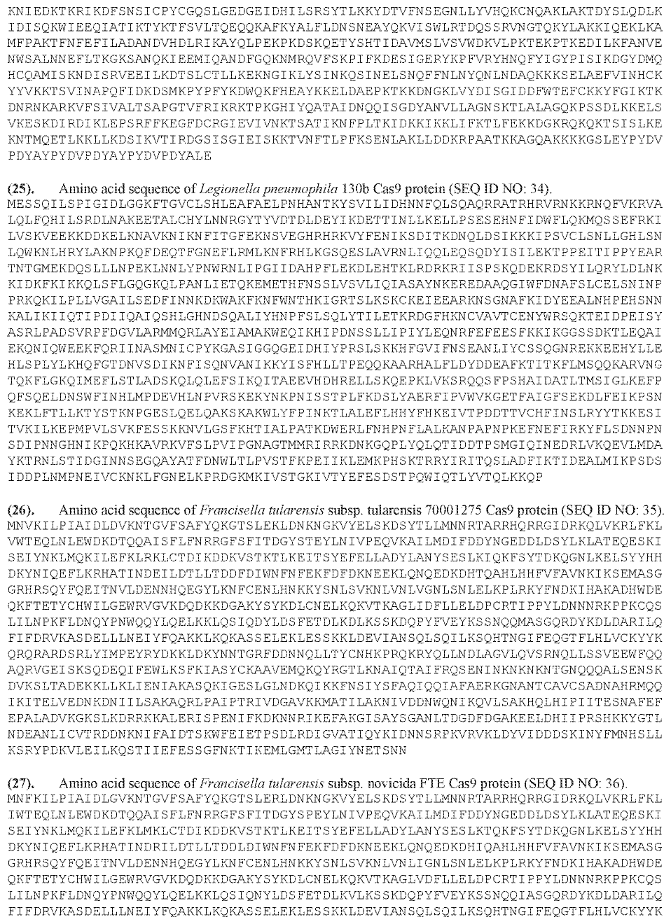

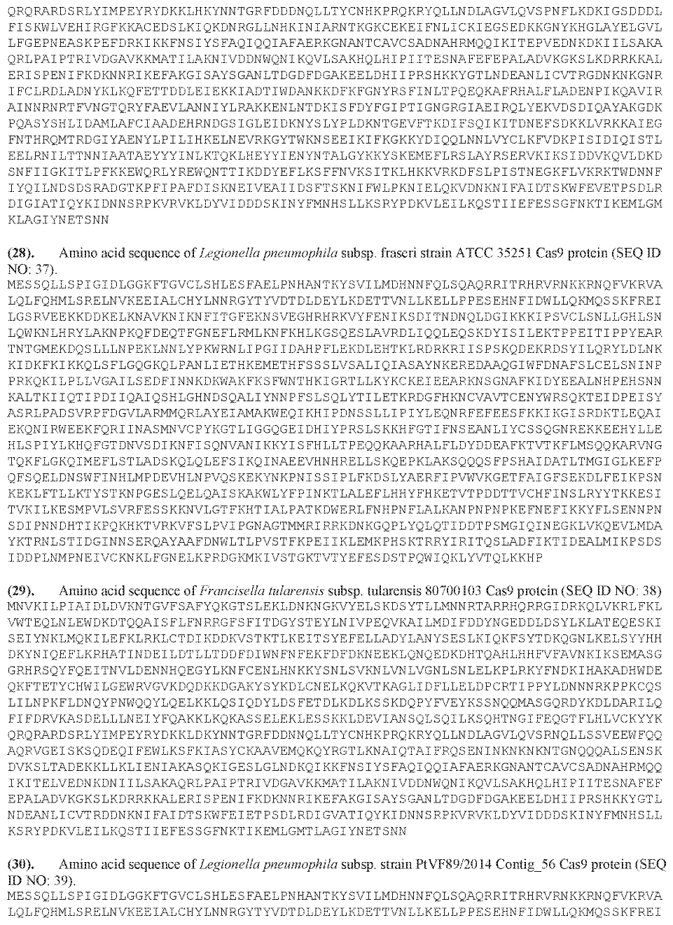

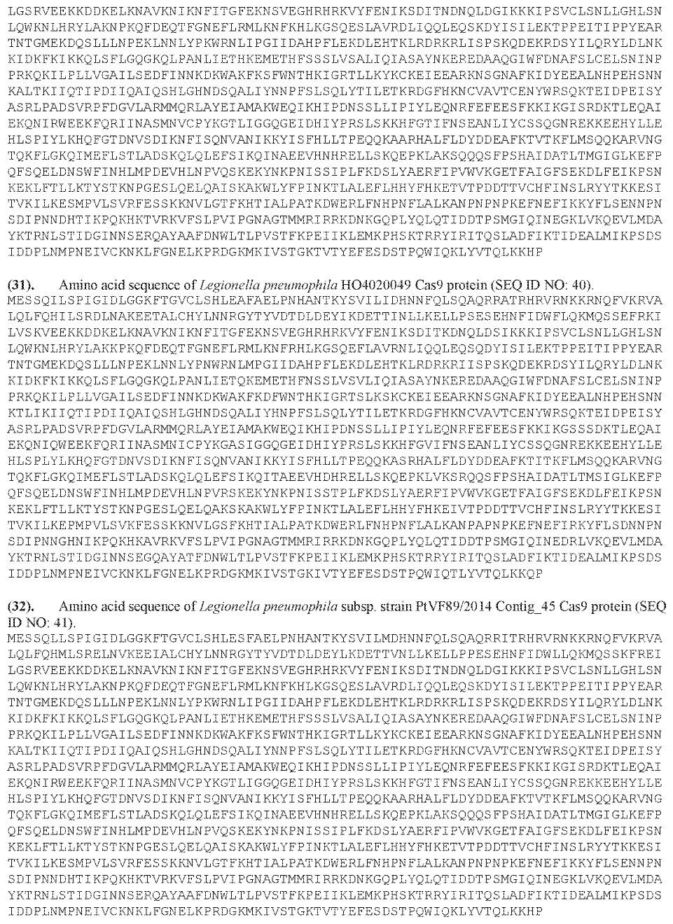

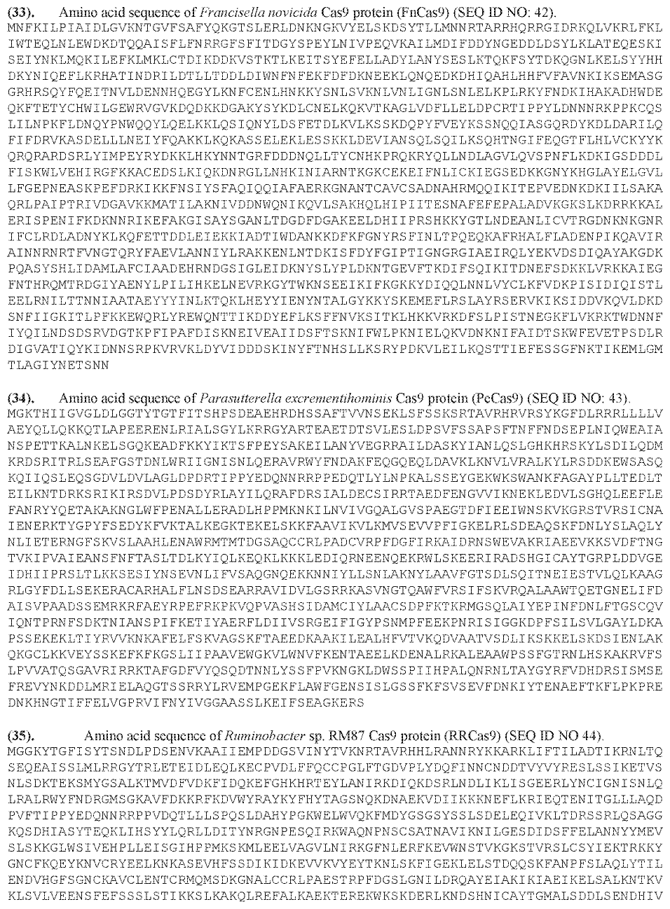

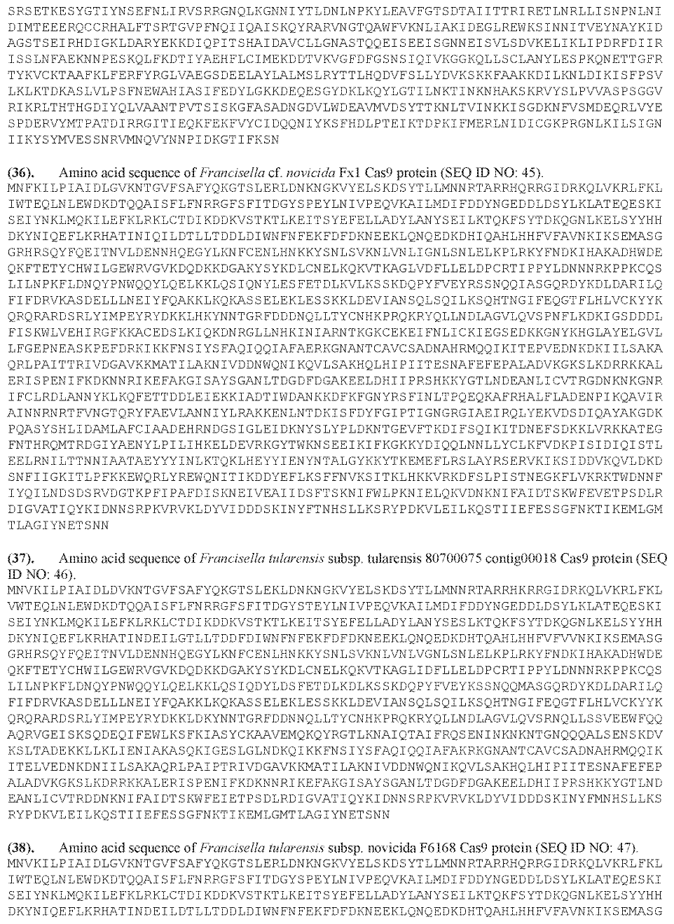

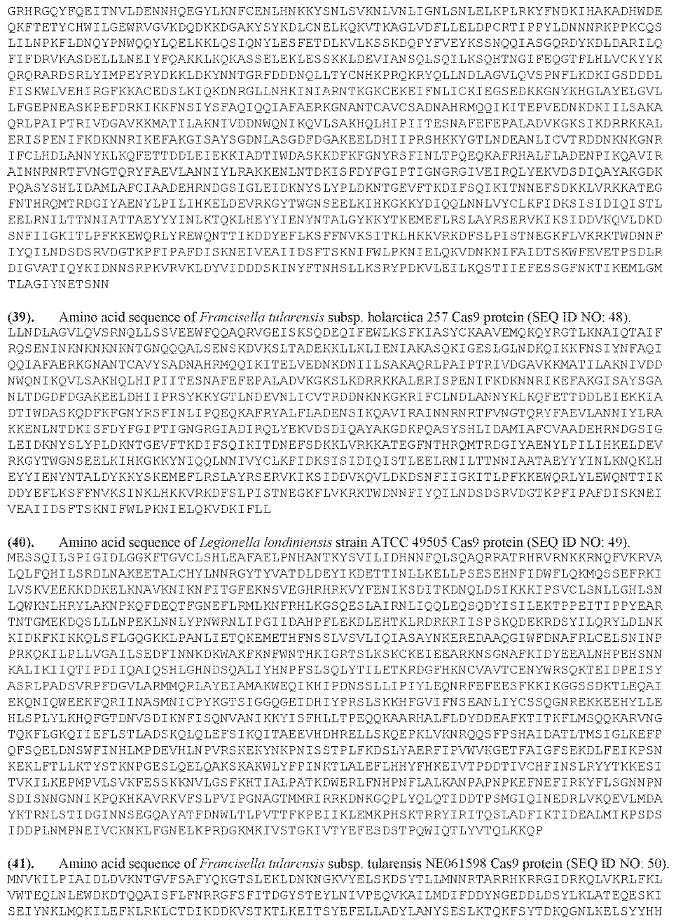

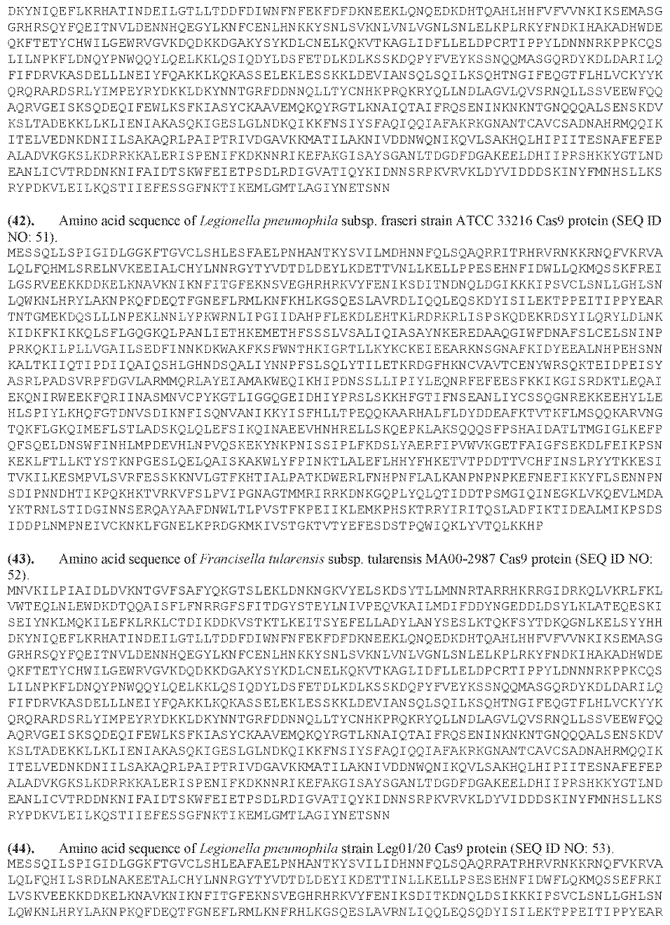

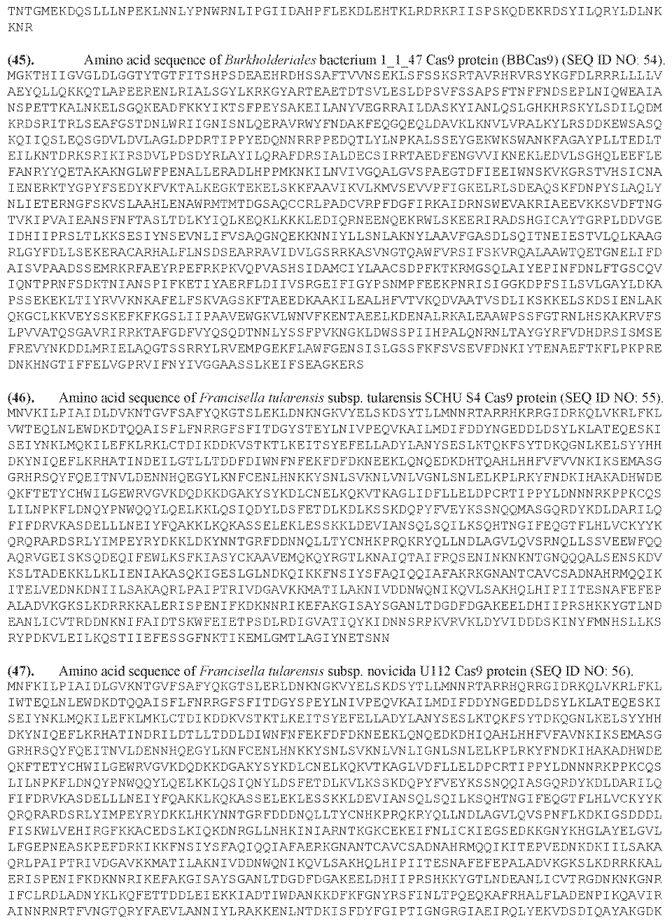

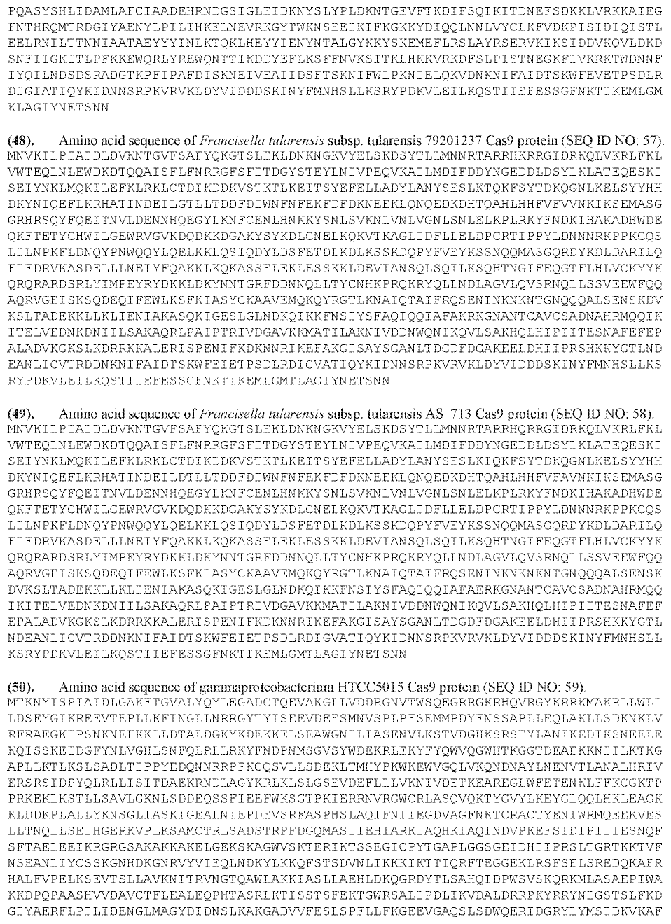

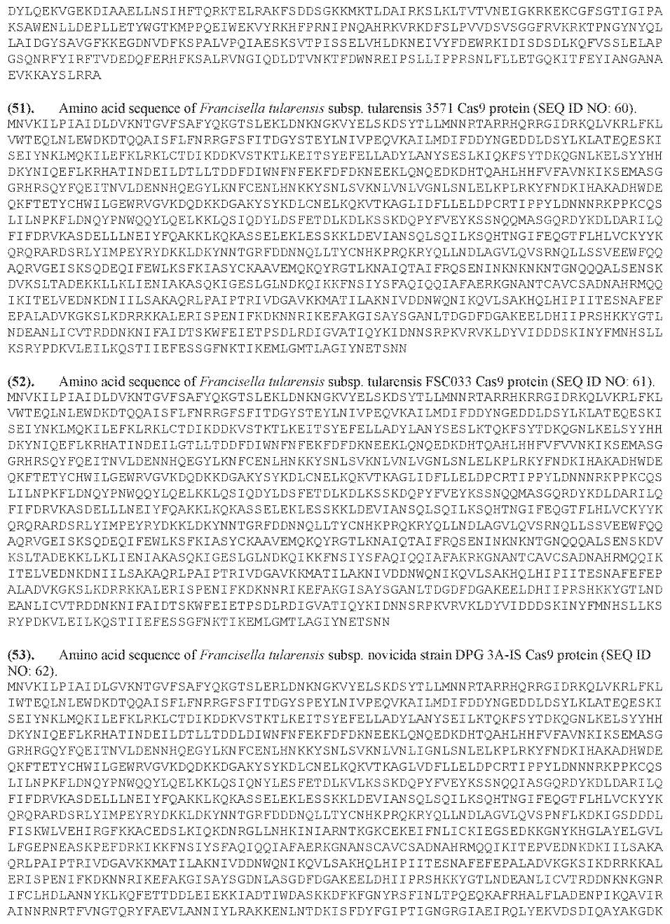

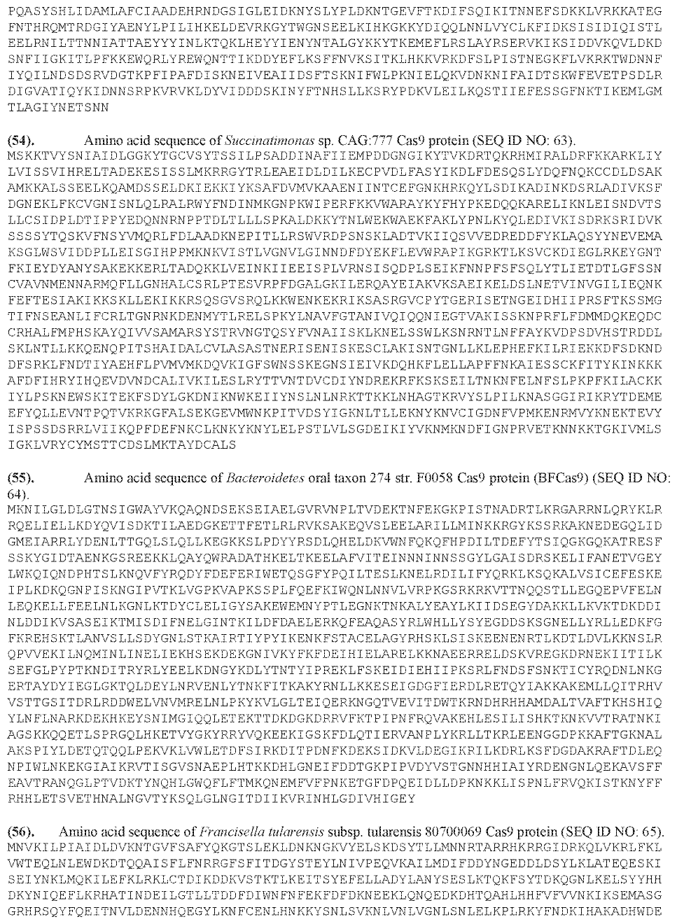

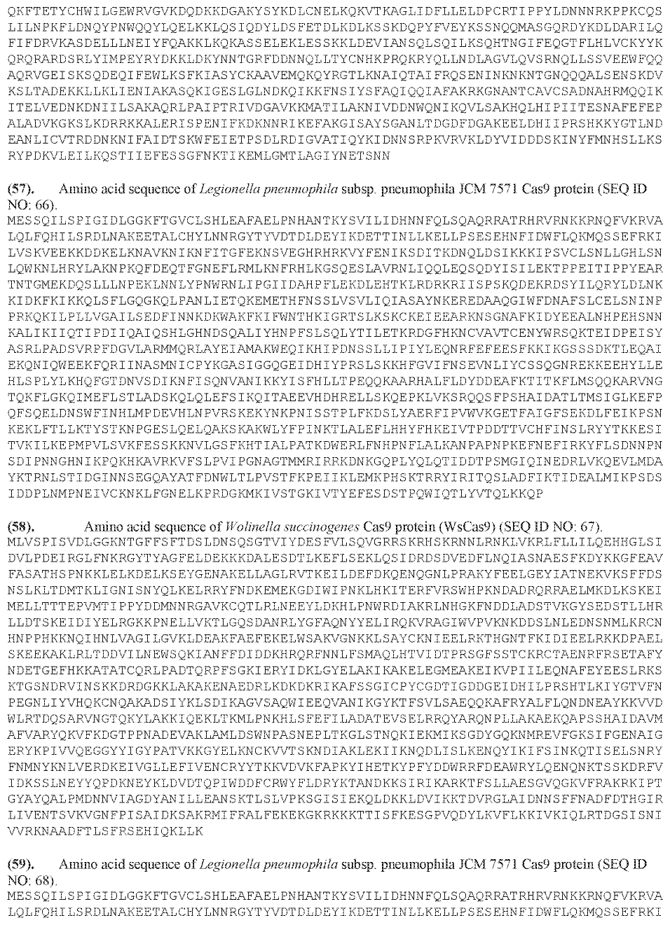

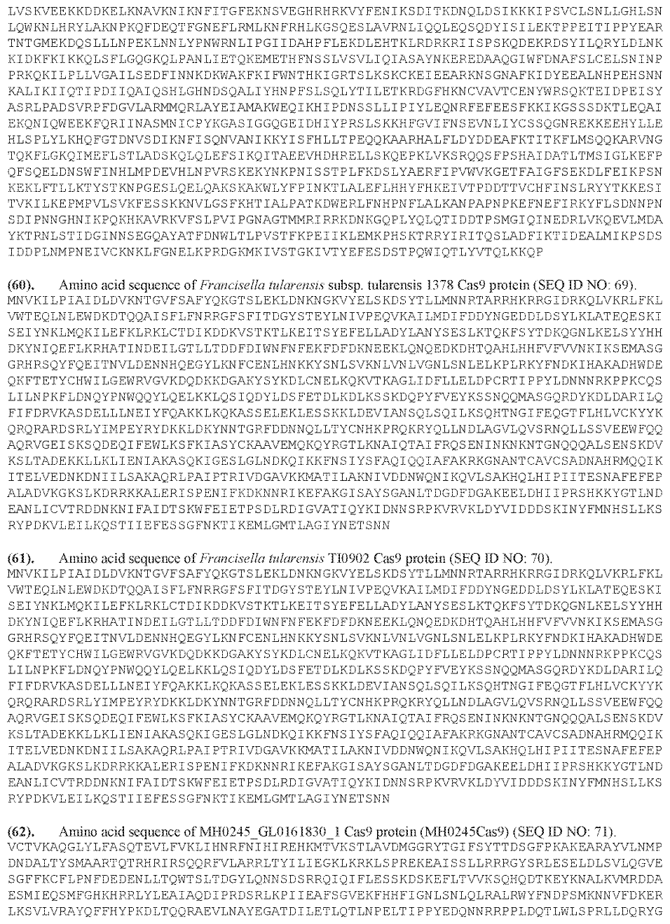

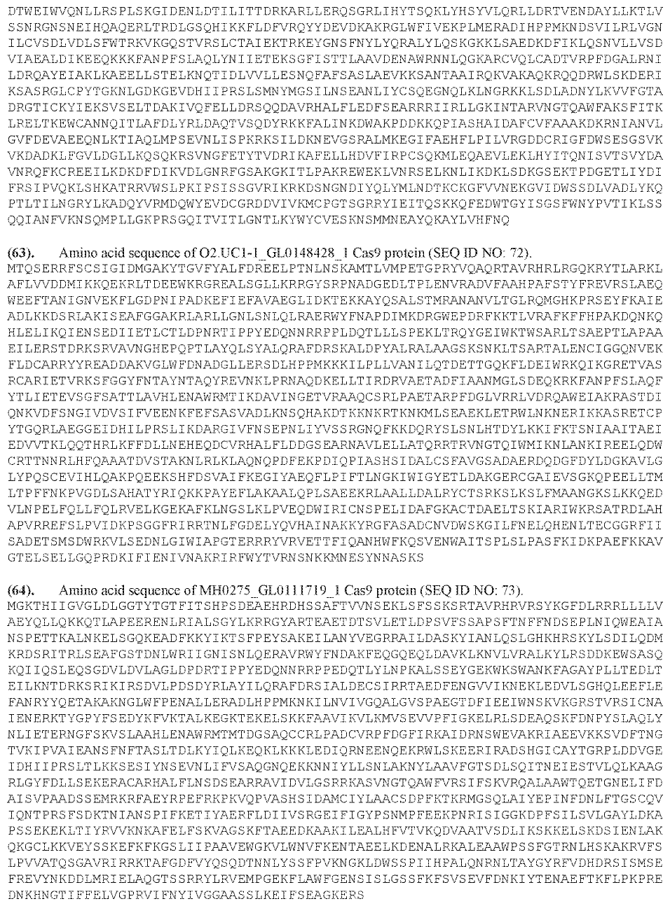

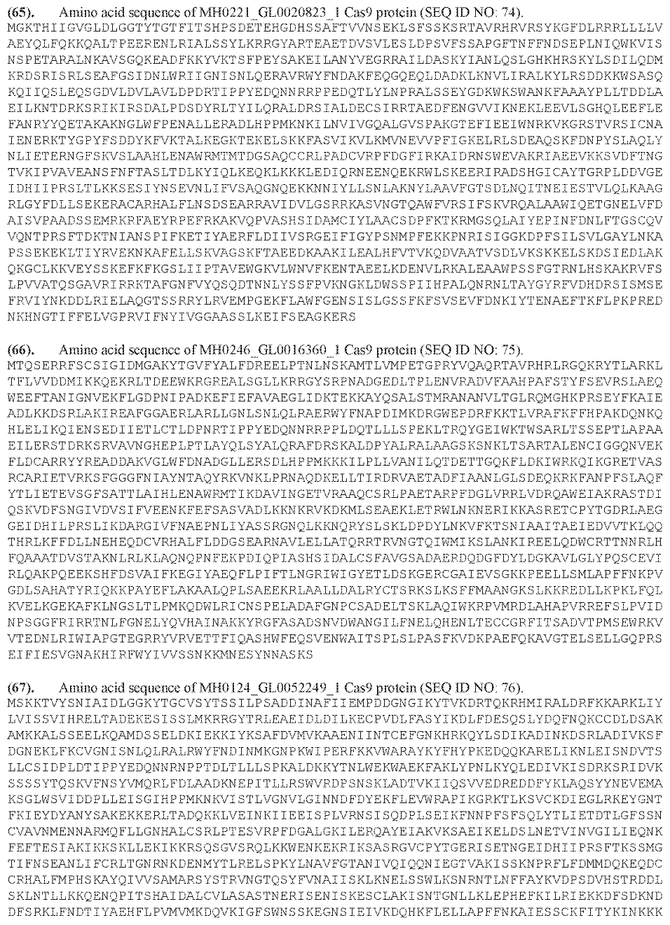

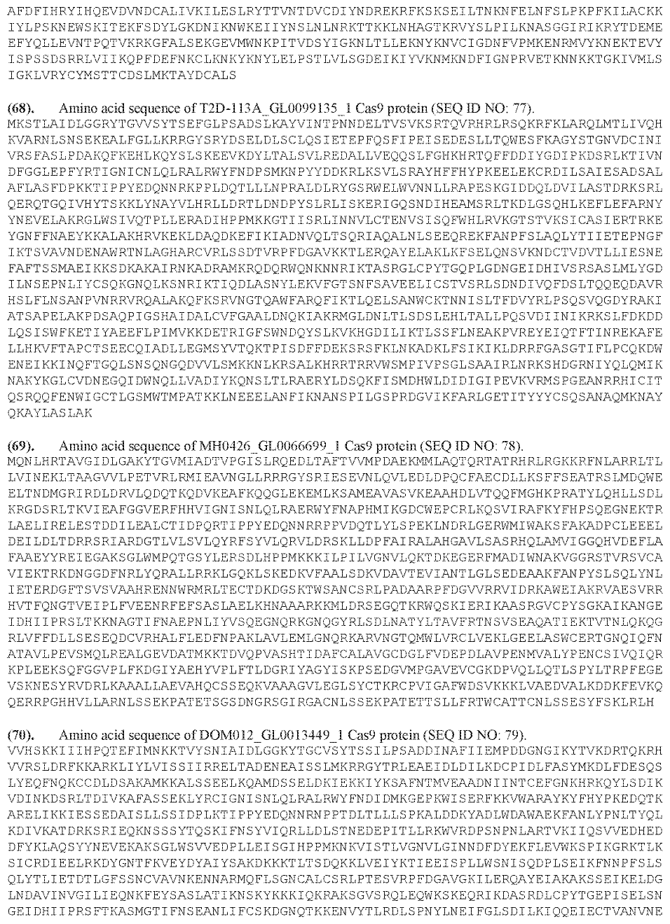

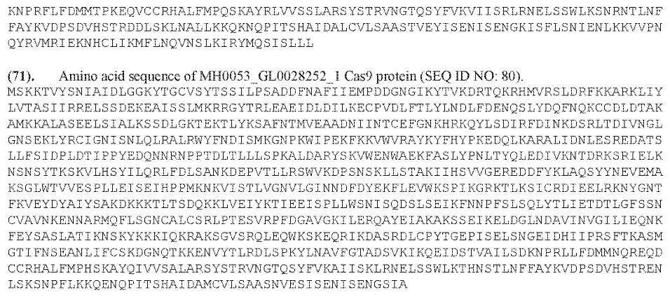

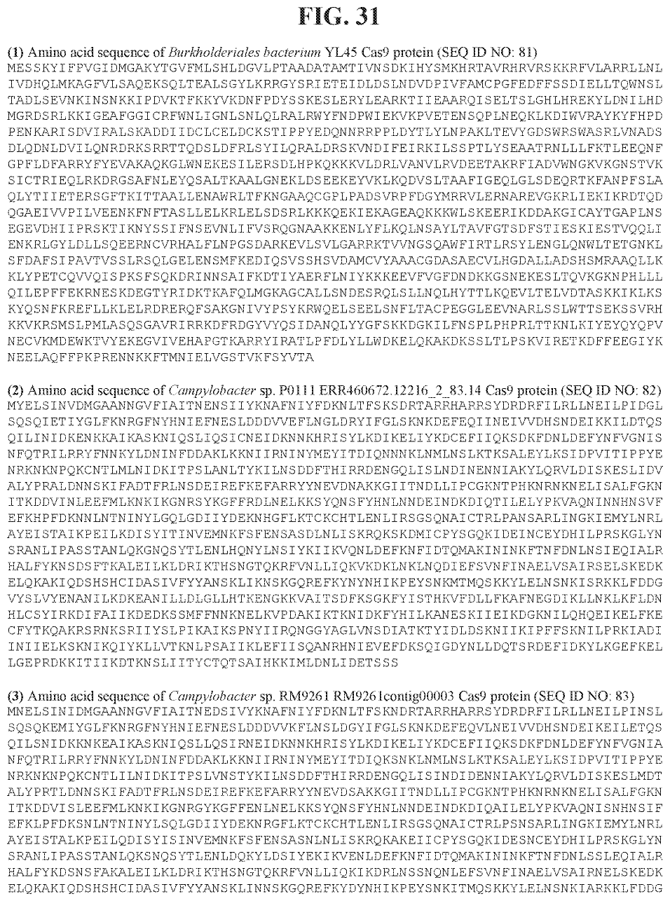

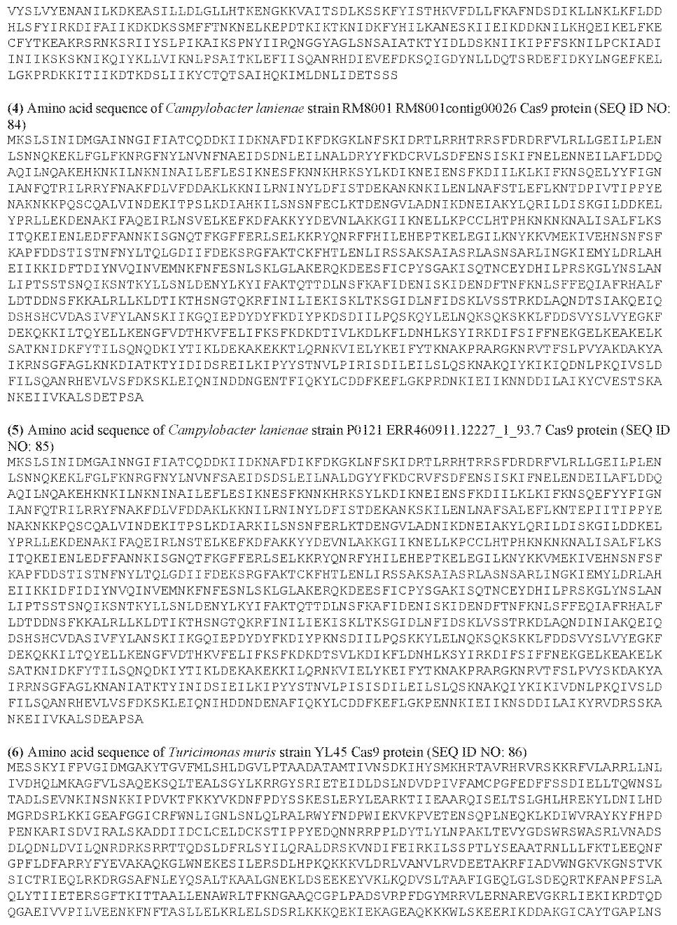

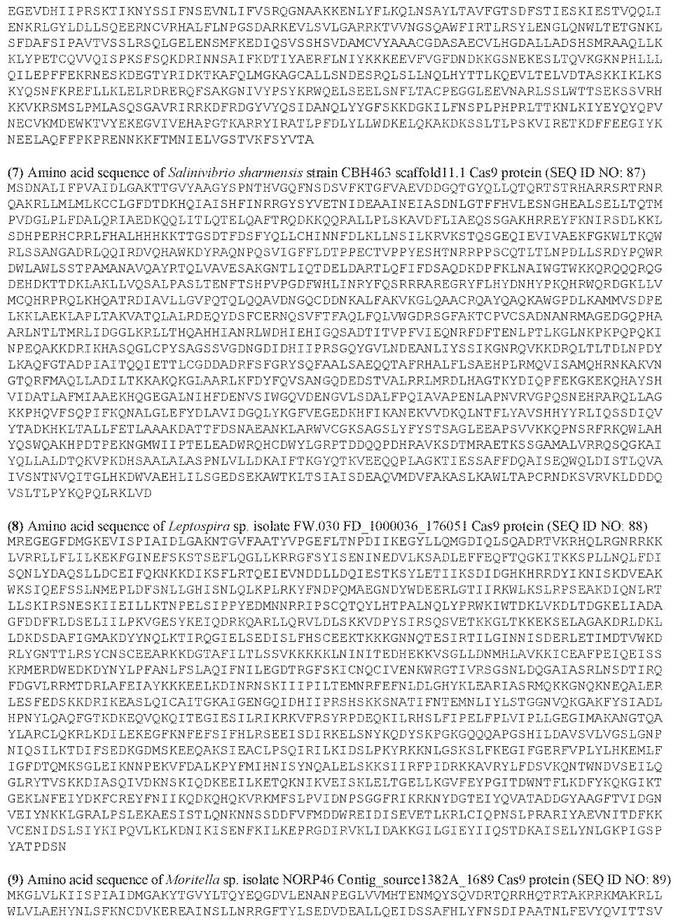

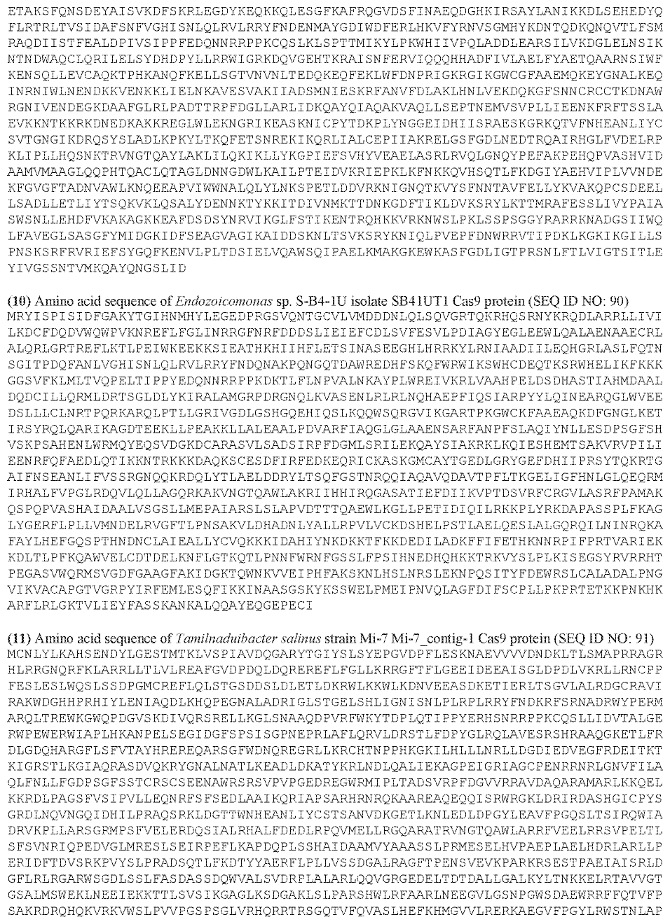

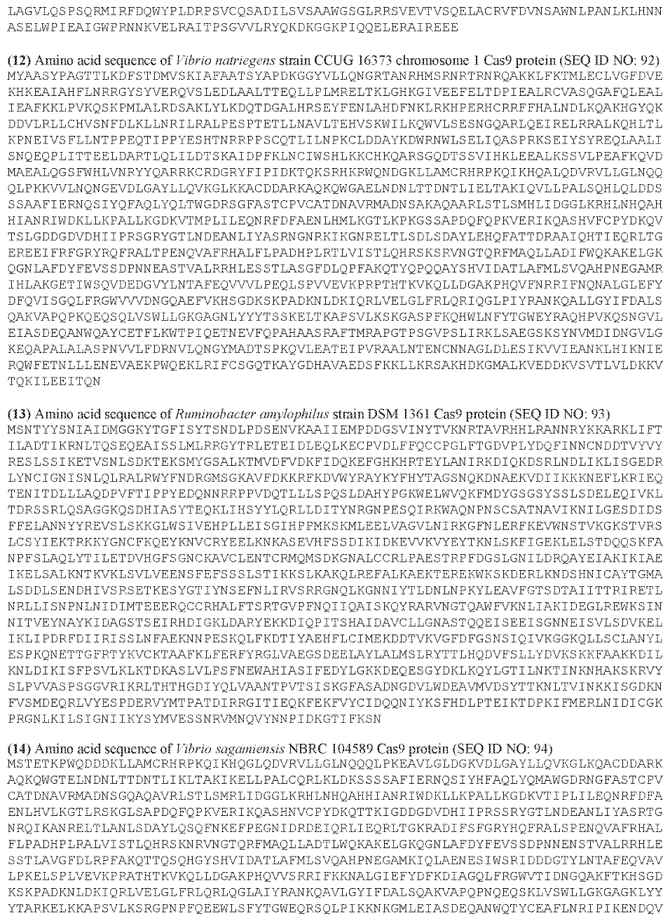

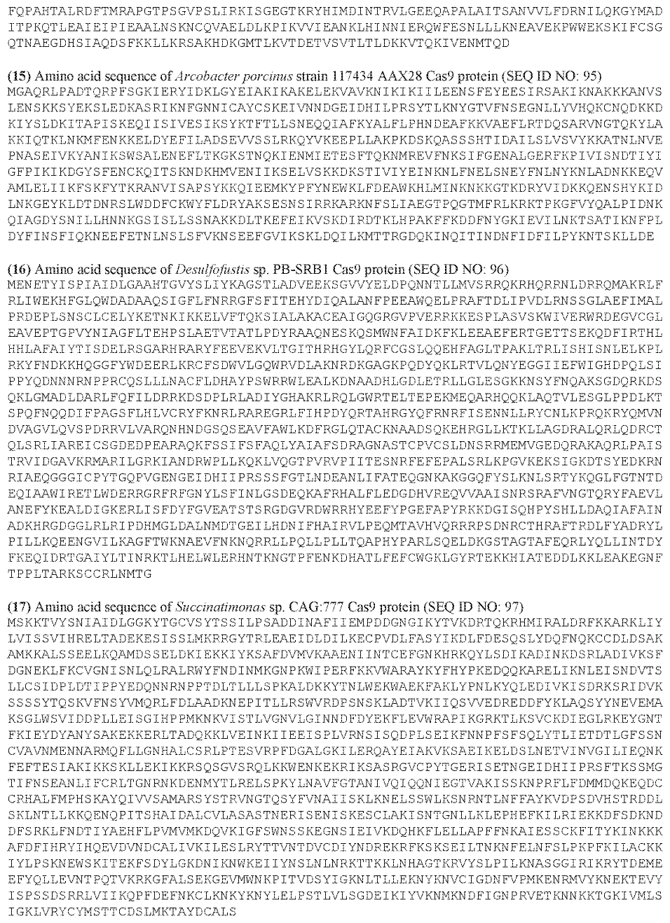

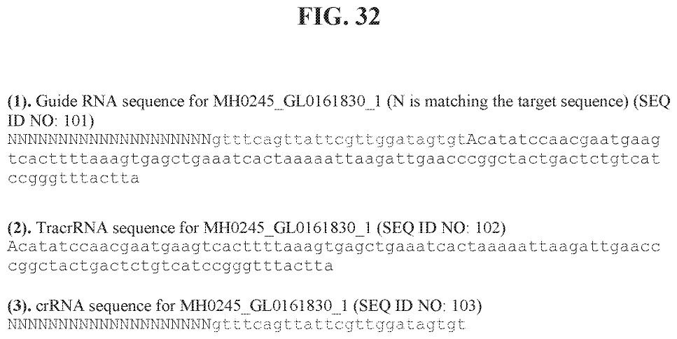

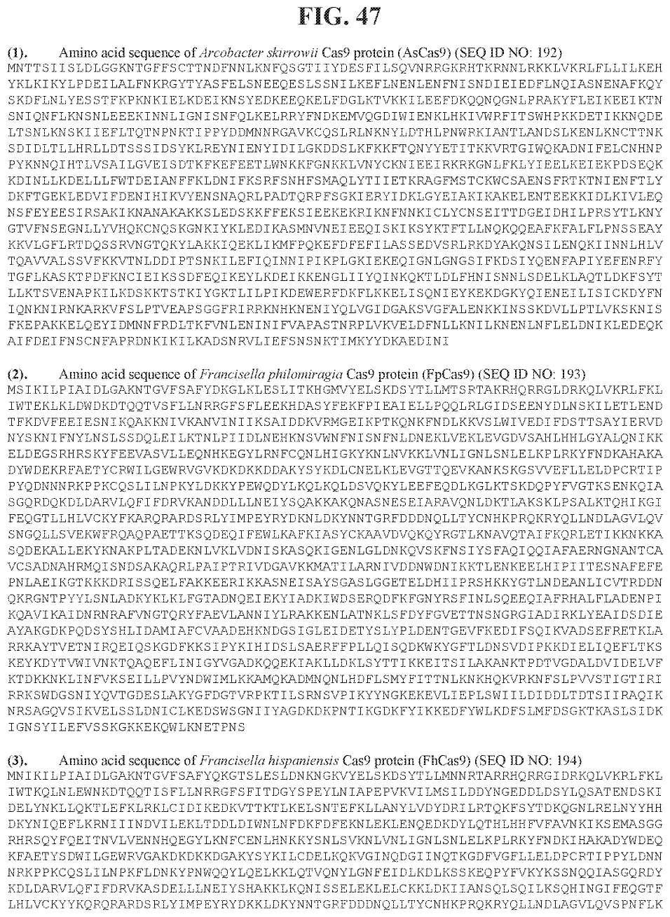

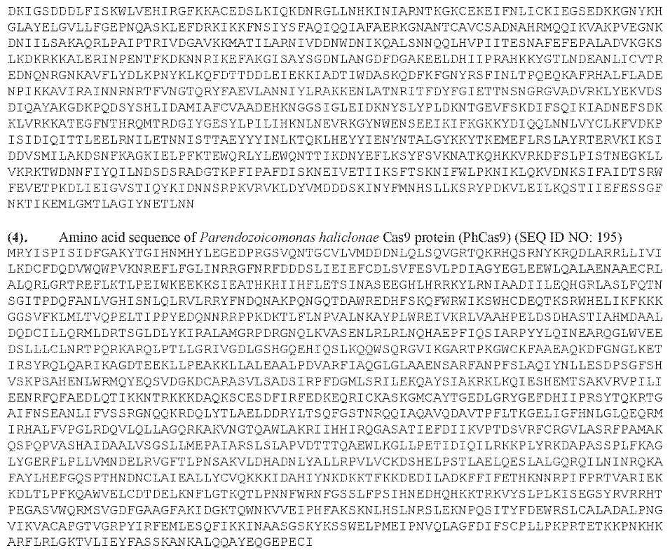

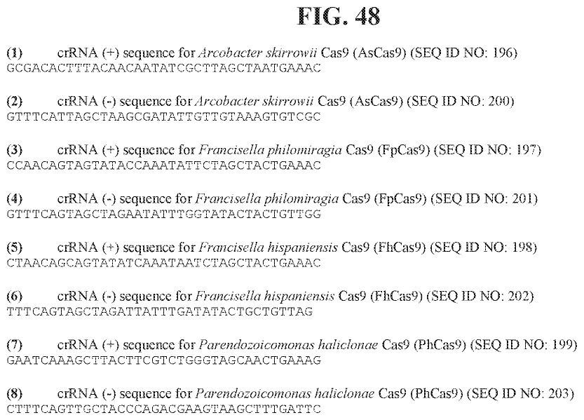

In some embodiments, the term Cas9 refers to a polypeptide comprising the amino acid sequence of Legionella pneumophila Cas9 protein. In some embodiments, the term Cas9 refers to a polypeptide comprising the amino acid sequence of Francisella novicida Cas9 protein. In some embodiments, the term Cas9 refers to a polypeptide comprising the amino acid sequence of gamma proteobacterium HTCC5015 Cas9 protein. In some embodiments, the term Cas9 refers to a polypeptide comprising the amino acid sequence of Parasutterella excrementihominis Cas9 protein. In some embodiments, the term Cas9 refers to a polypeptide comprising the amino acid sequence of Sutterella wadsworthensis Cas9 protein. In some embodiments, the term Cas9 refers to a polypeptide comprising the amino acid sequence of Sulfurospirillum sp. SCADC Cas9 protein. In some embodiments, the term Cas9 refers to a polypeptide comprising the amino acid sequence of Ruminobacter sp. RM87 Cas9 protein. In some embodiments, the term Cas9 refers to a polypeptide comprising the amino acid sequence of Burkholderiales bacterium 1_1_47 Cas9 protein. In some embodiments, the term Cas9 refers to a polypeptide comprising the amino acid sequence of Bacteroidetes oral taxon 274 str. F0058 Cas9 protein. In some embodiments, the term Cas9 refers to a polypeptide comprising the amino acid sequence of Wolinella succinogenes Cas9 protein. In some embodiments, the term Cas9 refers to a polypeptide comprising the amino acid sequence of Burkholderiales bacterium YL45 Cas9 protein. In some embodiments, the term Cas9 refers to a polypeptide comprising the amino acid sequence of Ruminobacter amylophilus strain DSM 1361 Cas9 protein. In some embodiments, the term Cas9 refers to a polypeptide comprising the amino acid sequence of Campylobacter sp. P0111 Cas9 protein. In some embodiments, the term Cas9 refers to a polypeptide comprising the amino acid sequence of Campylobacter sp. RM9261 Cas9 protein. In some embodiments, the term Cas9 refers to a polypeptide comprising the amino acid sequence of Campylobacter lanienae strain RM8001 Cas9 protein. In some embodiments, the term Cas9 refers to a polypeptide comprising the amino acid sequence of Camplylobacter lanienae strain P0121 Cas9 protein. In some embodiments, the term Cas9 refers to a polypeptide comprising the amino acid sequence of Turicimonas muris Cas9 protein. In some embodiments, the term Cas9 refers to a polypeptide comprising the amino acid sequence of Legionella londiniensis Cas9 protein. In some embodiments, the term Cas9 refers to a polypeptide comprising the amino acid sequence of Salinivibrio sharmensis Cas9 protein. In some embodiments, the term Cas9 refers to a polypeptide comprising the amino acid sequence of Leptospira sp. isolate FW.030 Cas9 protein. In some embodiments, the term Cas9 refers to a polypeptide comprising the amino acid sequence of Moritella sp. isolate NORP46 Cas9 protein. In some embodiments, the term Cas9 refers to a polypeptide comprising the amino acid sequence of Endozoicomonassp. S-B4-1U Cas9 protein. In some embodiments, the term Cas9 refers to a polypeptide comprising the amino acid sequence of Tamilnaduibacter salinus Cas9 protein. In some embodiments, the term Cas9 refers to a polypeptide comprising the amino acid sequence of Vibrio natriegens Cas9 protein. In some embodiments, the term Cas9 refers to a polypeptide comprising the amino acid sequence of Arcobacter skirrowii Cas9. In some embodiments, the term Cas9 refers to a polypeptide comprising the amino acid sequence of Francisella philomiragia Cas9. In some embodiments, the term Cas9 refers to a polypeptide comprising the amino acid sequence of Francisella hispaniensis Cas9. In some embodiments, the term Cas9 refers to a polypeptide comprising the amino acid sequence of Parendozoicomonas haliclonae Cas9. In some embodiments, the term Cas9 refers to a Cas9 polypeptide from a metagenomic sequence catalog. In some embodiments, the term Cas9 refers to a polypeptide comprising any of SEQ ID NOs: 10-97 or 192-195. See FIG. 30, SEQ ID NOs: 10-80; FIG. 31, SEQ ID NOs: 81-97; and FIG. 47, SEQ ID NOs: 192-195.

In some embodiments, the stiCas9 protein comprises a domain having a sequence of at least 70%, at least 75%, at least 80%, at least 85%, at least 90%, at least 95%, at least 96%, at least 97%, at least 98%, at least 99%, or about 100% identity with the amino acid sequence of any one of SEQ ID NOs: 10-97 or 192-195. In some embodiments, the stiCas9 protein is at least 70%, at least 75%, at least 80%, at least 85%, at least 90%, at least 95%, at least 96%, at least 97%, at least 98%, at least 99%, or about 100% identical with the amino acid sequence of any one of SEQ ID NOs: 10-97 or 192-195.

As used herein, the term “cohesive ends,” “staggered ends,” or “sticky ends” refer to a nucleic acid fragment with strands of unequal length. In contrast to “blunt ends,” cohesive ends are produced by a staggered cut on the nucleic acid, typically DNA. A sticky or cohesive end has protruding single-stranded strands with unpaired nucleotides, or “overhangs,” e.g., a 3′ or a 5′ overhang. Each overhang can anneal with another complementary overhang to form base pairs. The two complementary cohesive ends can anneal together via interactions such as hydrogen-bonding. The stability of the annealed cohesive ends depends on the melting temperature of the paired overhangs. The two complementary cohesive ends can be joined together by chemical or enzymatic ligation, for example, by DNA ligase.

Cas9 proteins were previously known to generate double-stranded DNA breaks with blunt ends (See, e.g., Jinek et al., 2012). The present disclosure provides a Cas9 protein capable of generating cohesive ends, herein also termed “stiCas9” or “sticky Cas9.” DNA fragments with cohesive ends provide an advantage over blunt ends in further applications such as, for example, inserting a nucleic acid in between the fragments and re-joining the fragments together. A DNA sequence with blunt ends does not provide specificity for inserting the nucleic acid, i.e., the nucleic acid could be inserted at either blunt end. A cohesive end, on the other hand, will only pair with a complementary cohesive end and thus enables the integration of the transgene with a preferable orientation. In some embodiments, cohesive ends facilitate the insertion of DNA through non-homologous end-joining and microhomology mediated end joining methods.

In some embodiments, the cohesive ends generated by the stiCas9 comprise a single-stranded polynucleotide overhang of 3 to 40 nucleotides. In some embodiments, the cohesive ends generated by the stiCas9 comprise a single-stranded polynucleotide overhang of 4 to 20 nucleotides. In some embodiments, the cohesive ends generated by the stiCas9 comprise a single-stranded polynucleotide overhang of 5 to 15 nucleotides. In some embodiments, the cohesive ends generated by the stiCas9 comprise a single-stranded polynucleotide overhang of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. In some embodiments, the cohesive ends generated by the stiCas9 is a 5′ overhang. In some embodiments, the cohesive ends generated by the stiCas9 is a 3′ overhang.

The compositions and methods described herein can comprise a guide polynucleotide. In some embodiments, the guide polynucleotide is an RNA molecule. The RNA molecule that binds to CRISPR-Cas components and targets them to a specific location within the target DNA is referred to herein as “guide RNA,” “gRNA,” or “small guide RNA” and may also be referred to herein as a “DNA-targeting RNA.” A guide polynucleotide, e.g., guide RNA, comprises at least two nucleotide segments: at least one “DNA-binding segment” and at least one “polypeptide-binding segment.” By “segment” is meant a part, section, or region of a molecule, e.g., a contiguous stretch of nucleotides of guide polynucleotide molecule. The definition of “segment,” unless otherwise specifically defined, is not limited to a specific number of total base pairs.

In some embodiments, the DNA-binding segment of the guide polynucleotide hybridizes with a target sequence in a eukaryotic cell, but not a sequence in a bacterial cell. A sequence in a bacterial cell, as used herein, refers to a polynucleotide sequence that is native to a bacterial organism, i.e., a naturally-occurring bacterial polynucleotide sequence, or a sequence of bacterial origin. For example, the sequence can be a bacterial chromosome or bacterial plasmid, or any other polynucleotide sequence that is found naturally in bacterial cells.

In some embodiments, the polypeptide-binding segment of the guide polynucleotide binds to Cas9. In some embodiments, the polypeptide-binding segment of the guide polynucleotide binds to stiCas9.

In some embodiments, the guide polynucleotide is 10 to 150 nucleotides. In some embodiments, the guide polynucleotide is 20 to 120 nucleotides. In some embodiments, the guide polynucleotide is 30 to 100 nucleotides. In some embodiments, the guide polynucleotide is 40 to 80 nucleotides. In some embodiments, the guide polynucleotide is 50 to 60 nucleotides. In some embodiments, the guide polynucleotide is 10 to 35 nucleotides. In some embodiments, the guide polynucleotide is 15 to 30 nucleotides. In some embodiments, the guide polynucleotide is 20 to 25 nucleotides.

The guide polynucleotide, e.g., guide RNA, can be introduced into the target cell as an isolated molecule, e.g., RNA molecule, or is introduced into the cell using an expression vector containing DNA encoding the guide polynucleotide, e.g., guide RNA.

The “DNA-binding segment” (or “DNA-targeting sequence”) of the guide polynucleotide, e.g., guide RNA, comprises a nucleotide sequence that is complementary to a specific sequence within a target DNA.

The guide polynucleotide, e.g., guide RNA, of the present disclosure can include a polypeptide-binding sequence/segment. The polypeptide-binding segment (or “protein-binding sequence”) of the guide polynucleotide, e.g., guide RNA, interacts with the polynucleotide-binding domain of a Cas protein of the present disclosure. Such polypeptide-binding segments or sequences are known to those of skill in the art, e.g., those disclosed in U.S. patent application publications 2014/0068797, 2014/0273037, 2014/0273226, 2014/0295556, 2014/0295557, 2014/0349405, 2015/0045546, 2015/0071898, 2015/0071899, and 2015/0071906, the disclosures of which are incorporated herein in their entireties.

In some embodiments of the present disclosure, the stiCas9 and the guide polynucleotide can form a complex. A “complex” is a group of two or more associated nucleic acids and/or polypeptides. In some embodiments, a complex is formed when all the components of the complex are present together, i.e., a self-assembling complex. In some embodiments, a complex is formed through chemical interactions between different components of the complex such as, for example, hydrogen-bonding. In some embodiments, a guide polynucleotide forms a complex with a stiCas9 through secondary structure recognition of the guide polynucleotide by the stiCas9. In some embodiments, a stiCas9 protein is inactive, i.e., does not exhibit nuclease activity, until it forms a complex with a guide polynucleotide. Binding of guide RNA induces a conformational change in stiCas9 to convert the stiCas9 from the inactive form to an active, i.e., catalytically active, form. In embodiments of the present disclosure, the complex of the stiCas9 and guide polynucleotide does not occur in nature.

In some embodiments, the present disclosure provides a non-naturally occurring CRISPR-Cas system comprising: a Cas9 effector protein capable of generating cohesive ends (stiCas9) and comprises a nuclear localization signal (NLS), and a guide polynucleotide that forms a complex with the stiCas9 and comprises a guide sequence, wherein the complex does not occur in nature.

In some embodiments, the stiCas9 comprises one or more nuclear localization signals. A “nuclear localization signal” or “nuclear localization sequence” (NLS) is an amino acid sequence that “tags” a protein for import into the cell nucleus by nuclear transport, i.e., a protein having an NLS is transported into the cell nucleus. Typically, the NLS comprises positively-charged Lys or Arg residues exposed on the protein surface. Exemplary nuclear localization sequences include, but are not limited to the NLS from: SV40 Large T-Antigen, w, EGL-13, c-Myc, and TUS-protein. In some embodiments, the NLS comprises the sequence PKKKRKV (SEQ ID NO: 1). In some embodiments, the NLS comprises the sequence AVKRPAATKKAGQAKKKKLD (SEQ ID NO: 2). In some embodiments, the NLS comprises the sequence PAAKRVKLD (SEQ ID NO: 3). In some embodiments, the NLS comprises the sequence MSRRRKANPTKLSENAKKLAKEVEN (SEQ ID NO: 4). In some embodiments, the NLS comprises the sequence KLKIKRPVK (SEQ ID NO: 5). Other nuclear localization sequences include, but are not limited to, the acidic M9 domain of hnRNP A1, the sequence KIPIK (SEQ ID NO: 6) in yeast transcription repressor Matα2, and PY-NLSs.

In some embodiments, the present disclosure provides a non-naturally occurring CRISPR-Cas system comprising: (a) one or more nucleotides encoding a Cas9 effector protein capable of generating cohesive ends (stiCas9); and (b) a nucleotide sequence encoding a guide polynucleotide that forms a complex with the stiCas9 and comprising a guide sequence, wherein the guide sequence hybridizes with a target sequence in a eukaryotic cell but does not hybridize to a sequence in a bacterial cell, and wherein the complex does not occur in nature.

In some embodiments, the stiCas9 protein is encoded by one or more polynucleotides. In some embodiments, the polynucleotide is DNA. In some embodiments, the polynucleotide is RNA.

In some embodiments, the stiCas9 is encoded by one or more polynucleotides derived from Legionella pneumophila Cas9 protein. In some embodiments, the stiCas9 is encoded by one or more polynucleotides derived from Francisella novicida Cas9 protein. In some embodiments, the stiCas9 is encoded by one or more polynucleotides derived from gamma proteobacterium HTCC5015 Cas9 protein. In some embodiments, the stiCas9 is encoded by one or more polynucleotides derived from Parasutterella excrementihominis Cas9 protein. In some embodiments, the stiCas9 is encoded by one or more polynucleotides derived from Sutterella wasworthensis Cas9 protein. In some embodiments, the stiCas9 is encoded by one or more polynucleotides derived from Sulfurospirillum sp. SCADC Cas9 protein. In some embodiments, the stiCas9 is encoded by one or more polynucleotides derived from Ruminobacter sp. RM87 Cas9 protein. In some embodiments, the stiCas9 is encoded by one or more polynucleotides derived from Burkholderiales bacterium 1_1_47 Cas9 protein. In some embodiments, the stiCas9 is encoded by one or more polynucleotides derived from Bacteroidetes oral taxon 274 str. F0058 Cas9 protein. In some embodiments, the stiCas9 is encoded by one or more polynucleotides derived from Wolinella succinogenes Cas9 protein. In some embodiments, the stiCas9 is encoded by one or more polynucleotides derived from Burkholderiales bacterium YL45 Cas9 protein. In some embodiments, the stiCas9 is encoded by one or more polynucleotides derived from Ruminobacter amylophilus strain DSM 1361 Cas9 protein. In some embodiments, the stiCas9 is encoded by one or more polynucleotides derived from Campylobacter sp. P0111 Cas9 protein. In some embodiments, the stiCas9 is encoded by one or more polynucleotides derived from Campylobacter sp. RM9261 Cas9 protein. In some embodiments, the stiCas9 is encoded by one or more polynucleotides derived from Campylobacter lanienae strain RM8001 Cas9 protein. In some embodiments, the stiCas9 is encoded by one or more polynucleotides derived from Camplylobacter lanienae strain P0121 Cas9 protein. In some embodiments, the stiCas9 is encoded by one or more polynucleotides derived from Turicimonas muris Cas9 protein. In some embodiments, the stiCas9 is encoded by one or more polynucleotides derived from Legionella londiniensis Cas9 protein. In some embodiments, the stiCas9 is encoded by one or more polynucleotides derived from Salinivibrio sharmensis Cas9 protein. In some embodiments, the stiCas9 is encoded by one or more polynucleotides derived from Leptospira sp. isolate FW.030 Cas9 protein. In some embodiments, the stiCas9 is encoded by one or more polynucleotides derived from Moritella sp. isolate NORP46 Cas9 protein. In some embodiments, the stiCas9 is encoded by one or more polynucleotides derived from Endozoicomonassp. S-B4-1U Cas9 protein. In some embodiments, the stiCas9 is encoded by one or more polynucleotides derived from Tamilnaduibacter salinus Cas9 protein. In some embodiments, the stiCas9 is encoded by one or more polynucleotides derived from Vibrio natriegens Cas9 protein. In some embodiments, the stiCas9 is encoded by one or more polynucleotides derived from Arcobacter skirrowii Cas9 protein. In some embodiments, the stiCas9 is encoded by one or more polynucleotides derived from Francisella philomiragia Cas9 protein. In some embodiments, the stiCas9 is encoded by one or more polynucleotides derived from Francisella hispaniensis Cas9 protein. In some embodiments, the stiCas9 is encoded by one or more polynucleotides derived from Parendozoicomonas haliclonae Cas9 protein.

In some embodiments, the stiCas9 of the present disclosure comprises a domain that matches the TIGR03031 protein family with an E-value cut-off of at least 1E-10, at least 1E-9, at least 1E-8, at least 1E-7, at least 1E-6, at least 1E-5, at least 1E-4, at least 1E-3, at least 1E-2, or at least 1E-1.

In some embodiments, the guide polynucleotide of the CRISPR-Cas system is encoded by a nucleotide sequence. In some embodiments, the nucleotide sequence is DNA. In some embodiments, the guide polynucleotide is guide RNA. In some embodiments, the guide sequence of the guide polynucleotide is a DNA-targeting sequence.

In some embodiments, the nucleotide sequence encoding a stiCas9 is codon optimized. An example of a codon optimized sequence is, in this instance, a sequence optimized for expression in a eukaryote, e.g., humans (i.e., being optimized for expression in humans), or for another eukaryote, animal, or mammal as discussed herein; see, e.g., SaCas9 human codon optimized sequence in WO 2014/093622 as an example of a codon optimized sequence (from knowledge in the art and this disclosure, codon optimizing coding nucleic acid molecule(s), especially as to effector protein (e.g., Cas9) is within the ambit of the skilled artisan). Other examples are possible and codon optimization for a host species other than human, or for codon optimization for specific organs is known. In some embodiments, an enzyme coding sequence encoding a DNA/RNA-targeting Cas protein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, are excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” (www.kazusa.orjp/codon/), and these tables can be adapted in a number of ways. See Nakamura et al., “Codon usage tabulated from the international DNA sequence databases: status for the year 2000,” Nucleic Acids Research 28: 292 (2000). Computer algorithms for codon optimizing a particular sequence for expression in a particular host cell are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database (www.yeastgenome.org/community/codon_usage.shtml), or Bennetzen and Hall, “Codon selection in yeast,” Journal of Biological Chemistry, 257(6): 3026-31 (1982). As to codon usage in plants including algae, reference is made to Campbell and Gowri, “Codon usage in higher plants, green algae, and cyanobacteria,” Plant Physiology 92(1): 1-11 (1990); as well as Murray et al., “Codon usage in plant genes,” Nucleic Acids Research 17(2): 477-98 (1989); or Morton, “Selection on the codon bias of chloroplast and cyanelle genes in different plant and algal lineages,” Molecular Evolution 46(4): 449-59 (1998). In some embodiments, one or more of SEQ ID NOS: 10-97 or 192-195 are codon optimized.

In some embodiments, the nucleotide sequence encoding a stiCas9 is codon optimized for expression in a eukaryotic cell. In some embodiments, the nucleotide sequence encoding a stiCas9 is codon optimized for expression in an animal cell. In some embodiments, the nucleotide sequence encoding a stiCas9 is codon optimized for expression in a human cell. The nucleotide sequence encoding a stiCas9 is codon optimized for expression in a plant cell. Codon optimization is the adjustment of codons to match the expression host's tRNA abundance in order to increase yield and efficiency of recombinant or heterologous protein expression. Codon optimization methods are routine in the art and may be performed using software programs such as, for example, Integrated DNA Technologies' Codon Optimization tool, Entelechon's Codon Usage Table analysis tool, GENEMAKER's Blue Heron software, Aptagen's Gene Forge software, DNA Builder Software, General Codon Usage Analysis software, the publicly available OPTIMIZER software, and Genscript's OptimumGene algorithm.

In some embodiments, the CRISPR-Cas systems of the present disclosure further comprise a tracrRNA. A “tracrRNA,” or trans-activating CRISPR-RNA, forms an RNA duplex with a pre-crRNA, or pre-CRISPR-RNA, and is then cleaved by the RNA-specific ribonuclease RNase III to form a crRNA/tracrRNA hybrid. In some embodiments, the guide RNA comprises the crRNA/tracrRNA hybrid. In some embodiments, the tracrRNA component of the guide RNA activates the Cas9 protein.

In some embodiments of the present disclosure, the stiCas9, guide polynucleotide, and tracrRNA are capable of forming a complex. In some embodiments, the complex of the stiCas9, guide polynucleotide, and tracrRNA does not occur in nature.

In some embodiments, the present disclosure provides a non-naturally occurring CRISPR-Cas system comprising one or more vectors comprising: (a) a regulatory element operably linked to one or more nucleotide sequences encoding a Cas9 effector protein capable of generating cohesive ends (stiCas9); (b) a guide polynucleotide that forms a complex with the stiCas9 and comprising a guide sequence, wherein the guide sequence is capable of hybridizing with a target sequence in a eukaryotic cell but does not hybridize to a sequence in a bacterial cell; wherein the complex does not occur in nature. It is understood by the skilled artisan that a vector comprising “a guide polynucleotide that forms a complex with the stiCas9 and comprising a guide sequence” would also include a vector comprising a polynucleotide sequence which can be transcribed to the guide polynucleotide. For example, the DNA vector can be transcribed to produce a guide RNA sequence.

In some embodiments, the present disclosure provides a non-naturally occurring CRISPR-Cas system comprising one or more vectors comprising: a regulatory element operably linked to one or more nucleotide sequences encoding a Cas9 effector protein capable of generating cohesive ends (stiCas9), wherein the regulatory element is a eukaryotic regulatory element, and a guide polynucleotide that forms a complex with the stiCas9 and comprises a guide sequence, wherein the complex does not occur in nature.

In some embodiments, the regulatory element is a promoter. In some embodiments, the regulatory element is a bacterial promoter. In some embodiments, the regulatory element is a viral promoter. In some embodiments, the regulatory element is a eukaryotic regulatory element, i.e., a eukaryotic promoter. In some embodiments, the eukaryotic regulatory element is a mammalian promoter.

“Operably linked” means that the nucleotide of interest, i.e., the nucleotide encoding a Cas9 protein, is linked to the regulatory element in a manner that allows for expression of the nucleotide sequence. Thus, in some embodiments, the vector is an expression vector.

In some embodiments, the guide polynucleotide of the vector comprising the CRISPR-Cas system is encoded by a nucleotide sequence. In some embodiments, the nucleotide sequence is DNA. In some embodiments, the guide polynucleotide is guide RNA. In some embodiments, the guide sequence of the guide polynucleotide is a DNA-targeting sequence.

In some embodiments, the stiCas9 and guide polynucleotide are capable of forming a complex. In some embodiments, the complex of the stiCas9 and guide polynucleotide does not occur in nature.

In some embodiments, the vector further comprises a nucleotide sequence comprising a tracrRNA sequence. In some embodiments, the guide RNA comprises the crRNA/tracrRNA hybrid. In some embodiments, the tracrRNA component of the guide RNA activates the Cas9 protein.

In some embodiments, the CRISPR-Cas system as described herein is capable of cleaving at a site within 10 nucleotides of a Protospacer Adjacent Motif. A Protospacer Adjacent Motif, or PAM, is a 2-6 base pair nucleotide sequence located within one nucleotide of the region complementary to the guide RNA. When Cas9 protein is activated (for example, by formation of a complex with the guide polynucleotide), it searches for target DNA by binding with sequences that match its PAM sequence. See, e.g., Sternberg et al., “DNA interrogation by the CRISPR RNA-guided endonuclease Cas9,” Nature 507(7490): 62-67 (2014), which is incorporated by reference herein in its entirety. Upon recognition of a potential target sequence with the appropriate PAM, and the guide RNA pairs properly with the target region, the nuclease domains of Cas9 (i.e., the RuvC and HNH domains) cut the target DNA.

In some embodiments, the RuvC and HNH domains of the Cas9 proteins of the present disclosure each cut one strand of the target DNA sequence. In embodiments, the cut sites of the RuvC and HNH domains of a stiCas9 protein are offset, i.e., each domain cuts at a different position on its respective strand of the target DNA, resulting in an overhang. In embodiments, the RuvC and HNH domains of the stiCas9 protein cut at a 3-nucleotide offset. In embodiments, the RuvC and HNH domains of the stiCas9 protein cut at a 4-nucleotide offset. In embodiments, the RuvC and HNH domains of the stiCas9 protein cut at a 5-nucleotide offset. In embodiments, the RuvC and HNH domains of the stiCas9 protein cut at an offset of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides.

In some embodiments, the RuvC and HNH domains of a Cas9 effector protein of the present disclosure cleaves at different positions on each strand of the double-stranded target DNA. In some embodiments, the RuvC domain of the Cas9 effector protein cleaves one strand of the double-stranded target DNA (which can be referred to, for example, as the “non-target strand”) at from about −10, about −9, about −8, about −7, or about −6 nucleotides from the PAM, and the HNH domain of the Cas9 effector protein cleaves the other strand of the double-stranded target DNA (which can be referred to, for example, as the “target strand”) at −5, about −4, about −3, about −2, or about −1 nucleotides from the PAM.

In some embodiments, the RuvC domain cleaves one strand of the double-stranded target DNA at about −8 nucleotides from the PAM. In some embodiments, the RuvC domain cleaves one strand of the double-stranded target DNA at about −7 nucleotides from the PAM. In some embodiments, the RuvC domain cleaves one strand of the double-stranded target DNA at about −6 nucleotides from the PAM. In some embodiments, the HNH domain cleaves one strand of the double-stranded target DNA at about −4 nucleotides from the PAM. In some embodiments, the HNH domain cleaves one strand of the double-stranded target DNA at about −3 nucleotides from the PAM. In some embodiments, the HNH domain cleaves one strand of the double-stranded target DNA at about −2 nucleotides from the PAM.

In some embodiments of the present disclosure, the complex comprising stiCas9 and a guide polynucleotide is capable of cleaving at a site within 10 nucleotides of a Protospacer Adjacent Motif (PAM). In some embodiments, the complex comprising stiCas9 and a guide polynucleotide is capable of cleaving at a site within 5 nucleotides of a PAM. In some embodiments, the complex comprising stiCas9 and a guide polynucleotide is capable of cleaving at a site within 3 nucleotides of a PAM. In some embodiments, the PAM is downstream (i.e., 3′ direction) of the target sequence. In some embodiments, the PAM is upstream (i.e., 5′ direction) of the target sequence. In some embodiments, the PAM is located within the target sequence.

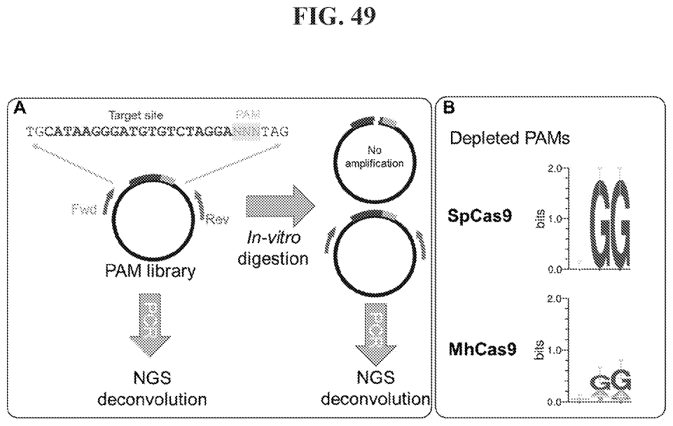

Different bacterial species recognize different PAM sequences. One method of identifying the preferred PAM sequence for a Cas9 protein of the present disclosure is illustrated in FIG. 49A and includes, for example, generating a plasmid library of various PAM sequences adjacent to a target sequence, contacting the plasmid library with a Cas9 protein, then sequencing the plasmid library to determine which PAM sequences have been “depleted” (i.e., not detected in the sequencing results). The “depleted” PAM sequences are the ones that are recognized and effected upon (i.e., cleaved) by the Cas9 protein.

For example, the PAM sequence recognized by the Cas9 of Streptococcus pyogenes is 5′-NGG-3′, wherein N is any nucleotide. Different PAMs are associated with the Cas9 proteins of Neisseria meningitidis, Treponema denticola, and Streptococcus thermophilus. The Cas9 protein of Francisella novicida has been engineered to recognize the PAM 5′-YG-3′, wherein Y is a pyrimidine.

In some embodiments, the PAM comprises a 3′ G-rich motif. In some embodiments, the PAM sequence is NGG, wherein N is A, C, T, U, or G. In some embodiments, the PAM sequence is NGA, wherein N is A, C, T, U, or G. In some embodiments, the PAM sequence is YG, wherein Y is a pyrimidine (i.e., C, T, or U).

In some embodiments, the target sequence is 5′ of a PAM and the PAM comprises a 3′ G-rich motif. In some embodiments, the target sequence is 5′ of a PAM and the PAM sequence is NGG, wherein N is A, C, T, U, or G. In some embodiments, the target sequence is 5′ of a PAM, the PAM sequence is YG, wherein Y is a pyrimidine, and the stiCas9 is derived from the bacterial species Francisella novicida.

In some embodiments, the stiCas9 comprises one or more nuclear localization signals. A “nuclear localization signal” or “nuclear localization sequence” (NLS) is an amino acid sequence that “tags” a protein for import into the cell nucleus by nuclear transport, i.e., a protein having an NLS is transported into the cell nucleus. Typically, the NLS comprises positively-charged Lys or Arg residues exposed on the protein surface. Exemplary nuclear localization sequences include, but are not limited to the NLS from: SV40 Large T-Antigen, nucleoplasmin, EGL-13, c-Myc, and TUS-protein. In some embodiments, the NLS comprises the sequence PKKKRKV (SEQ ID NO: 1). In some embodiments, the NLS comprises the sequence AVKRPAATKKAGQAKKKKLD (SEQ ID NO: 2). In some embodiments, the NLS comprises the sequence PAAKRVKLD (SEQ ID NO: 3). In some embodiments, the NLS comprises the sequence MSRRRKANPTKLSENAKKLAKEVEN (SEQ ID NO: 4). In some embodiments, the NLS comprises the sequence KLKIKRPVK (SEQ ID NO: 5). Other nuclear localization sequences include, but are not limited to, the acidic M9 domain of hnRNP A1, the sequence KIPIK (SEQ ID NO: 6) in yeast transcription repressor Matα2, and PY-NLSs.

In some embodiments, the guide polynucleotide of the present disclosure has a guide sequence that hybridizes to a target sequence in a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as, for example, EBx® cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the human cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes, plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the guide polynucleotide of the CRISPR-Cas system is linked to a direct repeat sequence. A direct repeat, or DR, sequence is an array of repetitive sequences in the CRISPR locus, interspaced by short stretches of non-repetitive sequences (spacers). The spacer sequences target the Protospacer Adjacent Motifs (PAM) on the target sequence. When the non-coding portion of the CRISPR locus (i.e., the guide polynucleotide and the tracrRNA) is transcribed, the transcript is cleaved at the DR sequences into short crRNAs containing individual spacer sequences, which direct the Cas9 nuclease to the PAM. In some embodiments, the DR sequence is RNA. In some embodiments, the DR sequence is encoded by a nucleic acid. In some embodiments, the DR sequence is linked to the guide polynucleotide. In some embodiments, the DR sequence is linked to the guide sequence of the guide polynucleotide. In some embodiments, the DR sequence comprises a secondary structure. In some embodiments, the DR sequence comprises a stem loop structure. In some embodiments, the DR sequence is 10 to 20 nucleotides. In some embodiments, the DR sequence is at least 16 nucleotides. In some embodiments, the DR sequence is at least 16 nucleotides and comprises a single stem loop. In some embodiments, the DR sequence comprises an RNA aptamer. In some embodiments, the secondary structure or stem loop in the DR is the recognized by a nuclease for cleavage. In some embodiments, the nuclease is a ribonuclease. In some embodiments, the nuclease is RNase III.

Various means are known in the art for delivery of CRISPR-Cas systems. In some embodiments, the CRISPR-Cas system of the present disclosure is delivered by a delivery particle. A delivery particle is a biological delivery system or formulation which includes a particle. A “particle,” as defined herein, is an entity having a maximum diameter of about 100 microns (μm). In some embodiments, the particle has a maximum diameter of about 10 μm. In some embodiments, the particle has a maximum diameter of about 2000 nanometers (nm). In some embodiments, the particle has a maximum diameter of about 1000 nm. In some embodiments, the particle has a maximum diameter of about 900 nm, about 800 nm, about 700 nm, about 600 nm, about 500 nm, about 400 nm, about 300 nm, about 200 nm, or about 100 nm. In some embodiments, the particle has a diameter of about 25 nm to about 200 nm. In some embodiments, the particle has a diameter of about 50 nm to about 150 nm. In some embodiments, the particle has a diameter of about 75 nm to about 100 nm.

Delivery particles may be provided in any form, including but not limited to: solid, semi-solid, emulsion, or colloidal particles. In some embodiments, the delivery particle is a lipid-based system, a liposome, a micelle, a microvesicle, an exosome, or a gene gun. In some embodiments, the delivery particle comprises a CRISPR-Cas system. In some embodiments, the delivery particle comprises a CRISPR-Cas system comprising a stiCas9 and a guide polynucleotide. In some embodiments, the delivery particle comprises a CRISPR-Cas system comprising a stiCas9 and a guide polynucleotide, wherein the stiCas9 and the guide polynucleotide are in a complex. In some embodiments, the delivery particle comprises a CRISPR-Cas system comprising a stiCas9, a guide polynucleotide, and polynucleotide comprising a tracrRNA. In some embodiments, the delivery particle comprises a CRISPR-Cas system comprising a stiCas9, a guide polynucleotide, and a tracrRNA.

In some embodiments, the delivery particle further comprises a lipid, a sugar, a metal or a protein. In some embodiments, the delivery particle is a lipid envelope. Delivery of mRNA using lipid envelopes or delivery particles comprising lipids is described, for example, in Su et al., “In vitro and in vivo mRNA delivery using lipid-enveloped pH-responsive polymer nanoparticles,” Molecular Pharmacology 8(3): 774-784 (2011).

In some embodiments, the delivery particle is a sugar-based particle, for example, GalNAc. Sugar-based particles are described in WO 2014/118272 and Nair et al., Journal of the American Chemical Society 136(49): 16958-16961 (2014), each of which is incorporated by reference herein in its entirety.

In some embodiments, the delivery particle is a nanoparticle. Nanoparticles encompassed in the present disclosure may be provided in different forms, e.g., as solid nanoparticles (e.g., metal such as silver, gold, iron, titanium), non-metal, lipid-based solids, polymers, suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Preparation of delivery particles is further described in U.S. Patent Publication Nos. 2011/0293703, 2012/0251560, and 2013/0302401; and U.S. Pat. Nos. 5,543,158, 5,855,913, 5,895,309, 6,007,845, and 8,709,843, each of which is incorporated by reference herein in its entirety.

In some embodiments, a vesicle comprises the CRISPR-Cas system of the present disclosure. A “vesicle” is a small structure within a cell having a fluid enclosed by a lipid bilayer. In some embodiments, the CRISPR-Cas system of the present disclosure is delivered by a vesicle. In some embodiments, the vesicle comprises a stiCas9 and a guide polynucleotide. In some embodiments, the vesicle comprises a stiCas9 and a guide polynucleotide, wherein the stiCas9 and the guide polynucleotide are in a complex. In some embodiments, the vesicle comprises a CRISPR-Cas system comprising a stiCas9, a guide polynucleotide, and polynucleotide comprising a tracrRNA. In some embodiments, the vesicle comprises a CRISPR-Cas system comprising a stiCas9, a guide polynucleotide, and a tracrRNA.

In some embodiments, the vesicle comprising the stiCas9 and guide polynucleotide is an exosome or a liposome. In some embodiments, the vesicle is an exosome. In some embodiments, the exosome is used to deliver the CRISPR-Cas systems of the present disclosure. Exosomes are endogenous nano-vesicles (i.e., having a diameter of about 30 to about 100 nm) that transport RNAs and proteins, and which can deliver RNA to the brain and other target organs. Engineered exosomes for delivery of exogenous biological materials into target organs is described, for example, by Alvarez-Erviti et al., Nature Biotechnology 29: 341 (2011), El-Andaloussi et al., Nature Protocols 7: 2112-2116 (2012), and Wahlgren et al., Nucleic Acids Research 40(17): e130 (2012), each of which is incorporated by reference herein in its entirety.

In some embodiments, the vesicle comprising the stiCas9 and guide polynucleotide is a liposome. In some embodiments, the liposome is used to deliver the CRISPR-Cas systems of the present disclosure. Liposomes are spherical vesicle structures having at least one lipid bilayer and can be used as a vehicle for administration of nutrients and pharmaceutical drugs. Liposomes are often composed of phospholipids, in particular phosphatidylcholine, but also other lipids such as egg phosphatidylethanolamine. Types of liposomes include, but are not limited to, multilamellar vesicle, small unilamellar vesicle, large unilamellar vesicle, and cochleate vesicle. See, e.g., Spuch and Navarro, “Liposomes for Targeted Delivery of Active Agents against Neurodegenerative Diseases (Alzheimer's Disease and Parkinson's Disease), Journal of Drug Delivery 2011, Article ID 469679 (2011). Liposomes for delivery of biological materials such as CRISPR-Cas components are described, for example, by Morrissey et al., Nature Biotechnology 23(8): 1002-1007 (2005), Zimmerman et al., Nature Letters 441: 111-114 (2006), and Li et al., Gene Therapy 19: 775-780 (2012), each of which is incorporated by reference herein in its entirety.

In some embodiments, the nucleotide encoding a Cas9 and a guide polynucleotide is on a single vector. In some embodiments, a nucleotide encoding a Cas9, a guide polynucleotide (or nucleotide that can be transcribed into a guide polynucleotide), and a tracrRNA are on a single vector. In some embodiments, the nucleotide encoding a Cas9, a guide polynucleotide (or nucleotide that can be transcribed into a guide polynucleotide), a tracrRNA, and a direct repeat sequence are on a single vector. In some embodiments, the vector is an expression vector. In some embodiments, the vector is a mammalian expression vector. In some embodiments, the vector is a human expression vector. In some embodiments, the vector is a plant expression vector.

In some embodiments, the nucleotide encoding a Cas9 and a guide polynucleotide is a single nucleic acid molecule. In some embodiments, the nucleotide encoding a Cas9, a guide polynucleotide, and a tracrRNA is a single nucleic acid molecule. In some embodiments, the nucleotide encoding a Cas9, a guide polynucleotide, a tracrRNA, and a direct repeat sequence is a single nucleic acid molecule. In some embodiments, the single nucleic acid molecule is an expression vector. In some embodiments, the single nucleic acid molecule is a mammalian expression vector. In some embodiments, the single nucleic acid molecule is a human expression vector. In some embodiments, the single nucleic acid molecule is a plant expression vector.

In some embodiments, a viral vector comprises the CRISPR-Cas systems of the present disclosure. In some embodiments, the CRISPR-Cas system of the present disclosure is delivered by a viral vector. In some embodiments, the viral vector comprises a stiCas9 and a guide polynucleotide. In some embodiments, the viral vector comprises a stiCas9 and a guide polynucleotide, wherein the stiCas9 and the guide polynucleotide are in a complex. In some embodiments, the viral vector comprises a CRISPR-Cas system comprising a stiCas9, a guide polynucleotide, and polynucleotide comprising a tracrRNA. In some embodiments, the viral vector comprises a CRISPR-Cas system comprising a stiCas9, a guide polynucleotide, and a tracrRNA. In some embodiments, the viral vector is of an adenovirus, a lentivirus, or an adeno-associated virus. Examples of viral vectors are provided herein.

In some embodiments, adeno-associated virus (AAV) and/or lentiviral vectors can be used as a viral vector comprising the elements of the CRISPR-Cas systems as described herein. In some embodiments of the present disclosure, the Cas protein is expressed intracellularly by cells transduced by a viral vector.

For many therapeutic strategies, included those envisaged by the present disclosure, Cas protein expression may only be required transiently. As a result, in some embodiments of the present disclosure, delivery of the Cas protein into cells is achieved using non-integrative viral vectors. In other embodiments, the expression of CRISPR-Cas system components is required for extended periods—for example, when used in gene circuits which are permanently integrated into the genome of target cells. Such applications have been discussed by Agustín-Pavón, et al., “Synthetic biology and therapeutic strategies for the degenerating brain,” Bioessays 36(10): 979-990 (2014), which is incorporated by reference herein in its entirety.

In some embodiments, the Cas proteins and methods of the present disclosure are used in ex vivo gene editing, such as CAR-T type therapies. These embodiments may involve modification of cells from human donors. In these instances, viral vectors can be also used; however, there is the additional option to directly transfect the Cas protein (along with in vitro transcribed guide RNA and donor DNA) into cultured cells.

In some embodiments, the present disclosure provides a eukaryotic cell comprising a CRISPR-Cas system comprising: (a) a Cas9 effector protein capable of generating cohesive ends (stiCas9), and (b) a guide polynucleotide that forms a complex with the stiCas9 and comprising a guide sequence, wherein the guide sequence is capable of hybridizing with a target sequence in the eukaryotic cell wherein the complex does not occur in nature. In some embodiments, the eukaryotic cell comprises a vector comprising the CRISPR-Cas system of the present disclosure.

In some embodiments, the eukaryotic cell is an animal or human cell. In some embodiments, the eukaryotic cell is an animal cell. In some embodiments, the eukaryotic cell is a human cell, including human stem cell. In some embodiments, the eukaryotic cell is a plant cell. Examples of various types of eukaryotic cells are provided herein.

In some embodiments, the present disclosure provides a eukaryotic cell comprising a CRISPR-Cas system comprising a Cas9 effector protein capable of generating cohesive ends (stiCas9), wherein the Cas9 effector protein is derived from a bacterial species having a Type II-B CRISPR system. In some embodiments, the eukaryotic cell comprises a stiCas9 comprising a domain that matches the TIGR03031 protein family with an E-value cut-off of at least 1E-10, at least 1E-9, at least 1E-8, at least 1E-7, at least 1E-6, at least 1E-5, at least 1E-4, at least 1E-3, at least 1E-2, or at least 1E-1. In some embodiments, the eukaryotic cell comprises a stiCas9 comprising a polypeptide sequence of at least 50%, at least 60%, at least 70%, at least 80%, at least 90%, at least 95%, at least 96%, at least 97%, at least 98%, or at least 99% sequence similarity to any one of SEQ ID NOs: 10-97 or 192-195. In some embodiments, the eukaryotic cell comprises a stiCas9 comprising a polypeptide sequence having at least 70%, at least 75%, at least 80%, at least 85%, at least 90%, at least 95%, at least 96%, at least 97%, at least 98%, at least 99%, or 100% identity with any one of SEQ ID NOs: 10-97 or 192-195.

In some embodiments, the Cas9 proteins of the present disclosure are part of a fusion protein comprising one or more heterologous protein domains (e.g., about or at least about 1, 2, 3, 4, 5, 6, 7, 8, 9, or 10 or more domains in addition to the Cas9 protein). A Cas9 fusion protein can comprise any additional protein sequence, and optionally a linker sequence between any two domains. Examples of protein domains that may be fused to a Cas9 protein include, without limitation: epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include: histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), autofluorescent proteins including blue fluorescent protein (BFP), and mCherry. In some embodiments, a Cas9 protein is fused to a protein or a fragment of a protein that binds DNA molecules or bind other cellular molecules, including but not limited to: maltose binding protein (MBP), S-tag, Lex A DNA binding domain (DBD), GAL4 DNA binding domain, and herpes simplex virus (HSV) BP16 protein. Additional domains that may form part of a fusion protein comprising a Cas9 protein are described in US20110059502, incorporated herein by reference in its entirety. In some embodiments, a tagged Cas9 protein is used to identify the location of a target sequence.

In some embodiments, a Cas9 protein may form a component of an inducible system. The inducible nature of the system allows for spatiotemporal control of gene editing or gene expression using a form of energy. The form of energy can include, but is not limited to: electromagnetic radiation, sound energy, chemical energy, and thermal energy. Non-limiting examples of inducible system include: tetracycline inducible promoters (Tet-On or Tet-Off), small molecule two-hybrid transcription activations systems (FKBP, ABA, etc), or light inducible systems (Phytochrome, LOV domains, or cryptochrome). In some embodiments, the Cas9 protein is a part of a Light Inducible Transcriptional Effector (LITE) to direct changes in transcriptional activity in a sequence-specific manner. The components of a light may include a Cas9 protein, a light-responsive cytochrome heterodimer (e.g., from Arabidopsis thaliana), and a transcriptional activation/repression domain. Further examples of inducible DNA binding proteins and methods for their use are provided in International Application Publication Nos. WO 2014/018423 and WO 2014/093635; U.S. Pat. Nos. 8,889,418 and 8,895,308; and U.S. Patent Publication Nos. 2014/0186919, 2014/0242700, 2014/0273234, and 2014/0335620; each of which is hereby incorporated by reference in its entirety.

### Methods for Site-Specific Modifications

In some embodiments, the present disclosure presents a method for providing site-specific modification of a target sequence in a eukaryotic cell, the method comprising: (1) introducing into the cell: (a) a Cas9 effector protein capable of generating cohesive ends (stiCas9), and (b) a guide polynucleotide that forms a complex with the stiCas9 and comprises a guide sequence, wherein the guide sequence is capable of hybridizing with the target sequence in the eukaryotic cell but does not hybridize to a sequence in a bacterial cell, wherein the complex does not occur in nature; (2) generating cohesive ends in the target sequence with the Cas9 effector protein and the guide polynucleotide; and (3) ligating: (a) the cohesive ends together, or (b) a polynucleotide sequence of interest (SoI) to the cohesive ends, thereby modifying the target sequence.

A “modification” of a target sequence encompasses single-nucleotide substitutions, multiple-nucleotide substitutions, insertions (i.e., knock-in) and deletions (i.e., knock-out) of a nucleic acid, frameshift mutations, and other nucleic acid modifications.

In some embodiments, the modification is a deletion of at least part of the target sequence. A target sequence can be cleaved at two different sites and generate complementary cohesive ends, and the complementary cohesive ends can be re-ligated, thereby removing the sequence portion in between the two sites.

In some embodiments, the modification is a mutation of the target sequence. Site-specific mutagenesis in eukaryotic cells is achieved by the use of site-specific nucleases that promote homologous recombination of an exogenous polynucleotide template (also called a “donor polynucleotide” or “donor vector”) containing a mutation of interest. In some embodiments, a sequence of interest (SoI) comprises a mutation of interest.

In some embodiments, the modification is inserting a sequence of interest (SoI) into the target sequence. The SoI can be introduced as an exogenous polynucleotide template. In some embodiments, the exogenous polynucleotide template comprises cohesive ends. In some embodiments, the exogenous polynucleotide template comprises cohesive ends complementary to cohesive ends in the target sequence.

The exogenous polynucleotide template can be of any suitable length, such as about or at least about 10, 15, 20, 25, 50, 75, 100, 150, 200, 250, 500 or 1000 or more nucleotides in length. In some embodiments, the exogenous polynucleotide template is complementary to a portion of a polynucleotide comprising the target sequence. When optimally aligned, the exogenous polynucleotide template overlaps with one or more nucleotides of a target sequence (e.g., about or at least about 1, 5, 10, 15, 20, 25, 30, 35, 40, 45, 50, 60, 70, 80, 90, or 100 or more nucleotides). In some embodiments, when the exogenous polynucleotide template and a polynucleotide comprising the target sequence are optimally aligned, the nearest nucleotide of the exogenous polynucleotide template is within about 1, 5, 10, 15, 20, 25, 50, 75, 100, 200, 300, 400, 500, 100, 1500, 2000, 2500, 5000, 10000 or more nucleotides from the target sequence.

In some embodiments, the exogenous polynucleotide is DNA, such as, e.g., a DNA plasmid, a bacterial artificial chromosome (BAC), a yeast artificial chromosome (YAC), a viral vector, a linear piece of single-stranded or double-stranded DNA, an oligonucleotide, a PCR fragment, a naked nucleic acid, or a nucleic acid complexed with a delivery vehicle such as a liposome.

In some embodiments, the exogenous polynucleotide is inserted into the target sequence using an endogenous DNA repair pathway of the cell. Endogenous DNA repair pathways include the Non-Homologous End Joining (NHEJ) pathway, Microhomology-Mediated End Joining (MMEJ) pathway, and the Homology-Directed Repair (HDR) pathway. NHEJ, MMEJ, and HDR pathways repair double-stranded DNA breaks. In NHEJ, a homologous template is not required for repairing breaks in the DNA. NHEJ repair can be error-prone, although errors are decreased when the DNA break comprises compatible overhangs. NHEJ and MMEJ are mechanistically distinct DNA repair pathways with different subsets of DNA repair enzymes involved in each of them. Unlike NHEJ, which can be precise as well as error-prone, MMEJ is always error-prone and results in both deletion and insertions at the site under repair. MMEI-associated deletions are due to the micro-homologies (2-10 base pairs) at both sides of a double-strand break. In contrast, HDR requires a homologous template to direct repair, but HDR repairs are typically high-fidelity and less error-prone. In some embodiments, the error-prone nature of NHEJ and MMEJ repairs is exploited to introduce non-specific nucleotide substitutions in the target sequence. In some embodiments, stiCas9 cuts the target sequence in a manner that facilitates HDR repair.

During the repair process, an exogenous polynucleotide template comprising the SoI can be introduced into the target sequence. In some embodiments, an exogenous polynucleotide template comprising the SoI flanked by an upstream sequence and a downstream sequence is introduced into the cell, wherein the upstream and downstream sequences share sequence similarity with either side of the site of integration in the target sequence. In some embodiments, the exogenous polynucleotide comprising the SoI comprises, for example, a mutated gene. In some embodiments, the exogenous polynucleotide comprises a sequence endogenous or exogenous to the cell. In some embodiments, the SoI comprises polynucleotides encoding a protein, or a non-coding sequence such as, e.g., a microRNA. In some embodiments, the SoI is operably linked to a regulatory element. In some embodiments, the SoI is a regulatory element. In some embodiments, the SoI comprises a resistance cassette, e.g., a gene that confers resistance to an antibiotic. In some embodiments, the SoI comprises a mutation of the wild-type target sequence. In some embodiments, the SoI disrupts or corrects the target sequence by creating a frameshift mutation or nucleotide substitution. In some embodiments, the SoI comprises a marker. Introduction of a marker into a target sequence can make it easy to screen for targeted integrations. In some embodiments, the marker is a restriction site, a fluorescent protein, or a selectable marker. In some embodiments, the SoI is introduced as a vector comprising the SoI.

The upstream and downstream sequences in the exogenous polynucleotide template are selected to promote homologous recombination between the target sequence and the exogenous polynucleotide. The upstream sequence is a nucleic acid sequence that shares sequence similarity with the sequence upstream of the targeted site for integration (i.e., the target sequence). Similarly, the downstream sequence is a nucleic acid sequence that shares sequence similarity with the sequence downstream of the targeted site for integration. Thus, in some embodiments, the exogenous polynucleotide template comprising the SoI is inserted into the target sequence by homologous recombination at the upstream and downstream sequences. In some embodiments, the upstream and downstream sequences in the exogenous polynucleotide template have at least 70%, at least 75%, at least 80%, at least 85%, at least 90%, at least 95%, at least 96%, at least 97%, at least 98%, at least 99%, or 100% sequence identity with the upstream and downstream sequences of the targeted genome sequence, respectively. In some embodiments, the upstream or downstream sequence has about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In some embodiments, the upstream or downstream sequence has about 50, about 100, about 250, about 500, about 100, about 1250, about 1500, about 1750, about 2000, about 2250, or about 2500 base pairs.

In some embodiments, the modification in the target sequence is inactivation of expression of the target sequence in the cell. For example, upon the binding of a CRISPR complex to the target sequence, the target sequence is inactivated such that the sequence is not transcribed, the coded protein is not produced, or the sequence does not function as the wild-type sequence does. For example, a protein or microRNA coding sequence may be inactivated such that the protein is not produced.

In some embodiments, a regulatory sequence can be inactivated such that it no longer functions as a regulatory sequence. Examples of a regulatory sequence include a promoter, a transcription terminator, an enhancer, and other regulatory elements described herein. The inactivated target sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). In some embodiments, the inactivation of a target sequence results in “knockout” of the target sequence.

In some embodiments, the stiCas9 and guide polynucleotide form a complex, and the guide polynucleotide hybridizes to the target sequence to be modified. In some embodiments, the stiCas9 generates cohesive ends in the target sequence that is hybridized to the guide polynucleotide.

In embodiments of the method, the cohesive ends generated by the stiCas9 comprise a single-stranded polynucleotide overhang of 3 to 40 nucleotides. In some embodiments, the cohesive ends generated by the stiCas9 comprise a single-stranded polynucleotide overhang of 4 to 20 nucleotides. In some embodiments, the cohesive ends generated by the stiCas9 comprise a single-stranded polynucleotide overhang of 5 to 15 nucleotides. In some embodiments, the cohesive ends generated by the stiCas9 is a 5′ overhang.

In embodiments of the method, the stiCas9 is derived from a bacterial species having a Type II-B CRISPR system. As discussed herein, Type II-B Cas9 proteins belong to the TIGR03031 TIGRFAM protein family. Thus, in some embodiments, the stiCas9 of the present disclosure comprises a domain that matches the TIGR03031 protein family with a 1E-5 profile cut-off value. In some embodiments, the stiCas9 of the present disclosure comprises a domain that matches the TIGR03031 protein family with a 1E-10 profile cut-off value. In some embodiments, the stiCas9 of the present disclosure comprises a domain that matches the TIGR03031 protein family with an E-value cut-off of at least 1E-10, at least 1E-9, at least 1E-8, at least 1E-7, at least 1E-6, at least 1E-5, at least 1E-4, at least 1E-3, at least 1E-2, or at least 1E-1.

In embodiments of the method, the Type II-B Cas9 is derived from any species having a Type II-B CRISPR system. In some embodiments, the Type II-B Cas9 is derived from the following bacterial species: Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1_47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonassp. S-B4-1U, Tamilnaduibacter salinus, Vibrio natriegens, Arcobacter skirrowii, Francisella philomiragia, Francisella hispaniensis, or Parendozoicomonas haliclonae.

In embodiments of the method, the guide polynucleotide is guide RNA. In some embodiments, the guide polynucleotide comprises at least two nucleotide segments: at least one “DNA-binding segment” or “guide sequence” and at least one “polypeptide-binding segment.” In some embodiments, the DNA-binding segment of the guide polynucleotide hybridizes with a target sequence in a eukaryotic cell, but not a sequence in a bacterial cell. In some embodiments, the polypeptide-binding segment of the guide polynucleotide binds to Cas9. In some embodiments, the polypeptide-binding segment of the guide polynucleotide binds to stiCas9.

In embodiments of the method, the guide polynucleotide is 10 to 35 nucleotides. In some embodiments, the guide polynucleotide is 15 to 30 nucleotides. In some embodiments, the guide polynucleotide is 20 to 25 nucleotides.

In embodiments of the method, the stiCas9 and the guide polynucleotide are capable of forming a complex. In some embodiments, a complex is formed when all the components of the complex are present together, i.e., a self-assembling complex. In some embodiments, a complex is formed through chemical interactions between different components of the complex such as, for example, hydrogen-bonding. In some embodiments, a guide polynucleotide forms a complex with a stiCas9 through secondary structure recognition of the guide polynucleotide by the stiCas9. In some embodiments, a stiCas9 protein is inactive, i.e., does not exhibit nuclease activity, until it forms a complex with a guide polynucleotide. Binding of guide RNA induces a conformational change in stiCas9 to convert the stiCas9 from the inactive form to an active, i.e., catalytically active, form. In embodiments of the method, the complex of the stiCas9 and guide polynucleotide does not occur in nature.

In embodiments of the method, the cohesive ends generated by the stiCas9 are ligated together (i.e., joined together chemically). Ligation can be performed, for example, by DNA ligase such as T4 ligase or DNA ligase IV. In some embodiments, the cohesive ends are ligated together with an error prone ligase that introduces one or more nucleotide substitutions. In some embodiments, a polynucleotide sequence of interest (SoI) is ligated to the cohesive ends. In some embodiments, the SoI comprises a mutation of interest.

In embodiments of the method, cohesive ends are generated in the SoI complementary to the cohesive ends generated in the target sequence. In some embodiments, cohesive ends in the SoI are generated by a stiCas9. In some embodiments, the SoI is ligated into the cohesive ends using an endogenous DNA repair pathway of the cell. Endogenous DNA repair pathways are described herein.

In some embodiments, the present disclosure provides a method for providing site-specific modification of a target sequence in a eukaryotic cell, the method comprising: (1) introducing into the cell: (a) a nucleotide sequence encoding a Cas9 effector protein capable of generating cohesive ends (stiCas9), and (b) a guide polynucleotide that forms a complex with the stiCas9 and comprises a guide sequence, wherein the guide sequence is capable of hybridizing with the target sequence in the eukaryotic cell but does not hybridize to a sequence in a bacterial cell, wherein the complex does not occur in nature; (2) generating cohesive ends in the target sequence with the Cas9 effector protein and the guide polynucleotide; and (3) ligating: (a) the cohesive ends together, or (b) a polynucleotide sequence of interest (SoI) to the cohesive ends, thereby modifying the target sequence.

In embodiments of the method, the stiCas9 is encoded by a nucleotide sequence. In some embodiments, the nucleotide is DNA. In some embodiments, the stiCas9 protein comprises a domain comprising a sequence having at least 70%, at least 75%, at least 80%, at least 85%, at least 90%, at least 95%, at least 96%, at least 97%, at least 98%, at least 99%, or 100% identity with the nucleotide sequence of any of SEQ ID NOs: 10-97 or 192-195.

In embodiments of the method, the CRISPR-Cas systems of the present disclosure further comprise a tracrRNA. In some embodiments, the guide RNA comprises the crRNA/tracrRNA hybrid. In some embodiments, the tracrRNA component of the guide RNA activates the Cas9 protein. In embodiments of the method, the stiCas9, guide polynucleotide, and tracrRNA are capable of forming a complex. In some embodiments, the complex of the stiCas9, guide polynucleotide, and tracrRNA does not occur in nature.

In embodiments of the method, the complex comprising stiCas9 and a guide polynucleotide is capable of cleaving at a site within 10 nucleotides of a Protospacer Adjacent Motif (PAM). In some embodiments, the complex comprising stiCas9 and a guide polynucleotide is capable of cleaving at a site within 5 nucleotides of a PAM. In some embodiments, the complex comprising stiCas9 and a guide polynucleotide is capable of cleaving at a site within 3 nucleotides of a PAM. In some embodiments, the PAM is downstream (i.e., 3′ direction) of the target sequence. In some embodiments, the PAM is upstream (i.e., 5′ direction) of the target sequence. In some embodiments, the PAM is located within the target sequence.

In embodiments of the method, the PAM comprises a 3′ G-rich motif. In some embodiments, the PAM sequence is NGG, wherein N is A, C, T, U, or G. In some embodiments, the PAM sequence is NGA, wherein N is A, C, T, U, or G. In some embodiments, the PAM sequence is YG, wherein Y is a pyrimidine (i.e., C, T, or U). In embodiments of the method, the target sequence is 5′ of a PAM and the PAM comprises a 3′ G-rich motif. In some embodiments, the target sequence is 5′ of a PAM and the PAM sequence is NGG, wherein N is A, C, T, U, or G.

In embodiments of the method, the eukaryotic cell is an animal or human cell. In some embodiments, the eukaryotic cell is an animal cell. In some embodiments, the eukaryotic cell is a human cell, including human stem cell. In some embodiments, the eukaryotic cell is a plant cell. Examples of various types of eukaryotic cells are provided herein. In embodiments of the method, the stiCas9 and guide polynucleotide are introduced into the eukaryotic cell via a delivery particle. In embodiments of the method, the stiCas9 and guide polynucleotide are introduced into the eukaryotic cell via a vesicle. In embodiments of the method, the stiCas9 and guide polynucleotide are introduced into the eukaryotic cell via a vector. In embodiments of the method, the stiCas9 and the guide polynucleotide are introduced into the eukaryotic cell via a viral vector. In embodiments of the method, the polynucleotides encoding components of the complex comprising a stiCas9 and guide polynucleotide are introduced on one or more vectors. Examples of vectors and methods of vector delivery into cells (e.g., transfection) are provided herein.

In some embodiments, the methods of the present disclosure further comprise introducing into a eukaryotic cell an exonuclease to remove overhangs generated from the stiCas9. In some embodiments, the exonuclease is a 5′ to 3′ exonuclease. In some embodiments, the exonuclease is a 3′ to 5′ exonuclease. In some embodiments, the exonuclease is added prior to the ligation step of the method. In some embodiments, the exonuclease is added instead of the ligation step of the method. Non-limiting examples of 5′ to 3′ exonucleases include: Lambda Exonuclease, RecJ, Exonuclease V, Exonuclease VIII, T5 Exonuclease, T7 Exonuclease, Artemis, and Cas4. Non-limiting examples of 3′ to 5′ exonucleases include: TREX1, TREX2, Werner syndrome (WRN) protein, p53, MRE11, RAD1, RAD9, APE1, and VDJP protein. In some embodiments, the exonuclease is Cas4, Artemis, or TREX2.

Introduction of Cas4, Artemis, TREX2, or other similar exonucleases allows the end processing of cohesive ends before ligation occurs, thereby decreasing the chance of precise ligations and thus increasing the efficiency of mutagenesis, competing with endogenous DNA repair enzymes to bias the repair towards one of the other repair pathways (e.g., NHEJ or MMEJ), and modulating the mutation patterns. For example, Cas4, Artemis, or TREX2 may increase the efficiency of mutagenesis by competing with endogenous end processing enzymes, thus promoting error-prone repairs. Cas4, Artemis, or TREX2 may also facilitate HDR repair by elongating the single-strand overhangs. A further role for Cas4, Artemis, or TREX2 may, for example, involve changing mutation patterns towards more desirable indels.

### Methods for Site-Specific Gene Insertions (ObLiGaRe 2.0)

In some embodiments, the present disclosure provides a method of introducing a sequence of interest (SoI) into a chromosome in a cell based on a derivation of the ObLiGaRe method described in U.S. Pat. No. 9,567,608. ObLiGaRe (Obligated Ligation-Gated Recombination) reflects the etymologic meaning of the Latin verb obligare (to ligate head to head). It is broadly applicable in different cell lines and provides an additional approach for genetic engineering. Whereas U.S. Pat. No. 9,567,608 employed zinc finger nucleases to target and cleave the target sequence, the disclosure herein provides for the use of a first Cas9-endonuclease dimer, e.g., Cas9-FokI, and a second Cas9-endonuclease dimer. The methods for site-specific gene insertions described herein are informally referred to “ObLiGaRe 2.0” as a shorthand, to distinguish it from the ObLiGaRe method described in U.S. Pat. No. 9,567,608.

In some embodiments, the present disclosure provides a method of introducing a sequence of interest (SoI) into a chromosome in a cell, wherein the chromosome comprises a target sequence (TSC) comprising region 1 and region 2, the method comprising introducing into the cell: (a) a vector comprising a target sequence (TSV), the TSV comprising region 2 and region 1 and the SoI; (b) a first Cas9-endonuclease dimer capable of generating cohesive ends in the TSC, wherein a first monomer of the first Cas9-endonuclease dimer cleaves at region 1 and a second monomer of the first Cas9-endonuclease dimer cleaves at region 2 of the TSC; and (c) a second Cas9-endonuclease dimer capable of generating cohesive ends in the TSV, wherein a first monomer of the second Cas9-endonuclease dimer cleaves at region 2 and a second monomer of the second Cas9-endonuclease dimer cleaves at region 1 of the TSV, and wherein introduction of the vector of (a), the first Cas9-endonuclease dimer of (b) and the second Cas9-endonuclease dimer of (c) results in insertion of the SoI into the chromosome of the cell.

In some embodiments, the disclosure is directed to a method of introducing a sequence of interest (SoI) into a chromosome in a cell, wherein the chromosome comprises a target sequence (TSC) comprising region 1 and region 2, the method comprising introducing into the cell: (a) a vector comprising a target sequence (TSV), the TSV comprising region 2 and region 1 and the SoI, wherein the vector comprises cohesive ends; and (b) a first Cas9-endonuclease dimer capable of generating cohesive ends in the TSC, wherein a first monomer of the first Cas9-endonuclease dimer cleaves at region 1 and a second monomer of the first Cas9-endonuclease dimer cleaves at region 2 of the TSC; wherein introduction of the vector of (a) and the first Cas9-endonuclease dimer of (b) results in insertion of the SoI into the chromosome of the cell.

The method of the present disclosure provides efficient and precise gene targeting without homology in the vector (or “donor plasmid”). The method of the present disclosure provides a strategy of site-specific gene insertion using the Non-Homologous End Joining (NHEJ) or Microhomology-Mediated End Joining (MMEJ) pathways. The design and location of the cleavage sites (i.e., region 1 and region 2) in the vector is sufficient to achieve precise end joining of the vector in the cleavage sites (i.e., region 1 and region 2) in the genomic site, i.e., the target sequence in the chromosome of the cell (TSC).

In some embodiments, the TSV is a circular vector, i.e., a plasmid. In some embodiments, the TSV is a linearized vector or linear DNA, such as, for example, a PCR product, or an annealed oligonucleotide duplex with complementary ends to the TSC after cleavage. In some embodiments, the TSV comprises cohesive ends. In some embodiments, the cohesive ends in the TSV are generated by a Cas9-endonuclease dimer. In some embodiments, the cohesive ends in the TSV are generated prior to introduction of the TSV into a cell. In some embodiments, the cohesive ends in the TSV are generated after introduction of the TSV into a cell.

In some embodiments, the target sequence on the chromosome (TSC) comprises, in a 5′ to 3′ manner, region 1 and region 2. As used herein, the directionality of a sequence (e.g., 5′ to 3′) refers to the direction when reading the “coding” strand or “sense” strand of a double-stranded DNA sequence (typically presented as the top strand of a double-stranded DNA sequence).

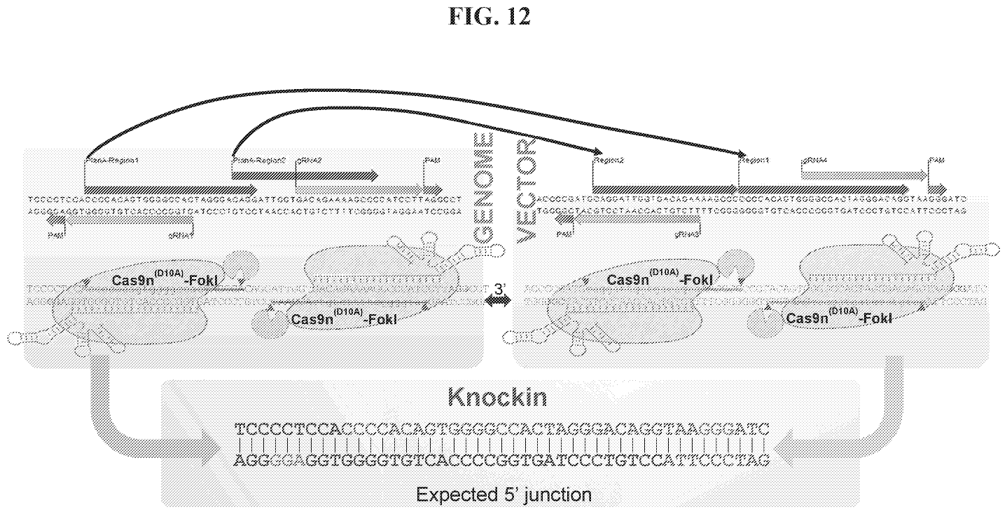

FIG. 12 represents an embodiment of the present disclosure. In FIG. 12, the TSC is represented by the sequence in the “Genome” box (left) and comprises: Region 1 and Region 2 (a portion of which is overlapping with Region 1) on the “coding” strand (shown as the top strand).

As shown in the “Genome” box of FIG. 12, upstream (i.e., 5′ with respect to the coding strand) of Region 1 and on the “non-coding” or “anti-sense” DNA strand (shown as the bottom strand), there is a first PAM sequence. The non-coding strand comprises a region that hybridizes to a first guide polynucleotide (“gRNA1”). gRNA1 hybridizes to a sequence upstream (i.e., 5′ with respect to the non-coding strand) of the first PAM sequence. This gRNA1 hybridization sequence includes a portion of Region 1 and additionally several nucleotides outside of Region 1. As indicated by the direction of the arrows, gRNA1 hybridizes with the non-coding strand of the target sequence.

As shown in the “Genome” box of FIG. 12, downstream (i.e., 3′ with respect to the coding strand) of Region 2 and on the coding strand, there is a second PAM sequence. The coding strand comprises a region that hybridizes to a second guide polynucleotide (“gRNA2”). gRNA2 hybridizes to a sequence upstream (i.e., 5′ with respect to the coding strand) of the second PAM sequence. This gRNA2 hybridization sequence includes a portion of Region 2 and additionally several nucleotides outside of Region 2. As indicated by the direction of the arrows, gRNA2 hybridizes with the coding strand of the target sequence.

In some embodiments, the target sequence on the vector (TSV) comprises, in a 5′ to 3′ manner, region 2, immediately followed by region 1, and the SoI. FIG. 12 represents an embodiment of the present disclosure. In FIG. 12, the TSV is represented by the sequence in the “Vector” box (right) and comprises: Region 2, followed by Region 1 (without any overlap between the two regions) on the “coding” strand.

As shown in the “Vector” box of FIG. 12, upstream (i.e., 5′ with respect to the coding strand) of Region 2 and on the “non-coding,” there is a third PAM sequence. The non-coding strand comprises a region that hybridizes to a third guide polynucleotide (“gRNA3”). gRNA3 hybridizes to a sequence upstream (i.e., 5′ with respect to the non-coding strand) of the third PAM sequence. This gRNA3 hybridization sequence includes a portion of Region 2 and additionally several nucleotides outside of Region 2. As indicated by the direction of the arrows, gRNA3 hybridizes with the non-coding strand of the target sequence.

As shown in the “Vector” box of FIG. 12, downstream (i.e., 3′ with respect to the coding strand) of Region 1 and on the coding strand, there is a fourth PAM sequence. The coding strand comprises a region that hybridizes to a fourth guide polynucleotide (“gRNA4”). gRNA4 hybridizes to a sequence upstream (i.e., 5′ with respect to the coding strand) of the fourth PAM sequence. This gRNA4 hybridization sequence includes a portion of Region 1 and additionally several nucleotides outside of Region 1. As indicated by the direction of the arrows, gRNA4 hybridizes with the coding strand of the target sequence.

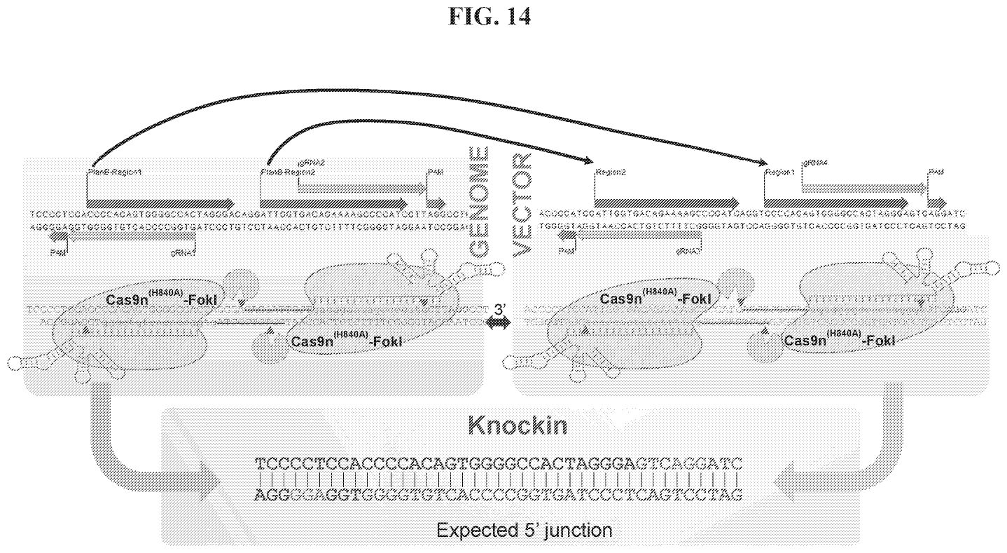

FIG. 14 represents another embodiment of the present disclosure. FIG. 14 is similar to FIG. 14, except that there is a gap of several nucleotides between Region 1 and Region 2 on the TSC, and that there is a gap of several nucleotides between Region 2 and Region 1 on the TSV. However, the arrangement of the regions relative to one another, and the directionality of the guide polynucleotides are the same in FIG. 14 and FIG. 12.

Thus, in some embodiments, the target sequence on the chromosome (i.e., the TSC) comprises region 1 and region 2, wherein a portion of region 1 overlaps with a portion of region 2. In other embodiments, the TSC comprises region 1 and region 2, wherein region 1 and region 2 are separated by one or more nucleotides. In some embodiments, region 1 and region 2 overlap by 1, 2, 3, 4, 5, 6, 7, 8, 9, 10 or more nucleotides. In some embodiments, region 1 and region 2 are separated by 1, 2, 3, 4, 5, 6, 7, 8, 9, 10 or more nucleotides.

In some embodiments, the target sequence on the vector (i.e., the TSV) comprises region 2 and region 1, wherein region 2 immediately precedes region 1 without any nucleotides in between. In other embodiments, the TSV comprises region 2 and region 1, wherein region 2 and region 1 are separated by 1 or more nucleotides. In some embodiments, region 2 and region 1 are separated by 1, 2, 3, 4, 5, 6, 7, 8, 9, 10 or more nucleotides.

In embodiments of the method, a Cas9-endonuclease dimer generates cohesive ends in the target sequence. As described herein, Cas9 proteins generate site-specific breaks in a nucleic acid. In some embodiments, Cas9 proteins generate site-specific double-stranded breaks in DNA. The ability of Cas9 to target a specific sequence in a nucleic acid (i.e., site specificity) is achieved by the Cas9 complexing with a guide polynucleotide, e.g., guide RNA, that hybridizes with the specified sequence. Thus, a complex comprising a Cas9 and guide polynucleotide has at least two distinct functions: (1) specific targeting of a nucleic acid sequence, and (2) nuclease activity generating a break at or near the targeted nucleic acid sequence. In some embodiments, a Cas9-guide polynucleotide complex is modified such that it performs only one of the two functions. In some embodiments, a Cas9 is modified to remove nuclease activity, but retains the ability to complex with a guide polynucleotide such that the Cas9 can still target a specific nucleic acid sequence.

As described herein, wild-type Cas9 is a monomeric protein comprising a nucleic acid-binding domain (which interacts with a guide polynucleotide) and a cleavage domain (which cleaves the target nucleic acid). In certain instances, it is advantageous to use a dimeric nuclease, i.e., a nuclease which is not active until both monomers of the dimer are present at the target sequence, in order to achieve higher targeting specificity. Binding domains and cleavage domains of naturally-occurring nucleases (such as, e.g., Cas9), as well as modular binding domains and cleavage domains that can be fused to create nucleases binding specific target sites, are well known to those of skill in the art. For example, the binding domain of RNA-programmable nucleases (e.g., Cas9), or a Cas9 protein having an inactive DNA cleavage domain, can be used as a binding domain (e.g., that binds a gRNA to direct binding to a target site) to specifically bind a desired target site, and fused or conjugated to a cleavage domain, for example, the cleavage domain of the endonuclease FokI, to create an engineered nuclease cleaving the target site. Cas9-FokI fusion proteins are further described in, e.g., U.S. Patent Publication No. 2015/0071899 and Guilinger et al., “Fusion of catalytically inactive Cas9 to FokI nuclease improves the specificity of genome modification,” Nature Biotechnology 32: 577-582 (2014), each of which is incorporated by reference herein in its entirety.

In some embodiments, the engineered nuclease recognizes a palindromic, double-stranded target site, for example, a double-stranded DNA target site. The target sites of many naturally-occurring nucleases such as, for example, naturally-occurring DNA restriction nucleases, are well-known to those of skill in the art. In some embodiments, a DNA nuclease such as, e.g., EcoRI, HindIII, or BamHI, recognizes a palindromic, double-stranded DNA target site of 4 to 10 base pairs in length and cuts each of the two DNA strands at a specific position within the target site. In some embodiments, an endonuclease cuts a double-stranded nucleic acid target site symmetrically, i.e., cutting both strands at the same position so that the ends comprise base-paired nucleotides, also referred to herein as blunt ends. In some embodiments, an endonuclease cuts a double-stranded nucleic acid target site asymmetrically, i.e., cutting each strand at a different position so that the ends comprise unpaired nucleotides, i.e., cohesive ends or overhangs. In some embodiments, the overhangs are 5′-overhangs, i.e., the unpaired nucleotides form the 5′ end of the DNA strand. In some embodiments, the overhangs are 3′-overhangs, i.e., the unpaired nucleotides form the 3′ end of the DNA strand. Overhangs can “stick” to (i.e., joined with) other double-stranded DNA molecule ends comprising complementary unpaired nucleotides.

In some embodiments, fusion proteins are provided comprising two domains: (i) an RNA-programmable nuclease (e.g., Cas9 protein, or fragment thereof) domain fused or linked to (ii) a nuclease domain. For example, in some embodiments, the Cas9 protein (e.g., the Cas9 domain of the fusion protein) comprises a nuclease-inactivated Cas9 (e.g., a Cas9 lacking DNA cleavage activity; “dCas9”) that retains RNA (gRNA) binding activity and is thus able to bind a target site complementary to a gRNA. In some embodiments, the nuclease fused to the nuclease-inactivated Cas9 domain is any nuclease requiring dimerization (e.g., the coming together of two monomers of the nuclease) in order to cleave a target nucleic acid (e.g., DNA). In some embodiments, the nuclease fused to the nuclease-inactivated Cas9 is a monomer of the FokI DNA cleavage domain, thereby producing the Cas9 variant referred to as Cas9-FokI. The FokI DNA cleavage domain is known, and in embodiments corresponds to amino acids 388-583 of FokI (NCBI accession number J04623). In some embodiments, the FokI DNA cleavage domain corresponds to amino acids 300-583, 320-583, 340-583, or 360-583 of FokI. (See also Wah et al., “Structure of FokI has implications for DNA cleavage,” Proceedings of the National Academy of Sciences USA 95(18): 10564-9 (1996); Li et al., “TAL nucleases (TALNs): hybrid proteins composed of TAL effectors and FokI DNA-cleavage domain,” Nucleic Acids Research 39(1): 359-72 (2011); Kim et al., “Hybrid restriction enzymes: zinc finger fusions to FokI cleavage domain,” Proceedings of the National Academy of Sciences USA 93: 1156-1160 (1996); each of which is herein incorporated by reference in its entirety.)

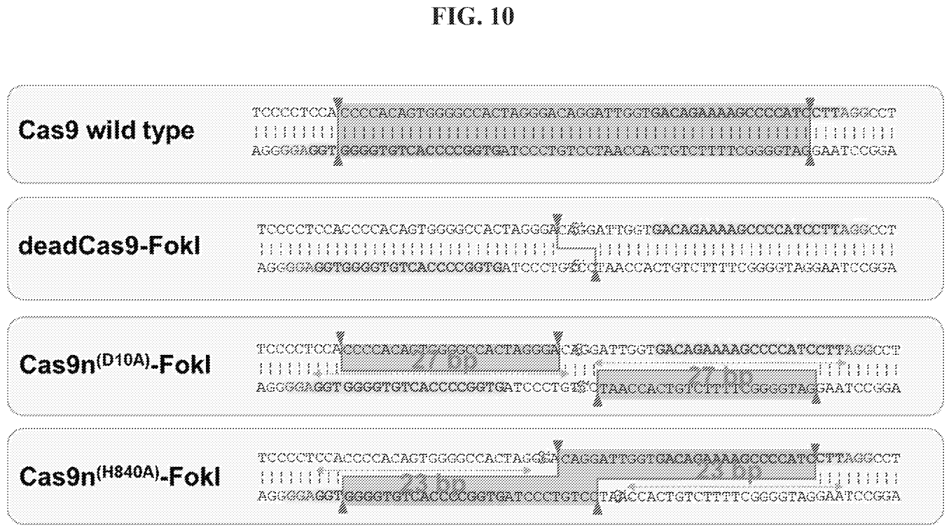

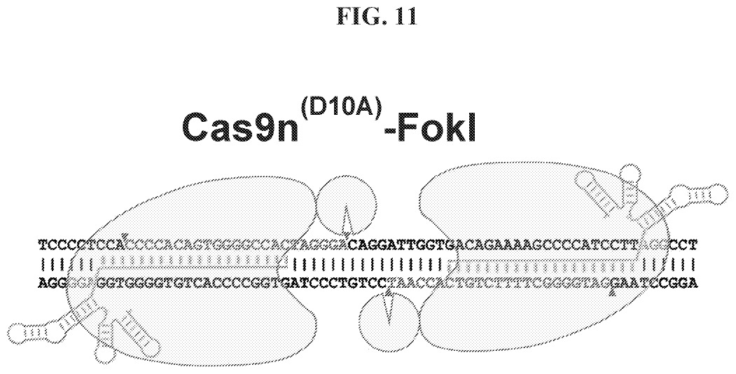

In some embodiments, a dimer of the Cas9-endonuclease fusion protein is provided, e.g., dimers of Cas9-FokI. For example, in some embodiments, the Cas9-FokI fusion protein forms a dimer with itself to mediate cleavage of the target nucleic acid. In some embodiments, the Cas9-endonuclease fusion proteins, or dimers thereof, are associated with one or more gRNAs. In some embodiments, because the dimer contains two fusion proteins, each having a Cas9 domain having gRNA binding activity, a target nucleic acid is targeted using two distinct gRNA sequences that complement two distinct regions of the nucleic acid target. See, e.g., FIGS. 10 and 11. Thus, in some embodiments, cleavage of the target nucleic acid does not occur until both fusion proteins bind the target nucleic acid (e.g., as specified by the gRNA:target nucleic acid base pairing), and the nuclease domains dimerize (e.g., the FokI DNA cleavage domains; as a result of their proximity based on the binding of the Cas9:gRNA domains of the fusion proteins) and cleave the target nucleic acid, e.g., in the region between the bound Cas9 fusion proteins. This is exemplified by the schematics shown in FIGS. 10 and 11. This approach represents a notable improvement over wild type Cas9 and other Cas9 variants, such as the nickases (Ran et al., “Double Nicking by RNA-Guided CRISPR Cas9 for Enhanced Genome Editing Specificity,” Cell 154: 1380-1389 (2013); Mali et al., “CAS9 transcriptional activators for target specificity screening and paired nickases for cooperative genome engineering,” Nature Biotechnology 31: 833-838 (2013)), which do not require the dimerization of nuclease domains to cleave a nucleic acid. These nickase variants can induce cleaving, or nicking upon binding of a single nickase to a nucleic acid, which can occur at on- and off-target sites, and nicking is known to induce mutagenesis. As the variants provided herein require the binding of two Cas9 variants in proximity to one another to induce target nucleic acid cleavage, the chances of inducing off-target cleavage is reduced. In some embodiments, a Cas9 variant fused to a nuclease domain (e.g., Cas9-FokI) has an on-target:off-target modification ratio that is at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, at least 150-fold, at least 175-fold, at least 200-fold, at least 250-fold, or more higher than the on-target:off-target modification ratio of a wild type Cas9 or other Cas9 variant (e.g., nickase). In some embodiments, a Cas9 variant fused to a nuclease domain (e.g., Cas9-FokI) has an on-target:off-target modification ratio that is between about 60- to 180-fold, between about 80- to 160-fold, between about 100- to 150-fold, or between about 120- to 140-fold higher than the on-target:off-target modification ratio of a wild type Cas9 or other Cas9 variant. Methods for determining on-target:off-target modification ratios are known. In some embodiments, the on-target:off-target modification ratios are determined by measuring the number or amount of modifications of known Cas9 off-target sites in certain genes. For example, the Cas9 off-target sites of the CLTA, EMX, and VEGF genes are known, and modifications at these sites can be measured and compared between test proteins and controls. The target site and its corresponding known off-target sites are amplified from genomic DNA isolated from cells (e.g., HEK293) treated with a particular Cas9 protein or variant. The modifications are then analyzed by high-throughput sequencing. Sequences containing insertions or deletions of two or more base pairs in potential genomic off-target sites and present in significantly greater numbers (p value <0.005, Fisher's exact test) in the target gRNA-treated samples versus the control gRNA-treated samples are considered Cas9 nuclease-induced genome modifications.

In some embodiments, the method of the present disclosure provides a dimer of Cas9-endonuclease comprising a first Cas9-endonuclease monomer and a second Cas9-endonuclease monomer. In embodiments of the method, the endonucleases of the Cas9-endonucleases are Type IIS endonucleases. In some embodiments, the endonuclease of the first monomer in the first Cas9-endonuclease dimer is a Type IIS endonuclease. In some embodiments, the endonuclease of the second monomer in the first Cas9-endonuclease dimer is a Type IIS endonuclease. In some embodiments, the endonuclease of the first monomer and the second monomer in the first Cas9-endonuclease dimer are Type IIS endonucleases. In some embodiments, the endonuclease of the first monomer in the second Cas9-endonuclease dimer is a Type IIS endonuclease. In some embodiments, the endonuclease of the second monomer in the second Cas9-endonuclease dimer is a Type IIS endonuclease. In some embodiments, the endonuclease of the first monomer and the second monomer in the second Cas9-endonuclease dimer are Type IIS endonucleases. In some embodiments, the endonucleases in the first Cas9-endonuclease dimer and the second Cas9-endonuclease dimer are Type IIS endonucleases.

Endonucleases, or restriction enzymes, are traditionally classified into four types on the basis of subunit composition, cleavage position, sequence specificity, and cofactor requirements. However, amino acid sequencing has uncovered extraordinary variety among restriction enzymes and revealed that at the molecular level, there are many more than four different types.

“Type IIS” endonucleases are those like FokI and AlwI that cleave outside of their recognition sequence to one side. Type IIS restriction enzymes are intermediate in size, 400-650 amino acids in length, and they recognize sequences that are continuous and asymmetric. They comprise two distinct domains, one for DNA binding, the other for DNA cleavage. They are thought to bind to DNA as monomers for the most part, but to cleave DNA cooperatively, through dimerization of the cleavage domains of adjacent enzyme molecules. For this reason, some Type IIS enzymes are much more active on DNA molecules that contain multiple recognition sites. Non-limiting examples of Type IIS endonucleases include: AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, CspCI, EarI, EciI, FauI, FokI, HgaI, HphI, HpyAV, MboII, MlyI, MmeI, MnlI, NmeAIII, PleI, SapI, and SfaNI. In some embodiments, the endonuclease in the first Cas9-endonuclease dimer and the second Cas9-endonuclease dimer are independently selected from the group consisting of: BbvI, BgcI, BfuAI, BmpI, BspMI, CspCI, FokI, MboII, MmeI, NmeAIII, and PleI. In some embodiments, the endonuclease in the first Cas9-endonuclease dimer and the second Cas9-endonuclease dimer are FokI. DNA cleavage by FokI only occurs upon dimerization of two FokI monomers. FokI cleavage of DNA generates cohesive ends with a 4 base-pair overhang.

Endonucleases in the Cas9-endonuclease fusion proteins can also be engineered FokI nucleases, e.g., engineered FokI dimers. In some embodiments, the engineered FokI dimers are obligatory heterodimers, i.e., two non-identical monomers are required to form a functional (catalytically active) dimer.

In some embodiments, the first and second Cas9-endonuclease dimers are the same. In some embodiments, the first and second Cas9-endonuclease dimers are different.

In some embodiments, the present method provides that the first, second, or both Cas9-endonuclease dimers comprise a modified Cas9. In some embodiments, the modified Cas9 is a catalytically inactive Cas9 (“deadCas9”). In some embodiments, the first, second, or both Cas9-endonuclease dimers comprise a catalytically inactive Cas9. Catalytically inactive Cas9 are incapable of cleaving DNA (i.e., the cleavage domain of Cas9 is inactivated); however, they retain the ability to target a nucleic acid sequence by forming a complex with a guide polynucleotide (e.g., guide RNA). Catalytically inactive Cas9 have been described in the art, e.g., by Jinek et al. (2012) and Qi et al., “Repurposing CRISPR as an RNA-guided platform for sequence-specific control of gene expression,” Cell 152(5): 1173-1183 (2013). In some embodiments, catalytically inactive Cas9 comprises a double amino-acid substitution relative to wild-type Cas9. In some embodiments, the Cas9-endonuclease dimer comprises a double amino-acid substitution relative to wild-type Cas9. In some embodiments, the double amino-acid substitution is D10A and H840A. In some embodiments, the endonuclease in the first, second, or both Cas9-endonuclease dimers is FokI and the Cas9 in the first, second, or both Cas9-endonuclease dimers is a catalytically inactive Cas9 (“deadCas9-FokI”). In some embodiments, the endonuclease in the first, second, or both Cas9-endonuclease dimers is FokI and the Cas9 in the first, second, or both Cas9-endonuclease dimers comprises the D10A/H840A double amino-acid substitution.

In some embodiments, the modified Cas9 is a Cas9 having nickase activity (“Cas9 nickase” or “Cas9n”). In some embodiments, the first, second, or both Cas9-endonuclease dimers comprise a Cas9 having nickase activity. Cas9 nickases are capable of cleaving only one strand of double-stranded DNA (i.e., “nicking” the DNA). Cas9 nickases are described in, e.g., Cho et al., “Analysis of off-target effects of CRISPR/Cas-derived RNA-guided endonucleases and nickases,” Genome Research 24: 132-141 (2013), Ran et al. (Cell 2013), and Mali et al. (Nature Biotechnology 2013). In some embodiments, Cas9 nickases comprise a single amino-acid substitution relative to wild-type Cas9. In some embodiments, the Cas9-endonuclease dimer comprises a single amino-acid substitution relative to wild-type Cas9. In some embodiments, the single amino-acid substitution is D10A (“Cas9n^{(D10A)}”). In some embodiments, the single amino-acid substitution is H840A (“Cas9n^{(H840A)}”). In some embodiments, the endonuclease in the first, second, or both Cas9-endonuclease dimers is FokI and the Cas9 in the first, second, or both Cas9-endonuclease dimers is a Cas9 nickase. In some embodiments, the endonuclease in the first, second, or both Cas9-endonuclease dimers is FokI and the Cas9 in the first, second, or both Cas9-endonuclease dimers comprises the D10A single amino-acid substitution (“Cas9n^{(D10A)}-FokI”). In some embodiments, the endonuclease in the first, second, or both Cas9-endonuclease dimers is FokI and the Cas9 in the first, second, or both Cas9-endonuclease dimers comprises the H8410A single amino-acid substitution (“Cas9n^{(H840A)}-FokI”).

In some embodiments, the wild-type Cas9 is derived from Streptococcus pyogenes, Staphylococcus aureus, Staphylococcus pseudintermedius, Planococcus antarcticus, Streptococcus sanguinis, Streptococcus thermophilus, Streptococcus mutans, Coribacterium glomerans, Lactobacillus farciminis, Catenibacterium mitsuokai, Lactobacillus rhamnosus, Bifidobacterium bifidum, Oenococcus kitahara, Fructobacillus fructosus, Finegoldia magna, Veillonella atyipca, Solobacterium moorei, Acidaminococcus sp. D21, Eubacterium yurri, Coprococcus catus, Fusobacterium nucleatum, Filifactor alocis, Peptoniphilus duerdenii, or Treponema denticola.

In some embodiments, the cohesive ends generated by the Cas9-endonuclease comprise a 5′ overhang. In some embodiments, the cohesive ends generated by the Cas9-endonuclease comprise a 3′ overhang. In some embodiments, the first, second, or both Cas9-endonuclease dimers generate cohesive ends comprising a single-stranded polynucleotide of 3 to 40 nucleotides. In some embodiments, the first, second, or both Cas9-endonuclease dimers generate cohesive ends comprising a single-stranded polynucleotide of 4 to 30 nucleotides. In some embodiments, the first, second, or both Cas9-endonuclease dimers generate cohesive ends comprising a single-stranded polynucleotide of 5 to 20 nucleotides. In some embodiments, the first, second, or both Cas9-endonuclease dimers generate cohesive ends comprising a single-stranded polynucleotide of about 5 nucleotides, about 10 nucleotides, about 15 nucleotides, about 20 nucleotides, about 25 nucleotides, or about 30 nucleotides. In some embodiments, a deadCas9-FokI dimer generates cohesive ends comprising a 4-nucleotide 5′ overhang. In some embodiments, a Cas9n^{(D10A)}-FokI dimer generates cohesive ends comprising a 27-nucleotide 5′ overhang. In some embodiments, a Cas9^{(H840A)}-FokI dimer generates cohesive ends comprising a 23-nucleotide 3′ -overhang.

In embodiments of the method, the sequence of interest (SoI) is comprised by a donor plasmid. The donor plasmid can be of any suitable length, such as about or at least about 10, 15, 20, 25, 50, 75, 100, 150, 200, 250, 500 or 1000 or more nucleotides in length. In some embodiments, the donor plasmid is complementary to a portion of the chromosome comprising the TSC. When optimally aligned, the donor plasmid template overlaps with one or more nucleotides of TSC (e.g., about or at least about 1, 5, 10, 15, 20, 25, 30, 35, 40, 45, 50, 60, 70, 80, 90, or 100 or more nucleotides). In some embodiments, when the donor plasmid and a chromosome comprising the TSC are optimally aligned, the nearest nucleotide of the donor plasmid is within about 1, 5, 10, 15, 20, 25, 50, 75, 100, 200, 300, 400, 500, 100, 1500, 2000, 2500, 5000, 10000 or more nucleotides from the TSC.

In some embodiments, the SoI is DNA, such as, e.g., a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome.

In some embodiments, the SoI is inserted into the TSC using an endogenous DNA repair pathway of the cell. In some embodiments, the SoI is inserted into the TSC using components of the Non-Homologous End Joining (NHEJ) repair pathway. During the repair process, a donor plasmid comprising the SoI can be introduced into the TSC.

In some embodiments, a donor plasmid comprising the SoI flanked by an upstream sequence and a downstream sequence is introduced into the cell, wherein the upstream and downstream sequences share sequence similarity with either side of the site of integration in the TSC. In some embodiments, the exogenous polynucleotide comprising the SoI comprises, for example, a mutated gene. In some embodiments, the exogenous polynucleotide comprises a sequence endogenous or exogenous to the cell. In some embodiments, the SoI comprises polynucleotides encoding a protein, or a non-coding sequence such as, e.g., a microRNA. In some embodiments, the SoI is operably linked to a regulatory element. In some embodiments, the SoI is a regulatory element. In some embodiments, the SoI comprises a resistance cassette, e.g., a gene that confers resistance to an antibiotic. In some embodiments, the SoI comprises a mutation of the wild-type target sequence. In some embodiments, the SoI disrupts the target sequence by creating a frameshift mutation or nucleotide substitution. In some embodiments, the SoI comprises a marker. Introduction of a marker into a target sequence can make it easy to screen for targeted integrations. In some embodiments, the marker is a restriction site, a fluorescent protein, or a selectable marker. In some embodiments, the SoI is introduced as a vector comprising the SoI.

The upstream and downstream sequences in the exogenous polynucleotide template are selected to promote homologous recombination between the target sequence and the exogenous polynucleotide. The upstream sequence is a nucleic acid sequence that shares sequence similarity with the sequence upstream of the targeted site for integration (i.e., the target sequence). Similarly, the downstream sequence is a nucleic acid sequence that shares sequence similarity with the sequence downstream of the targeted site for integration. Thus, in some embodiments, the exogenous polynucleotide template comprising the SoI is inserted into the target sequence by homologous recombination at the upstream and downstream sequences. In some embodiments, the upstream and downstream sequences in the exogenous polynucleotide template has at least 70%, at least 75%, at least 80%, at least 85%, at least 90%, at least 95%, at least 96%, at least 97%, at least 98%, at least 99%, or 100% sequence identity with the upstream and downstream sequences in targeted genome sequence, respectively. In some embodiments, the upstream or downstream sequence has about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In some embodiments, the upstream or downstream sequence has about 50, about 100, about 250, about 500, about 100, about 1250, about 1500, about 1750, about 2000, about 2250, or about 2500 base pairs.

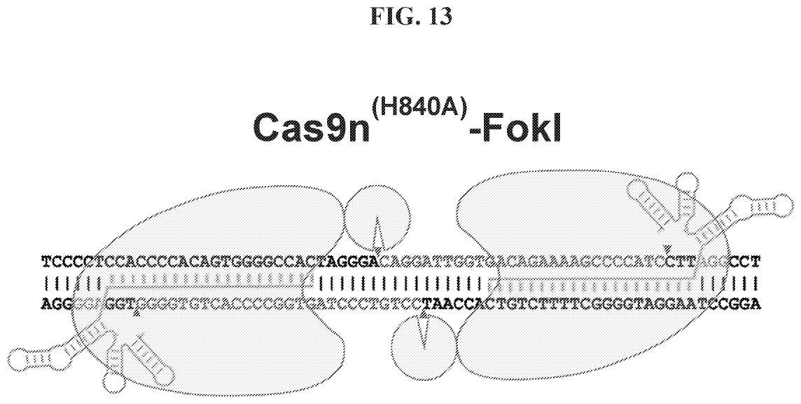

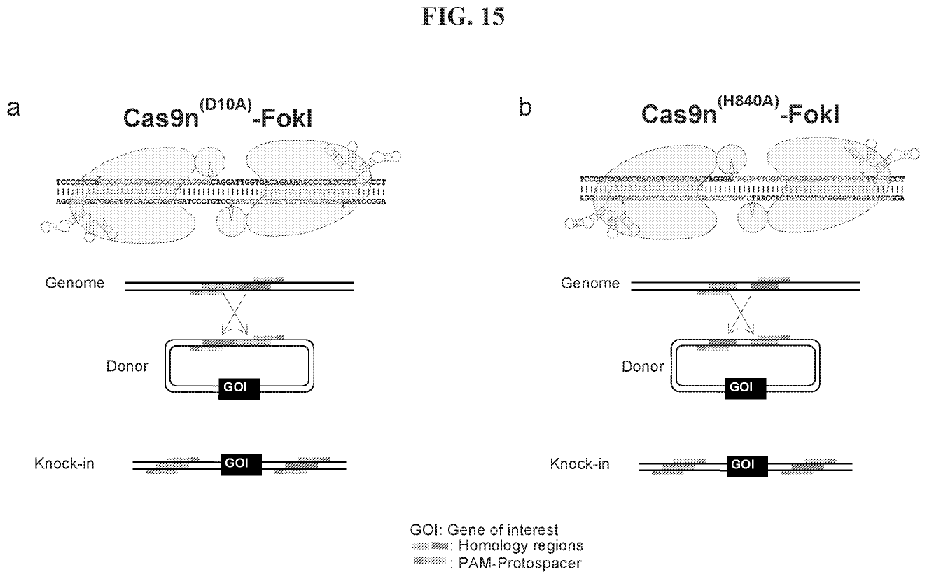

In some embodiments, upon the insertion of the SoI, the target sequence in the chromosome and the target sequence in the plasmid are not reconstituted. That is, in some embodiments, the resulting sequence in the chromosome (i.e., the resulting sequence from insertion of the SoI) does not hybridize to any of the first, second, third, or fourth guide polynucleotides. Thus, in some embodiments, the resulting sequence in the chromosome comprising the SoI is not susceptible to cleavage by the first or second Cas9-endonuclease dimers, or any of the monomers in the first or second Cas9-endonuclease dimers. As exemplified in FIGS. 13 and 15, the resulting “Knockin” sequence (“Expected 5′ junction”) is a different sequence from the “Genome” and “Vector” sequences, and the “Knockin” sequence does not have a hybridizable sequence to any of gRNA1, gRNA2, gRNA3, or gRNA4.

In some embodiments, the method of the present disclosure further comprises introducing into the cell a first guide polynucleotide that forms a complex with the first monomer of the first Cas9-endonuclease dimer and comprises a first guide sequence, wherein the first guide sequence hybridizes to the TSC comprising region 1 but does not hybridize to the vector. As exemplified by FIGS. 13 and 15, the first guide sequence (shown as “gRNA1”) binds to a portion of Region1 as well as several nucleotides outside of Region1 on the non-coding strand of the target DNA in the genome. gRNA1 does not hybridize to any other sequence in the genome or the vector. In some embodiments, the first guide polynucleotide forms a complex with the first monomer of the first Cas9-endonuclease dimer by interaction with the binding domain of the Cas9.

In some embodiments, the method of the present disclosure further comprises introducing into the cell a second guide polynucleotide that forms a complex with the second monomer of the first Cas9-endonuclease dimer and comprises a second guide sequence, wherein the second guide sequence hybridizes to the TSC comprising region 2 but does not hybridize to the vector. As exemplified by FIGS. 13 and 15, the second guide sequence (shown as “gRNA2”) binds to a portion of Region2 on the coding strand of the target DNA in the genome. gRNA2 does not hybridize to any other sequence in the genome or the vector. In some embodiments, the second guide polynucleotide forms a complex with the second monomer of the first Cas9-endonuclease dimer by interacting with the binding domain of the Cas9.

In some embodiments, the method of the present disclosure further comprises introducing into the cell a third guide polynucleotide that forms a complex with the first monomer of the second Cas9-endonuclease dimer and comprises a third guide sequence, wherein the third guide sequence hybridizes to the TSV comprising region 2 but does not hybridize to the genome. As exemplified by FIGS. 13 and 15, the third guide sequence (shown as “gRNA3”) binds to a portion of Region2 as well as several nucleotides outside of Region2 on the non-coding strand of the target DNA in the vector. gRNA3 does not hybridize to any other sequence in the genome or the vector. In some embodiments, the third guide polynucleotide forms a complex with the first monomer of the second Cas9-endonuclease dimer by interaction with the binding domain of the Cas9.

In some embodiments, the method of the present disclosure further comprises introducing into the cell a fourth guide polynucleotide that forms a complex with the second monomer of the second Cas9-endonuclease dimer and comprises a fourth guide sequence, wherein the fourth guide sequence hybridizes to the TSC comprising region 1 but does not hybridize to the genome. As exemplified by FIGS. 13 and 15, the fourth guide sequence (shown as “gRNA4”) binds to a portion of Region1 on the coding strand of the target DNA in the vector. gRNA4 does not hybridize to any other sequence in the genome or the vector. In some embodiments, the fourth guide polynucleotide forms a complex with the second monomer of the second Cas9-endonuclease dimer by interacting with the binding domain of the Cas9.

In some embodiments, a guide polynucleotide is capable of binding to both the TSC and the TSV. Thus, in some embodiments, the method further comprises introducing into the cell a first guide polynucleotide that forms a complex with the first monomer of the first Cas9-endonuclease dimer and comprises a first guide sequence, wherein the first guide sequence hybridizes to the TSC and the TSV.

In some embodiments, the method further comprises introducing into the cell a second guide polynucleotide that forms a complex with the second monomer of the first Cas9-endonuclease dimer and comprises a second guide sequence, wherein the second guide sequence hybridizes to the TSC and the TSV.

In some embodiments, the method further comprises introducing into the cell a third guide polynucleotide that forms a complex with the first monomer of the second Cas9-endonuclease dimer and comprises a third guide sequence, wherein the third guide sequence hybridizes to the TSC and the TSV.

In some embodiments, the method further comprises introducing into the cell a fourth guide polynucleotide that forms a complex with the second monomer of the second Cas9-endonuclease dimer and comprises a fourth guide sequence, wherein the fourth guide sequence hybridizes to the TSC and the TSV.

In some embodiments, the first, second, third, and/or fourth guide polynucleotides are the same. In some embodiments, the first, second, third, and/or fourth guide polynucleotides are different.

In some embodiments, the method of the present disclosure comprises introducing into the cell the first, second, third, and fourth guide polynucleotides. In some embodiments, the first monomer of the first Cas9-endonuclease dimer forms a complex with the first guide polynucleotide, and the second monomer of the first Cas9-endonuclease dimer forms a complex with the second guide polynucleotide. In some embodiments, the first monomer of the second Cas9-endonuclease dimer forms a complex with the third guide polynucleotide, and the second monomer of the second Cas9-endonuclease dimer forms a complex with the fourth guide polynucleotide.

In some embodiments, the first monomer of the first Cas9-endonuclease dimer forms a complex with the first guide polynucleotide, the second monomer of the first Cas9-endonuclease dimer forms a complex with the second guide polynucleotide, the first monomer of the second Cas9-endonuclease dimer forms a complex with the third guide polynucleotide, and the second monomer of the second Cas9-endonuclease dimer forms a complex with the fourth guide polynucleotide. In some embodiments, the first and second guide polynucleotides guide the first Cas9-endonuclease dimer to a target sequence on the chromosome of the cell, and the third and fourth guide polynucleotides guide the second Cas9-endonuclease dimer to a target sequence on the vector introduced into the cell.

In some embodiments, the method of the present disclosure further comprises introducing into the cell a tracrRNA. In some embodiments, the guide polynucleotide comprises a crRNA/tracrRNA hybrid. In some embodiments, the tracrRNA component of the guide polynucleotide activates the Cas9 of the Cas9-endonuclease. In some embodiments, a Cas9-endonuclease, guide polynucleotide, and tracrRNA are capable of forming a complex. In some embodiments, the complex comprises a Cas9-endonuclease, two guide polynucleotides, and two tracrRNA sequences. In some embodiments, the complex of Cas9-endonuclease, guide polynucleotide, and tracrRNA does not occur in nature.

In some embodiments, the first monomer of the first Cas9-endonuclease dimer forms a complex with the first guide polynucleotide sequence and a tracrRNA sequence, and the second monomer of the first Cas9-endonuclease dimer forms a complex with the second guide polynucleotide sequence and a tracrRNA sequence. In some embodiments, the first monomer of the second Cas9-endonuclease dimer forms a complex with the third guide polynucleotide sequence and a tracrRNA sequence, and the second monomer of the second Cas9-endonuclease dimer forms a complex with the fourth guide polynucleotide sequence and a tracrRNA sequence.

In some embodiments, the first monomer of the first Cas9-endonuclease dimer forms a complex with the first guide polynucleotide and a tracrRNA, the second monomer of the first Cas9-endonuclease dimer forms a complex with the second guide polynucleotide and a tracrRNA, the first monomer of the second Cas9-endonuclease dimer forms a complex with the third guide polynucleotide and a tracrRNA, and the second monomer of the second Cas9-endonuclease dimer forms a complex with the fourth guide polynucleotide and a tracrRNA. In some embodiments, the first guide polynucleotide and tracrRNA and second guide polynucleotide and tracrRNA guide the first Cas9-endonuclease dimer to a target sequence on the chromosome of the cell, and the third guide polynucleotide and tracrRNA and fourth guide polynucleotide and tracrRNA guide the second Cas9-endonuclease dimer to a target sequence on the vector introduced into the cell.

In embodiments of the method, the TSV, first and/or second Cas9-endonuclease dimers are introduced into the cell as polynucleotide(s) encoding the first and second Cas9-endonuclease dimers. In some embodiments, the polynucleotide encoding the TSV, first and/or second Cas9-endonuclease dimers are codon-optimized for expression in a eukaryotic cell. In some embodiments, the polynucleotide encoding the TSV, first and/or second Cas9-endonuclease dimers are codon-optimized for expression in a mammalian cell. Codon optimization methods and techniques are described herein.

In some embodiments, the TSV, first and/or second Cas9-endonuclease dimers are introduced into the cell as a single nucleic acid molecule. In some embodiments, the polynucleotide encoding the TSV, first and/or second Cas9-endonuclease dimers is on a single vector. In some embodiments, the polynucleotide encoding the first and second Cas9-endonuclease dimers, one or more guide polynucleotides, and one or more tracrRNA sequences is on a single vector. In some embodiments, the vector is an expression vector. In some embodiments, the vector is a eukaryotic expression vector. In some embodiments, the vector is a mammalian expression vector. In some embodiments, the vector is a human expression vector. In some embodiments, the vector is a plant expression vector.

In some embodiments, the polynucleotide encoding the TSV, first and/or second Cas9-endonuclease dimers is on more than one vector. In some embodiments, the polynucleotide encoding the TSV, first and/or second Cas9-endonuclease dimers, one or more guide polynucleotides, and one or more tracrRNA sequences is on more than one vector. In some embodiments, the vectors are expression vectors. In some embodiments, the vectors are eukaryotic expression vectors. In some embodiments, the vectors are mammalian expression vectors. In some embodiments, the vectors are human expression vectors. In some embodiments, the vectors are plant expression vectors.

In embodiments of the method, the cell is a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the human cell is a differentiated form of any of the cells described herein. In some embodiments, the eukaryotic cell is a cell derived from any primary cell in culture. In some embodiments, the cell is a stem cell or stem cell line.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, i.e., potatoes; plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In embodiments of the method, a first Cas9-endonuclease dimer capable of generating cohesive ends in the TSC and a second Cas9-endonuclease dimer capable of generating cohesive ends in the TSV are introduced into a cell via delivery particles, vesicles, or viral vectors.

In some embodiments, the TSV, first and/or second Cas9-endonuclease dimers are delivered into the cell via a delivery particle. Examples of delivery particles are provided herein. In some embodiments, the delivery particle is a lipid-based system, a liposome, a micelle, a microvesicle, an exosome, or a gene gun. In some embodiments, the delivery particle comprises both monomers of the Cas9-endonuclease dimer. In some embodiments, the delivery particle comprises both monomers of both Cas9-endonuclease dimers. In some embodiments, the delivery particle comprises a Cas9-endonuclease and a guide polynucleotide. In some embodiments, the delivery particle comprises a Cas9-endonuclease and a guide polynucleotide, wherein the Cas9-endonuclease and the guide polynucleotide are in a complex. In some embodiments, the delivery particle comprises a polynucleotide encoding a Cas9-endonuclease, a polynucleotide encoding a guide polynucleotide, and a polynucleotide comprising a tracrRNA. In some embodiments, the delivery particle comprises a Cas9-endonuclease, a guide polynucleotide, and a tracrRNA. In some embodiments, the delivery particle comprises the first and/or second Cas9-endonuclease dimers, the first, second, third, and/or fourth guide polynucleotides, and a tracrRNA. In some embodiments, the delivery particle comprises a polynucleotide encoding one or more Cas9-endonucleases, a polynucleotide encoding the first, second, third, and/or fourth guide polynucleotides, and a polynucleotide encoding a tracrRNA.

In some embodiments, the delivery particle further comprises a lipid, a sugar, a metal or a protein. In some embodiments, the delivery particle is a lipid envelope. In some embodiments, the delivery particle is a sugar-based particle, for example, GalNAc. In some embodiments, the delivery particle is a nanoparticle. Examples of nanoparticles are described herein. Preparation of delivery particles is further described in U.S. Patent Publication Nos. 2011/0293703, 2012/0251560, and 2013/0302401; and U.S. Pat. Nos. 5,543,158, 5,855,913, 5,895,309, 6,007,845, and 8,709,843, each of which is incorporated by reference herein in its entirety.

In some embodiments, the TSV, first and/or second Cas9-endonuclease dimers are delivered into the cell via a vesicle. A “vesicle” is a small structure within a cell having a fluid enclosed by a lipid bilayer. Examples of vesicles are provided herein. In some embodiments, the vesicle comprises both monomers of the Cas9-endonuclease dimer. In some embodiments, the vesicle comprises both monomers of both Cas9-endonuclease dimers. In some embodiments, the vesicle comprises a Cas9-endonuclease and a guide polynucleotide. In some embodiments, the vesicle comprises a Cas9-endonuclease and a guide polynucleotide, wherein the Cas9-endonuclease and the guide polynucleotide are in a complex. In some embodiments, the vesicle comprises a polynucleotide encoding a Cas9-endonuclease, a polynucleotide encoding a guide polynucleotide, and a polynucleotide comprising a tracrRNA. In some embodiments, the vesicle comprises a Cas9-endonuclease, a guide polynucleotide, and a tracrRNA. In some embodiments, the vesicle comprises the first and/or second Cas9-endonuclease dimers, the first, second, third, and/or fourth guide polynucleotides, and a tracrRNA. In some embodiments, the vesicle comprises a polynucleotide encoding one or more Cas9-endonucleases, a polynucleotide encoding the first, second, third, and/or fourth guide polynucleotides, and a polynucleotide encoding a tracrRNA.

In some embodiments, the vesicle is an exosome or a liposome. In some embodiments, the first and/or second Cas9-endonuclease dimer is delivered into the cell via an exosome. Exosomes are endogenous nano-vesicles (i.e., having a diameter of about 30 to about 100 nm) that transport RNAs and proteins, and which can deliver RNA to the brain and other target organs. Engineered exosomes for delivery of exogenous biological materials into target organs is described, for example, by Alvarez-Erviti et al., Nature Biotechnology 29: 341 (2011), El-Andaloussi et al., Nature Protocols 7: 2112-2116 (2012), and Wahlgren et al., Nucleic Acids Research 40(17): e130 (2012), each of which is incorporated by reference herein in its entirety.

In some embodiments, the TSV, first and/or second Cas9-endonuclease dimer is delivered into the cell via a liposome. Liposomes are spherical vesicle structures having at least one lipid bilayer and can be used as a vehicle for administration of nutrients and pharmaceutical drugs. Liposomes are often composed of phospholipids, in particular phosphatidylcholine, but also other lipids such as egg phosphatidylethanolamine. Types of liposomes include, but are not limited to, multilamellar vesicle, small unilamellar vesicle, large unilamellar vesicle, and cochleate vesicle. See, e.g., Spuch and Navarro, “Liposomes for Targeted Delivery of Active Agents against Neurodegenerative Diseases (Alzheimer's Disease and Parkinson's Disease), Journal of Drug Delivery 2011, Article ID 469679 (2011). Liposomes for delivery of biological materials such as CRISPR-Cas components are described, for example, by Morrissey et al., Nature Biotechnology 23(8): 1002-1007 (2005), Zimmerman et al., Nature Letters 441: 111-114 (2006), and Li et al., Gene Therapy 19: 775-780 (2012), each of which is incorporated by reference herein in its entirety.

In embodiments of the method, the TSV, first and/or second Cas9-endonuclease dimers are delivered into the cell by a viral vector. In some embodiments, the viral vector comprises both monomers of the Cas9-endonuclease dimer. In some embodiments, the viral vector comprises both monomers of both Cas9-endonuclease dimers. In some embodiments, the viral vector comprises the TSV. In some embodiments, the viral vector comprises a Cas9-endonuclease and a guide polynucleotide. In some embodiments, the viral vector comprises a Cas9-endonuclease and a guide polynucleotide, wherein the Cas9-endonuclease and the guide polynucleotide are in a complex. In some embodiments, the viral vector comprises a polynucleotide encoding a Cas9-endonuclease, a polynucleotide encoding a guide polynucleotide, and a polynucleotide comprising a tracrRNA. In some embodiments, the viral vector comprises the first and/or second Cas9-endonuclease dimers, the first, second, third, and/or fourth guide polynucleotides, and a tracrRNA. In some embodiments, the viral vector comprises a polynucleotide encoding one or more Cas9-endonucleases, a polynucleotide encoding the first, second, third, and/or fourth guide polynucleotides, and a polynucleotide encoding a tracrRNA. In some embodiments, the viral vector comprises the TSV, and a polynucleotide encoding one or more Cas9-endonucleases, a polynucleotide encoding the first, second, third, and/or fourth guide polynucleotides, and a polynucleotide encoding a tracrRNA.

In some embodiments, the viral vector is of an adenovirus, a lentivirus, or an adeno-associated virus. Examples of viral vectors are provided herein. Viral transduction with adeno-associated virus (AAV) and lentiviral vectors (where administration can be local, targeted or systemic) have been used as delivery methods for in vivo gene therapy. In embodiments of the present disclosure, the Cas protein is expressed intracellularly by transduced cells.

In some embodiments, the first, second, or both Cas9-endonuclease dimers comprise a nuclear localization signal. In some embodiments, the first, second, or both monomers of the first Cas9-endonuclease dimer comprise a nuclear localization signal. In some embodiments, the first, second, or both monomers of the second Cas9-endonuclease dimer comprise a nuclear localization signal. In some embodiments, the first, second, or both monomers of the first, second, or both Cas9-endonuclease dimers comprise a nuclear localization signal. Nuclear localization signals (“NLSs”) are described herein. Exemplary nuclear localization sequences include, but are not limited to the NLS from: SV40 Large T-Antigen, nucleoplasmin, EGL-13, c-Myc, and TUS-protein. In some embodiments, the NLS comprises the sequence PKKKRKV (SEQ ID NO: 1). In some embodiments, the NLS comprises the sequence AVKRPAATKKAGQAKKKKLD (SEQ ID NO: 2). In some embodiments, the NLS comprises the sequence PAAKRVKLD (SEQ ID NO: 3). In some embodiments, the NLS comprises the sequence MSRRRKANPTKLSENAKKLAKEVEN (SEQ ID NO: 4). In some embodiments, the NLS comprises the sequence KLKIKRPVK (SEQ ID NO: 5). Other nuclear localization sequences include, but are not limited to, the acidic M9 domain of hnRNP A1, the sequence KIPIK (SEQ ID NO: 6) in yeast transcription repressor Matα2, and PY-NLSs.

### Methods for Seamless Mutagenesis

In some embodiments, the present disclosure provides a method of seamlessly modifying one or more nucleotides in a target polynucleotide sequence in a cell. “Seamless mutagenesis” refers to site-directed mutagenesis (i.e., substitution, deletion, or insertion of one or more nucleotides) without any other nearby change, such as the presence of the selectable gene used to introduce the mutation. Seamless DNA engineering for mutagenesis in a protein coding region is advantageous because any extraneous sequence introduced during the mutagenic step could interfere with protein expression. The present disclosure provides seamless mutagenesis using a two-step selection/counter-selection strategy, which first involves insertion at the target site of a selectable cassette such as an antibiotic resistance gene accompanied by a counter-selectable gene. The cassette is then subsequently replaced seamlessly with the desired sequence by selecting against the counter-selectable gene usually involving the administration of a small molecule, such as streptomycin or a sugar. Popular options of counter-selectable markers include sacB, rpsL, as well as markers that can, in the right host background, both be selected for and against including galK, thyA and tolC. Previous methods of seamless mutagenesis were described in, e.g., Wang et al., “Improved seamless mutagenesis by recombineering using ccdB for counterselection,” Nucleic Acids Research 42(5): e37 (2014); Zhang et al., “A new logic for DNA engineering using recombination in Escherichia coli,” Nature Genetics 20(2): 123-128 (1998); Westenberg et al., “Counter-selection recombineering of the baculovirus genome: a strategy for seamless modification of repeat-containing BACs,” Nucleic Acids Research 38: e166 (2010); Wong et al., “Efficient and seamless DNA recombineering using a thymidylate synthase A selection system in Escherichia coli,” Nucleic Acids Research 33: e59 (2005), each of which is incorporated by reference herein in its entirety.

In some embodiments, the present disclosure provides a method of modifying one or more nucleotides in a target polynucleotide sequence in a cell, the method comprising: (1) introducing into the cell a vector comprising an insertion cassette (IC), the IC comprising, in a 5′ to 3′ direction: (a) a first region homologous to part of the target polynucleotide sequence, (b) a second region comprising a mutation of one or more nucleotides in the target polynucleotide sequence, (c) a first nuclease binding site, (d) a polynucleotide sequence encoding a marker gene, (e) a second nuclease binding site, (f) a third region comprising a mutation of one or more mutations in the target polynucleotide sequence, and (g) a fourth region homologous to part of the target polynucleotide sequence, wherein the first region and the fourth region are 95%-100% identical to their respective parts of the target polynucleotide sequence; (2) inserting the IC into the target polynucleotide sequence via homologous recombination to generate a first modified target polynucleotide; (3) selecting a cell which expresses the marker gene; (4) subjecting the first modified target polynucleotide to a site-specific nuclease to generate a second modified target polynucleotide having cohesive ends; and (5) subjecting the second modified target polynucleotide having cohesive ends to a ligase, wherein the ligase ligates the cohesive ends at the second region and the third region to create a ligated modified target nucleic acid comprising one or more modified nucleotides when compared to the target polynucleotide sequence.

In some embodiments, the modification of one or more nucleotides in a target polynucleotide sequence is a nucleotide substitution, i.e., a single-nucleotide substitution or multiple-nucleotide substitution. Modification of one or more nucleotides in a target polynucleotide sequence can result in a change in the polypeptide sequence encoded by the polynucleotide. Modification of one or more nucleotides in a target polynucleotide sequence can also result in inactivation of expression of a downstream polynucleotide sequence in the cell. For example, the downstream sequence is inactivated such that the sequence is not transcribed, the coded protein is not produced, or the sequence does not function as the wild-type sequence does. In some embodiments, the target polynucleotide sequence is a regulatory sequence. In some embodiments, a regulatory sequence can be inactivated such that it no longer functions as a regulatory sequence. Examples of regulatory sequences are described herein.

The method of modifying one or more nucleotides in a target polynucleotide sequence in a cell via seamless mutagenesis utilizes an insertion cassette. In some embodiments, the insertion cassette (IC) is on a vector. Examples of vectors are provided herein. The IC as described herein comprises:
(i) a first region homologous to part of the target polynucleotide sequence,
(ii) a second region comprising a mutation of the target polynucleotide sequence of one or more nucleotides,
(iii) a first nuclease binding site,
(iv) a polynucleotide sequence encoding a marker gene,
(v) a second nuclease binding site,
(vi) a third region comprising a mutation of the target polynucleotide sequence of one or more nucleotides, and
(vii) a fourth region homologous to part of the target polynucleotide sequence, wherein the first region and the fourth region are 95%-100% identical to their respective parts of the target polynucleotide sequence.

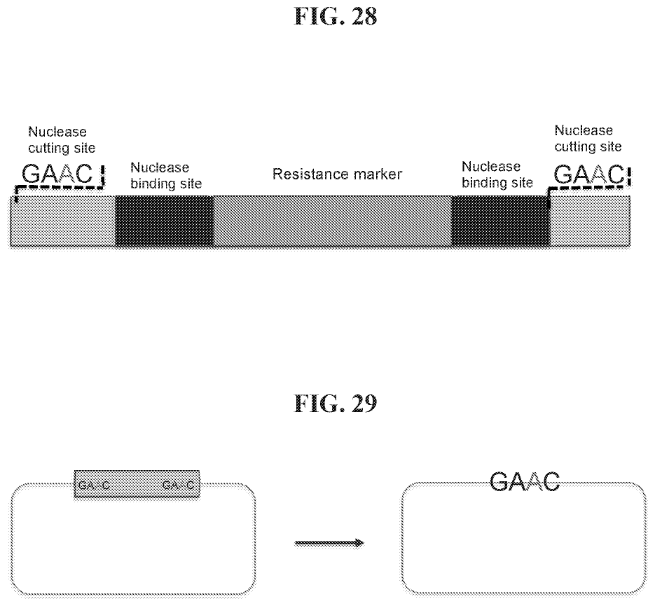

An exemplary IC is shown in FIG. 28. In FIG. 28, the IC comprises, in a 5′ to 3′ (with respect to the “top” or “coding” strand of double-stranded DNA) direction: a first nuclease cutting site, a first nuclease binding site, a resistance marker, a second nuclease binding site, and a second nuclease cutting site. The first and second nuclease cutting sites comprise the desired nucleotide mutation within the target polynucleotide sequence.

As shown in FIG. 27, “homology arms” (“HA”) are present upstream of the first nuclease cutting site and downstream of the second nuclease cutting site. The “homology arms” comprise regions homologous to part of the target polynucleotide sequence. In some embodiments, the first region of the IC homologous to part of the target polynucleotide sequence comprises the HA upstream of the first nuclease cutting site. In some embodiments, the fourth region of the IC homologous to part of the target polynucleotide sequence comprises the HA downstream of the second nuclease cutting site.

In some embodiments, the IC comprises a first region homologous to a part of a target polynucleotide sequence. In some embodiments, the IC comprises a fourth region homologous to a part of a target polynucleotide sequence. In some embodiments, the first and fourth regions in the IC have at least 70%, at least 75%, at least 80%, at least 85%, at least 90%, at least 95%, at least 96%, at least 97%, at least 98%, at least 99%, or 100% sequence identity with their respective parts of the target polynucleotide sequence. In some embodiments, the HA of the first and fourth regions in the IC have about 10 to 5000 base pairs, about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In some embodiments, the HA of the first and fourth regions in the IC have about 5, about 10, about 20, about 30, about 40, about 50, about 100, about 250, about 500, about 100, about 1250, about 1500, about 1750, about 2000, about 2250, or about 2500 base pairs.

In some embodiments, the IC comprises a second region comprising a mutation of the target polynucleotide sequence of one or more nucleotides. In some embodiments, the IC comprises a third region comprising a mutation of the target polynucleotide sequence of one or more nucleotides. As shown in FIGS. 28 and 29, the nuclease cutting sites comprise the mutation of one or more nucleotides within the target polynucleotide sequence. In some embodiments, the nuclease cutting site is the cleavage site of any suitable nuclease. For example, the nuclease cutting site can be the cleavage site of a restriction enzyme, such as, e.g., HindIII, BamHI, EcoRI, BbvI, FokI, MmeI, and the like. In some embodiments, the second region of the IC comprises a first nuclease cutting site comprising the desired mutation. In some embodiments, the third region of the IC comprises a second nuclease cutting site comprising the desired mutation. In some embodiments, the second and third regions of the IC are identical, or substantially identical.

In some embodiments, the IC comprises a first and second nuclease binding sites. The nuclease binding site can be the binding site of any suitable nuclease. For example, the nuclease binding site of a restriction enzyme, a zinc finger nuclease, a TALEN (transcription activator-like endonuclease), or a Cas9. For example, if the nuclease is Cas9, a guide RNA can be designed to hybridize to any sequence upstream (i.e., 5′ with respect to the relevant DNA strand) of a PAM. Thus, in some embodiments, the nuclease binding site is upstream of a PAM. In some embodiments, the first and second nuclease binding sites are identical, or substantially identical.

In some embodiments, the IC comprises a polynucleotide encoding a marker gene. “Marker” genes are used to determine whether a nucleic acid sequence has been successfully inserted into a target sequence. Marker genes can be selectable markers (e.g., resistance or selection markers) or screenable markers (e.g., fluorescent or colorimetric markers).

Non-limiting examples of resistance/selection markers include: antibiotic resistance genes (e.g., ampicillin-resistance genes, kanamycin resistance genes and the like) and other antibiotic resistance genes; auxotrophic markers (e.g., URA3, HIS3) and/or other host cell selection markers; nucleic acids to facilitate insertion into donor nucleic acid, e.g., transposase and inverted repeats, such as for transposition into a Mycoplasma genome; nucleic acids to support replication and segregation in the host cell, such as an autonomously replicated sequence (ARS) or centromere sequence (CEN).

Screenable markers will make cells containing the marker gene look different. Non-limiting examples of screenable markers include: green fluorescent protein (GFP) and its variants (e.g., yellow fluorescent protein, red fluorescent protein and the like); β-glucuronidase, used in the GUS assay to detect cells by staining it blue; and X-gal, used in the blue/white screen well-known to one of skill in the art.

The method of selection of cells which express the marker gene varies depending on the marker used. For example, if an antibiotic resistance marker is used, then selection involves growing a population of cells in a culture medium containing the antibiotic and collecting the cells which survive. If a screenable marker such as GFP is used, then selection involves collecting the cells which are green. Collecting the cells may be performed, for example, by manually picking colonies from a culture plate, or by sorting using a flow cytometry device, e.g. fluorescence-activated cell sorting (FACS).

In embodiments of the methods for seamless mutagenesis, the first step of the method comprises introducing into the cell a vector comprising the IC. The vector can be introduced into the cell using a method routine in the art, such as, for example, transfection, transduction, cell fusion, and lipofection. Introduction of vectors into a cell is further described herein.

In embodiments of the methods for seamless mutagenesis, the second step of the method comprises inserting the IC into the target polynucleotide sequence via homologous recombination to generate a first modified target polynucleotide. As exemplified in FIG. 27, the resistance cassette is inserted into the target polynucleotide sequence via homologous recombination (as indicated by the crosses on either side of the “GATC” sequence). As described herein, for specific homologous recombination, the vector will contain sufficiently long regions of homology (i.e., the first and fourth regions in the IC) to sequences of the chromosome to allow complementary binding and incorporation of the vector into the chromosome. As described herein, longer regions of homology, and greater degrees of sequence similarity, may increase the efficiency of homologous recombination.

In embodiments of the methods for seamless mutagenesis, the third step of the method comprises selecting a cell which expresses the marker gene. As described herein, the method of selection of a cell which expresses the marker gene depends on the selection marker. Selection methods, as well as various types of marker genes, are described herein.

In embodiments of the methods for seamless mutagenesis, the fourth step of the method comprises subjecting the first modified target polynucleotide (i.e., the first modified target polynucleotide generated from step (2) above) to a site-specific nuclease to generate a second modified target polynucleotide having cohesive ends. In some embodiments, the cohesive ends are in the second and third regions of the IC. The site-specific nuclease can be any site-specific nuclease which generates cohesive ends, including but not limited to restriction enzymes, Cas9-endonucleases described herein, or stiCas9 described herein. In some embodiments, the nuclease generates a double-stranded DNA break comprising cohesive ends. In some embodiments, the site-specific nuclease is exogenous to the cell, i.e., the site-specific nuclease does not occur naturally in the cell. In some embodiments, the site-specific nuclease is introduced into the cell. In some embodiments, the site-specific nuclease is introduced into the cell as a polynucleotide encoding the site-specific nuclease. Methods of introducing polynucleotides (such as, e.g., vectors) are described herein and include, for example, transfection, transduction, cell fusion, and lipofection. In some embodiments, the site-specific nuclease is a recombinant site-specific nuclease. As described herein, recombinant proteins refer to proteins not native to the cell producing them, or proteins with sequences which result from a new combination of genetic material that is not known to exist in nature such as, e.g., proteins expressed from an exogenous nucleic acid introduced into a cell. In some embodiments, the recombinant site-specific nuclease is expressed from a nucleic acid not native to the cell.

In some embodiments, the site-specific nuclease is a Cas9 effector protein. Cas9 proteins are described herein. In some embodiments, the Cas9 effector protein is a Type II-B Cas9. Type II-B Cas9 proteins are described herein and are capable of generating cohesive ends. As described herein, Type II-B CRISPR systems are identified, inter alia, by the presence of a cas4 gene on the cas operon, and Type II-B Cas9 proteins is of the TIGR03031 TIGRFAM protein family. Thus, in some embodiments, the site-specific nuclease is of the TIGR03031 TIGRFAM protein family. In some embodiments, the site-specific nuclease comprises a domain that matches the TIGR03031 protein family with an E-value cut-off of 1E-5. In some embodiments, the site-specific nuclease comprises a domain that matches the TIGR03031 protein family with an E-value cut-off of 1E-10. Type II-B CRISPR systems are found in bacterial species such as, e.g., Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1_47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonassp. S-B4-1U, Tamilnaduibacter salinus, Vibrio natriegens, Arcobacter skirrowii, Francisella philomiragia, Francisella hispaniensis, or Parendozoicomonas haliclonae.

In some embodiments, the site-specific nuclease is a Cas9-endonuclease fusion protein. Cas9-endonuclease proteins are described herein. In some embodiments, the Cas9-endonuclease fusion protein comprises the DNA-targeting domain of Cas9 and the nuclease domain of an endonuclease. In some embodiments, the endonuclease in the Cas9-endonuclease fusion protein is a Type IIS endonuclease. Examples of Type IIS endonucleases are provided herein and include: BbvI, BgcI, BfuAI, BmpI, BspMI, CspCI, FokI, MboII, MmeI, NmeAIII, and PleI. In some embodiments, the endonuclease in the Cas9-endonuclease fusion protein is FokI. DNA cleavage by FokI only occurs upon dimerization of two FokI monomers. FokI cleavage of DNA generates cohesive ends with a 4 base-pair overhang.

In some embodiments, the Cas9-endonuclease fusion protein comprises a modified Cas9. Modified Cas9 is described herein and comprises catalytically inactive Cas9 and Cas9 having nickase activity. In some embodiments, the modified Cas9 is a catalytically inactive Cas9 (“deadCas9”). Catalytically inactive Cas9 are incapable of cleaving DNA (i.e., the cleavage domain of Cas9 is inactivated); however, they retain the ability to target a nucleic acid sequence by forming a complex with a guide polynucleotide (e.g., guide RNA). Catalytically inactive Cas9 are described herein. In some embodiments, catalytically inactive Cas9 comprises a double amino-acid substitution relative to wild-type Cas9. In some embodiments, the double amino-acid substitution is D10A and H840A. In some embodiments, the Cas9-endonuclease fusion protein comprises a catalytically inactive Cas9, and the endonuclease is FokI.

In some embodiments, the modified Cas9 is a Cas9 having nickase activity (“Cas9 nickase” or “Cas9n”). Cas9 nickases are capable of cleaving only one strand of double-stranded DNA (i.e., “nicking” the DNA). Cas9 nickases are described herein. In some embodiments, Cas9 nickases comprise a single amino-acid substitution relative to wild-type Cas9. In some embodiments, the single amino-acid substitution is D10A (“Cas9n^{(D10A)}”). In some embodiments, the single amino-acid substitution is H840A (“Cas9n^{(H840A)}”). In some embodiments, the Cas9-endonuclease fusion protein comprises a Cas9 having nickase activity, and the endonuclease is FokI. In some embodiments, the Cas9-endonuclease fusion protein comprises a Cas9 having a D10A mutation, and the endonuclease is FokI. In some embodiments, the Cas9-endonuclease fusion protein comprises a Cas9 having an H840A mutation, and the endonuclease is FokI.

In some embodiments, the site-specific nuclease is Cpf1. Cpf1 (Centromere and Promoter Factor 1) is a single RNA-guided endonuclease found in CRISPR/Cpf1 systems capable of generating cohesive ends. A CRISPR/Cpf1 system is analogous to a CRISPR/Cas9 system. However, there are several significant differences between Cas9 and Cpf1. Cpf1 does not utilize a tracrRNA. Cpf1 proteins recognize a different PAM sequence than Cas9. The PAM sequence of Cpf1 is a 5′ T-rich motif, such as, e.g., 5′-TTTN-3′, wherein N is A, T, C, or G. Cpf1 cleaves at a different site from Cas9. While Cas9 cleaves at a sequence adjacent to the PAM, Cpf1 cleaves at a sequence further away from the PAM. Cp1 proteins are further described in, e.g., foreign patent publication GB 1506509.7, U.S. Pat. No. 9,580,701, U.S. Patent Publication 2016/0208243, and Zetsche et al., “Cpf1 Is a Single RNA-Guided Endonuclease of a Class 2 CRISPR-Cas System,” Cell 163(3): 759-771 (2015), each of which is incorporated by reference herein in its entirety.

In some embodiments, the site-specific nuclease is Cas9, Cpf1, or Cas9-FokI.

In some embodiments, the cohesive ends generated by the site-specific nuclease comprise a 5′ overhang. In some embodiments, the cohesive ends generated by the site-specific nuclease comprise a 3′ overhang. In some embodiments, the site-specific nuclease generates cohesive ends comprising a single-stranded polynucleotide of 3 to 40 nucleotides. In some embodiments, the site-specific nuclease generates cohesive ends comprising a single-stranded polynucleotide of 4 to 30 nucleotides. In some embodiments, the site-specific nuclease generates cohesive ends comprising a single-stranded polynucleotide of 5 to 20 nucleotides. In some embodiments, the site-specific nuclease generates cohesive ends comprising a single-stranded polynucleotide of about 5 nucleotides, about 10 nucleotides, about 15 nucleotides, about 20 nucleotides, about 25 nucleotides, or about 30 nucleotides. In some embodiments, a deadCas9-FokI dimer generates cohesive ends comprising a 4-nucleotide 5′ overhang. In some embodiments, a Cas9n^{(D10A)}-FokI dimer generates cohesive ends comprising a 27-nucleotide 5′ overhang. In some embodiments, a Cas9^{(H840A)}-FokI dimer generates cohesive ends comprising a 23-nucleotide 3′-overhang.

In embodiments of the method, the fifth step of the method comprises subjecting the second modified target polynucleotide having cohesive ends to a ligase, wherein the ligase ligates the cohesive ends at the second region and the third region to create a ligated modified target nucleic acid comprising one or more modified nucleotides when compared to the target polynucleotide sequence. A ligase is an enzyme that catalyzes the joining of two or more nucleic acid fragments by forming a chemical bond. In some embodiments, a ligase joins together two or more DNA fragments by catalyzing the formation of a phosphodiester bond. Any suitable ligase can be used, and the suitable ligase can be determined by one of skill in the art. Non-limiting examples of ligases include: E. coli ligase, T4 DNA ligase from bacteriophage T4, DNA ligase I, DNA ligase II, DNA ligase III, DNA ligase IV, and thermostable ligases such as Ampligase® DNA Ligase. Ligases can ligate blunt ends or cohesive ends. In some embodiments, the ligase ligates cohesive ends. In some embodiments, the ligase requires ATP in order to ligate DNA fragments.

In some embodiments, the ligase is exogenous to the cell, i.e., the ligase does not occur naturally in the cell. In some embodiments, the ligase is introduced into the cell. In some embodiments, the ligase is introduced into the cell as a polynucleotide encoding a ligase. Methods of introducing polynucleotides (such as, e.g., vectors) are described herein. In some embodiments, the ligase is a recombinant ligase, i.e., a ligase expressed from a nucleic acid not native to the cell.

In some embodiments, the ligated modified target nucleic acid comprises one or more modified nucleotides when compared with the target polynucleotide sequence, but does not comprise the marker gene or any additional nucleotides upstream or downstream of the target polynucleotide sequence, i.e., the target polynucleotide sequence was mutated seamlessly.

In embodiments of the method, the first modified target nucleic acid is isolated from the cell after the third step. Methods of isolating nucleic acids from cells are well-established in the art and include, for example, phenol/chloroform extraction, precipitation under low pH/high salt conditions, and solid phase extraction. Commercially available kits for isolation of nucleic acids, such as the QIAGEN Miniprep Kit, Bio-Rad Quantum Prep® Miniprep Kit, and Zymo Research ZYMOPURE Plasmid Miniprep Kit, may be used.

In embodiments of the method, the first modified target nucleic acid is in the cell after the third step, i.e., the nucleic acid is not isolated from the cell. In some embodiments, steps (1)-(5) of the method are performed within the same cell. In some embodiments, components of the method are introduced into the cell. In some embodiments, the vector comprising the insertion cassette, the site-specific nuclease, and the ligase are introduced into the cell. Methods of introducing vectors and proteins into cells are described herein and include, for example, delivery via delivery particles, vesicles, and/or vectors including viral vectors.

In embodiments of the method, the target polynucleotide sequence is in a plasmid. Plasmids and examples thereof are described herein. In some embodiments, the plasmid containing the target polynucleotide sequence is a native bacterial plasmid (i.e., a plasmid that occurs naturally in a bacterial cell). In some embodiments, the plasmid containing the target polynucleotide sequence is an exogenous plasmid introduced into a cell. In some embodiments, the cell is a bacterial cell. In some embodiments, the plasmid is an engineered plasmid. In some embodiments, modification of one or more nucleotides in a plasmid leads to a modified behavior of the cell. The modified behavior may be the expression of a modified protein, higher or lower levels of expression of one or more proteins, increased resistance or susceptibility to antibiotics, altered response to small molecules and/or proteins, altered production of small molecules and/or proteins, etc.

In embodiments of the method, the target polynucleotide sequence is in a chromosome. The chromosome may be a prokaryotic chromosome or eukaryotic chromosome. In some embodiments, the chromosome is of a eukaryotic cell. In some embodiments, the chromosome is of a human cell. In some embodiments, the chromosome is of an animal cell. In some embodiments, the chromosome is of a plant cell. In some embodiments, modification of one or more nucleotides in a chromosome leads to a modified behavior of the cell. The modified behavior may be the expression of a modified protein, higher or lower levels of expression of one or more proteins, increased resistance or susceptibility to antibiotics, altered response to small molecules and/or proteins, altered production of small molecules and/or proteins, etc.

Engineered Guide RNA (sgRNA)

In some embodiments, the disclosure provides an engineered guide RNA that forms a complex with a stiCas9 protein, comprising: (a) a guide sequence capable of hybridizing to a target sequence in a eukaryotic cell; and (b) a tracrRNA sequence capable of binding to the Cas9 protein, wherein the tracrRNA differs from a naturally-occurring tracrRNA sequence by at least 10 nucleotides, wherein the engineered guide RNA improves nuclease efficiency of the Cas9 protein.

As described herein, in some embodiments, a guide polynucleotide, e.g., guide RNA, forms a complex with a Cas9 protein, i.e., in some embodiments, a guide polynucleotide binds to Cas9. In some embodiments, the DNA-binding segment of the guide polynucleotide hybridizes with a target sequence in a eukaryotic cell, but not a sequence in a bacterial cell.

In some embodiments, the guide polynucleotide is 10 to 150 nucleotides. In some embodiments, the guide polynucleotide is 20 to 120 nucleotides. In some embodiments, the guide polynucleotide is 30 to 100 nucleotides. In some embodiments, the guide polynucleotide is 40 to 80 nucleotides. In some embodiments, the guide polynucleotide is 50 to 60 nucleotides. In some embodiments, the guide polynucleotide is 10 to 35 nucleotides. In some embodiments, the guide polynucleotide is 15 to 30 nucleotides. In some embodiments, the guide polynucleotide is 20 to 25 nucleotides.

The guide polynucleotide can be introduced into the target cell as an isolated molecule, e.g., RNA molecule, or is introduced into the cell using an expression vector containing DNA encoding the guide polynucleotide.

Naturally-occurring CRISPR systems utilize crRNA, which contains a region complementary to the target sequence, and tracrRNA, which binds to the Cas9 protein and also hybridizes with the crRNA. The crRNA/tracrRNA hybrid forms RNA secondary structures that allow binding of the crRNA portion to the target sequence and binding of the tracrRNA portion to the Cas9 protein. Non-limiting examples of RNA secondary structures include helices, stem loops, and pseudoknots. In some embodiments, the Cas9 protein recognizes at least one stem loop in the crRNA/tracrRNA hybrid for binding.

In engineered CRISPR-Cas systems, such as, for example, the CRISPR-Cas systems of the disclosure, it may be advantageous to utilize a single guide polynucleotide that can both complement the target sequence and bind the Cas9 protein. Thus, in some embodiments, the disclosure provides a non-naturally occurring CRISPR-Cas system comprising a Cas9 effector protein capable of generating cohesive ends (stiCas9); and a guide polynucleotide that forms a complex with the stiCas9 and comprises a guide sequence, wherein the guide sequence is capable of hybridizing with a target sequence in a eukaryotic cell but does not hybridize to a sequence in a bacterial cell; wherein the complex does not occur in nature, and wherein the system does not comprise a tracrRNA. In some embodiments, the guide polynucleotide forms at least one secondary structure. In some embodiments, the at least one secondary structure is one of a stem loop, a helix, or a pseudoknot.

It may be advantageous to optimize the engineered guide polynucleotides described herein, in order to improve binding affinity to the Cas9 protein and/or increase targeting efficiency to the target sequence. See, e.g., Dang et al., Genome Biology 16:280 (2015); Nowak et al., Nucleic Acids Res 44(20):9555-9564 (2016); and Vejnar et al., Cold Spring Harb Protoc, doi:10.1101/pdb.top090894 (2016). In some embodiments, the engineered guide polynucleotide, e.g., guide RNA, is shorter than the combination of the naturally-occurring crRNA and tracrRNA. In some embodiments, the engineered guide RNA is at least 5 nucleotides shorter, at least 6 nucleotides shorter, at least 7 nucleotides shorter, at least 8 nucleotides shorter, at least 8 nucleotides shorter, at least 9 nucleotides shorter, at least 10 nucleotides shorter, at least 11 nucleotides shorter, at least 12 nucleotides shorter, at least 13 nucleotides shorter, at least 14 nucleotides shorter, at least 15 nucleotides shorter, at least 16 nucleotides shorter, at least 17 nucleotides shorter, at least 18 nucleotides shorter, at least 19 nucleotides shorter, at least 20 nucleotides shorter, at least 21 nucleotides shorter, at least 22 nucleotides shorter, at least 23 nucleotides shorter, at least 24 nucleotides shorter, at least 25 nucleotides shorter, at least 26 nucleotides shorter, at least 27 nucleotides shorter, at least 28 nucleotides shorter, at least 29 nucleotides shorter, or at least 30 nucleotides shorter than the combination of the naturally-occurring crRNA and tracrRNA.

In some embodiments, the tracrRNA sequence is at least 5 nucleotides shorter, at least 6 nucleotides shorter, at least 7 nucleotides shorter, at least 8 nucleotides shorter, at least 8 nucleotides shorter, at least 9 nucleotides shorter, at least 10 nucleotides shorter, at least 11 nucleotides shorter, at least 12 nucleotides shorter, at least 13 nucleotides shorter, at least 14 nucleotides shorter, at least 15 nucleotides shorter, at least 16 nucleotides shorter, at least 17 nucleotides shorter, at least 18 nucleotides shorter, at least 19 nucleotides shorter, at least 20 nucleotides shorter, at least 21 nucleotides shorter, at least 22 nucleotides shorter, at least 23 nucleotides shorter, at least 24 nucleotides shorter, at least 25 nucleotides shorter, at least 26 nucleotides shorter, at least 27 nucleotides shorter, at least 28 nucleotides shorter, at least 29 nucleotides shorter, or at least 30 nucleotides shorter than the naturally-occurring tracrRNA sequence.

In some embodiments, the engineered guide polynucleotide is 5 nucleotides to 40 nucleotides shorter, 6 nucleotides to 40 nucleotides shorter, 7 nucleotides to 40 nucleotides shorter, 8 nucleotides to 40 nucleotides shorter, 9 nucleotides to 40 nucleotides shorter, 10 nucleotides to 40 nucleotides shorter, 11 nucleotides to 40 nucleotides shorter, 12 nucleotides to 40 nucleotides shorter, 13 nucleotides to 40 nucleotides shorter, 14 nucleotides to 40 nucleotides shorter, 15 nucleotides to 40 nucleotides shorter, 16 nucleotides to 40 nucleotides shorter, 17 nucleotides to 40 nucleotides shorter, 18 nucleotides to 40 nucleotides shorter, 19 nucleotides to 40 nucleotides shorter, 20 nucleotides to 40 nucleotides shorter, 21 nucleotides to 40 nucleotides shorter, 22 nucleotides to 40 nucleotides shorter, 23 nucleotides to 40 nucleotides shorter, 24 nucleotides to 40 nucleotides shorter, 25 nucleotides to 40 nucleotides shorter, 26 nucleotides to 40 nucleotides shorter, 27 nucleotides to 40 nucleotides shorter, 28 nucleotides to 40 nucleotides shorter, 29 nucleotides to 40 nucleotides shorter, 30 nucleotides to 40 nucleotides shorter, 31 nucleotides to 40 nucleotides shorter, 32 nucleotides to 40 nucleotides shorter, 33 nucleotides to 40 nucleotides shorter, 34 nucleotides to 40 nucleotides shorter, 35 nucleotides to 40 nucleotides shorter, 36 nucleotides to 40 nucleotides shorter, 37 nucleotides to 40 nucleotides shorter, 38 nucleotides to 40 nucleotides shorter, or 39 nucleotides to 40 nucleotides shorter than the combination of the naturally-occurring crRNA and tracrRNA.

In some embodiments, the engineered tracrRNA is 5 nucleotides to 40 nucleotides shorter, 6 nucleotides to 40 nucleotides shorter, 7 nucleotides to 40 nucleotides shorter, 8 nucleotides to 40 nucleotides shorter, 9 nucleotides to 40 nucleotides shorter, 10 nucleotides to 40 nucleotides shorter, 11 nucleotides to 40 nucleotides shorter, 12 nucleotides to 40 nucleotides shorter, 13 nucleotides to 40 nucleotides shorter, 14 nucleotides to 40 nucleotides shorter, 15 nucleotides to 40 nucleotides shorter, 16 nucleotides to 40 nucleotides shorter, 17 nucleotides to 40 nucleotides shorter, 18 nucleotides to 40 nucleotides shorter, 19 nucleotides to 40 nucleotides shorter, 20 nucleotides to 40 nucleotides shorter, 21 nucleotides to 40 nucleotides shorter, 22 nucleotides to 40 nucleotides shorter, 23 nucleotides to 40 nucleotides shorter, 24 nucleotides to 40 nucleotides shorter, 25 nucleotides to 40 nucleotides shorter, 26 nucleotides to 40 nucleotides shorter, 27 nucleotides to 40 nucleotides shorter, 28 nucleotides to 40 nucleotides shorter, 29 nucleotides to 40 nucleotides shorter, 30 nucleotides to 40 nucleotides shorter, 31 nucleotides to 40 nucleotides shorter, 32 nucleotides to 40 nucleotides shorter, 33 nucleotides to 40 nucleotides shorter, 34 nucleotides to 40 nucleotides shorter, 35 nucleotides to 40 nucleotides shorter, 36 nucleotides to 40 nucleotides shorter, 37 nucleotides to 40 nucleotides shorter, 38 nucleotides to 40 nucleotides shorter, or 39 nucleotides to 40 nucleotides shorter than the naturally-occurring tracrRNA.

In some embodiments, the engineered guide polynucleotide, e.g., guide RNA, is longer than the combination of the naturally-occurring crRNA and tracrRNA. In some embodiments, the engineered guide RNA is at least 5 nucleotides longer, at least 6 nucleotides longer, at least 7 nucleotides longer, at least 8 nucleotides longer, at least 8 nucleotides longer, at least 9 nucleotides longer, at least 10 nucleotides longer, at least 11 nucleotides longer, at least 12 nucleotides longer, at least 13 nucleotides longer, at least 14 nucleotides longer, at least 15 nucleotides longer, at least 16 nucleotides longer, at least 17 nucleotides longer, at least 18 nucleotides longer, at least 19 nucleotides longer, at least 20 nucleotides longer, at least 21 nucleotides longer, at least 22 nucleotides longer, at least 23 nucleotides longer, at least 24 nucleotides longer, at least 25 nucleotides longer, at least 26 nucleotides longer, at least 27 nucleotides longer, at least 28 nucleotides longer, at least 29 nucleotides longer, or at least 30 nucleotides longer than the combination of the naturally-occurring crRNA and tracrRNA.

In some embodiments, the tracrRNA sequence is at least 5 nucleotides longer, at least 6 nucleotides longer, at least 7 nucleotides longer, at least 8 nucleotides longer, at least 8 nucleotides longer, at least 9 nucleotides longer, at least 10 nucleotides longer, at least 11 nucleotides longer, at least 12 nucleotides longer, at least 13 nucleotides longer, at least 14 nucleotides longer, at least 15 nucleotides longer, at least 16 nucleotides longer, at least 17 nucleotides longer, at least 18 nucleotides longer, at least 19 nucleotides longer, at least 20 nucleotides longer, at least 21 nucleotides longer, at least 22 nucleotides longer, at least 23 nucleotides longer, at least 24 nucleotides longer, at least 25 nucleotides longer, at least 26 nucleotides longer, at least 27 nucleotides longer, at least 28 nucleotides longer, at least 29 nucleotides longer, or at least 30 nucleotides longer than the naturally-occurring tracrRNA sequence.

In some embodiments, the engineered guide polynucleotide is 5 nucleotides to 40 nucleotides longer, 6 nucleotides to 40 nucleotides longer, 7 nucleotides to 40 nucleotides longer, 8 nucleotides to 40 nucleotides longer, 9 nucleotides to 40 nucleotides longer, 10 nucleotides to 40 nucleotides longer, 11 nucleotides to 40 nucleotides longer, 12 nucleotides to 40 nucleotides longer, 13 nucleotides to 40 nucleotides longer, 14 nucleotides to 40 nucleotides longer, 15 nucleotides to 40 nucleotides longer, 16 nucleotides to 40 nucleotides longer, 17 nucleotides to 40 nucleotides longer, 18 nucleotides to 40 nucleotides longer, 19 nucleotides to 40 nucleotides longer, 20 nucleotides to 40 nucleotides longer, 21 nucleotides to 40 nucleotides longer, 22 nucleotides to 40 nucleotides longer, 23 nucleotides to 40 nucleotides longer, 24 nucleotides to 40 nucleotides longer, 25 nucleotides to 40 nucleotides longer, 26 nucleotides to 40 nucleotides longer, 27 nucleotides to 40 nucleotides longer, 28 nucleotides to 40 nucleotides longer, 29 nucleotides to 40 nucleotides longer, 30 nucleotides to 40 nucleotides longer, 31 nucleotides to 40 nucleotides longer, 32 nucleotides to 40 nucleotides longer, 33 nucleotides to 40 nucleotides longer, 34 nucleotides to 40 nucleotides longer, 35 nucleotides to 40 nucleotides longer, 36 nucleotides to 40 nucleotides longer, 37 nucleotides to 40 nucleotides longer, 38 nucleotides to 40 nucleotides longer, or 39 nucleotides to 40 nucleotides longer than the combination of the naturally-occurring crRNA and tracrRNA.

In some embodiments, the engineered tracrRNA is 5 nucleotides to 40 nucleotides longer, 6 nucleotides to 40 nucleotides longer, 7 nucleotides to 40 nucleotides longer, 8 nucleotides to 40 nucleotides longer, 9 nucleotides to 40 nucleotides longer, 10 nucleotides to 40 nucleotides longer, 11 nucleotides to 40 nucleotides longer, 12 nucleotides to 40 nucleotides longer, 13 nucleotides to 40 nucleotides longer, 14 nucleotides to 40 nucleotides longer, 15 nucleotides to 40 nucleotides longer, 16 nucleotides to 40 nucleotides longer, 17 nucleotides to 40 nucleotides longer, 18 nucleotides to 40 nucleotides longer, 19 nucleotides to 40 nucleotides longer, 20 nucleotides to 40 nucleotides longer, 21 nucleotides to 40 nucleotides longer, 22 nucleotides to 40 nucleotides longer, 23 nucleotides to 40 nucleotides longer, 24 nucleotides to 40 nucleotides longer, 25 nucleotides to 40 nucleotides longer, 26 nucleotides to 40 nucleotides longer, 27 nucleotides to 40 nucleotides longer, 28 nucleotides to 40 nucleotides longer, 29 nucleotides to 40 nucleotides longer, 30 nucleotides to 40 nucleotides longer, 31 nucleotides to 40 nucleotides longer, 32 nucleotides to 40 nucleotides longer, 33 nucleotides to 40 nucleotides longer, 34 nucleotides to 40 nucleotides longer, 35 nucleotides to 40 nucleotides longer, 36 nucleotides to 40 nucleotides longer, 37 nucleotides to 40 nucleotides longer, 38 nucleotides to 40 nucleotides longer, or 39 nucleotides to 40 nucleotides longer than the naturally-occurring tracrRNA.

In some embodiments, the engineered guide polynucleotide differs from the combination of the naturally-occurring crRNA and tracrRNA by at least one nucleotide, such that the binding affinity and/or the targeting efficiency of the engineered guide polynucleotide is higher than that of the naturally-occurring crRNA/tracrRNA hybrid. In some embodiments, the engineered guide polynucleotide differs from crRNA/tracrRNA hybrid by at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the engineered tracrRNA differs from naturally occurring tracrRNA by at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides.

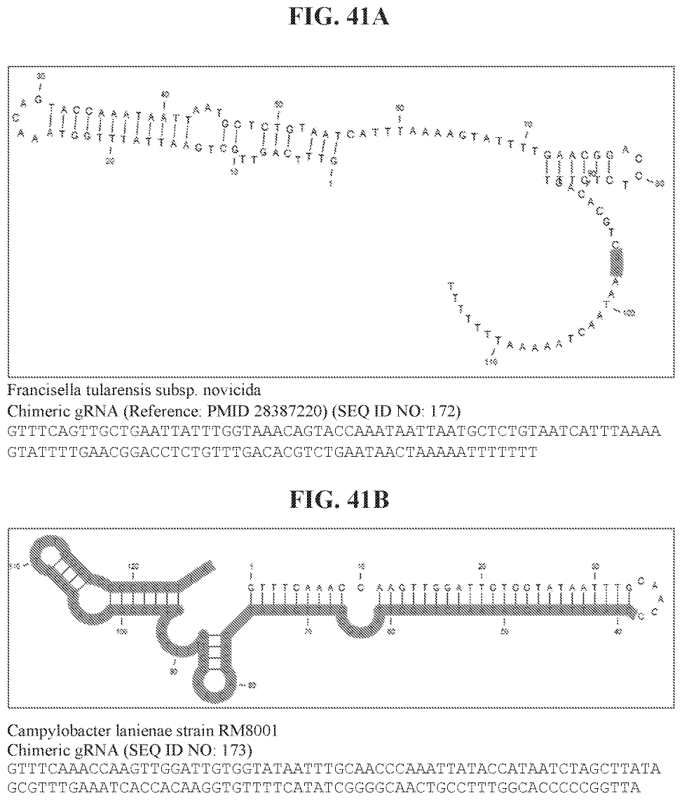

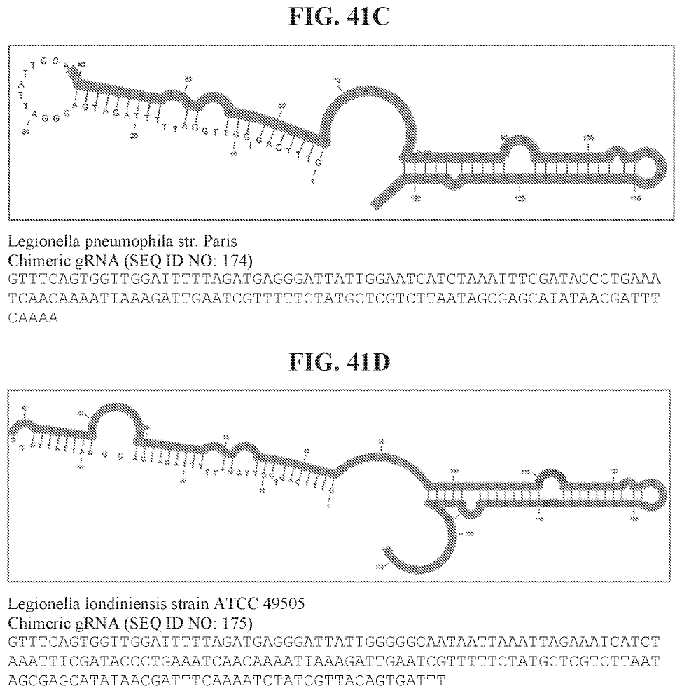

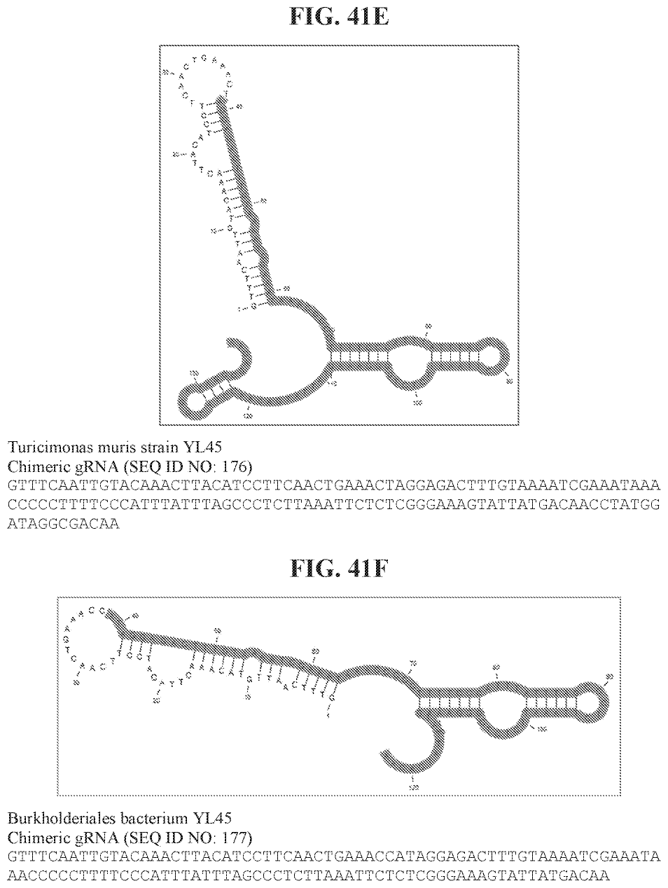

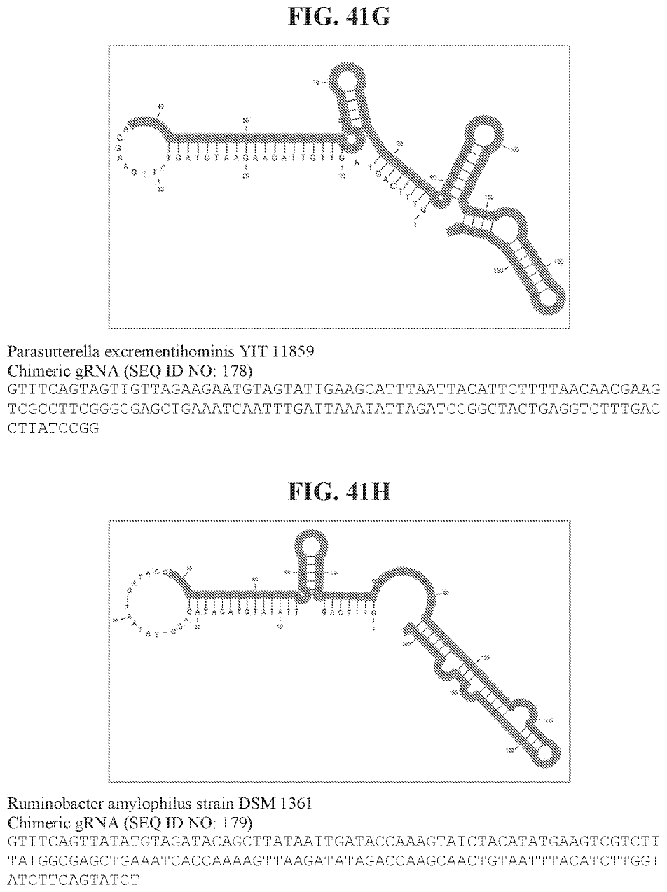

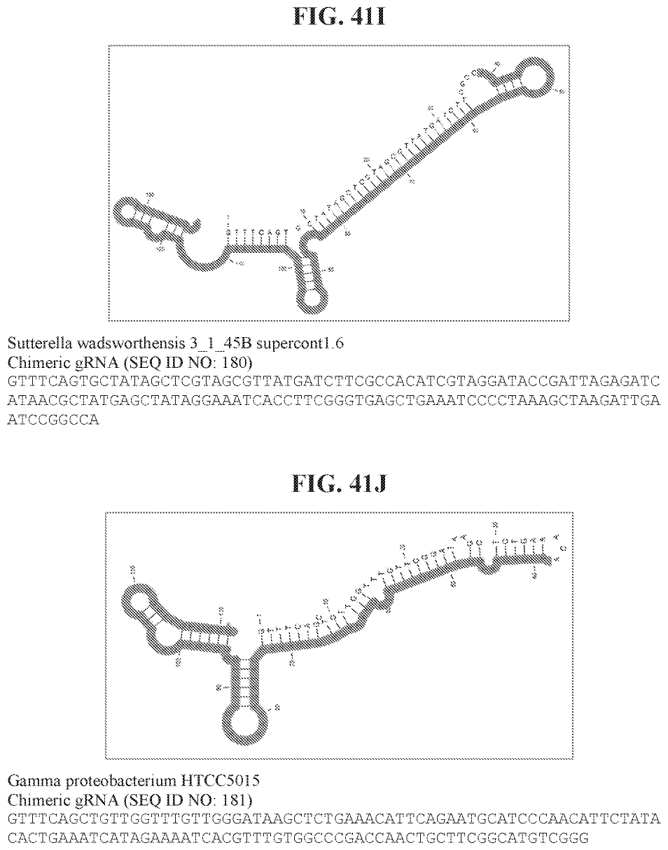

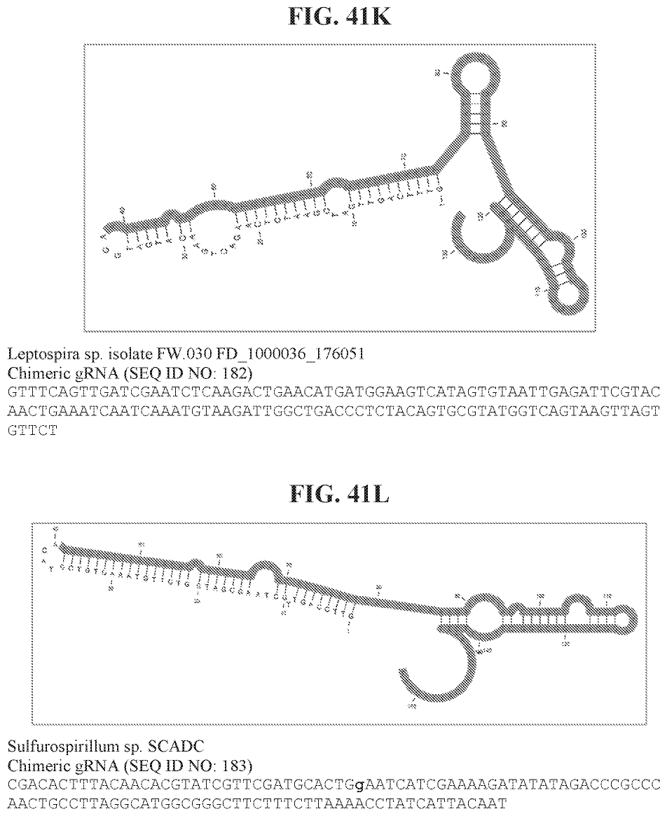

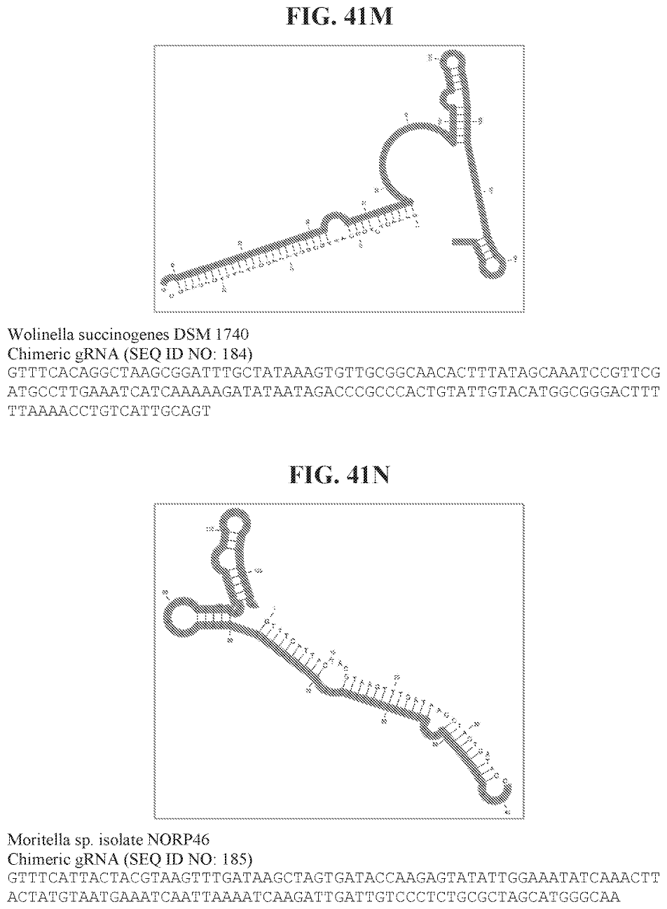

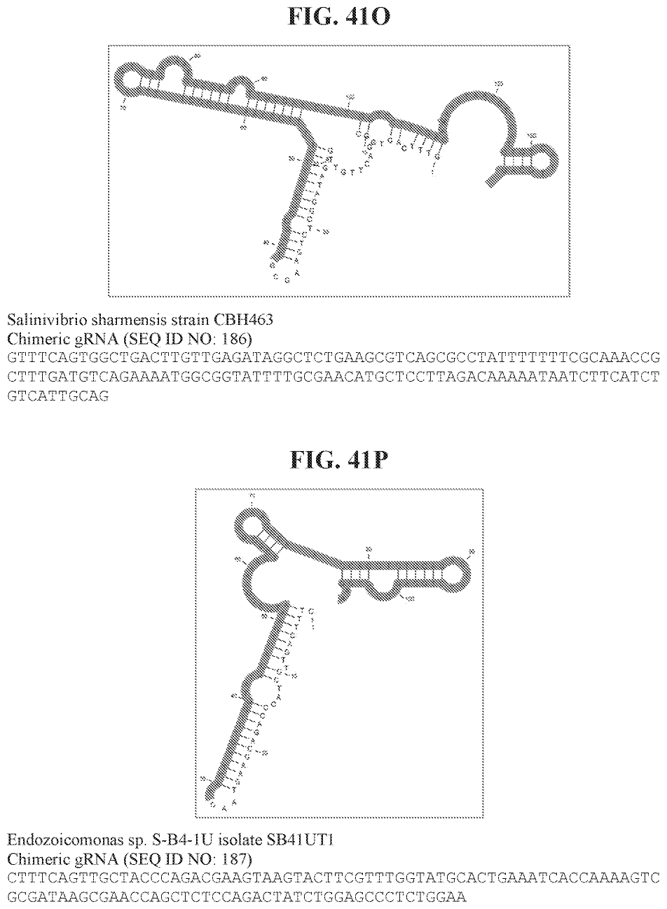

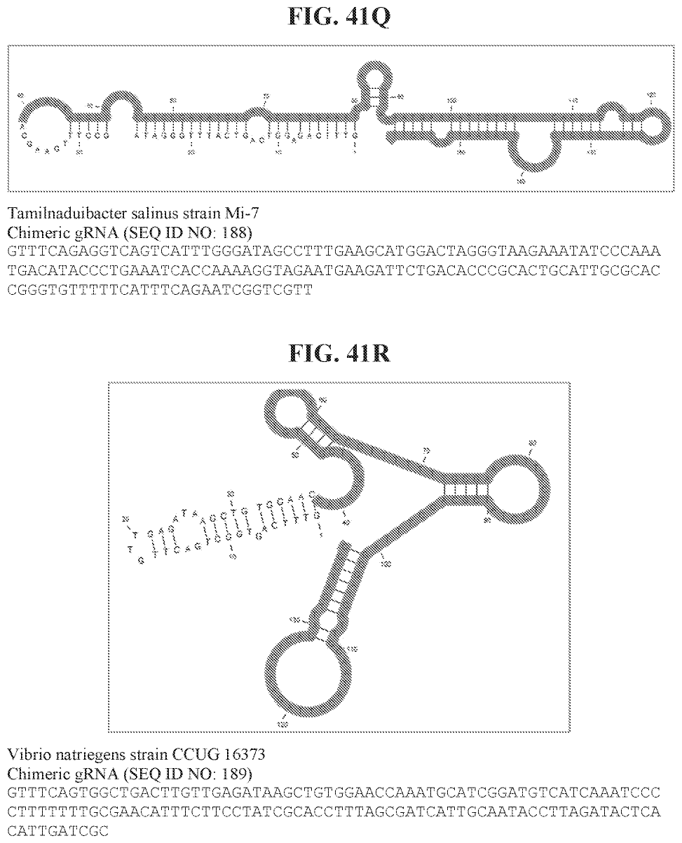

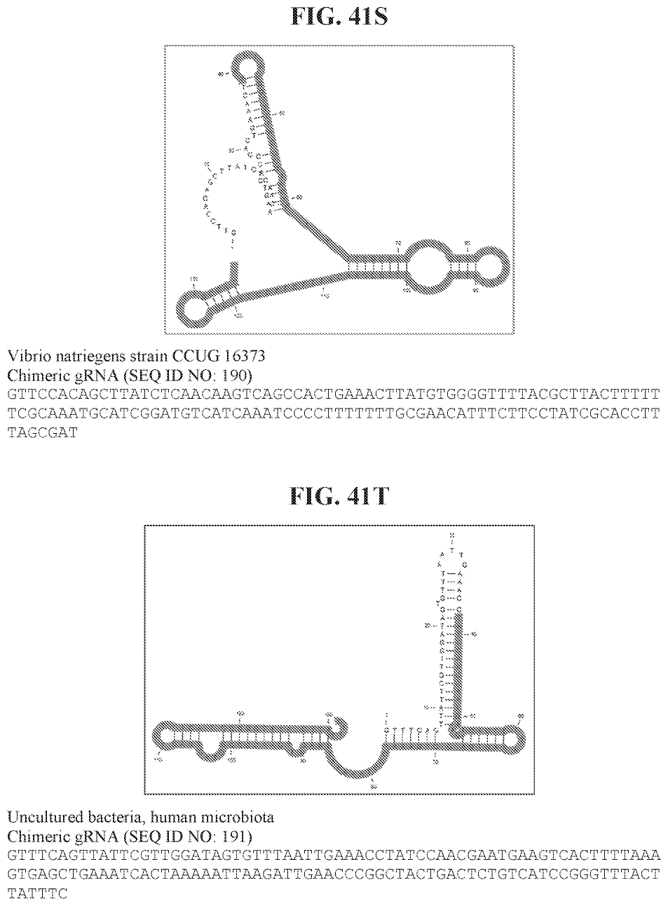

In some embodiments, modifications are made to a naturally-occurring tracrRNA to improve nuclease efficiency of a Cas9 protein. In some embodiments, the modification is in a stem loop of the tracrRNA. In some embodiments, the modification is elongation of the stem loop. In some embodiments, the modification is shortening of the stem loop. In some embodiments, the modification is one or more nucleotide substitutions in the stem loop. In some embodiments, the modification is to a stem-loop as shown in FIG. 41.

In some embodiments, the nuclease efficiency of the Cas9 protein, with the engineered guide RNA, improves by at least about 30%, at least about 40%, at least about 50%, at least about 60%, at least about 70%, at least about 80%, at least about 90%, or at least about 100%. In some embodiments, the nuclease efficiency of the Cas9 protein, with the engineered guide RNA, improves by at least about two-fold, at least about three-fold, at least about four-fold, at least about five-fold, at least about six-fold, at least about seven-fold, at least about eight-fold, at least about nine-fold, or at least about ten-fold.

The nuclease efficiency of the Cas9 protein can be measured, for example, in order to compare the nuclease efficiency of a Cas9 protein complexed with a naturally-occurring guide RNA, with a Cas9 protein complexed with the engineered guide RNA described herein. In some embodiments, the measurement method is a biochemical assay, such as, for example, measurement of the rate of in vitro Cas9 nuclease activity against a linear or circular template. In some embodiments, the measurement method measures targeting efficiency of the Cas9 protein using, for example, next-generation sequencing, T7 endonuclease I assay, and/or Cell assay. In some embodiments, the measurement method is an affinity test between the Cas9 protein and the tracrRNA using, for example, the BIACORE system.

In some embodiments, the guide sequence comprises at least 90% sequence identity to any one of SEQ ID NOs: 104-125 or 196-199. In some embodiments, the tracrRNA sequence comprises at least 90% sequence identity to any one of SEQ ID NOs: 148-171. In some embodiments, the guide RNA comprises at least 90% sequence identity to any one of SEQ ID NOs: 172-191.

In some embodiments, the engineered guide RNA, or the crRNA portion of the guide RNA, has at least 90% sequence identity to any one of SEQ ID NO: 104-125 or 196-199. In some embodiments, the guide RNA, or the crRNA portion of the guide RNA, has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 104-125 or 196-199.

In some embodiments, the protein-binding segment, or the tracrRNA sequence, of engineered guide polynucleotide has at least 90% sequence identity to any one of SEQ ID NOs: 102 and 148-171. In some embodiments, the protein-binding segment of the engineered guide polynucleotide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 102 and 148-171.

In some embodiments, the disclosure provides an engineered guide polynucleotide for a Cas9 protein, having at least 90% sequence identity to any one of SEQ ID NOs: 172-191. In some embodiments, the engineered guide polynucleotide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 172-191.

Guide polynucleotides described herein may be designed using bioinformatics tools with biochemical validation. An exemplary process for designing a guide polynucleotide is as follows: (1) find a relevant CRISPR operon using protein BLAST; (2) search for crRNAs which are already annotated in the genome, or annotate the CRISPR using, e.g., CRISPR-Finder; (3) determine the possible location of tracrRNA using an alignment tool, e.g., the CLC Genomics Workbench (QIAGEN); (4) search for a TATAA Box in the vicinity of the regions with similarity to the crRNA; (5) test the secondary structure of the crRNA and all possible tracrRNAs found during the alignment and select the crRNA/tracrRNA hybrid that makes the desired secondary structure; and (6) trim the crRNA and the tracrRNA to create a short guide RNA (sgRNA). For example, the crRNA and tracrRNA sequences described herein may be combined to generate a sgRNA. In some embodiments, the crRNA and tracrRNA sequences are combined as shown in Table 1 to generate a sgRNA.

TABLE 1
Short Guide RNA Sequences (sgRNA) for Cas9 Proteins

| Cas9 Protein | crRNA SEQ ID NO | tracrRNA SEQ ID NO |
|---|---|---|
| LpCas9 | 104 | 148 |
| SsCas9 | 105 | 149 |
| WsCas9 | 106 | 150 |
| BbCas9 | 107 | 151 |
| PeCas9 | 108 | 152 |
| SwCas9 | 109 | 153 |
| RaCas9 | 110 | 154 |
| Csp1Cas9 | 111 | 155 |
| Csp2Cas9 | 112 | 156 |
| Cl 1Cas9 | 113 | 157 |
| C12Cas9 | 114 | 158 |
| MH0245Cas9 | 115 | 159 |
| FnCas9 | 116 | 160 |
| GpCas9 | 117 | 161; 162 |
| TmCas9 | 118 | 163 |
| L1Cas9 | 119 | 164 |
| SshCas9 | 120 | 165 |
| Lept.Cas9 | 121 | 166 |
| MoritellaCas9 | 122 | 167 |
| ExCas9 | 123 | 168 |
| TsCas9 | 124 | 169 |
| VnCas9 | 125 | 170; 171 |

### EXAMPLES

### Example 1

### Targeted Gene Insertion at the AAVS1 Locus

This Example verified gene insertion into the AAVS1 locus using seamless mutagenesis as disclosed herein (ObLiGaRe 2.0 system).

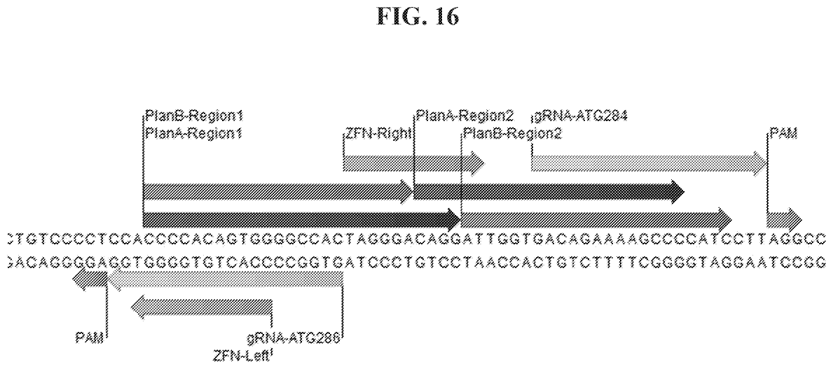

Two Cas9n-FokI variants, Cas9n^{D10A} and Cas9n^{H840A} were generated as shown in FIGS. 12 and 14. Two donor vectors were generated as shown in FIGS. 13 and 15, containing ObLiGaRe 2.0 target sites (denoted as Region2 and Region1 in the figures) upstream of a SA-2A-Puro selection cassette. The size of the donor vector was 6 kb. The ObLiGaRe 2.0 target sites were designed based on the AAVS1 locus, as shown in FIG. 16.

A plasmid encoding one of the Cas9n-FokI variants, 4 separately-cloned guide RNAs (gRNA), and the corresponding donor vector were co-transfected into HEK293 cells. Genomic insertion of the puromycin resistance cassette (gene of interest on the donor plasmid) was shown schematically in FIG. 15.

Cells which had puromycin resistance were selected, and genomic DNA of the puromycin-resistant cells were collected and subjected to junction PCR. The PCR products were TOPO-cloned and sequenced by Sanger sequencing to determine the precision at the junctions.

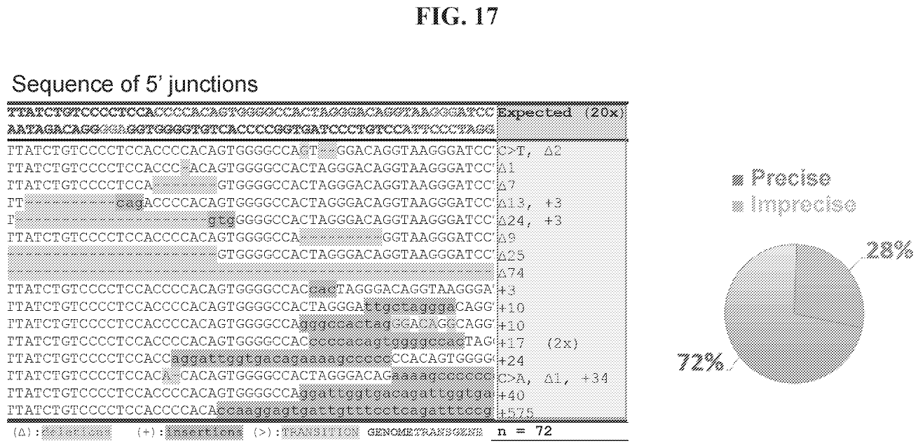

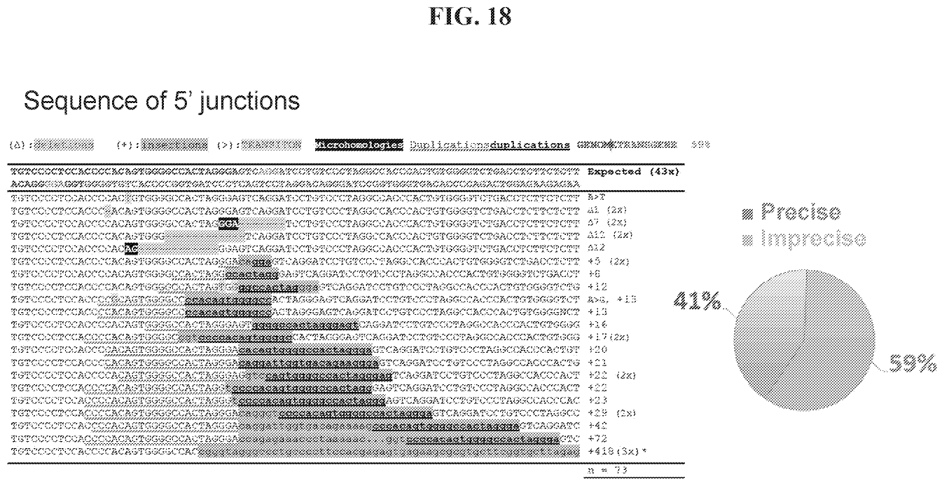

The sequence of 5′ junctions for gene insertion using Cas9n^{D10A}-FokI were shown in FIG. 17. The sequence of 5′ junctions for gene insertion using Cas9n^{H840A}-FokI were shown in FIG. 18. Thus, transgene cassettes were successfully knocked into AAVS1 locus using the ObLiGaRe 2.0 system, with high precision on the expected junctions.

### Example 2

### Evaluating the Efficiency of Targeted Insertion without Antibiotic Selection, and the Influence of Spacer Length on Gene Insertion Efficiency

In this Example, the influence of spacer length (the off-set sequence between two gRNAs) on the gene insertion efficiency was tested using an experimental set-up that did not require antibiotic selection.

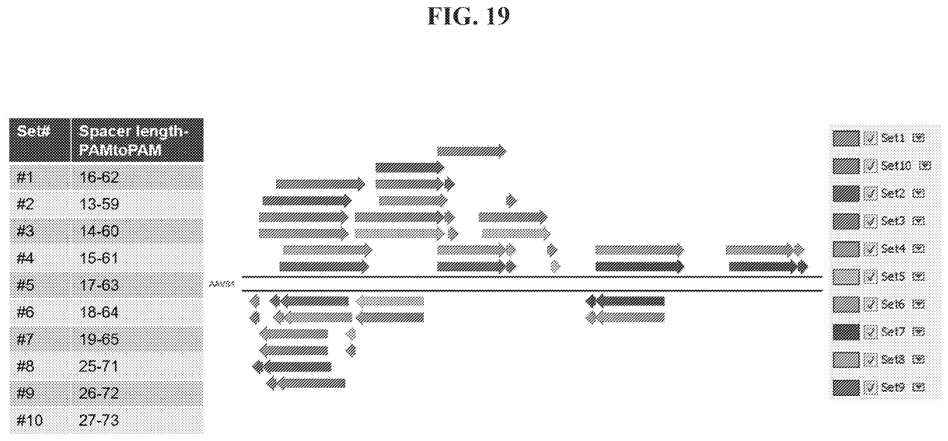

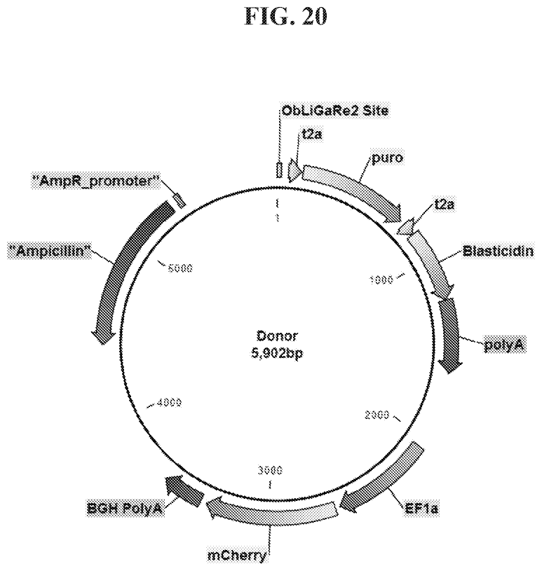

The AAVS1-Exon2 locus was selected as the target site. Required gRNAs for targeting 10 target sites, differing in the length of the spacer, were designed and cloned as shown in FIG. 19. Accordingly, 10 donor vectors containing the designed ObLiGaRe 2.0 target site and mCherry (under the control of a EF1a promoter) were generated as shown in FIG. 20.

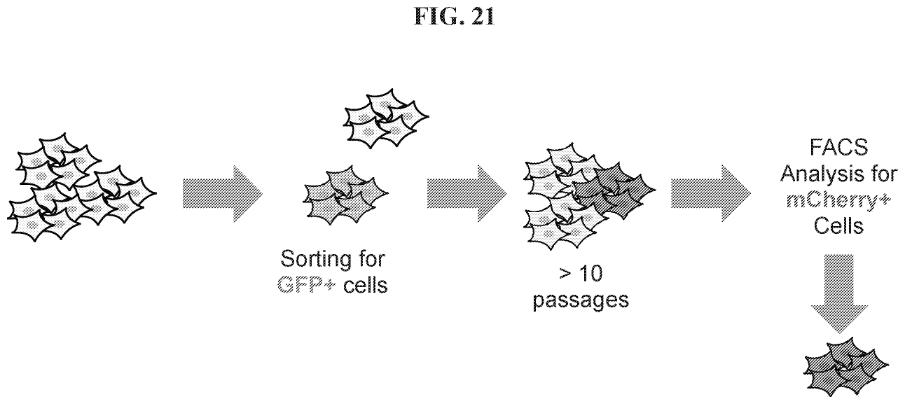

A plasmid encoding Cas9n^{H840A}-FokI and 2AGFP, 2 of the gRNAs, and the donor vector were co-transfected into HEK293 cells. Selection was carried out as follows: cells were first sorted by FACS for GFP expression, indicating introduction of active Cas9n-FokI. Then, cells were passaged for at least 10 passages, and then sorted by FACS for mCherry expression, indicating insertion of mCherry at the target site. This schematic was shown in FIG. 21.

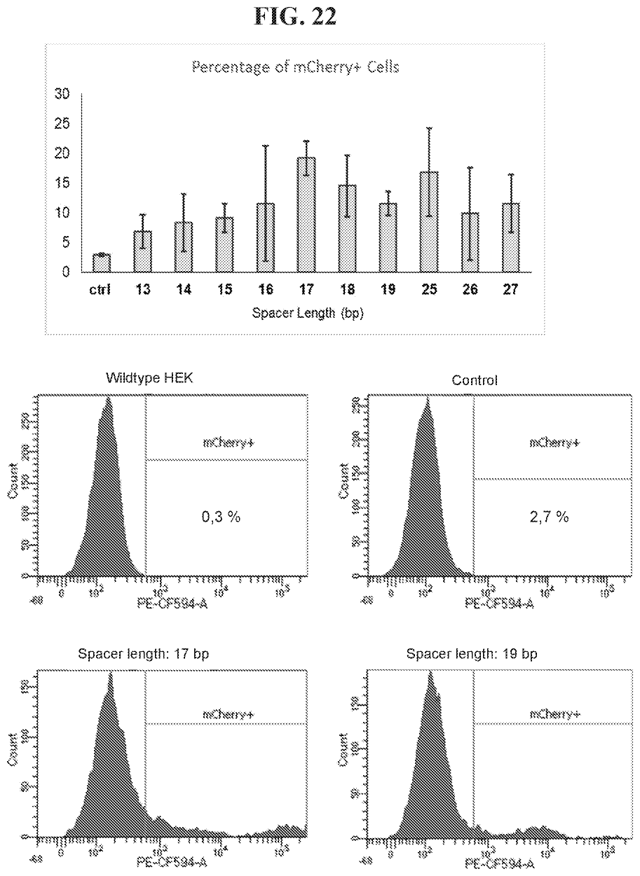

Results for the percentage of cells with mCherry vs. the spacer length (indicated in base pairs) were shown in FIG. 22. A spacer length of 17 bp indicated the highest efficiency of mCherry insertion (˜20%). Thus, high efficiency of transgene insertions with ObLiGaRe 2.0 without applying antibiotic selection was achieved.

### Example 3

### Comparison of the Efficiencies of Different Gene Insertions Methods

In this Example, gene insertion using ObLiGaRe (using zinc finger nucleases), and ObLiGaRe 2.0 were compared.

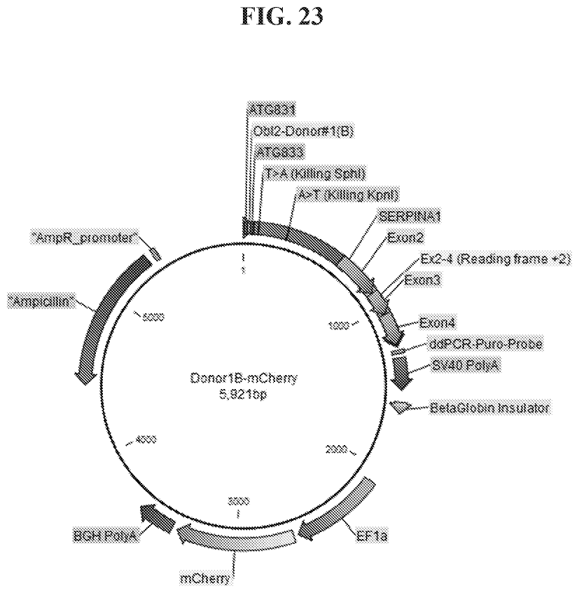

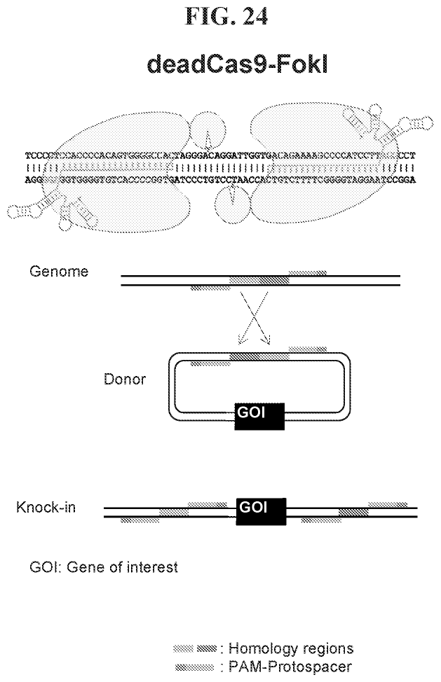

ObLiGaRe gene insertion was used for gene insertion into the AAVS1-int1 locus. ObLiGaRe 2.0 using Cas9n-FokI variants were used with 2 or 4 gRNAs, targeting AAVS1-int1 and three sites in SERPINA1-intron1 loci. ObLiGaRe 2.0 using deadCas9-FokI was also tested. The experimental procedure was carried out as described in Example 2 (no antibiotic selection, and cell selection based on FACS measurements of mCherry-positive cells). The donor plasmid for the SERPINA1 loci is shown in FIG. 23. Genomic insertion of the gene of interest on the donor plasmid using deadCas9-FokI was shown in FIG. 24.

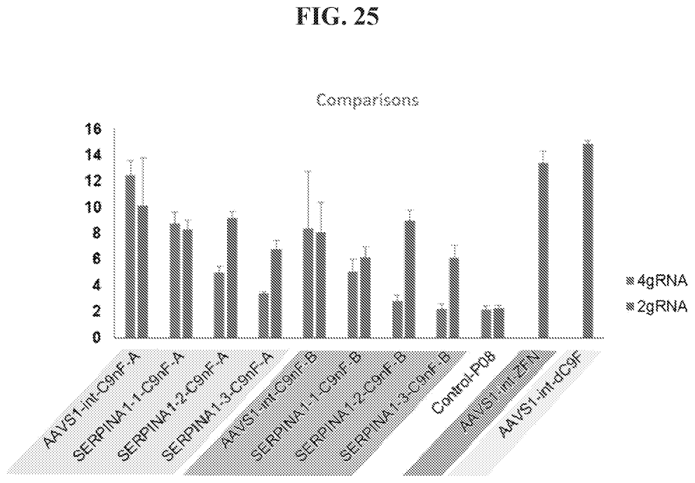

The results obtained for each of the gene insertion methods tested were shown in FIG. 25. The results were obtained from three independent biological replications in one experiment. Error bars indicated the S.E.M. The efficiency for the zinc finger nuclease-based ObLiGaRe (“AAVS1-int-ZFN”) and Cas9n^{D10A}-FokI (AAVS1-int-C9nF-A″) at the AAVS1-int1 locus were comparable. Variation in ObLiGaRe 2.0 efficiencies across different loci could be due to the efficiency of gRNAs. Obtaining a high gene insertion efficiency is achieved by evaluating a combination of target sites and different spacer lengths.

### Example 4

### Seamless Mutagenesis

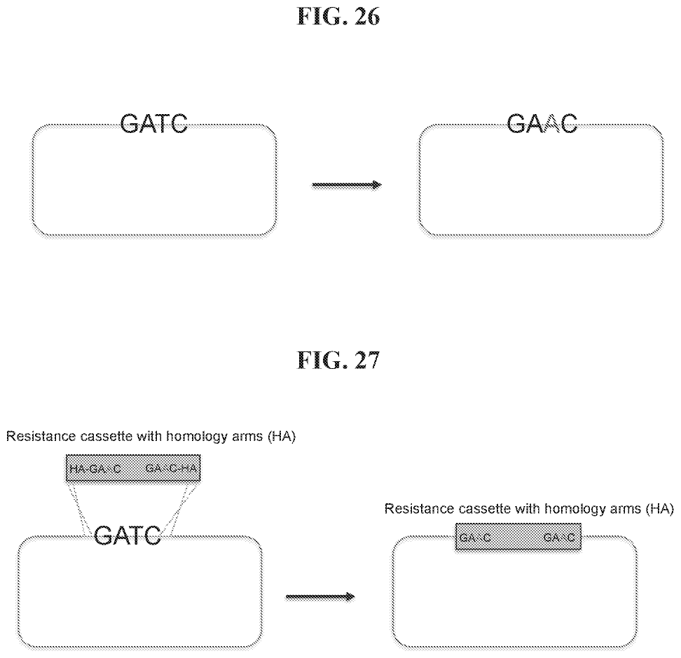

In this Example, a general process for seamless mutagenesis as provided in the disclosure herein is described. The desired result for seamless mutagenesis is shown in FIG. 26, wherein a mutation is made at a target site without changing any sequence in the target.

Step 1 of the process is shown in FIG. 27. A resistance cassette flanked by homology arms is introduced into a cell with the target sequence and inserted into the target region by homologous recombination. Cells containing the resistance cassette are selected.

A close-up of the resistance cassette is shown in FIG. 28. A nuclease cutting site and nuclease binding site are present on both sides of the resistance cassette. A nuclease such as Cpf1or Cas9 capable of generating overhangs cleaves at the nuclease cutting site, generating overhangs that include the desired point mutation.

Step 2 of the process is shown in FIG. 29. In vitro or in vivo ligation uses the compatible overhangs generated by the nuclease to remove the resistance cassette. The point mutation is thus inserted without leaving any “scar,” i.e., any extra sequences. A protocol for nucleic acid digestion and ligation is described in Example 5.

### Example 5

### Protocol for Seamless Mutagenesis using Cpf1

In this Example, nucleic acid digestion and ligation is performed as follows:

Digestion
1. Add together in a RNase-free 0.5 mL tube:

| | |
|---|---|
| 1 μL | Cas9 10 × Buffer |
| 1 μL | Cpf1 protein (10 μg/μL) |
| 1 μL | gRNA |

Up to 10 μL RNase-free H₂O (this amount is determined by the amount of DNA added in step 3).
2. Incubate at room temperature for 5 minutes.
3. Add 2-2.5 μg plasmid DNA to be cut (this volume will vary depending on the concentration; adjust the amount of water in step 1 accordingly).
4. Incubate at 37° C. for 2 hours.
5. After digestion, perform gel electrophoresis with 1.5% agarose gel at 150V.

Gel Extraction
6. Cut the DNA with the appropriate length from the gel.
7. Use a Gel Extraction Kit (e.g., from QIAGEN) to extract DNA from the gel.
8. Measure the DNA concentration on a NANODROP.

Ligation
9. Add together in a PCR tube:

| | | |
|---|---|---|
| 25-30 | ng | plasmid DNA (this volume will vary |
| | | depending on the concentration) |
| 1 | μL | DTT |
| 1 | μL | 10 × T4 ligase buffer |
| 1 | μL | T4 ligase |

Up to 10 μL H₂O
10. Incubate at 16° C. for 2 hours.
11. Use 10 μl for transformation.

Transformation
12. Thaw NEB10β cells (NEW ENGLAND BIOLABS) from −80° C. freezer by placing them on ice for 10 minutes. Each vial contains 50 μL (sufficient for 3 transformations). Thaw SOC medium.
13. Add 10 μL of the ligation reaction to a 1.5 mL EPPENDORF tube and place on ice to cool down.
14. After thawing, add 15 μL NEB10β cells to the ligation reaction.
15. Leave on ice for 30 minutes. Warm up 42° C. water bath.
16. Heat-shock cells by placing them at 42° C. in the water bath for 30 seconds, and then on ice for 2 minutes.
17. Add 300 μL SOC medium to the cells and incubate for 45 minutes at 37° C.
18. Plate 100 μL of the cells on ⅓ of a plate, or 300 μL on a whole plate; the plate contains the appropriate antibiotic.

### Example 6

### Cas9 In Vitro Digestion Protocol

In this Example, in vitro digestion of substrate DNA by Cas9 is performed as follows (for a 30 μL reaction):
1. Assemble the reaction at room temperature in the following order:

| | |
|---|---|
| 20 μL | Nuclease-free water |
| 3 μL | 10 × Cas9 Nuclease Reaction Buffer |
| 3 μL | 300 nM sgRNA (30 nM final concentration) |
| 1 μL | 1 μM Cas9 Nuclease (~30 nM final concentration) |

Pre-incubate for 10 minutes at 25° C., then add:

| | |
|---|---|
| 3 μL | 30 nM substrate DNA |

2. Mix thoroughly and pulse-spin in a microfuge.
3. Incubate at 37° C. for 15 minutes.
4. Add 1 μL of Proteinase K to each sample. Mix thoroughly and pulse-spin in a microfuge.
5. Incubate at room temperature for 10 minutes.
6. Proceed with fragment analysis.

### Example 7

### Analysis of DNA Repair Profiles Following Cas9 Cleavage

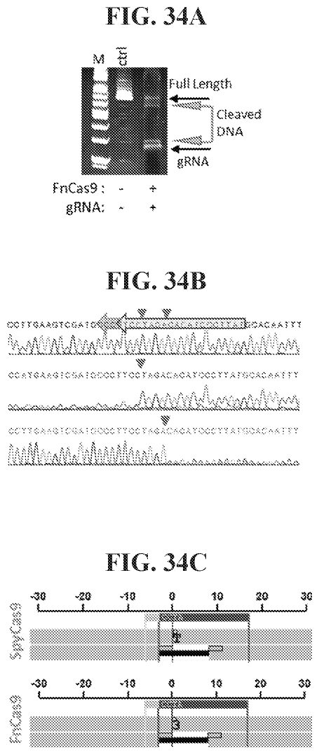

In this Example, computational analysis was used to identify Type II-B Cas9 operons by searching for presence of cas4 in the operon. The Cas9 protein from Francisella novicida (FnCas9) was chosen for production. Nuclease activity was demonstrated in an in vitro cleavage assay as shown in FIG. 34A. Sanger sequencing of cleaved products revealed that FnCas9 generates 5′ cohesive ends in vitro, as shown in FIG. 34B. The protein expression construct was validated in a HEK293 human cell line. RIMA was used to compare mutation patterns in FnCas9 and the Cas9 protein from Streptococcus pyogenes (SpyCas9), as shown in FIG. 34C.

### Example 8

### Analysis of DNA Cut Profiles Following Cas9 Treatment

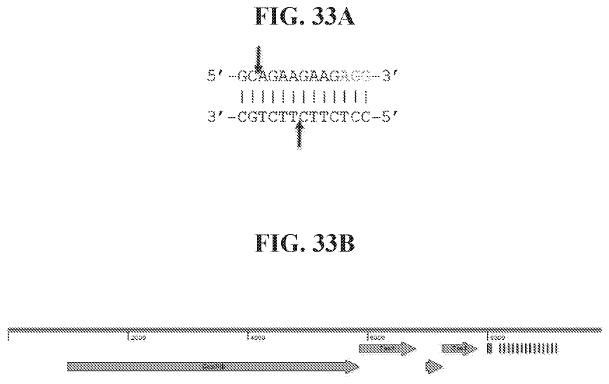

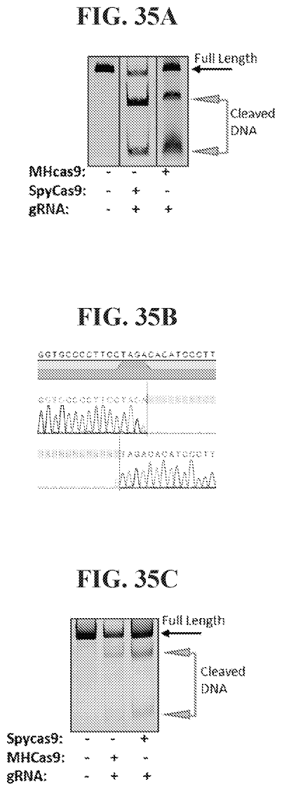

A Type II-B Cas9 variant from Francisella novicida (FnCas9) was shown to form cohesive ends with a low editing efficiency in mammalian cells, as described in Example 7. Other members of the Type II-B Cas9 family were tested for generating cohesive ends. A new Cas9 variant from the sequenced gut metagenome MH0245 was identified (MHCas9). Sequences of the guide RNA, tracrRNA, and crRNA designed for MHCas9 are shown in FIG. 33. In vitro assays showed that MHCas9 is capable of cleaving a DNA fragment, as shown in FIG. 35A. Sanger sequencing revealed that MHCas9 generates 5′ overhangs in vitro, as shown in FIG. 35B. Furthermore, a Cell1 assay was performed to validate that MHCas9 is also functional in a HEK293-REMINDEL human cell line, as shown in FIG. 35C.

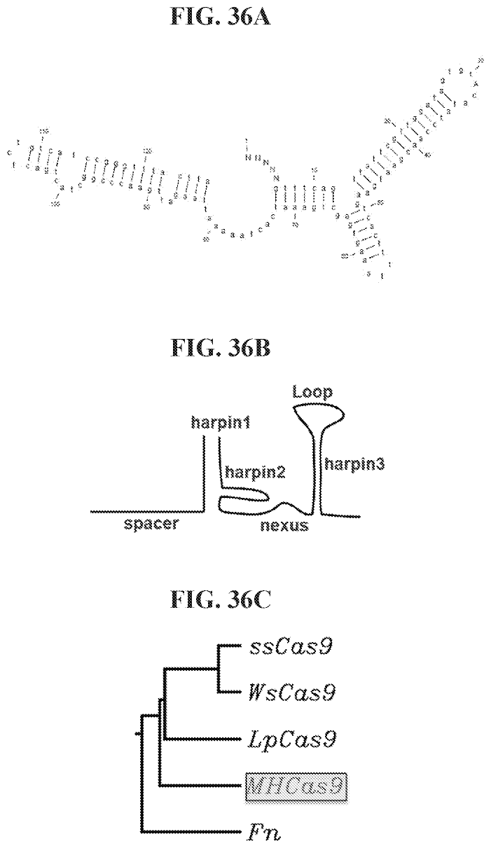

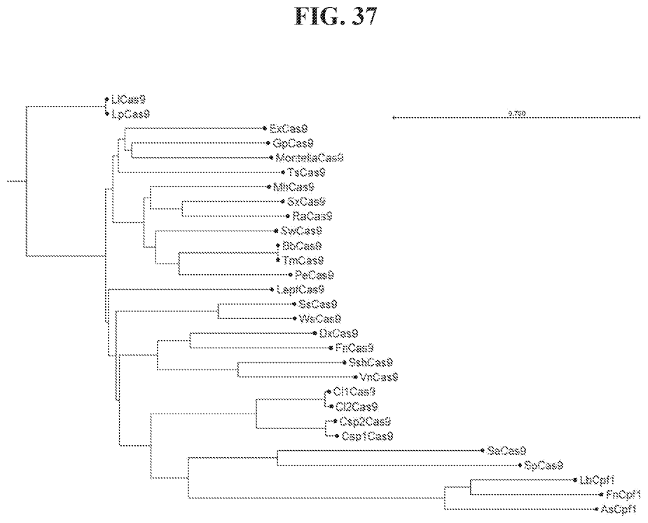

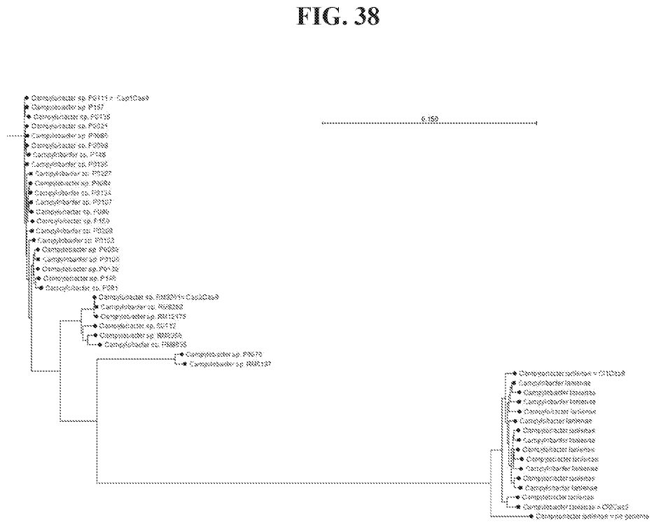

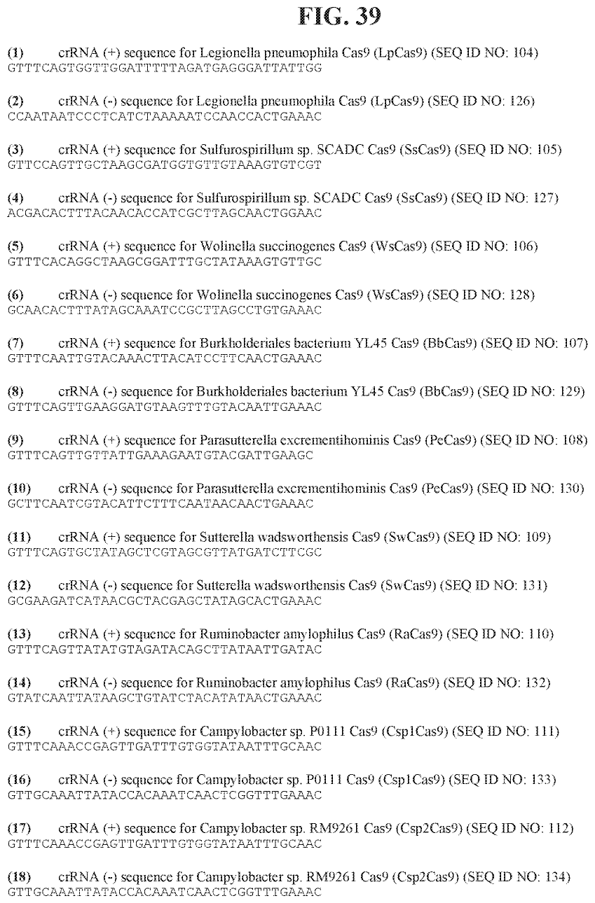

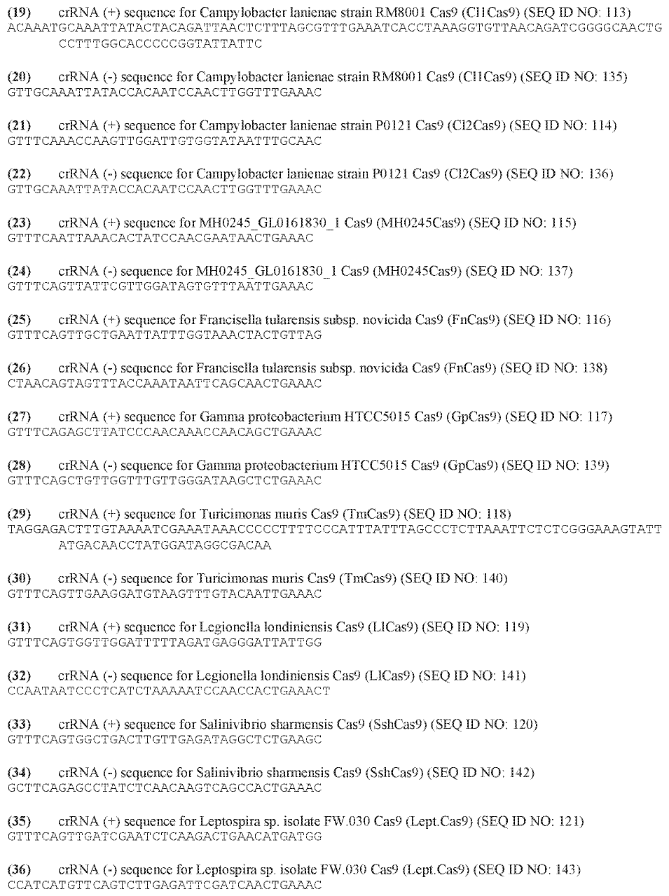

The sequence of the crRNA/tracrRNA from MHCas9 is shown in FIG. 36A. A scheme of the crRNA/tracrRNA, indicating the secondary structures, is shown in FIG. 36B. A truncated phylogenetic tree in FIG. 36C shows alignment of MHCas9 with other Type II-B Cas9, including Cas9 from Sulfurospirillum sp. SCADCh (ssCas9), Wolinella succinogenes (WsCas9), Legionella pneumophila (LpCas9) and FnCas9. As indicated by the phylogenetic tree, FnCas9 and MHCas9 are fairly divergent. However, experimental results described in Example 7 and this example show that MHCas9 and FnCas9 share the same mechanism of cleavage.

### Example 9

### Design of sgRNAs

In this Example, the methodology for design of a sgRNA is described:
1. Find the relevant CRISPR operons using Protein BLAST (NCBI, blast.ncbi.nlm.nih.gov/Blast.cgi?PAGE=Proteins). For each of the species appeared in the search, one of the RefSeq is selected for further analysis. BLAST is run several times with various inputs and different settings.
2. Check for the CRISPR RNAs (crRNAs) that are already annotated. Otherwise, annotate the crRNAs using CRISPR-Finder (crispr.i2bc.paris-saclay.fr/Server/).
3. Find the possible location of tracrRNA using “Create Alignment” in CLC Genomics Workbench v.9.5 (QIGEN). Both strands of the crRNA are aligned to the sequence between Cas4 and the CRISPR repeat sequences.
4. Look for a TATAA Box in the vicinity of the regions which show similarity with the crRNA.
5. Test the secondary structure of the crRNA with all possible tracrRNAs (found in the alignment) and select the ones that make a desirable structure.
6. Trim the crRNA and tracrRNA to make a short guide RNA (sgRNA).

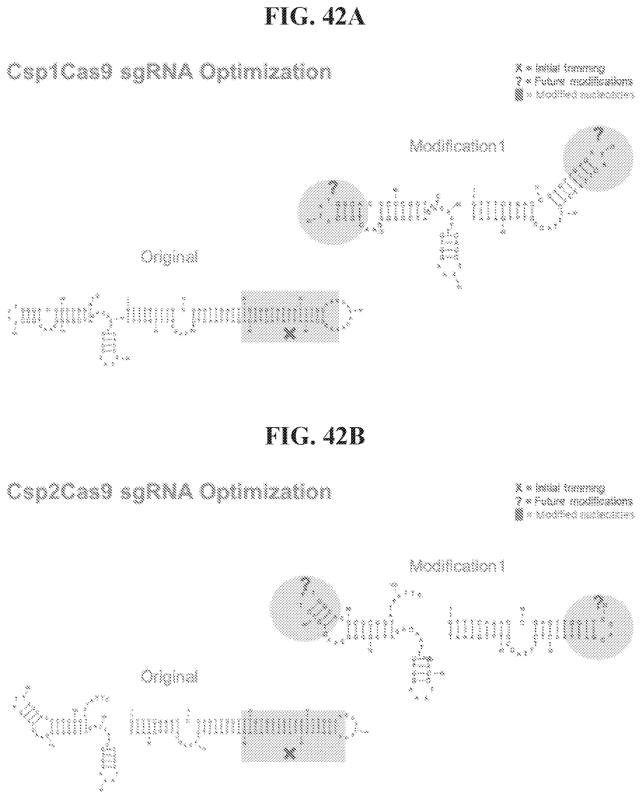

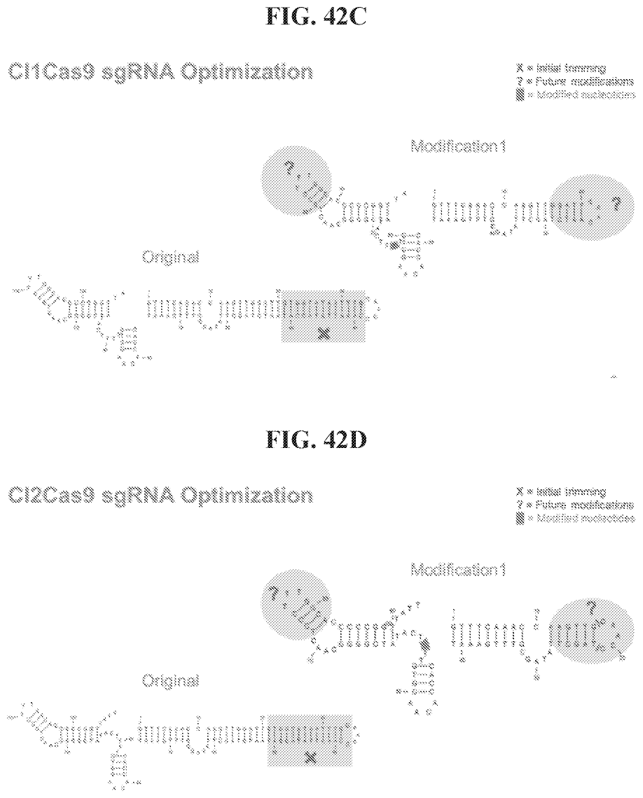

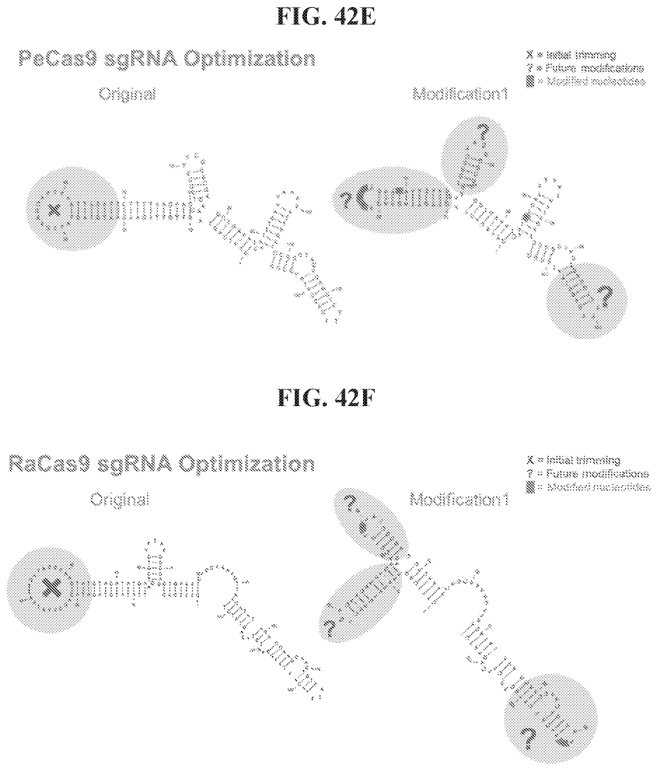

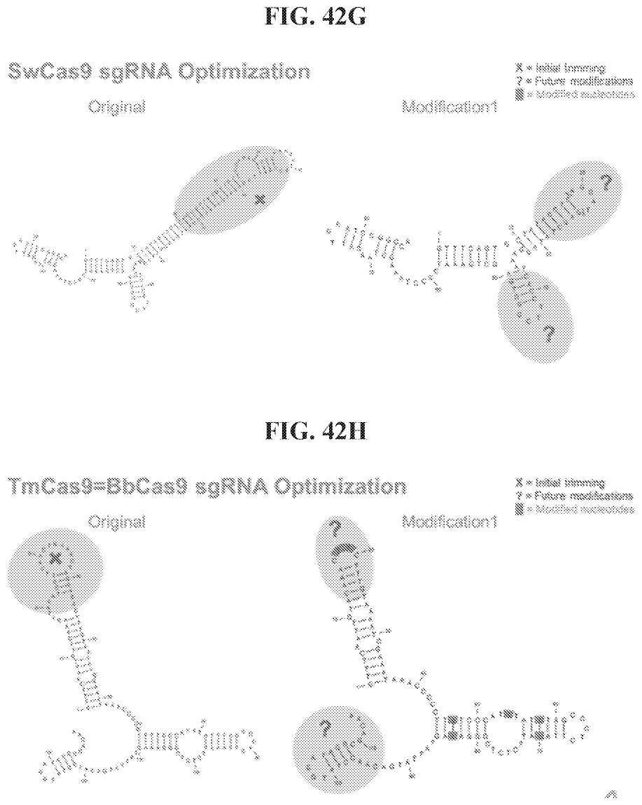

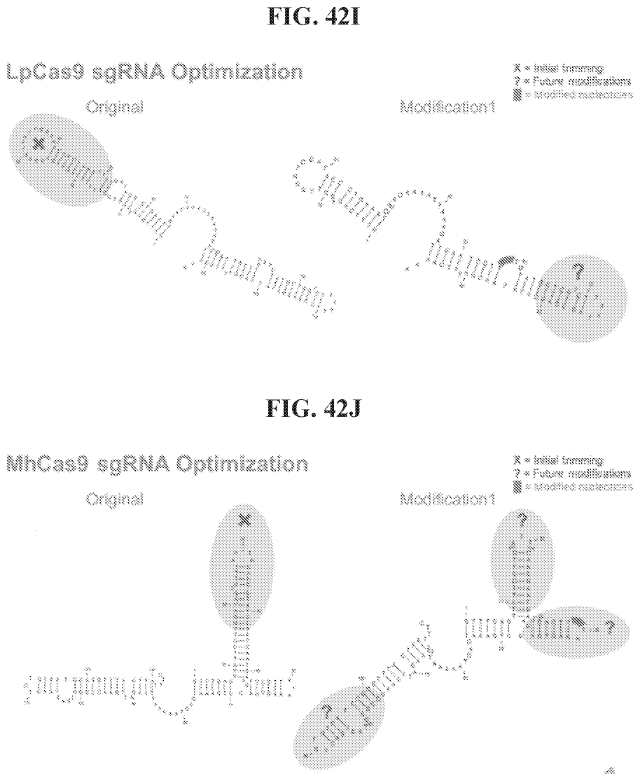

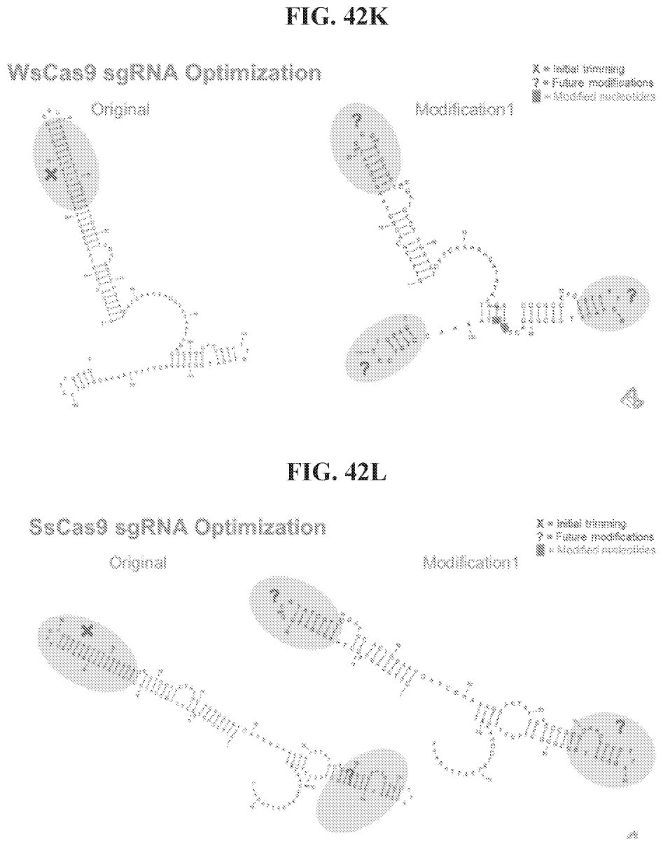

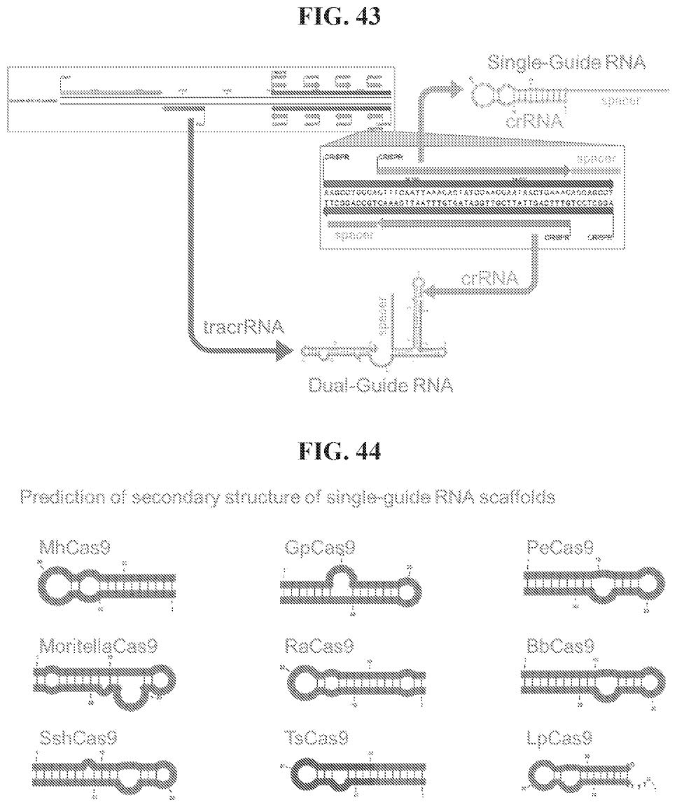

FIGS. 41A-T illustrate various sgRNAs designed by the method described herein. FIGS. 42A-L illustrate the optimization of sgRNAs (also termed “chimeric gRNA) by trimming, and possible target sites for further modifications.

### Example 10

### In Vitro Digestion Assays of Modified sgRNA

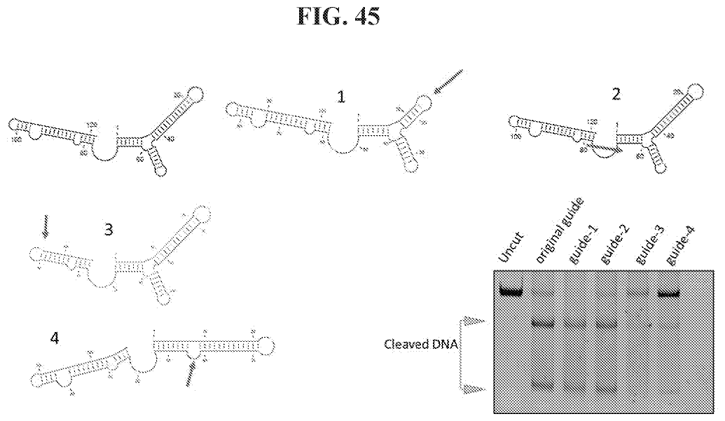

Four different guide RNA were engineered as outlined in FIG. 45 (guide-1, guide-2, guide-3, guide-4) by removing various nucleotides. The modified guide RNA were then compared to the original guide RNA in an in vitro digestion assay. FIG. 45 demonstrates that some modifications improved the digestion efficiency of MHCas9.

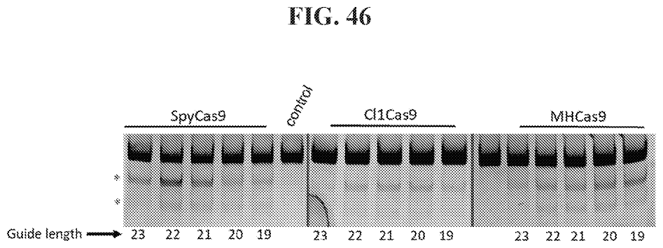

Guide RNA length was further investigated in three different Cas9 systems: SpyCas9, Cl1Cas9 and MHCas9. Guide RNA of lengths 19-23 were prepared, then the new Cas9 variants and engineered guide RNA were transfected into a reporter cell line and subjected to Surveyor™ nuclease assay (Integrated DNA Technologies, Skokie, Ill.). FIG. 46 demonstrates the cutting efficiency and functionality of new Cas9 variances Cl1 and MH in vitro.

### Example 11

### PAM Sequences for MHCas9

The preferred PAM sequence for MHCas9 was investigated using the method shown schematically in FIG. 49A. A pooled library of 64 plasmids was generated covering various PAM sequence combinations and a target cleavage site. SpCas9 and MHCas9 were used to separately digest the library. Forward and reverse primers for the plasmid were used to amplify the region containing the target cleavage site and the PAM, and the amplified regions were then sequenced by next-generation sequencing. The plasmids containing the preferred PAM sequences for either SpCas9 or MHCas9 were digested and thus not amplified or sequenced. On the other hand, the plasmids containing non-preferred PAM sequences for SpCas9 or MHCas9 were not digested and could be amplified.

Results for the “depleted” PAM sequences for SpCas9 and MHCas9 are shown in FIG. 49B. Compared with SpCas9, MHCas9 has a less stringent preference for the “NGG” PAM sequence.

### Example 12

### Coupling Cas9 Proteins with Exonucleases

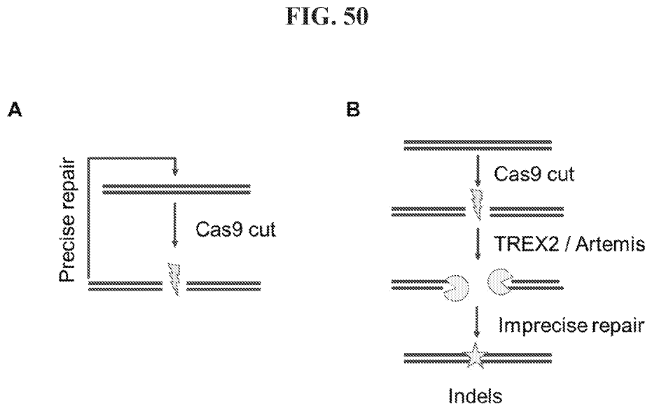

Cleavage by Type II-B Cas9 proteins was coupled with an end processing exonuclease enzyme to increase editing efficiency. A schematic of the method is illustrated in FIG. 50. As shown in FIG. 50A, overhangs generated from cleavage by Type II-B Cas9 can be repaired precisely by the cell to revert to the original sequence, thus limiting the editing efficiency when insertion-deletion or substitution modifications are desired. In FIG. 50B, after cleavage by Type II-B Cas9, the end processing exonuclease enzyme Artemis or TREX2 is introduced, which further processes the cleaved overhangs at the Type II-B Cas9 cut site. Cellular repair of these processed ends results in imprecise repair (i.e., increased number of insertion-deletion or substitution modifications) relative to the original sequence, thereby increasing the editing efficiency.

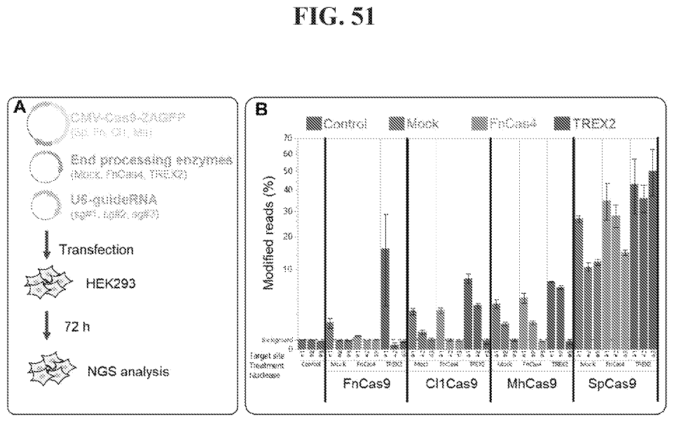

To test the effects of coupling Cas9 with exonucleases, Type II-B Cas9 with or without an end processing enzyme were tested for activity in human cell lines. FIG. 51A shows a schematic overview of the experimental procedure. Plasmids encoding various Type II-B Cas9 proteins (FnCas9, Cl1Cas9, MHCas9) and the Type II-A SpCas9 were introduced into HEK293 cells, along with plasmids encoding end processing enzymes FnCas4 or TREX2 and plasmids encoding three different guide RNA sequences. Genomic DNA from the HEK293 cells were harvested 72 hours after transfection and analyzed by next-generation sequencing.

Results are shown in FIG. 51B. Cells transfected with control plasmids showed only background levels of modification (attributed to natural variation in sequencing). FnCas9, MHCas9, and SpCas9 all showed varying amounts of genome modification either in the presence or absence of an end processing enzyme. Generally, introduction of Cas9 with an end processing enzyme showed increased number of modifications relative to no end processing enzyme.

### Example 13

### Mutation Pattern Analysis of Cas9 Proteins

Mutation pattern analysis for cuts made by different Cas9 was conducted. HEK293 cells were transfected with SpCas9, Cl1Cas9, or MHCas9 and their respective guide RNA's. Cells were lysed after 72 hours, and genomic DNA was extracted and subjected to next-generation amplicon sequencing. Sequencing reads were analyzed using bioinformatic tools to quantify the relative frequency of each mutation among the detected modified reads.

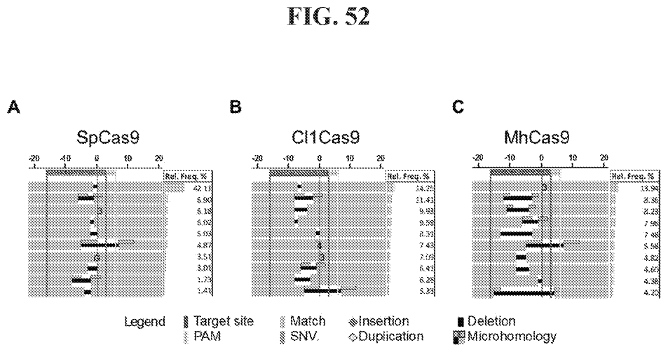

Results are shown in FIG. 52. FIGS. 52A, 52B, and 52C show the mutation patterns for the same target sequence after inducing a cut using, respectively, SpCas9, Cl1Cas9, and MHCas9. The target sequence is shown at the top of each of the panels. These results indicate that mutation patterns at the same locus after inducing a cut using different Cas9 protein are different, indicating different modes nuclease activity for different Cas9s.

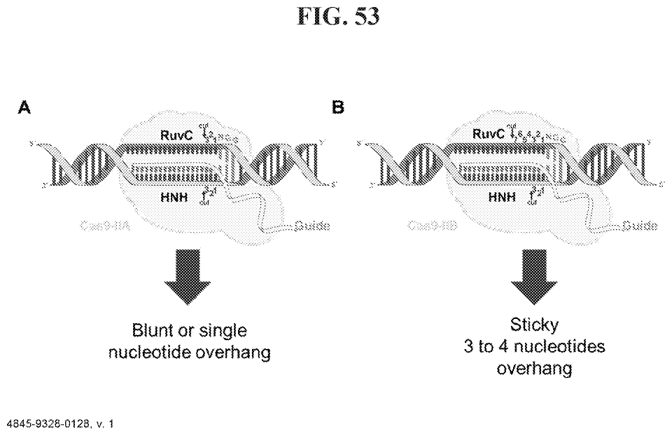

One non-limiting hypothesis for the difference in nuclease activity may be that the RuvC and HNH nuclease domain configurations differ between Type II-A and Type II-B Cas9 proteins. As illustrated in FIG. 53, a Type II-A Cas9 (panel A) indicates the same cut site for its RuvC and HNH domains (e.g., approximately 3 nucleotides upstream of the NGG PAM sequence), which leads to blunt ends or a single nucleotide overhang. On the other hand, a Type II-B Cas9 (panel B) indicates offset cut sites for RuvC and HNH (e.g., approximately 7 and 3 nucleotides, respectively, upstream of the NGG PAM sequence), which results in “sticky” ends, i.e., a 3-4 nucleotide overhang.

What is claimed is:

1. A non-naturally occurring CRISPR-Cas system comprising:
a) a Cas9 effector protein capable of generating cohesive ends (stiCas9); and
b) a guide polynucleotide that forms a complex with the stiCas9 and comprises a guide sequence, wherein the guide sequence is capable of hybridizing with a target sequence in a eukaryotic cell but does not hybridize to a sequence in a bacterial cell;
wherein the complex does not occur in nature.

2. A non-naturally occurring CRISPR-Cas system comprising:
a) a Cas9 effector protein capable of generating cohesive ends (stiCas9) and comprises a nuclear localization sequence (NLS); and
b) a guide polynucleotide that forms a complex with the stiCas9 and comprises a guide sequence;
wherein the complex does not occur in nature.

3. A non-naturally occurring CRISPR-Cas system comprising:
a) one or more nucleotide sequences encoding a Cas9 effector protein capable of generating cohesive ends (stiCas9); and
b) a nucleotide sequence encoding a guide polynucleotide that forms a complex with the stiCas9 and comprises a guide sequence, wherein the guide sequence is capable of hybridizing with a target sequence in a eukaryotic cell but does not hybridize to a sequence in a bacterial cell;
wherein the complex does not occur in nature.

4. A non-naturally occurring CRISPR-Cas system comprising:
a) one or more nucleotide sequences encoding a Cas9 effector protein capable of generating cohesive ends (stiCas9); and
b) a nucleotide sequence encoding a guide polynucleotide that forms a complex with the stiCas9 and comprises a guide sequence;
wherein the nucleotide sequences of (a) and (b) are under control of a eukaryotic promoter, and wherein the complex does not occur in nature.

5. The CRISPR-Cas system of any one of claims 1 to 4, wherein the guide polynucleotide comprises a tracrRNA sequence.

6. The CRISPR-Cas system of any one of claims 1 to 4, further comprising a separate polynucleotide comprising a tracrRNA sequence.

7. The CRISPR-Cas system of claim 6, wherein the guide polynucleotide, tracrRNA sequence and the stiCas9 are capable of forming a complex, and wherein the complex does not occur in nature.

8. A non-naturally occurring CRISPR-Cas system comprising one or more vectors comprising:
a) a regulatory element operably linked to one or more nucleotide sequences encoding a Cas9 effector protein capable of generating cohesive ends (stiCas9); and
b) a guide polynucleotide that forms a complex with the stiCas9 and comprises a guide sequence, wherein the guide sequence is capable of hybridizing with a target sequence in a eukaryotic cell but does not hybridize to a sequence in a bacterial cell;
wherein the complex does not occur in nature.

9. A non-naturally occurring CRISPR-Cas system comprising one or more vectors comprising:
a) a regulatory element operably linked to one or more nucleotide sequences encoding a Cas9 effector protein capable of generating cohesive ends (stiCas9), wherein the regulatory element is a eukaryotic regulatory element; and
b) a guide polynucleotide that forms a complex with the stiCas9 and comprising a guide sequence;
wherein the complex does not occur in nature.

10. The non-naturally occurring vector of claim 8 or claim 9, wherein the guide polynucleotide further comprises a tracrRNA sequence.

11. The non-naturally occurring vector of claim 9 or claim 10, further comprising a nucleotide sequence comprising a tracrRNA sequence.

12. The system of any one of claims 1 to 11, wherein the complex is capable of cleaving at a site within 10 nucleotides of a Protospacer Adjacent Motif (PAM).

13. The system of any one of claims 1 to 12, wherein the complex is capable of cleavage at a site within 5 nucleotides of a Protospacer Adjacent Motif (PAM).

14. The system of any of any one of claims 1 to 13, wherein the complex is capable of cleavage at a site within 3 nucleotides of a Protospacer Adjacent Motif (PAM).

15. The system of any one of claims 1 to 14, wherein the target sequence is 5′ of a Protospacer Adjacent Motif (PAM) and the PAM comprises a 3′ G-rich motif

16. The system of any one of claims 1 to 15, wherein the target sequence is 5′ of a Protospacer Adjacent Motif (PAM) and the PAM sequence is NGG, wherein N is A, C, G, or T.

17. The system of any one of claims 1 to 16, wherein the cohesive ends comprise a single-stranded polynucleotide overhang of 3 to 40 nucleotides.

18. The system of any one of claims 1 to 17, wherein the cohesive ends comprise a single-stranded polynucleotide overhang of 4 to 20 nucleotides.

19. The system of any one of claims 1 to 18, wherein the cohesive ends comprise a single-stranded polynucleotide overhang of 5 to 15 nucleotides.

20. The system of any one of claims 1 to 19, wherein the stiCas9 is derived from a bacterial species having a Type II-B CRISPR system.

21. The system of any one of claims 1 to 20, wherein the stiCas9 comprises a domain having at least 95% identity to any one of SEQ ID NOs: 10-97 or 192-195.

22. The system of any of one of claims 1 to 21, wherein the stiCas9 comprises a domain that matches a TIGR03031 protein family with an E-value cut-off of 1E-5.

23. The system of any one of claims 1 to 22, wherein the stiCas9 comprises a domain that matches the TIGR03031 protein family with an E-value cut-off of 1E-10.

24. The system of claim 23, wherein the bacterial species is Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1-47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonassp. S-B4-1U, Tamilnaduibacter salinus, Vibrio natriegens, Arcobacter skirrowii, Francisella philomiragia, Francisella hispaniensis, or Parendozoicomonas haliclonae.

25. The system of claim 24, wherein the target sequence is 5′ of a Protospacer Adjacent Motif (PAM) and the PAM sequence is YG, wherein Y is a pyrimidine and the stiCas9 is derived from the bacterial species F. novicida.

26. The system of any of any one of claims 1 to 25, wherein the stiCas9 comprises one or more nuclear localization signals.

27. The system of any of one of claims 1 to 26, wherein the eukaryotic cell is an animal or human cell.

28. The system of any one of claims 1 to 27, wherein the eukaryotic cell is a human cell.

29. The system of any one of claims 1 to 26, wherein the eukaryotic cell is a plant cell.

30. The system of any one of claims 1 to 29, wherein the guide sequence is linked to a direct repeat sequence.

31. A delivery particle comprising the system according to any one of claims 1 to 30.

32. The delivery particle of claim 31, wherein the stiCas9 and the guide polynucleotide are in a complex.

33. The delivery particle of claim 32, wherein the complex further comprises a polynucleotide comprising a tracrRNA sequence.

34. The delivery particle of claim 32 or 22, further comprising a lipid, a sugar, a metal, or a protein.

35. A vesicle comprising the system according to any one of claims 1 to 30.

36. The vesicle of claim 35, wherein the stiCas9 and the guide polynucleotide are in a complex.

37. The vesicle of claim 36, further comprising a polynucleotide comprising a tracrRNA sequence.

38. The vesicle of any one of claims 35 to 37, wherein the vesicle is an exosome or a liposome.

39. The system of any one of claims 5 to 9, wherein the one or more nucleotide sequences encoding the stiCas9 is codon optimized for expression in a eukaryotic cell.

40. The system of any one of claim 5 to 30 or 39, wherein the nucleotide sequence encoding a Cas9 effector protein and the guide polynucleotide are on a single vector.

41. The system of any one of claim 5 to 30 or 39, wherein the nucleotide sequence encoding a Cas9 effector protein and the guide polynucleotide are a single nucleic acid molecule.

42. A viral vector comprising the system according to any one of claims 5 to 30 or 39 to 41.

43. The viral vector of claim 42, wherein the viral vector is of an adenovirus, a lentivirus, or an adeno-associated virus.

44. A eukaryote cell comprising a CRISPR-Cas system comprising
a) a Cas9 effector protein capable of generating cohesive ends (stiCas9), and
b) a guide polynucleotide that forms a complex with the stiCas9 and comprises a guide sequence, wherein the guide sequence is capable of hybridizing with a target sequence in a eukaryotic cell;
wherein the complex does not occur in nature.

45. A eukaryote cell comprising a CRISPR-Cas system comprising a Cas9 effector protein capable of generating cohesive ends (stiCas9), wherein the Cas9 effector protein is derived from a bacterial species having a Type II-B CRISPR system.

46. A method for providing site-specific modification of a target sequence in a eukaryotic cell, the method comprising:
a) introducing into the cell:
i. a Cas9 effector protein capable of generating cohesive ends (stiCas9); and
ii. a guide polynucleotide that forms a complex with the stiCas9 and comprises a guide sequence, wherein the guide sequence is capable of hybridizing with the target sequence in the eukaryotic cell but does not hybridize to a sequence in a bacterial cell;
wherein the complex does not occur in nature; and
b) generating cohesive ends in the target sequence with the Cas9 effector protein and the guide polynucleotide; and
c) ligating
i. the cohesive ends together, or
ii. a polynucleotide sequence of interest (SoI) to the cohesive ends;
thereby modifying the target sequence.

47. A method for providing site-specific modification of a target sequence in a eukaryotic cell, the method comprising:
a) introducing into the cell:
i. a nucleotide sequence encoding a Cas9 effector protein capable of generating cohesive ends (stiCas9); and
ii. a guide polynucleotide that forms a complex with the stiCas9 and comprising a guide sequence, wherein the guide sequence is capable of hybridizing with the target sequence in the eukaryotic cell but does not hybridize to a sequence in a bacterial cell;
wherein the complex does not occur in nature; and
b) generating cohesive ends in the target sequence with the Cas9 effector protein and the guide polynucleotide; and
c) ligating
i. the cohesive ends together, or
ii. a polynucleotide sequence of interest (SoI) to the cohesive ends;
thereby modifying the target sequence.

48. The method of claim 46 or 47, wherein the guide polynucleotide further comprises a tracrRNA sequence.

49. The method of claim 46 or 47, further comprising introducing into the cell a polynucleotide comprising a tracrRNA sequence.

50. The method of claim 49, wherein the guide polynucleotide, tracrRNA sequence, and the stiCas9 are capable of forming a complex, and wherein the complex does not occur in nature.

51. The method of any one of claims 46 to 50, wherein the complex is capable of cleaving at a site within 10 nucleotides of a Protospacer Adjacent Motif (PAM).

52. The method of any one of claims 46 to 51, wherein the complex is capable of cleaving at a site within 5 nucleotides of a Protospacer Adjacent Motif (PAM).

53. The method of any one of claims 46 to 52, wherein the complex is capable of cleaving at a site within 3 nucleotides of a Protospacer Adjacent Motif (PAM).

54. The method of any one of claims 46 to 53, wherein the target sequence is 5′ of a Protospacer Adjacent Motif (PAM) and the PAM comprises a 3′ G-rich motif

55. The method of any one of claims 46 to 54, wherein the target sequence is 5′ of a PAM and the PAM sequence is NGG, wherein N is A, C, G, or T.

56. The method of any one of claims 46 to 55, wherein the cohesive ends comprise a single-stranded polynucleotide overhang of 3 to 40 nucleotides.

57. The method of any one of claims 46 to 56, wherein the cohesive ends comprise a single-stranded polynucleotide overhang of 4 to 20 nucleotides.

58. The method of any one of claims 46 to 57, wherein the cohesive ends comprise a single-stranded polynucleotide overhang of 5 to 15 nucleotides.

59. The method of any one of claims 46 to 58, wherein the stiCas9 is derived from a bacterial species having a Type II-B CRISPR system.

60. The method of any one of claims 46 to 59, wherein the eukaryotic cell is an animal or human cell.

61. The method of any one of claims 46 to 60, wherein the eukaryotic cell is a human cell.

62. The method of any one of claims 46 to 59, wherein the eukaryotic cell is a plant cell.

63. The method of any one of claims 46 to 62, wherein the modification is deletion of at least part of the target sequence.

64. The method of any one of claims 46 to 62, wherein the modification is mutation of the target sequence.

65. The method of any one of claims 46 to 62, wherein the modification is inserting a sequence of interest into the target sequence.

66. The method of any one of claims 46 to 65, further comprising introducing an exonuclease to remove overhangs generated by the stiCas9.

67. The method of claim 66, wherein the exonuclease is Cas4, Artemis, or TREX2.

68. The method of claim 67, wherein the Cas4 is derived from a bacterial species having a Type II-B CRISPR system.

69. The method of any one of claims 46 to 68, wherein polynucleotides encoding components of the complex are introduced on one or more vectors.

70. A method of introducing a sequence of interest (SoI) into a chromosome in a cell, wherein the chromosome comprises a target sequence (TSC) comprising region 1 and region 2, the method comprising introducing into the cell:
a) a vector comprising a target sequence (TSV), the TSV comprising region 2 and region 1 and the SoI;
b) a first Cas9-endonuclease dimer capable of generating cohesive ends in the TSC, wherein a first monomer of the first Cas9-endonuclease dimer cleaves at region 1 and a second monomer of the first Cas9-endonuclease dimer cleaves at region 2 of the TSC; and
c) a second Cas9-endonuclease dimer capable of generating cohesive ends in the TSV, wherein a first monomer of the second Cas9-endonuclease dimer cleaves at region 2 and a second monomer of the second Cas9-endonuclease dimer cleaves at region 1 of the TSV;
wherein introduction of the vector of (a), the first Cas9-endonuclease dimer of (b) and the second Cas9-endonuclease dimer of (c) into the cell results in insertion of the SoI into the chromosome of the cell.

71. A method of introducing a sequence of interest (SoI) into a chromosome in a cell, wherein the chromosome comprises a target sequence (TSC) comprising region 1 and region 2, the method comprising introducing into the cell:
a) a vector comprising a target sequence (TSV), the TSV comprising region 2 and region 1 and the SoI, wherein the vector comprises cohesive ends;
b) a first Cas9-endonuclease dimer capable of generating cohesive ends in the TSC, wherein a first monomer of the Cas9-endonuclease dimer cleaves at region 1 and a second monomer of the Cas9-endonuclease dimer cleaves at region 2 of the TSC;
wherein introduction of the vector of (a) and the first Cas9-endonuclease dimer of (b) into the cell results in insertion of the SoI into the chromosome of the cell.

72. The method of claim 70 or claim 71, wherein the first and second Cas9-endonuclease dimers are the same.

73. The method of claim 70 or claim 71, wherein the first and second Cas9-endonuclease dimers are different.

74. The method of any one of claims 70 to 73, further comprising introducing into the cell a first guide polynucleotide that forms a complex with the first monomer of the first Cas9-endonuclease dimer and comprises a first guide sequence, wherein the first guide sequence hybridizes to the TSC comprising region 1 but does not hybridize to the vector.

75. The method of any one of claims 70 to 73, further comprising introducing into the cell a first guide polynucleotide that forms a complex with the first monomer of the first Cas9-endonuclease dimer and comprises a first guide sequence, wherein the first guide sequence hybridizes to the TSC and the TSV.

76. The method of any one of claims 70 to 75, further comprising introducing into the cell a second guide polynucleotide that forms a complex with the second monomer of the first Cas9-endonuclease dimer and comprises a second guide sequence, wherein the second guide sequence hybridizes to the TSC comprising region 2 but does not hybridize to the vector.

77. The method of any one of claims 70 to 75, further comprising introducing into the cell a second guide polynucleotide that forms a complex with the second monomer of the first Cas9-endonuclease dimer and comprises a second guide sequence, wherein the second guide sequence hybridizes to the TSC and the TSV.

78. The method of any one of claims 70 to 77, further comprising introducing into the cell a third guide polynucleotide that forms a complex with the first monomer of the second Cas9-endonuclease dimer and comprises a third guide sequence, wherein the third guide sequence hybridizes to the TSV comprising region 2 but does not hybridize to the chromosome.

79. The method of claims 70 to 78, further comprising introducing into the cell a third guide polynucleotide that forms a complex with the first monomer of the second Cas9-endonuclease dimer and comprises a third guide sequence, wherein the third guide sequence hybridizes to the TSC and the TSV.

80. The method of any one of claims 70 to 79, further comprising introducing into the cell a fourth guide polynucleotide that forms a complex with the second monomer of the second Cas9-endonuclease dimer and comprises a fourth guide sequence, wherein the fourth guide sequence hybridizes to the TSV comprising region 1 but does not hybridize to the chromosome.

81. The method of any one of claims 70 to 80, further comprising introducing into the cell a fourth guide polynucleotide that forms a complex with the second monomer of the second Cas9-endonuclease dimer and comprises a fourth guide sequence, wherein the fourth guide sequence hybridizes to the TSC and the TSV.

82. The method of any one of claims 70 to 81, comprising introducing into the cell the first, second, third, and fourth guide polynucleotides.

83. The method of any one of claims 70 to 82, further comprising introducing into the cell a polynucleotide comprising a tracrRNA sequence.

84. The method of any one of claims 70 to 83, wherein the endonucleases in the first monomer and the second monomer of the first Cas9-endonuclease dimer are Type IIS endonucleases.

85. The method of any one of claims 70 to 83, wherein the endonucleases in the first monomer and the second monomer of the second Cas9-endonuclease dimer are Type IIS endonucleases.

86. The method of any one of claims 70 to 85, wherein the endonucleases in the first Cas9-endonuclease dimer and the second Cas9-endonuclease dimer are Type IIS endonucleases.

87. The method of any one of claims 70 to 86, wherein the endonucleases in the first Cas9-endonuclease dimer and the second Cas9-endonuclease dimer, are independently selected from the group consisting of BbvI, BgcI, BfuAI, BmpI, BspMI, CspCI, FokI, MboII, MmeI, NmeAIII, and PleI.

88. The method of any one of claims 70 to 87, wherein the endonucleases in the first Cas9-endonuclease dimer and the second Cas9-endonuclease dimer are FokI.

89. The method of any one of claims 70 to 88, wherein the first and second Cas9-endonuclease dimers are introduced into the cell as polynucleotides encoding the first and second Cas9-endonuclease dimers.

90. The method of claim 89, wherein the polynucleotide encoding the first and second Cas9-endonuclease dimers are on one vector.

91. The method of claim 89, wherein the polynucleotide encoding the first and second Cas9-endonuclease dimers are on more than one vector.

92. The method of any one of claims 70 to 91, wherein the first, second or both Cas9-endonuclease dimers comprise a modified Cas9.

93. The method of claim 92, wherein the first, second or both Cas9-endonuclease dimers comprise a catalytically inactive Cas9.

94. The method of claim 93, wherein the endonuclease in the first, second or both Cas9-endonuclease dimers is FokI.

95. The method of claim 92, wherein the first, second or both Cas9-endonuclease dimers comprise a Cas9 having nickase activity.

96. The method of claim 95, wherein the endonuclease in the first, second or both Cas9-endonuclease dimers is FokI.

97. The method of claim 92, wherein the Cas9-endonuclease dimer comprises a single amino-acid substitution in Cas9 relative to a wild-type Cas9.

98. The method of claim 97, wherein the endonuclease in the first, second or both Cas9-endonuclease dimers are FokI.

99. The method of claim 97 or 98, wherein the single amino-acid substitution is D10A or H840A.

100. The method of claim 97 or 98, wherein the single amino-acid substitution is D10A.

101. The method of claim 97 or 98, wherein the single amino-acid substitution is H840A.

102. The method of claim 92, wherein the Cas9-endonuclease dimer comprises a double amino-acid substitution relative to a wild-type Cas9.

103. The method of claim 102, wherein the double amino-acid substitution is D10A and H840A.

104. The method of claim 97, wherein the wild-type Cas9 is derived from Streptococcus pyogenes, Staphylococcus aureus, Staphylococcus pseudintermedius, Planococcus antarcticus, Streptococcus sanguinis, Streptococcus thermophilus, Streptococcus mutans, Coribacterium glomerans, Lactobacillus farciminis, Catenibacterium mitsuokai, Lactobacillus rhamnosus, Bifidobacterium bifidum, Oenococcus kitahara, Fructobacillus fructosus, Finegoldia magna, Veillonella atyipca, Solobacterium moorei, Acidaminococcus sp. D21, Eubacterium yurri, Coprococcus catus, Fusobacterium nucleatum, Filifactor alocis, Peptoniphilus duerdenii, or Treponema denticola.

105. The method of any one of claims 70 to 104, wherein the cohesive ends comprise a 5′ overhang.

106. The method of any one of claims 70 to 104, wherein the cohesive ends comprise a 3′ overhang.

107. The method of any one of claims 70 to 106, wherein the first, second or both Cas9-endonuclease dimers generate cohesive ends comprising a single-stranded polynucleotide of 3 to 40 nucleotides.

108. The method of any one of claims 70 to 106, wherein the first, second or both Cas9-endonuclease dimers generate cohesive ends comprising a single-stranded polynucleotide of 4 to 30 nucleotides.

109. The method of any one of claims 70 to 106, wherein the first, second or both Cas9-endonuclease dimers generate cohesive ends comprising a single-stranded polynucleotide of 5 to 20 nucleotides.

110. The method of any one of claims 70 to 109, wherein upon the insertion, the target sequence in the chromosome and the target sequence in the plasmid are not reconstituted.

111. The method of any one of claims 70 to 110, wherein the cell is a eukaryotic cell.

112. The method of any one of claims 70 to 111, wherein the cell is an animal or human cell.

113. The method of any one of claims 70 to 112, wherein the cell is a plant cell.

114. The method of any one of claims 70 to 113, wherein the vector of (a), the first Cas9-endonuclease dimer of (b), the second Cas9-endonuclease dimer of (c) or combinations thereof are introduced into the cell via delivery particles, vesicles, or viral vectors.

115. The method of any one of claims 70 to 114, wherein the vector of (a), the first Cas9-endonuclease dimer of (b), the second Cas9-endonuclease dimer of (c) or combinations thereof are introduced into the cell via delivery particles.

116. The method of claim 115, wherein the delivery particles comprise a lipid, a sugar, a metal, or a protein.

117. The method of any one of claims 70 to 114, wherein the vector of (a), the first Cas9-endonuclease dimer of (b), the second Cas9-endonuclease dimer of (c) or combinations thereof are introduced into the cell via vesicles.

118. The method of claim 117, wherein the vesicles are exosomes or liposomes.

119. The method of any one of claims 70 to 113, wherein polynucleotides capable or expressing (b), (c) or combinations thereof are introduced into the cell via a viral vector.

120. The method of any one of claims 70 to 113, wherein the vector of (a) is a viral vector.

121. The method of claim 119 or 120, wherein the viral vector is an adenovirus, lentivirus, or adeno-associated virus.

122. The method of any one of claims 70 to 121, wherein the first monomer of the first Cas9-endonuclease dimer forms a complex with the first guide polynucleotide, and the second monomer of the first Cas9-endonuclease dimer forms a complex with the second guide polynucleotide.

123. The method of any one of claims 70 to 122, wherein the first monomer of the second Cas9-endonuclease dimer forms a complex with the third guide polynucleotide, and the second monomer of the second Cas9-endonuclease dimer forms a complex with the fourth guide polynucleotide.

124. The method of any one of claims 70 to 121, wherein the first monomer of the first Cas9-endonuclease dimer forms a complex with the first guide polynucleotide sequence and a tracrRNA sequence, and the second monomer of the first Cas9-endonuclease dimer forms a complex with the second guide polynucleotide sequence and a tracrRNA sequence.

125. The method of any one of claims 70 to 122, wherein the first monomer of the second Cas9-endonuclease dimer forms a complex with the third guide polynucleotide sequence and a tracrRNA sequence, and the second monomer of the second Cas9-endonuclease dimer forms a complex with the fourth guide polynucleotide sequence and a tracrRNA sequence.

126. The method of any one of claims 70 to 125, wherein the first, second or both Cas9-endonuclease dimers comprise a nuclear localization signal.

127. The method of any one of claims 70 to 126, wherein the cell comprises a stem cell or stem cell line.

128. A method of modifying one or more nucleotides in a target polynucleotide sequence in a cell, the method comprising:
a) introducing into the cell a vector comprising an insertion cassette (IC), the IC comprising, in a 5′ to 3′ direction,
i. a first region homologous to part of the target polynucleotide sequence,
ii. a second region comprising a mutation of one or more nucleotides in the target polynucleotide sequence,
iii. a first nuclease binding site,
iv. a polynucleotide sequence encoding a marker gene,
v. a second nuclease binding site,
vi. a third region comprising a mutation of one or more nucleotides in the target polynucleotide sequence, and
vii. a fourth region homologous to part of the target polynucleotide sequence, wherein the first region and the fourth region are 95%-100% identical to their respective parts of the target polynucleotide sequence;
b) inserting the IC into the target polynucleotide sequence via homologous recombination to generate a first modified target polynucleotide;
c) selecting a cell which expresses the marker gene;
d) subjecting the first modified target polynucleotide to a site-specific nuclease to generate a second modified target polynucleotide having cohesive ends; and
e) subjecting the second modified target polynucleotide having cohesive ends to a ligase, wherein the ligase ligates the cohesive ends at the second region and the third region to create a ligated modified target nucleic acid comprising one or more modified nucleotides when compared to the target polynucleotide sequence.

129. The method of claim 128, wherein the first modified target nucleic acid is isolated from the cell after (c).

130. The method of claim 128 or 129, wherein the site-specific nuclease is exogenous to the cell.

131. The method of any one of claims 128 to 130, wherein the ligase is exogenous to the cell.

132. The method of claim 128, wherein the first modified target protein is in the cell after (c).

133. The method of claim 132, wherein the site-specific nuclease is introduced into the cell as a polynucleotide encoding the site-specific nuclease.

134. The method of claim 132 or 133, wherein the ligase is introduced into the cell as a polynucleotide encoding a ligase.

135. The method of any one of claims 128 to 134, wherein the site-specific nuclease is a recombinant site-specific nuclease.

136. The method of any one of claims 128 to 135, wherein the ligase is a recombinant ligase.

137. The method of any one of claims 128 to 136, wherein the site-specific nuclease is a Cas9 effector protein.

138. The method of claim 137, wherein the Cas9 effector protein is a Type II-B Cas9.

139. The method of any one of claims 128 to 131, wherein the site-specific nuclease is a Cas9-endonuclease fusion protein.

140. The method of claim 139, wherein the endonuclease in the Cas9-endonuclease fusion protein is a Type IIS endonuclease.

141. The method of claim 139, wherein the endonuclease in the Cas9-endonuclease fusion protein is FokI.

142. The method of any one of claims 139 to 141, wherein the Cas9-endonuclease fusion protein comprises a modified Cas9.

143. The method of claim 142, wherein the modified Cas9 comprises a catalytically inactive Cas9.

144. The method of claim 143, wherein the endonuclease is FokI.

145. The method of claim 142, wherein the Cas9-endonuclease fusion protein comprises a Cas9 having nickase activity, and the endonuclease is FokI.

146. The method of claim 143, wherein the Cas9-endonuclease fusion protein comprises a Cas9 having a D10A substitution.

147. The method of claim 143, wherein the Cas9-endonuclease fusion protein comprises a Cas9 having a H840A substitution.

148. The method of claim 128, wherein the site-specific nuclease is Cas9, Cpf1, or Cas9-FokI.

149. The method of claim 128, wherein the site-specific nuclease is a Cpf1 effector protein.

150. The method of any one of claims 128 to 149, wherein the cohesive ends of the second modified target polynucleotide of (d) comprise a 5′ overhang.

151. The method of any one of claims 128 to 149, wherein the cohesive ends of the second modified target polynucleotide of (d) comprise a 3′ overhang.

152. The method of any one of claims 128 to 151, wherein the site-specific nuclease is capable of generating cohesive ends comprising a single-stranded polynucleotide of 3 to 40 nucleotides.

153. The method of any one of claims 128 to 151, wherein the nuclease is capable of generating cohesive ends comprising a single-stranded polynucleotide of 4 to 30 nucleotides.

154. The method of any one of claims 128 to 151, wherein the nuclease is capable of generating cohesive ends comprising a single-stranded polynucleotide of 5 to 20 nucleotides.

155. The method of any one of claims 128 to 154, wherein the target polynucleotide sequence is in a plasmid.

156. The method of any one of claims 128 to 155, wherein the target polynucleotide sequence is in a chromosome.

157. An engineered guide RNA that forms a complex with a stiCas9 protein, comprising:
a) a guide sequence capable of hybridizing to a target sequence in a eukaryotic cell; and
b) a tracrRNA sequence capable of binding to the Cas9 protein, wherein the tracrRNA differs from a naturally-occurring tracrRNA sequence by at least 10 nucleotides,
wherein the engineered guide RNA improves nuclease efficiency of the Cas9 protein.

158. The engineered guide RNA of claim 157, wherein the tracrRNA sequence has at least 10 fewer nucleotides than a naturally-occurring tracrRNA.

159. The engineered guide RNA of claim 157, wherein the tracrRNA sequence has at least 10 more nucleotides than a naturally-occurring tracrRNA.

160. The engineered guide RNA of claim 157, wherein the guide sequence comprises at least 90% sequence identity to any one of SEQ ID NOs: 104-125 or 196-199.

161. The engineered guide RNA of claim 157, wherein the tracrRNA sequence comprises at least 90% sequence identity to any one of SEQ ID NOs: 148-171.

162. The engineered guide RNA of claim 157, wherein the guide RNA comprises at least 90% sequence identity to any one of SEQ ID NOs: 172-191.

163. The engineered guide RNA of any one of claims 157 to 159, wherein the tracrRNA comprises one or more modifications in a stem loop of the tracrRNA.

164. The engineered guide RNA of claim 163, wherein the modification comprises elongation of the stem loop.

165. The engineered guide RNA of claim 163, wherein the modification comprises shortening of the stem loop.

166. The engineered guide RNA of claim 163, wherein the modification comprises one or more nucleotide substitutions in the stem loop.

167. The engineered guide RNA of any one of claims 157 to 166, wherein the improved nuclease efficiency of the Cas9 protein is determined by a biochemical assay, a sequencing assay, and/or an affinity test.

168. A CRISPR-Cas system comprising an engineered guide RNA of any one of claims 157 to 163.

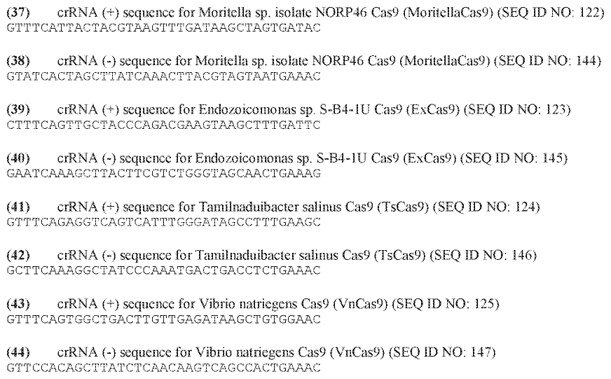

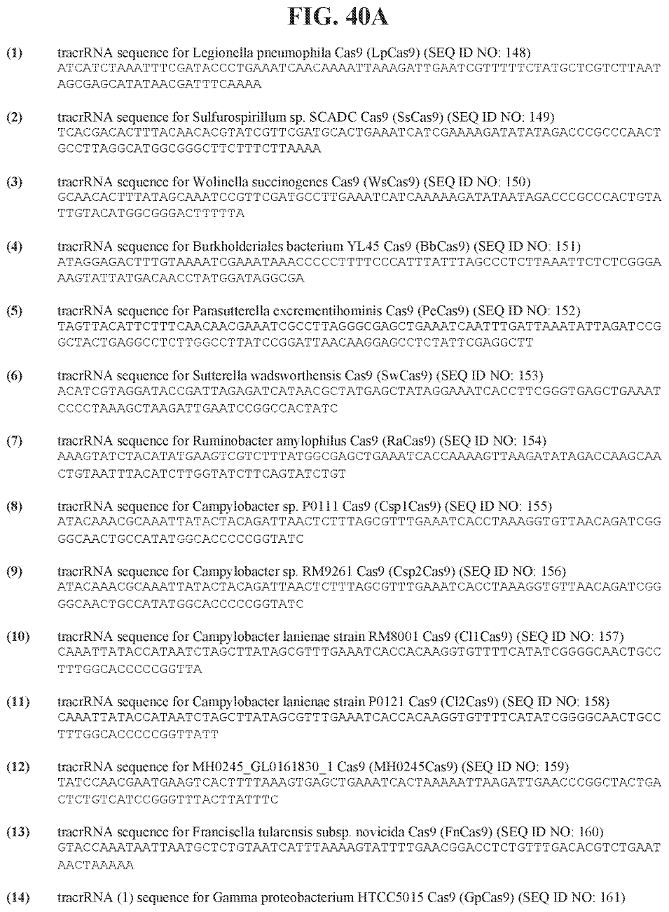

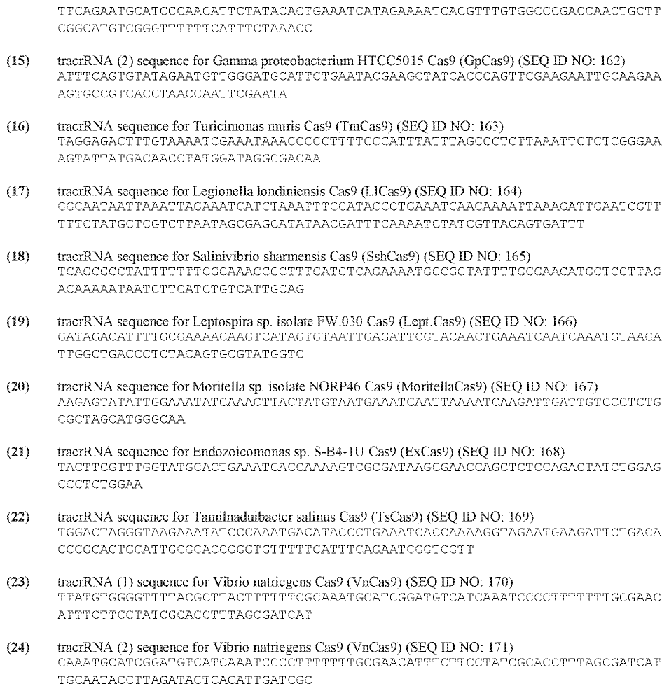

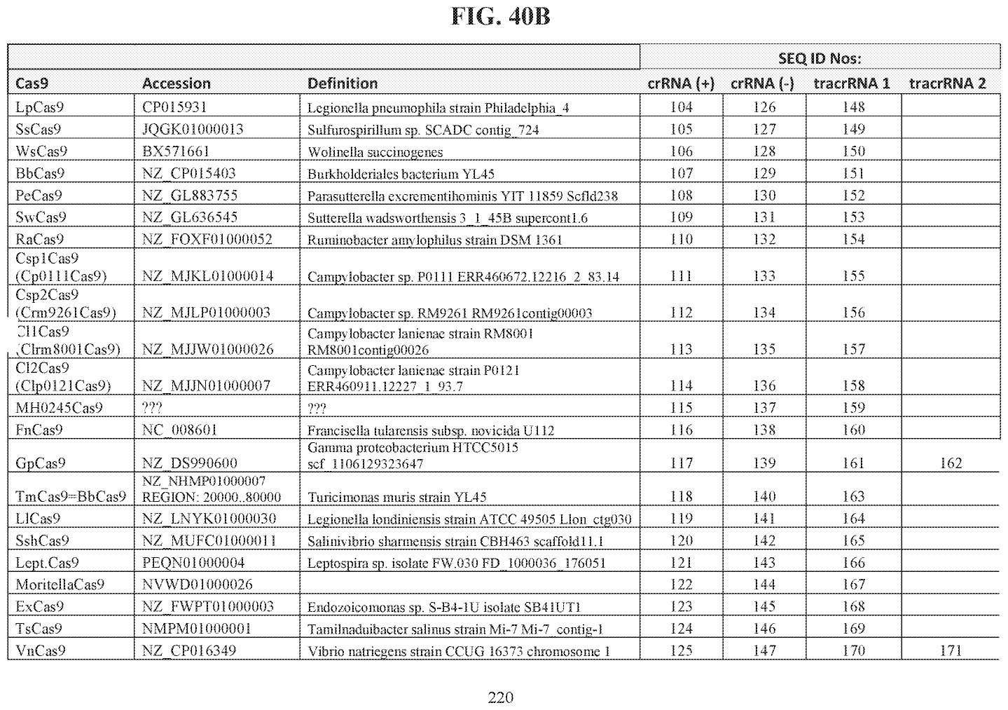

169. An engineered Cas9-guide RNA complex, comprising any combination of Cas9, guide sequence, and tracrRNA sequence as found in FIG. 40B.

170. The CRISPR-Cas system of claim 163, wherein the system does not comprise a tracrRNA sequence on a separate polynucleotide.

171. A method of producing an engineered guide RNA that binds to a Cas9 protein, comprising:
a. providing a guide sequence capable of hybridizing to a target sequence in a eukaryotic cell;
b. modifying a naturally-occurring tracrRNA sequence by removing at least ten nucleotides from the tracrRNA sequence to form a modified tracrRNA sequence; and
c. linking the guide sequence to the modified tracrRNA sequence to generate the engineered guide RNA.

172. A non-naturally occurring CRISPR-Cas system comprising:
a) a Cas9 effector protein capable of generating cohesive ends (stiCas9); and
b) a guide RNA that forms a complex with the stiCas9 and comprises a guide sequence, wherein the guide sequence is capable of hybridizing with a target sequence in a eukaryotic cell but does not hybridize to a sequence in a bacterial cell;
wherein the complex does not occur in nature, and
wherein the system does not comprise a tracrRNA sequence on a separate polynucleotide.
